SEQUENCE LISTING (830 sequences)

SEQ ID NO: 1 - protein, 1001 aa, unidentified
KMQDSVSKMKYRLGIDLGTTSLGWAMLRLDEQNEPYAVIRAGVRIFNNGRDPKTEASLAVARRLARQQRRTRDRKIRRKERLIGELVDMGFFPKDPVKRRQLASLDPFKLRTEALDRALSPEEFARAIFHLARRRGFKSNRKTDSGDTESSKMKEAIKRTLNELQNKGFRTVGEWLNMRHQQRLGTRSRIKNVPTGSGKQTTAYDFYLNRFMIEYEFDRIWEKQSQMNPGLFTNERKAILKDIIFYQRPLRPVEPGRCTFMPDNPRAPLALPQQQDFRIYQEVNNLRKIDPTSLLEVNLTLPERDRIVELLQRKPALTFDAVRKALCFNGTFNLEGENRSELKGNLTNCALAKKKLFGESWYSFDAHKRFEIVEHLLQEESEENLVSWLQKECNLSEEYAKNVASVRLPAGYGALCQEALDLILPYLKAEVITYDKAVQKAGMNHSELTLAQETGEILPELPYYGQYLKRHVGFGTGKPEDSAEKRYGKIPNPTVHIALNQLRTVVNALIRRYGKPTQIVIELARELKQNKKAKDQYRIEMNHNQNRNERIRADISMILGINPENVKRKDIEKQILWEELNLKDATARCCPYSGKQISAEMLFTDEVEIDHILPFSRTLDDSKNNKVVCIREANRIKGNRTPWEARKDFEKRGWSVEAMTARAQAMPKAKRFRFAEDGYKVWLKDFDGFEARALTDTQYMSRVAREYLQLICPGQTWSVPGQLTGMLRRFLGLNDILGVNGEKNRDDHRHHAVDACVIALTDRSMLQRISTASARAENKHLTRLLESFPAPWATFYEHVTRAVKSICVSHKPEHAYQGAMNEQTAYGLRPDGYVKYRQNGKVEHKKLNVIPQVSVKGTWRHGLNSDGSLKAYKGLKGGSNFCIEIVMGEGGRWEGDVITTYEAYQIVRAKGEAALYGSVSRSGKPLVMRLMQKDIVEMTLADGRCKMLLYIITQNKQMFFYRIENAGGGREDVSKRPGSLQKALAKKIIVSPIGDFRKEKL

SEQ ID NO: 2 - protein, 1002 aa, unidentified
MKMQDSVSKMKYRLGIDLGTTSLGWAMLRLDEQNEPYAVIRAGVRIFNNGRDPKTEASLAVARRLARQQRRTRDRKIRRKERLIGELVDMGFFPKDPVKRRQLASLDPFKLRTEALDRALSPEEFARAIFHLARRRGFKSNRKTDSGDTESSKMKEAIKRTLNELQNKGFRTVGEWLNMRHQQRLGTRSRIKNVPTGSGKQTTAYDFYLNRFMIEYEFDRIWEKQSQMNPGLFTNERKAILKDIIFYQRPLRPVEPGRCTFMPDNPRAPLALPQQQDFRIYQEVNNLRKIDPTSLLEVNLTLPERDRIVELLQRKPALTFDAVRKALCFNGTFNLEGENRSELKGNLTNCALAKKKLFGESWYSFDAHKRFEIVEHLLQEESEENLVSWLQKECNLSEEYAKNVASVRLPAGYGALCQEALDLILPYLKAEVITYDKAVQKAGMNHSELTLAQETGEILPELPYYGQYLKRHVGFGTGKPEDSAEKRYGKIPNPTVHIALNQLRTVVNALIRRYGKPTQIVIELARELKQNKKAKDQYRIEMNHNQNRNERIRADISMILGINPENVKRKDIEKQILWEELNLKDATARCCPYSGKQISAEMLFTDEVEIDHILPFSRTLDDSKNNKVVCIREANRIKGNRTPWEARKDFEKRGWSVEAMTARAQAMPKAKRFRFAEDGYKVWLKDFDGFEARALTDTQYMSRVAREYLQLICPGQTWSVPGQLTGMLRRFLGLNDILGVNGEKNRDDHRHHAVDACVIALTDRSMLQRISTASARAENKHLTRLLESFPAPWATFYEHVTRAVKSICVSHKPEHAYQGAMNEQTAYGLRPDGYVKYRQNGKVEHKKLNVIPQVSVKGTWRHGLNSDGSLKAYKGLKGGSNFCIEIVMGEGGRWEGDVITTYEAYQIVRAKGEAALYGSVSRSGKPLVMRLMQKDIVEMTLADGRCKMLLYIITQNKQMFFYRIENAGGGREDVSKRPGSLQKALAKKIIVSPIGDFRKEKL

SEQ ID NO: 3 - protein, 1052 aa, synthetic construct
MGKPIPNPLLGLDSTKRTADGSEFESPKKKRKVKMQDSVSKMKYRLGIDLGTTSLGWAMLRLDEQNEPYAVIRAGVRIFNNGRDPKTEASLAVARRLARQQRRTRDRKIRRKERLIGELVDMGFFPKDPVKRRQLASLDPFKLRTEALDRALSPEEFARAIFHLARRRGFKSNRKTDSGDTESSKMKEAIKRTLNELQNKGFRTVGEWLNMRHQQRLGTRSRIKNVPTGSGKQTTAYDFYLNRFMIEYEFDRIWEKQSQMNPGLFTNERKAILKDIIFYQRPLRPVEPGRCTFMPDNPRAPLALPQQQDFRIYQEVNNLRKIDPTSLLEVNLTLPERDRIVELLQRKPALTFDAVRKALCFNGTFNLEGENRSELKGNLTNCALAKKKLFGESWYSFDAHKRFEIVEHLLQEESEENLVSWLQKECNLSEEYAKNVASVRLPAGYGALCQEALDLILPYLKAEVITYDKAVQKAGMNHSELTLAQETGEILPELPYYGQYLKRHVGFGTGKPEDSAEKRYGKIPNPTVHIALNQLRTVVNALIRRYGKPTQIVIELARELKQNKKAKDQYRIEMNHNQNRNERIRADISMILGINPENVKRKDIEKQILWEELNLKDATARCCPYSGKQISAEMLFTDEVEIDHILPFSRTLDDSKNNKVVCIREANRIKGNRTPWEARKDFEKRGWSVEAMTARAQAMPKAKRFRFAEDGYKVWLKDFDGFEARALTDTQYMSRVAREYLQLICPGQTWSVPGQLTGMLRRFLGLNDILGVNGEKNRDDHRHHAVDACVIALTDRSMLQRISTASARAENKHLTRLLESFPAPWATFYEHVTRAVKSICVSHKPEHAYQGAMNEQTAYGLRPDGYVKYRQNGKVEHKKLNVIPQVSVKGTWRHGLNSDGSLKAYKGLKGGSNFCIEIVMGEGGRWEGDVITTYEAYQIVRAKGEAALYGSVSRSGKPLVMRLMQKDIVEMTLADGRCKMLLYIITQNKQMFFYRIENAGGGREDVSKRPGSLQKALAKKIIVSPIGDFRKEKLKRTADGSEFESPKKKRKV

SEQ ID NO: 4 - DNA, 3009 bp, unidentified
  source 1..3009: mol_type = unassigned DNA
atgaaaatgcaagactctgtctcaaaaatgaaatacagactcggaatcgaccttggaaccacttccttgggctgggctatgttgcggcttgacgaacaaaacgaaccttacgccgtaattcgagccggggttcgtatctttaataacggtcgagacccgaaaacggaagcctctttggccgtagcgcgccgtcttgcacgtcaacaaagaaggactcgtgacagaaaaatcaggcgtaaagaacgccttatcggggagctggtggacatggggttcttcccgaaagatcccgttaaacggcgtcaactcgcctcgttagatccttttaaacttcgaactgaagccttagatagggctctttctccggaagaatttgccagagcgattttccatttagccagacgacgcggcttcaaaagcaatcggaaaacagattccggcgataccgaatcgagcaagatgaaagaggctatcaaacgcactttaaatgaactacaaaacaagggctttcggaccgttggtgagtggctcaatatgcgccatcaacaacgcctcggcacgcgttcgcgcattaaaaatgtccccaccggttccggtaagcaaaccaccgcatacgacttctacttaaatcgattcatgattgagtatgaatttgatcgtatttgggaaaagcagtcccaaatgaatcccggccttttcaccaatgaacgcaaggccatcttaaaagacattatcttttaccaaagaccccttcgtcctgtggaacccggacgttgcacctttatgccggacaatccacgagcaccgctagctcttcctcaacaacaagactttcgtatttatcaagaagttaacaacttgaggaaaattgacccaacctccctgcttgaagttaacctcacgctgcccgaaagagatcgaatcgtcgaattgcttcaacgaaaacctgctctgacctttgatgccgttagaaaggcgctttgctttaacggaacatttaatttagaaggagaaaatcgttccgagttaaaaggcaatctcacaaactgtgcgctcgctaagaaaaaactatttggagaaagttggtattccttcgatgcccataaacgatttgaaatcgttgaacatcttttgcaagaggaatccgaagaaaacctcgtttcctggttgcaaaaagaatgcaatctttctgaagagtatgcaaagaatgtcgcttccgtgcgactccctgcaggatacggagccttgtgtcaagaggccttagatttaatccttccatatctgaaagctgaagtcatcacatacgacaaagcggtccaaaaagccgggatgaaccacagcgaactgacattagcacaagaaaccggtgaaatccttccagagcttccttactacggtcagtatctcaaacgtcacgtgggtttcgggaccgggaaacccgaggattccgcagagaagcgttacggaaaaattcccaacccgacggtacacatcgccctcaatcaattgaggaccgttgtcaatgctttaatccgcagatatggaaaaccgacccagattgttatcgagcttgctcgagaactcaaacaaaacaaaaaggcaaaagatcaatatcgaatcgagatgaatcacaaccagaatcggaatgaacggattcgcgcggatatttcgatgattctcggaatcaatccagaaaatgtgaaacggaaagatattgaaaaacagattttatgggaagaacttaatctgaaggacgcaacggctcgttgttgtccatatagcggaaagcaaatcagcgcagaaatgctttttaccgatgaagttgaaatcgatcatattctcccgttttccaggactttggacgattcaaagaataataaagtcgtttgtatccgagaagccaaccgcattaaagggaatcgaaccccttgggaagcacgaaaggattttgagaagcgaggatggtcggtagaagcgatgacggcacgcgcccaagcaatgccgaaagccaagcgatttcgcttcgccgaggacggatataaagtttggttaaaagatttcgatggcttcgaggcacgcgcgttgacagacacacaatacatgagtcgagtcgctcgcgaatatcttcagttaatttgccccggtcaaacctggtcggttcccggacaactcaccggaatgttaagaagatttttagggctaaatgacattttgggggttaatggtgagaaaaaccgcgatgaccaccgccaccatgccgttgatgcctgcgtgattgccctcacggatcgttccatgttacaaaggatttcgacggcaagtgcgagggctgaaaataaacatcttactcgtctgttggaatctttcccggctccatgggctacgttctatgagcatgttacccgcgccgtgaaatcgatttgtgtcagtcacaaaccggagcacgcctatcaaggggccatgaacgaacaaacagcctacggcttaagaccggacggatatgtcaaatacagacaaaacggaaaagttgaacataagaagttaaatgttatccctcaggtatcggtcaagggaacctggagacatggtcttaactctgacggatcattaaaagcgtacaaaggactaaagggaggaagcaatttttgtattgaaattgtaatgggagagggcggtcgctgggaaggcgacgttattacaacatacgaggcctaccaaatcgtacgggcgaaaggagaagctgcgctttatgggagtgtgagtcgttccggaaaaccgcttgtaatgcgcttgatgcaaaaggatatcgttgaaatgactcttgcggatggccgatgcaaaatgcttctttacataatcacccaaaacaaacaaatgttcttttaccgcatcgaaaatgccggcggtggaagagaagatgtttccaagagaccaggatccttacaaaaggcacttgcgaaaaaaatcatagtctctccgataggggatttccgtaaggaaaaattatga

SEQ ID NO: 5 - DNA, 3009 bp, synthetic construct
  source 1..3009: mol_type = other DNA
atgaagatgcaagacagcgtcagcaagatgaaatatagactcggcatcgatctcggaacaacatctctgggatgggccatgctgagactggacgagcagaacgaaccctacgccgtgattagggctggagtgagaatttttaacaacggaagggaccccaagaccgaagcctctctggctgtggctaggagactggccagacaacagagaaggacaagagatagaaaaattagaaggaaggaaagactcatcggcgagctggtcgacatgggcttcttccctaaagaccccgtgaagaggagacagctggcttctctggaccccttcaagctcagaaccgaggccctcgatagagctctgagccccgaggagttcgctagagccatcttccatctggctagaaggagaggcttcaagagcaatagaaagacagacagcggcgacaccgagagcagcaaaatgaaggaagccattaaaaggacactgaacgagctccaaaacaagggatttagaaccgtgggcgagtggctcaacatgagacatcagcaaaggctcggcacaagatctagaatcaaaaacgtgcccaccggatccggaaagcagaccacagcctacgacttctatctgaatagattcatgattgagtacgagtttgatagaatctgggagaaacagagccagatgaaccccggactgttcacaaatgaaaggaaagctattctgaaagatatcattttctaccaaagacctctcagacccgtggagcccggaagatgcaccttcatgcccgacaaccccagagcccctctggctctcccccaacagcaagactttagaatctatcaagaggtgaataatctgagaaaaatcgaccccacctctctgctggaagtcaatctgacactccccgaaagagatagaatcgtggagctgctgcagagaaagcccgctctgaccttcgacgccgtcagaaaggccctctgcttcaatggcaccttcaacctcgagggagagaatagaagcgaactcaagggcaacctcaccaattgcgccctcgctaagaagaagctctttggcgagagctggtatagcttcgacgcccacaagaggttcgaaatcgtggaacatctgctgcaagaggagagcgaagagaatctggtgagctggctgcagaaagagtgcaatctcagcgaggagtacgccaagaatgtcgctagcgtgagactgcccgccggatacggcgctctctgccaagaagccctcgatctcatcctcccctacctcaaggccgaggtgatcacctacgataaagccgtgcagaaagccggcatgaaccactccgagctcacactggcccaagaaaccggcgaaatcctccccgagctgccctattatggccaatacctcaagaggcacgtcggctttggaaccggcaagcccgaagatagcgctgagaagagatatggcaagatccccaatcccacagtccatattgctctgaaccagctgagaacagtggtgaatgccctcatcagaaggtatggaaagcccacacaaatcgtgatcgaactcgctagggaactgaagcagaacaagaaggccaaggatcagtataggatcgaaatgaatcacaatcagaacagaaacgagaggattagagccgacatcagcatgattctgggcatcaatcccgagaacgtgaagaggaaggacatcgagaagcaaattctgtgggaggagctgaatctgaaagacgccaccgctagatgctgcccttacagcggaaaacagatttccgccgaaatgctcttcacagacgaagtggagatcgaccacattctgcccttcagcagaacactggacgacagcaagaataacaaggtggtgtgcattagggaggccaacagaatcaagggcaacagaaccccttgggaggccagaaaagacttcgagaaaaggggatggagcgtggaggctatgacagctagggcccaagccatgcccaaggccaagagattcagattcgccgaggatggctacaaggtgtggctgaaggactttgatggattcgaagccagagctctgaccgacacccagtacatgtctagagtcgccagagagtatctgcaactgatctgccccggccagacatggtccgtgcccggccagctgaccggcatgctgagaagatttctgggactgaacgacatcctcggagtcaacggcgagaagaatagagatgaccacagacatcacgccgtggacgcttgcgtgattgctctcacagatagaagcatgctgcaaagaatctccaccgccagcgctagagctgagaacaagcacctcacaagactgctggagtccttccccgccccttgggccaccttctatgaacacgtgaccagagccgtgaagagcatctgcgtgtcccataaacccgagcacgcctaccaaggcgctatgaacgagcagacagcctacggcctcagacccgacggatatgtgaagtataggcagaacggcaaggtcgaacacaagaagctgaacgtgatcccccaagtgtccgtcaaaggaacatggaggcatggactgaattccgacggctctctgaaagcttacaagggactgaaaggcggatccaacttctgcatcgagatcgtgatgggcgagggaggaagatgggagggagatgtgatcaccacctacgaggcctaccagattgtgagagccaaaggagaggctgctctctacggctccgtctctagaagcggaaagcccctcgtcatgaggctcatgcagaaggatatcgtcgagatgacactggccgacggcagatgcaagatgctgctgtacatcatcacccagaataaacagatgttcttttatagaattgagaacgccggcggaggaagagaagatgtcagcaaaagacccggcagcctccagaaagctctggccaagaagattatcgtgagccccatcggcgactttagaaaggagaagctgtga

SEQ ID NO: 6 - DNA, 3159 bp, synthetic construct
  source 1..3159: mol_type = other DNA
atgggaaaacctatccctaaccctctgctgggactcgatagcacaaagagaaccgccgatggaagcgagttcgagtcccctaagaagaagaggaaagtcaagatgcaagacagcgtcagcaagatgaaatatagactcggcatcgatctcggaacaacatctctgggatgggccatgctgagactggacgagcagaacgaaccctacgccgtgattagggctggagtgagaatttttaacaacggaagggaccccaagaccgaagcctctctggctgtggctaggagactggccagacaacagagaaggacaagagatagaaaaattagaaggaaggaaagactcatcggcgagctggtcgacatgggcttcttccctaaagaccccgtgaagaggagacagctggcttctctggaccccttcaagctcagaaccgaggccctcgatagagctctgagccccgaggagttcgctagagccatcttccatctggctagaaggagaggcttcaagagcaatagaaagacagacagcggcgacaccgagagcagcaaaatgaaggaagccattaaaaggacactgaacgagctccaaaacaagggatttagaaccgtgggcgagtggctcaacatgagacatcagcaaaggctcggcacaagatctagaatcaaaaacgtgcccaccggatccggaaagcagaccacagcctacgacttctatctgaatagattcatgattgagtacgagtttgatagaatctgggagaaacagagccagatgaaccccggactgttcacaaatgaaaggaaagctattctgaaagatatcattttctaccaaagacctctcagacccgtggagcccggaagatgcaccttcatgcccgacaaccccagagcccctctggctctcccccaacagcaagactttagaatctatcaagaggtgaataatctgagaaaaatcgaccccacctctctgctggaagtcaatctgacactccccgaaagagatagaatcgtggagctgctgcagagaaagcccgctctgaccttcgacgccgtcagaaaggccctctgcttcaatggcaccttcaacctcgagggagagaatagaagcgaactcaagggcaacctcaccaattgcgccctcgctaagaagaagctctttggcgagagctggtatagcttcgacgcccacaagaggttcgaaatcgtggaacatctgctgcaagaggagagcgaagagaatctggtgagctggctgcagaaagagtgcaatctcagcgaggagtacgccaagaatgtcgctagcgtgagactgcccgccggatacggcgctctctgccaagaagccctcgatctcatcctcccctacctcaaggccgaggtgatcacctacgataaagccgtgcagaaagccggcatgaaccactccgagctcacactggcccaagaaaccggcgaaatcctccccgagctgccctattatggccaatacctcaagaggcacgtcggctttggaaccggcaagcccgaagatagcgctgagaagagatatggcaagatccccaatcccacagtccatattgctctgaaccagctgagaacagtggtgaatgccctcatcagaaggtatggaaagcccacacaaatcgtgatcgaactcgctagggaactgaagcagaacaagaaggccaaggatcagtataggatcgaaatgaatcacaatcagaacagaaacgagaggattagagccgacatcagcatgattctgggcatcaatcccgagaacgtgaagaggaaggacatcgagaagcaaattctgtgggaggagctgaatctgaaagacgccaccgctagatgctgcccttacagcggaaaacagatttccgccgaaatgctcttcacagacgaagtggagatcgaccacattctgcccttcagcagaacactggacgacagcaagaataacaaggtggtgtgcattagggaggccaacagaatcaagggcaacagaaccccttgggaggccagaaaagacttcgagaaaaggggatggagcgtggaggctatgacagctagggcccaagccatgcccaaggccaagagattcagattcgccgaggatggctacaaggtgtggctgaaggactttgatggattcgaagccagagctctgaccgacacccagtacatgtctagagtcgccagagagtatctgcaactgatctgccccggccagacatggtccgtgcccggccagctgaccggcatgctgagaagatttctgggactgaacgacatcctcggagtcaacggcgagaagaatagagatgaccacagacatcacgccgtggacgcttgcgtgattgctctcacagatagaagcatgctgcaaagaatctccaccgccagcgctagagctgagaacaagcacctcacaagactgctggagtccttccccgccccttgggccaccttctatgaacacgtgaccagagccgtgaagagcatctgcgtgtcccataaacccgagcacgcctaccaaggcgctatgaacgagcagacagcctacggcctcagacccgacggatatgtgaagtataggcagaacggcaaggtcgaacacaagaagctgaacgtgatcccccaagtgtccgtcaaaggaacatggaggcatggactgaattccgacggctctctgaaagcttacaagggactgaaaggcggatccaacttctgcatcgagatcgtgatgggcgagggaggaagatgggagggagatgtgatcaccacctacgaggcctaccagattgtgagagccaaaggagaggctgctctctacggctccgtctctagaagcggaaagcccctcgtcatgaggctcatgcagaaggatatcgtcgagatgacactggccgacggcagatgcaagatgctgctgtacatcatcacccagaataaacagatgttcttttatagaattgagaacgccggcggaggaagagaagatgtcagcaaaagacccggcagcctccagaaagctctggccaagaagattatcgtgagccccatcggcgactttagaaaggagaagctgaagagaaccgctgacggcagcgaattcgaaagccccaaaaagaagagaaaggtgtga

SEQ ID NO: 7 - protein, 1003 aa, unidentified
EITINREIGKLGLPRHLVLGMDPGIASCGFALIDTANREILDLGVRLFDSPTHPKTGQSLAVIRRGFRSTRRNIDRTQARLKHCLQILKAYGLIPQDATKEYFHTTKGDKQPLKLRVDGLDRLLNDREWALVLYSLCKRRGYIPHGEGNQDKSSEGGKVLSALAANKEAIAETSCRTVGEWLAQQPQSRNRGGNYDKCVTHAQLIEETHILFDAQRSFGSKYASPEFEAAYIEVCDWERSRKDFDRRTYDLVGHCSYFPTEKRAARCTLTSELVSAYGALGNITIIHDDGTSRALSATERDECIAILFSCEPIRGNKDCAVKFGALRKALDLSSGDYFKGVPAADEKTREVYKPKGWRVLRNTLNAANPILLQRLRDDRNLADAVMEAVAYSSALPVLQEQLQGLPLSEAEIEALCRLPYSSKALNGYGNRSKKALDMLLDCLEEPEVLNLTQAENDCGLLGLRIAGTQLERSDRLMPYETWIERTGRTNNNPVVIRAMSQMRKVVNAICRKWGVPNEIHVELDRELRLPQRAKDEIAKANKKNEKNRERIAGQIAELRGCTADEVTGKQIEKYRLWEEQECFDLYTGAKIEVDRLISDDTYTQIDHILPFSRTGENSRNNKVLVLAKSNQDKREQTPYEWMSHDGAPSWDAFERRVQENQKLSRRKKNFLLEKDLDTKEGEFLARSFTDTAYMSREVCAYLADCLLFPDDGAKAHVVPTTGRATAWLRRRWGLNFGSNGEKDRSDDRHHATDACVIAACSRSLVIKTARINQETHWSITRGMNETQRRDAIMKALESVMPWETFANEVRAAHDFVVPTRFVPRKGKGELFEQTVYRYAGVNAQGKDIARKASSDKDIVMGNAVVSADEKSVIKVSEMLCLRLWHDPEAKKGQGAWYADPVYKADIPALKDGTYVPRIAKQKYGRKVWKAVPNSALTQKPLEIYLGDLIKVGDKLGRYNGYNIATANWSFVDALTKKEIAFPSVGMLSNELQPIIIRESILDN

SEQ ID NO: 8 - protein, 1004 aa, unidentified
MEITINREIGKLGLPRHLVLGMDPGIASCGFALIDTANREILDLGVRLFDSPTHPKTGQSLAVIRRGFRSTRRNIDRTQARLKHCLQILKAYGLIPQDATKEYFHTTKGDKQPLKLRVDGLDRLLNDREWALVLYSLCKRRGYIPHGEGNQDKSSEGGKVLSALAANKEAIAETSCRTVGEWLAQQPQSRNRGGNYDKCVTHAQLIEETHILFDAQRSFGSKYASPEFEAAYIEVCDWERSRKDFDRRTYDLVGHCSYFPTEKRAARCTLTSELVSAYGALGNITIIHDDGTSRALSATERDECIAILFSCEPIRGNKDCAVKFGALRKALDLSSGDYFKGVPAADEKTREVYKPKGWRVLRNTLNAANPILLQRLRDDRNLADAVMEAVAYSSALPVLQEQLQGLPLSEAEIEALCRLPYSSKALNGYGNRSKKALDMLLDCLEEPEVLNLTQAENDCGLLGLRIAGTQLERSDRLMPYETWIERTGRTNNNPVVIRAMSQMRKVVNAICRKWGVPNEIHVELDRELRLPQRAKDEIAKANKKNEKNRERIAGQIAELRGCTADEVTGKQIEKYRLWEEQECFDLYTGAKIEVDRLISDDTYTQIDHILPFSRTGENSRNNKVLVLAKSNQDKREQTPYEWMSHDGAPSWDAFERRVQENQKLSRRKKNFLLEKDLDTKEGEFLARSFTDTAYMSREVCAYLADCLLFPDDGAKAHVVPTTGRATAWLRRRWGLNFGSNGEKDRSDDRHHATDACVIAACSRSLVIKTARINQETHWSITRGMNETQRRDAIMKALESVMPWETFANEVRAAHDFVVPTRFVPRKGKGELFEQTVYRYAGVNAQGKDIARKASSDKDIVMGNAVVSADEKSVIKVSEMLCLRLWHDPEAKKGQGAWYADPVYKADIPALKDGTYVPRIAKQKYGRKVWKAVPNSALTQKPLEIYLGDLIKVGDKLGRYNGYNIATANWSFVDALTKKEIAFPSVGMLSNELQPIIIRESILDN

SEQ ID NO: 9 - protein, 1054 aa, synthetic construct
MGKPIPNPLLGLDSTKRTADGSEFESPKKKRKVEITINREIGKLGLPRHLVLGMDPGIASCGFALIDTANREILDLGVRLFDSPTHPKTGQSLAVIRRGFRSTRRNIDRTQARLKHCLQILKAYGLIPQDATKEYFHTTKGDKQPLKLRVDGLDRLLNDREWALVLYSLCKRRGYIPHGEGNQDKSSEGGKVLSALAANKEAIAETSCRTVGEWLAQQPQSRNRGGNYDKCVTHAQLIEETHILFDAQRSFGSKYASPEFEAAYIEVCDWERSRKDFDRRTYDLVGHCSYFPTEKRAARCTLTSELVSAYGALGNITIIHDDGTSRALSATERDECIAILFSCEPIRGNKDCAVKFGALRKALDLSSGDYFKGVPAADEKTREVYKPKGWRVLRNTLNAANPILLQRLRDDRNLADAVMEAVAYSSALPVLQEQLQGLPLSEAEIEALCRLPYSSKALNGYGNRSKKALDMLLDCLEEPEVLNLTQAENDCGLLGLRIAGTQLERSDRLMPYETWIERTGRTNNNPVVIRAMSQMRKVVNAICRKWGVPNEIHVELDRELRLPQRAKDEIAKANKKNEKNRERIAGQIAELRGCTADEVTGKQIEKYRLWEEQECFDLYTGAKIEVDRLISDDTYTQIDHILPFSRTGENSRNNKVLVLAKSNQDKREQTPYEWMSHDGAPSWDAFERRVQENQKLSRRKKNFLLEKDLDTKEGEFLARSFTDTAYMSREVCAYLADCLLFPDDGAKAHVVPTTGRATAWLRRRWGLNFGSNGEKDRSDDRHHATDACVIAACSRSLVIKTARINQETHWSITRGMNETQRRDAIMKALESVMPWETFANEVRAAHDFVVPTRFVPRKGKGELFEQTVYRYAGVNAQGKDIARKASSDKDIVMGNAVVSADEKSVIKVSEMLCLRLWHDPEAKKGQGAWYADPVYKADIPALKDGTYVPRIAKQKYGRKVWKAVPNSALTQKPLEIYLGDLIKVGDKLGRYNGYNIATANWSFVDALTKKEIAFPSVGMLSNELQPIIIRESILDNKRTADGSEFESPKKKRKV

SEQ ID NO: 10 - DNA, 3015 bp, unidentified
  source 1..3015: mol_type = unassigned DNA
atggagatcaccatcaatcgcgaaattgggaagctcggacttcccaggcatcttgtgcttggcatggatccaggaattgcaagctgcggattcgcacttatcgacacagccaatcgtgaaatcctggatttgggcgtcagattatttgactctccaactcatcctaaaacgggccaaagtcttgcggttattcgcaggggcttccgctctacccgtcgaaacattgaccgtacccaggcgcgcttgaagcactgtctccaaatcctcaaggcttatggcctcatcccccaagacgccaccaaagagtacttccacaccacaaaaggcgacaagcagccgctcaagcttcgcgttgatgggcttgaccgcctgctcaacgatcgcgagtgggcgctagtcctatactccctctgcaagcgccgtggatacatcccccacggagaaggcaatcaggataaatcaagcgaaggcggcaaggttctatctgcccttgcggccaacaaggaagcaattgcggagacctcgtgccgcaccgttggcgaatggctcgctcagcagcctcaaagtcgcaatcgtggcggcaattacgacaagtgtgtaacgcacgcccagcttatcgaagaaactcatatcctatttgatgctcaacgctcctttggctccaaatacgcttcgccggaatttgaggccgcatatatcgaggtttgcgattgggagcgttcgcgcaaagacttcgaccgccgcacgtacgacctcgttggacactgctcatacttcccaacagaaaaacgagccgcacgctgcacgcttacgagcgaacttgtttcagcctatggcgcactcggcaacatcaccatcatccacgatgacgggacctctcgcgccttgagcgcaacggagcgcgatgaatgcattgcaatcctgttctcgtgcgagccaattcgaggcaacaaagattgtgctgttaaattcggcgccctcagaaaagcgctcgaccttagttcaggcgattacttcaaaggggttccagctgccgacgaaaaaacgcgcgaggtgtacaagcccaagggatggcgcgtgctccgcaataccctcaatgcagccaaccccattctcctgcagcgtttacgcgatgaccgcaatctcgccgatgccgttatggaggcggtagcatattcctcggcccttcccgtactccaagagcagcttcaggggttaccgctctcggaagcggagatcgaggcgctttgtaggcttccctattcatccaaagctcttaacggctatggcaaccgttccaaaaaagcactcgacatgctgctcgattgcctcgaggagcctgaggtcctcaaccttacgcaggccgaaaatgactgcggtctgctgggactgcgcatcgctggcacccagctcgagcgctccgatcgcctgatgccctatgagacctggatcgaacgtaccggtcgaacaaataacaatcccgtcgtcattcgcgccatgtcgcaaatgcgaaaagtggtcaacgccatctgccgcaagtggggcgtgccaaacgaaatccacgttgagcttgatcgagagctcaggttgcctcagcgcgcaaaagacgagattgccaaggccaataagaagaacgagaaaaatcgcgagcgcattgccgggcaaatcgctgagctgcgtggctgcacggcagatgaggtcacgggcaaacagatagagaagtaccgcctgtgggaagagcaggaatgcttcgacctttacacgggcgctaaaatcgaagtcgatcgcctaattagcgacgacacctacacgcagattgaccacatcctgccgttctctcgcacgggagaaaactcgcgcaacaataaagtcctggtcctcgccaaaagcaatcaggataaacgcgaacagacaccttacgaatggatgtcccacgacggtgcgccttcatgggatgcttttgagcgtcgcgttcaggaaaaccagaaactcagccgtcgcaaaaagaacttcctgctggaaaaagaccttgataccaaggaaggcgaattcttagcacgcagcttcaccgacaccgcctatatgtcgcgagaagtatgcgcttacctcgccgactgcctgctgttccccgatgatggcgcaaaggcacatgttgttcctaccactggcagagcgaccgcatggctgcgtcgcaggtgggggcttaactttggttcgaatggcgaaaaagaccgctcggacgatcgtcaccatgccaccgatgcttgtgtgattgcagcatgtagtcgaagcctcgtgattaaaaccgctcgaatcaaccaagagacacactggagcataaccagaggtatgaacgagacccaacgccgcgatgccatcatgaaggctctcgaaagtgttatgccctgggaaacctttgcgaacgaagtacgcgcggcgcacgatttcgtcgtacccacccgctttgttccgcgtaagggaaagggcgagttgttcgagcagacggtctatcgctatgccggcgttaatgcacagggcaaagacattgctcgcaaggcgagctccgataaggacatcgtcatgggcaacgccgttgtgtcggcagacgagaagtcggtcatcaaggtgagcgaaatgctgtgtctgaggctctggcatgacccggaggccaagaaggggcagggcgcttggtacgcagaccccgtctataaggcggatattcctgcacttaaggatgggacgtatgtgcccaggattgcgaagcaaaaatatgggcggaaggtctggaaagccgttcccaatagcgctttaactcaaaaaccactcgaaatatatctgggtgacctcattaaagtcggggataagctcggtcgctacaatggctacaacattgcaacggccaattggtcctttgttgacgctctcacgaaaaaggagattgcattcccctcggttggaatgctctcaaacgaattgcaaccgataattattcgcgagagcattctcgataattaa

SEQ ID NO: 11 - DNA, 3015 bp, synthetic construct
  source 1..3015: mol_type = other DNA
atggagatcacaattaatagggaaattggcaagctgggactccctagacatctggtgctgggcatggatcccggcattgccagctgcggattcgctctgatcgacacagccaatagagaaattctggatctgggcgtgaggctgttcgattcccctacccatcctaagaccggccagtctctggctgtcatcagaaggggcttcagatccacaagaaggaatatcgacagaacccaagctagactcaagcactgcctccagatcctcaaagcctatggcctcattccccaagacgccaccaaagagtacttccacaccaccaagggagataagcagcctctgaaactgagagtggacggactggatagactgctgaacgatagagaatgggctctggtgctgtacagcctctgtaagagaaggggctacatccctcacggcgagggaaatcaagacaagtccagcgaaggaggcaaagtgctgagcgccctcgccgccaacaaggaagctatcgccgagacaagctgtagaaccgtgggcgaatggctcgctcaacagcctcagagcagaaatagaggcggaaactatgacaagtgcgtcacccatgctcagctcattgaggaaacccatatcctcttcgacgctcagagaagctttggcagcaagtacgccagccccgagttcgaagccgcctacattgaagtgtgtgactgggaaaggtctagaaaggattttgatagaaggacatacgatctggtcggccactgcagctacttccctaccgaaaagagagccgctagatgcacactgaccagcgagctggtcagcgcctacggagctctcggcaacatcaccatcatccacgacgacggaaccagcagagctctgagcgccaccgaaagggatgagtgcatcgccatcctctttagctgcgagcccattagaggcaataaggattgtgccgtgaagttcggagctctgagaaaggctctggacctctcctccggagattacttcaagggagtccccgccgccgacgaaaagaccagagaggtctacaagcccaagggctggagagtgctgagaaacaccctcaacgccgccaaccctattctgctccagagactgagagatgacagaaacctcgctgacgctgtgatggaagctgtcgcttacagcagcgctctgcccgtgctccaagagcagctgcaaggactgcctctctccgaggctgagatcgaggctctgtgcagactgccttacagctccaaagctctgaacggctacggcaatagaagcaaaaaagctctggacatgctgctcgattgtctggaggaacccgaagtgctgaacctcacccaagccgaaaacgattgtggactgctcggactgagaatcgccggaacccagctggagagatccgatagactcatgccttatgaaacatggatcgagagaaccggaagaaccaataacaaccccgtggtcatcagagccatgagccaaatgagaaaggtggtcaacgccatctgcagaaagtggggcgtgcccaacgaaattcatgtggagctggatagagagctgagactgccccaaagggctaaggacgagatcgccaaggctaataagaagaatgaaaagaacagagagagaatcgccggccagattgctgaactgagaggctgtacagctgacgaggtgaccggcaagcagattgagaagtatagactctgggaggagcaagagtgctttgatctgtacaccggagccaagatcgaggtggataggctcatcagcgatgatacatacacccagatcgaccacattctgccttttagcagaaccggcgagaactctagaaacaacaaagtgctggtgctcgctaagtccaatcaagacaagagggagcagaccccctatgaatggatgagccatgatggcgctcccagctgggacgccttcgaaaggagggtgcaagagaaccagaagctgtctagaaggaagaagaactttctgctcgagaaggatctggacaccaaggagggagagtttctcgctagaagcttcaccgacacagcctatatgtctagagaggtgtgtgcctatctggccgactgtctgctcttccccgacgatggagctaaggcccatgtggtgcctacaaccggaagagccaccgcttggctcagaaggagatggggactgaacttcggctccaatggcgagaaagatagatccgacgacagacatcacgctacagatgcttgcgtgatcgctgcttgctccagatctctggtgattaagaccgctagaatcaaccaagagacacattggagcatcacaagaggaatgaacgaaacccagaggagggatgccatcatgaaggctctggagtccgtcatgccttgggaaaccttcgccaacgaggtgagagctgcccacgattttgtcgtgcctaccagattcgtgcccagaaaaggcaaaggcgagctcttcgagcagaccgtgtatagatacgctggcgtgaacgcccaaggcaaggatatcgctagaaaggcctcctccgataaagacatcgtcatgggaaacgccgtggtgagcgccgatgaaaagagcgtgatcaaagtgagcgagatgctgtgtctgagactgtggcacgatcccgaggccaagaagggccaaggcgcttggtatgctgaccccgtgtacaaggctgatatccccgctctgaaagatggcacatacgtccctagaatcgccaaacagaaatatggaagaaaggtctggaaggccgtgcccaatagcgctctgacccaaaaacctctggagatctacctcggagatctgattaaggtcggcgataagctgggcagatacaacggctacaacatcgccaccgccaattggtcctttgtcgacgctctgaccaagaaggaaattgctttccctagcgtcggcatgctgagcaatgaactccaacctatcatcatcagagaaagcattctggacaactga

SEQ ID NO: 12 - DNA, 3165 bp, synthetic construct
  source 1..3165: mol_type = other DNA
atgggaaagcctattcccaaccctctgctgggactggactccacaaaaagaacagccgacggcagcgagttcgaaagccccaagaagaagagaaaagtggagatcacaattaatagggaaattggcaagctgggactccctagacatctggtgctgggcatggatcccggcattgccagctgcggattcgctctgatcgacacagccaatagagaaattctggatctgggcgtgaggctgttcgattcccctacccatcctaagaccggccagtctctggctgtcatcagaaggggcttcagatccacaagaaggaatatcgacagaacccaagctagactcaagcactgcctccagatcctcaaagcctatggcctcattccccaagacgccaccaaagagtacttccacaccaccaagggagataagcagcctctgaaactgagagtggacggactggatagactgctgaacgatagagaatgggctctggtgctgtacagcctctgtaagagaaggggctacatccctcacggcgagggaaatcaagacaagtccagcgaaggaggcaaagtgctgagcgccctcgccgccaacaaggaagctatcgccgagacaagctgtagaaccgtgggcgaatggctcgctcaacagcctcagagcagaaatagaggcggaaactatgacaagtgcgtcacccatgctcagctcattgaggaaacccatatcctcttcgacgctcagagaagctttggcagcaagtacgccagccccgagttcgaagccgcctacattgaagtgtgtgactgggaaaggtctagaaaggattttgatagaaggacatacgatctggtcggccactgcagctacttccctaccgaaaagagagccgctagatgcacactgaccagcgagctggtcagcgcctacggagctctcggcaacatcaccatcatccacgacgacggaaccagcagagctctgagcgccaccgaaagggatgagtgcatcgccatcctctttagctgcgagcccattagaggcaataaggattgtgccgtgaagttcggagctctgagaaaggctctggacctctcctccggagattacttcaagggagtccccgccgccgacgaaaagaccagagaggtctacaagcccaagggctggagagtgctgagaaacaccctcaacgccgccaaccctattctgctccagagactgagagatgacagaaacctcgctgacgctgtgatggaagctgtcgcttacagcagcgctctgcccgtgctccaagagcagctgcaaggactgcctctctccgaggctgagatcgaggctctgtgcagactgccttacagctccaaagctctgaacggctacggcaatagaagcaaaaaagctctggacatgctgctcgattgtctggaggaacccgaagtgctgaacctcacccaagccgaaaacgattgtggactgctcggactgagaatcgccggaacccagctggagagatccgatagactcatgccttatgaaacatggatcgagagaaccggaagaaccaataacaaccccgtggtcatcagagccatgagccaaatgagaaaggtggtcaacgccatctgcagaaagtggggcgtgcccaacgaaattcatgtggagctggatagagagctgagactgccccaaagggctaaggacgagatcgccaaggctaataagaagaatgaaaagaacagagagagaatcgccggccagattgctgaactgagaggctgtacagctgacgaggtgaccggcaagcagattgagaagtatagactctgggaggagcaagagtgctttgatctgtacaccggagccaagatcgaggtggataggctcatcagcgatgatacatacacccagatcgaccacattctgccttttagcagaaccggcgagaactctagaaacaacaaagtgctggtgctcgctaagtccaatcaagacaagagggagcagaccccctatgaatggatgagccatgatggcgctcccagctgggacgccttcgaaaggagggtgcaagagaaccagaagctgtctagaaggaagaagaactttctgctcgagaaggatctggacaccaaggagggagagtttctcgctagaagcttcaccgacacagcctatatgtctagagaggtgtgtgcctatctggccgactgtctgctcttccccgacgatggagctaaggcccatgtggtgcctacaaccggaagagccaccgcttggctcagaaggagatggggactgaacttcggctccaatggcgagaaagatagatccgacgacagacatcacgctacagatgcttgcgtgatcgctgcttgctccagatctctggtgattaagaccgctagaatcaaccaagagacacattggagcatcacaagaggaatgaacgaaacccagaggagggatgccatcatgaaggctctggagtccgtcatgccttgggaaaccttcgccaacgaggtgagagctgcccacgattttgtcgtgcctaccagattcgtgcccagaaaaggcaaaggcgagctcttcgagcagaccgtgtatagatacgctggcgtgaacgcccaaggcaaggatatcgctagaaaggcctcctccgataaagacatcgtcatgggaaacgccgtggtgagcgccgatgaaaagagcgtgatcaaagtgagcgagatgctgtgtctgagactgtggcacgatcccgaggccaagaagggccaaggcgcttggtatgctgaccccgtgtacaaggctgatatccccgctctgaaagatggcacatacgtccctagaatcgccaaacagaaatatggaagaaaggtctggaaggccgtgcccaatagcgctctgacccaaaaacctctggagatctacctcggagatctgattaaggtcggcgataagctgggcagatacaacggctacaacatcgccaccgccaattggtcctttgtcgacgctctgaccaagaaggaaattgctttccctagcgtcggcatgctgagcaatgaactccaacctatcatcatcagagaaagcattctggacaacaagaggacagctgacggaagcgagttcgagagccccaagaaaaagagaaaagtctga

SEQ ID NO: 13 - RNA, 36 nt, synthetic construct
  source 1..36: mol_type = other RNA
gttctggtctaagttcatttcctaactgataaaatc

SEQ ID NO: 14 - RNA, 88 nt, synthetic construct
  source 1..88: mol_type = other RNA
tcagttaggaaatgggctttctccactaacaagctgagagatgcacaagatgcggggtcgctatatgcgaccatttttcgtatccaaa

SEQ ID NO: 15 - RNA, 121 nt, synthetic construct
  source 1..121: mol_type = other RNA
gttctggtctaagttcatttcctaactgagaaatcagttaggaaatgggctttctccactaacaagctgagagatgcacaagatgcggggtcgctatatgcgaccatttttcgtatccaaa

SEQ ID NO: 16 - RNA, 121 nt, synthetic construct
  source 1..121: mol_type = other RNA
gttctggtctaagttcatttcctaactgagaaatcagttaggaaatgggctttctccactaacaagctgagagatgcacaagatgcggggtcgctatatgcgaccattattcgtatccaaa

SEQ ID NO: 17 - RNA, 89 nt, synthetic construct
  source 1..89: mol_type = other RNA
gttctggtctaaggaaactttctccactaacaagctgagagatgcacaagatgcggggtcgctatatgcgaccattattcgtatccaaa

SEQ ID NO: 18 - RNA, 117 nt, synthetic construct
  source 1..117: mol_type = other RNA
gttctggtctaagttcatttcctaactgagaaatcagttaggaaatgggctttctccactaacaagcgaaagcacaagatgcgggctcgctatatgcgagcattattcgtatccaaa

SEQ ID NO: 19 - RNA, 85 nt, synthetic construct
  source 1..85: mol_type = other RNA
gttctggtctaaggaaactttctccactaacaagcgaaagcacaagatgcgggctcgctatatgcgagcattattcgtatccaaa

SEQ ID NO: 20 - RNA, 36 nt, synthetic construct
  source 1..36: mol_type = other RNA
gtcttgagcacgcgcccttccccaaggtgatacgct

SEQ ID NO: 21 - RNA, 105 nt, synthetic construct
  source 1..105: mol_type = other RNA
tcaccttggggaagggcgcggctccagacaaggggagccacttaagtggcttacccgtaaagtaacccccgttcaatcttcggattgggcggggcgaactttttt

SEQ ID NO: 22 - RNA, 133 nt, synthetic construct
  source 1..133: mol_type = other RNA
gtcttgagcacgcgcccttccccaaggtgagaaatcaccttggggaagggcgcggctccagacaaggggagccacttaagtggcttacccgtaaagtaacccccgttcaatcttcggattgggcggggcgaac

SEQ ID NO: 23 - RNA, 133 nt, synthetic construct
  source 1..133: mol_type = other RNA
gtcttgagcacgcgcccttccgcaaggtgagaaatcaccttgcggaagggcgcggctccagacaagcggagccacttaagtggcttacgcgtaaagtaaccgccgttcaatcttcggattgggcggcgcgaac

SEQ ID NO: 24 - RNA, 99 nt, synthetic construct
  source 1..99: mol_type = other RNA
gtcttgagcacgcgaaagcggctccagacaaggggagccacttaagtggcttacccgtaaagtaacccccgttcaatcttcggattgggcggggcgaac

SEQ ID NO: 25 - RNA, 99 nt, synthetic construct
  source 1..99: mol_type = other RNA
gtcttgagcacgcgaaagcggctccagacaagcggagccacttaagtggcttacgcgtaaagtaaccgccgttcaatcttcggattgggcggcgcgaac

SEQ ID NO: 26 - RNA, 139 nt, synthetic construct
  source 1..139: mol_type = other RNA
gtcttgagcacgcgcccttccccaaggtgagaaatcaccttggggaagggcgcggctccagacaaggggagccacttaagtggcttacccgtaaagtaacccccgttcaatcttcggattgggcggggcgaactttttt

SEQ ID NO: 27 - RNA, 139 nt, synthetic construct
  source 1..139: mol_type = other RNA
gtcttgagcacgcgcccttccgcaaggtgagaaatcaccttgcggaagggcgcggctccagacaagcggagccacttaagtggcttacgcgtaaagtaaccgccgttcaatcttcggattgggcggcgcgaactttttt

SEQ ID NO: 28 - RNA, 105 nt, synthetic construct
  source 1..105: mol_type = other RNA
gtcttgagcacgcgaaagcggctccagacaaggggagccacttaagtggcttacccgtaaagtaacccccgttcaatcttcggattgggcggggcgaactttttt

SEQ ID NO: 29 - RNA, 105 nt, synthetic construct
  source 1..105: mol_type = other RNA
gtcttgagcacgcgaaagcggctccagacaagcggagccacttaagtggcttacgcgtaaagtaaccgccgttcaatcttcggattgggcggcgcgaactttttt

SEQ ID NO: 30 - protein, 1004 aa, unidentified
KKKIFGFDLGIASIGWAVIDHSDENFDPETGEIIEGKVVGCGVRCFPVAENPKDGSSLAAPRREKRLLRRITRRKARRMLGIKRLFVAKGLAASTAELETLYAAQTGGDVWNLRAEALRRPLSKEELLRVLTHLAKHRGFKSYRKAAEEADKESGRILTAIAENRKETAGFQTLAQMIVERAKHSDDHKMRNYTSQEGENKGVAVYVNSIPREEIEKETKLIFEYQKQFGLFTEDLYRDFCKIASRYREAGSVGHMVGRCRFEPEQPRAPKEAPSAELFVALSKINNLKVTVDGERRFLNGEERKALLELLKNTKEVKYLTIKNKLFKGREVFFDDVNYAQKTKKGKSGEEKAVNPEDAKFYAMKGWHKLKAAFSPEQWKEVGSNLPLLDLGMTAVVCEKNDAGIERFLSEKGIPEDYREVFKKLTGSEFINLSLKALYKLNPYLAEGLKYNQACEKAGYDFREDGIKLAEEKGLLLPPIADDKLTTVPVVNRAVAQFRKVYNAMVRTYGAPDQINLEIGRDLKKSRDERNQIMRRQKENEAERKEAEDWLEKEGLAANGKNMLKYRLYRQQNGKCIYSGKAIDLRRLDENGYCDVDHIIPYSRSLDDGQNNKVLCLAEENRKKGSQTPYEYLEPLGRWEEFETVVNTTPSINRYKRNNLLNKDYKEKENDLEFRERNANDNSYIARYVKRYLEDAIDFSASSCTIGNRVQVRTGSLTDYLRHQWGLIKDRDASDRHHAQDAVVVACATQGMVQKLSKLSAIFENKDDFRRKKAEELGHEEAEAWYKYVKQQIREPWSGFRAEVLASLEKVFVSRPPRKNATGEIHQETIRTVNPKRKKYNEKEILSGIKIRGGLAKNGLMLRTDVFVKKNKKGKDEFYLVPVYLSDMGKELPNKAMVPGKKENEWIELDETCQFKFSFYMDDLIKIKKKENEIFGYFRGTNRATASVSVTTHDRSHTFEGIGVKTQDGIEKYQVDPLGRIAKVKKEIRLPLTMMKKNRHKKEE

SEQ ID NO: 31 - protein, 1005 aa, unidentified
MKKKIFGFDLGIASIGWAVIDHSDENFDPETGEIIEGKVVGCGVRCFPVAENPKDGSSLAAPRREKRLLRRITRRKARRMLGIKRLFVAKGLAASTAELETLYAAQTGGDVWNLRAEALRRPLSKEELLRVLTHLAKHRGFKSYRKAAEEADKESGRILTAIAENRKETAGFQTLAQMIVERAKHSDDHKMRNYTSQEGENKGVAVYVNSIPREEIEKETKLIFEYQKQFGLFTEDLYRDFCKIASRYREAGSVGHMVGRCRFEPEQPRAPKEAPSAELFVALSKINNLKVTVDGERRFLNGEERKALLELLKNTKEVKYLTIKNKLFKGREVFFDDVNYAQKTKKGKSGEEKAVNPEDAKFYAMKGWHKLKAAFSPEQWKEVGSNLPLLDLGMTAVVCEKNDAGIERFLSEKGIPEDYREVFKKLTGSEFINLSLKALYKLNPYLAEGLKYNQACEKAGYDFREDGIKLAEEKGLLLPPIADDKLTTVPVVNRAVAQFRKVYNAMVRTYGAPDQINLEIGRDLKKSRDERNQIMRRQKENEAERKEAEDWLEKEGLAANGKNMLKYRLYRQQNGKCIYSGKAIDLRRLDENGYCDVDHIIPYSRSLDDGQNNKVLCLAEENRKKGSQTPYEYLEPLGRWEEFETVVNTTPSINRYKRNNLLNKDYKEKENDLEFRERNANDNSYIARYVKRYLEDAIDFSASSCTIGNRVQVRTGSLTDYLRHQWGLIKDRDASDRHHAQDAVVVACATQGMVQKLSKLSAIFENKDDFRRKKAEELGHEEAEAWYKYVKQQIREPWSGFRAEVLASLEKVFVSRPPRKNATGEIHQETIRTVNPKRKKYNEKEILSGIKIRGGLAKNGLMLRTDVFVKKNKKGKDEFYLVPVYLSDMGKELPNKAMVPGKKENEWIELDETCQFKFSFYMDDLIKIKKKENEIFGYFRGTNRATASVSVTTHDRSHTFEGIGVKTQDGIEKYQVDPLGRIAKVKKEIRLPLTMMKKNRHKKEE

SEQ ID NO: 32 - DNA, 3015 bp, unidentified
  source 1..3015: mol_type = unassigned DNA
atgaaaaagaaaatttttggttttgatttggggattgcttcgatcggttgggcggttattgatcatagtgatgaaaatttcgatccggaaacaggagagattattgaaggaaaagtcgttggctgcggagtgcgctgttttccggtagcggaaaacccgaaggacggttcctcgctggcggcgccccggcgggaaaaacgcttgttgcgccggatcacccgccgaaaggccaggagaatgctcggtattaagcgtctgtttgtcgccaagggactggctgcttcaacggcggaattggaaacgctttatgccgcacaaacaggcggcgacgtttggaatctgcgggcagaggccttgcggcgtccgctgtcaaaagaggaactgctgcgagttttgactcatttggcaaaacatcgcggtttcaagtcttaccgtaaggctgctgaagaggcggacaaggaaagcggccgcatcttgactgcgattgcggaaaaccggaaggaaacggccggttttcaaacgctggcgcagatgatcgttgaacgggccaaacattccgacgatcataaaatgcgcaactatacgtcgcaggaaggcgaaaacaagggcgtagccgtttatgtcaattccattccgcgggaagaaattgaaaaggaaacaaaactgatttttgagtatcagaagcaattcggtttgtttaccgaggacttataccgcgatttttgcaagattgcctcccgttaccgggaagcggggagtgtcgggcacatggttggcagatgccggtttgaaccggaacagccgcgggcgccgaaagaagccccttcggcggagctcttcgttgctttaagcaaaattaataacctgaaagtgaccgttgacggcgaacgccgtttcttaaacggagaagagcggaaggctttgcttgaattgctgaaaaacaccaaggaagtcaaatatttgactatcaaaaacaaattgtttaaaggccgggaagtcttttttgacgacgttaactatgcgcaaaaaacaaaaaaaggaaaaagcggagaagaaaaagcggtaaatcccgaagacgcaaagttttacgccatgaaaggctggcataagctgaaagccgctttttcaccggagcagtggaaagaggcggttcgaatttgccgttgctggatctcggcatgaccgcggtcgtctgcgaaaaaaacgacgccggaatagagcgctttttaagcgaaaaaggaataccggaggattatcgggaagtttttaaaaagctgaccggcagcgagtttattaatctgtcactgaaagccctttataagctcaatccttatctggcggaaggtttgaagtataaccaagcctgcgaaaaggccggatacgacttccgcgaggacggcatcaaactggcggaggaaaaaggtttgttgctgccgccgattgcagatgacaaactgacgacggtgccggtcgttaaccgtgcggttgcccagttccgcaaagtatataacgccatggttcggacatatggtgcgccggatcagataaatctggaaatcggccgggatttgagaaaagccgtgacgagcgcaatcagatcatgcggcggcaaaaggaaaacgaggccgaacggaaagaagccgaggactggttggaaaaggaaggacttgccgcaaacggtaaaaatatgttgaaataccgtttgtaccggcagcaaaacggcaaatgcatctattccggaaaagcgattgacctccgccgcctggacgaaaacggttattgcgacgttgaccacatcatcccttattcccgttcgcttgacgacggtcagaacaacaaagtgctttgtctggccgaagaaaaccgcaagaaaggaagccaaactccttatgaatatctggagccgctcggacgatgggaagagtttgaaaccgttgttaacaccacgccgtcgattaaccgttacaaaagaaacaacctgttgaacaaagattataaagaaaaagaaaacgatttggaatttcgcgaaagaaacgccaatgacaactcctatattgcccgctatgtcaaacggtatttggaagatgccattgatttttccgccagttcctgcacaatcggaaaccgggttcaggtgcgcaccggttcgttaaccgattacctccgccatcagtgggggctgataaaagatcgtgacgcaagcgacaggcatcatgctcaggacgcggttgtcgttgcctgcgccacgcagggaatggtgcagaaactgtcaaaactttccgcgatttttgaaaacaaggacgatttccgcagaaagaaagcggaagaactcgggcacgaggaggccgaagcctggtacaaatacgtcaaacagcaaattcgggaaccctggagcggttttcgggctgaagtactggccagcctggaaaaggttttcgtttcccgtccgccgcgcaaaaacgcaaccgagagattcaccaggaaacgattcgcacggttaatccgaaacgtaaaaaatataatgaaaaggaaattctgtccggcatcaaaatccgcggcgggctggccaaaaacggcctgatgctgcgaacggacgtttttgtaaaaaagaacaaaaagggaaaagacgaattttacctggtgccggtttatctttccgatatgggaaaagagctgccgaacaaggcgatggttccgggtaaaaaagaaaacgaatggattgaactggatgaaacctgtcagtttaaattcagcttttatatggacgatttgataaaaatcaaaaaaaaggaaaatgagattttcggctatttcagaggaacaaacagggcgacggcgtcagtatccgttaccacccatgaccgcagtcatacttttgaaggcatcggcgtcaaaactcaggacggtatcgaaaaatatcaggtggatccgctgggacgtattgccaaagtcaaaaaagaaatccggctcccgctgacgatgatgaaaaagaaccggcataaaaaggaggagtga

SEQ ID NO: 33 - DNA, 3168 bp, synthetic construct
  source 1..3168: mol_type = other DNA
atgggcaagcccatccctaatcctctgctgggactggacagcaccaaaagaaccgctgacggatccgagttcgagagccccaagaaaaagaggaaggtcaagaaaaagatttttggcttcgatctcggaattgctagcatcggatgggccgtgattgaccactccgacgagaacttcgaccccgaaaccggcgagattatcgagggcaaggtggtcggctgcggagtgagatgtttccccgtggccgagaatcccaaggacggaagctccctcgctgcccctaggagggagaagaggctgctgagaaggatcaccagaagaaaggccagaaggatgctgggcatcaaaaggctgttcgtggccaaaggactggccgctagcacagctgagctggagacactctacgccgctcagaccggcggagatgtgtggaatctgagggctgaggccctcagaaggcctctgagcaaggaggaactgctcagagtgctcacccatctggccaagcatagaggatttaagagctatagaaaagccgccgaggaagccgacaaagagtccggaagaatcctcaccgctatcgccgagaataggaaggaaaccgccggctttcaaacactggcccagatgattgtggaaagagccaagcacagcgatgaccacaagatgaggaattacacctcccaagagggcgagaacaaaggcgtggccgtgtacgtcaactccattcctagagaggagatcgaaaaggaaaccaaactgattttcgaataccagaagcagttcggactgttcaccgaagatctgtatagagacttctgcaagatcgccagcagatatagagaggctggctccgtgggacacatggtcggaaggtgcagatttgagcccgagcaacccagagctcccaaggaggccccttccgccgaactgttcgtggctctgtccaagatcaacaacctcaaagtgacagtggatggcgagagaagatttctgaacggcgaggagagaaaagccctcctcgagctgctcaaaaacaccaaggaagtcaagtacctcaccattaagaataagctcttcaagggcagagaggtcttcttcgatgacgtgaactacgcccagaaaaccaaaaaaggcaagagcggcgaggaaaaggctgtgaaccccgaggacgccaagttttacgctatgaagggatggcacaagctcaaggctgccttttcccccgaacagtggaaagaggtgggcagcaatctgcccctcctcgatctgggaatgacagccgtggtctgcgagaagaacgacgctggcatcgagagatttctgtccgaaaagggcattcccgaagactatagagaggtgtttaaaaaactgaccggctccgagttcatcaacctctctctgaaggctctctataagctgaacccctatctggccgagggactgaaatacaaccaagcttgcgaaaaagccggctacgactttagagaggacggcattaagctggccgaagaaaaaggactgctgctgccccccattgccgatgataaactgaccaccgtgcccgtggtcaacagagccgtggcccagttcagaaaagtgtataacgctatggtgagaacatacggagcccccgaccaaatcaatctggaaattggaagagatctgaagaagtctagagatgagaggaaccaaatcatgaggaggcaaaaagagaacgaggccgagaggaaggaagccgaggattggctcgaaaaggagggactggctgccaacggaaaaaacatgctcaagtacagactgtataggcagcagaacggcaagtgcatctacagcggcaaagctatcgatctgagaaggctggacgaaaatggatactgcgacgtggatcacatcatcccttactccagatctctggacgacggacaaaacaacaaagtgctgtgtctcgccgaggaaaatagaaagaagggcagccaaaccccttacgagtatctggagcctctgggcagatgggaggaattcgaaaccgtggtgaacaccacaccctccatcaatagatataagagaaataatctgctcaataaagattataaggaaaaggagaacgacctcgagtttagggagaggaacgccaacgacaacagctacatcgctagatacgtgaagaggtatctggaggacgccatcgactttagcgcttccagctgcaccatcggcaatagagtgcaagtgagaaccggcagcctcaccgactatctgagacaccaatggggactcattaaggatagagacgctagcgacagacaccatgcccaagacgctgtggtcgtcgcttgcgccacccaaggaatggtgcagaagctctccaaactcagcgctatctttgaaaataaagatgattttagaagaaagaaggccgaggaactgggacacgaagaggctgaggcttggtacaagtacgtgaagcagcagattagagaaccttggtccggatttagggccgaggtgctggcctctctggagaaggtgttcgtctctagacctcccagaaagaacgctaccggagagatccaccaagaaaccattaggaccgtcaaccccaagagaaaaaaatacaacgagaaagagatcctctccggcatcaagattagaggaggcctcgccaagaacggcctcatgctgagaaccgatgtctttgtgaagaaaaataaaaagggcaaggacgaattctacctcgtccccgtgtatctgtccgacatgggcaaagagctgcctaataaggctatggtgcccggcaagaaggaaaacgagtggatcgaactggacgagacatgccaattcaaattttccttctacatggatgacctcatcaagattaagaaaaaggaaaatgagatcttcggctattttagaggaacaaacagagccaccgctagcgtctccgtgaccacccacgatagaagccacacatttgagggcatcggagtgaagacccaagacggaattgagaagtaccaagtggaccctctcggaagaatcgccaaggtgaaaaaggagattagactgcctctgaccatgatgaaaaagaacagacataagaaggaggagaagagaacagctgatggcagcgagttcgaatcccctaagaagaagaggaaggtgtga

SEQ ID NO: 34 - protein, 1003 aa, unidentified
EITINREIGKLGLPRHLVLGMDPGIASCGFALIDTANHEILDLGVRLFDSPTHPKTGQSLAVIRRGFRSTRRNIDRTQARLKHCLQVLKAYGLIPQDATKEYLHTTKGDKQPLKLRVDGLDRLLNDREWALVLYSLCKRRGYIPHGEGNQDKSSEGGKVLSALAANKEAIAETSCRTVGEWLAWQPQSRNRGGNYDKCVTHAQLIEETHILFDAQRSFGSKYASPEFEAAYIEVCDWERSRKDFDRRTYDLVGHCSYFPTEKRAARCTLTSELVSAYGALGNITIIHENGTSRALSATERDECIAILFSCEPIRGNKDCAVKFGALRKALDLSSGDYFKGVPAADEKTREVYKPKGWRVLRNTLNAANPILLQRLRDDRNLADAVMEAVAYSSALPVLQEQLQGLPFSEAEIEALCRLPYSSKALNGYGNRSKKALDMLLDCLEEPEVLNLTQAENDCGLLGLRIAGAQLERSDRLMPYETWIELTGRTNNNPVVIRSMSQMRKVVNAVCRKWGVPNEIHVELDRELRLPQRAKDEIAKANKKNEKNRERIAGQIAELRGCTADEVTGKQIEKYRLWEEQECFDLYTGAKIEVDRLISDDTYTQIDHILPFSRTGENSRNNKVLVLAKSNQDKREQTPYEWMSHDGAPSWDAFERRVQENQKLSRRKKNFLLEKDLDTKEGEFLARSFTDTAYMSREACAYLADCLLFPDDGAKAHVVPTTGRATAWLRRRWGLNFGSNGEKDRSDDRHHATDACVIAACSRSLVIKTARINQETHWSITRGMNETQRRDAIMKALESVMPWETFANEVRAAHDFVVPTRFVPRKGKGELFEQTVYRYAGVNAQGKDIARKASSDKDIVMGNAVVSLDEKSVIKVSEMLCLRLWHDPEAKKGQGAWYADPVYKADIPALKDGTYVPRIAKAHTGRKAWKPVPESAMKKPPLEIYLGDLVQIGDFMGRFSGYNIANANWSFVDRLTKEALGCPTVVKLDNKLAPAIIRESIIMH

SEQ ID NO: 35 - protein, 1004 aa, unidentified
MEITINREIGKLGLPRHLVLGMDPGIASCGFALIDTANHEILDLGVRLFDSPTHPKTGQSLAVIRRGFRSTRRNIDRTQARLKHCLQVLKAYGLIPQDATKEYLHTTKGDKQPLKLRVDGLDRLLNDREWALVLYSLCKRRGYIPHGEGNQDKSSEGGKVLSALAANKEAIAETSCRTVGEWLAWQPQSRNRGGNYDKCVTHAQLIEETHILFDAQRSFGSKYASPEFEAAYIEVCDWERSRKDFDRRTYDLVGHCSYFPTEKRAARCTLTSELVSAYGALGNITIIHENGTSRALSATERDECIAILFSCEPIRGNKDCAVKFGALRKALDLSSGDYFKGVPAADEKTREVYKPKGWRVLRNTLNAANPILLQRLRDDRNLADAVMEAVAYSSALPVLQEQLQGLPFSEAEIEALCRLPYSSKALNGYGNRSKKALDMLLDCLEEPEVLNLTQAENDCGLLGLRIAGAQLERSDRLMPYETWIELTGRTNNNPVVIRSMSQMRKVVNAVCRKWGVPNEIHVELDRELRLPQRAKDEIAKANKKNEKNRERIAGQIAELRGCTADEVTGKQIEKYRLWEEQECFDLYTGAKIEVDRLISDDTYTQIDHILPFSRTGENSRNNKVLVLAKSNQDKREQTPYEWMSHDGAPSWDAFERRVQENQKLSRRKKNFLLEKDLDTKEGEFLARSFTDTAYMSREACAYLADCLLFPDDGAKAHVVPTTGRATAWLRRRWGLNFGSNGEKDRSDDRHHATDACVIAACSRSLVIKTARINQETHWSITRGMNETQRRDAIMKALESVMPWETFANEVRAAHDFVVPTRFVPRKGKGELFEQTVYRYAGVNAQGKDIARKASSDKDIVMGNAVVSLDEKSVIKVSEMLCLRLWHDPEAKKGQGAWYADPVYKADIPALKDGTYVPRIAKAHTGRKAWKPVPESAMKKPPLEIYLGDLVQIGDFMGRFSGYNIANANWSFVDRLTKEALGCPTVVKLDNKLAPAIIRESIIMH

SEQ ID NO: 36 - DNA, 3015 bp, unidentified
  source 1..3015: mol_type = unassigned DNA
atggagatcaccatcaatcgcgaaattgggaagctcggacttcccaggcatcttgtgcttggcatggatccaggaattgcaagctgcggattcgcacttatcgacacggccaatcatgaaatcctggatttgggcgtcagattatttgactctccaactcatcctaaaacgggacaaagccttgcggttattcgcaggggattccgctctacccgtcgaaacattgaccgtacccaggcgcgcttgaagcactgtctccaagtcctcaaggcttatggcctcatcccccaagacgccaccaaagagtacctccacaccacaaaaggcgacaagcagccgctcaagcttcgtgttgatggccttgaccgcctgctcaacgatcgcgagtgggcactagtcctatactccctctgcaagcgccgtggatacatcccccacggagaaggcaatcaggataaatcaagcgaaggcggcaaggttctatccgcccttgcggccaacaaggaggcaattgcggagacctcgtgccgcaccgttggcgaatggctcgcttggcaacctcaaagtcgcaatcgtggcggcaattacgacaagtgtgtaacgcacgcccagcttatcgaagaaactcatatcctatttgatgctcaacgctcctttggctccaaatacgcttcgccggaatttgaggccgcatatatcgaggtttgcgattgggagcgttcgcgcaaagacttcgaccgccgcacgtacgacctcgttggccactgctcatacttcccaacagaaaaacgagccgcacgctgcacgcttacgagcgaacttgtttcagcctatggagcactcggcaacatcaccatcatccacgaaaacgggacctctcgcgccctgagcgcaacggagcgtgatgagtgcattgcaatcctgttctcgtgcgaaccaattcgaggcaacaaagattgtgctgttaaattcggcgccctcagaaaagcgctcgaccttagttccggcgattactttaagggagttccagccgccgacgaaaaaacgcgagaggtgtacaagcccaagggatggcgcgtgctccgcaataccctcaatgcggccaaccccattctcctgcagcgtttgcgcgatgaccgcaatctcgccgatgccgttatggaggcggtggcatattcctcggcccttcccgtactccaagagcagcttcaggggttgccgttctcggaagcggagatcgaggcgctttgtaggcttccctattcatccaaagctcttaacggctatggcaaccgttccaaaaaagcactcgacatgctgctcgattgcctcgaggagcccgaggtcctcaaccttacacaggccgaaaatgactgcggcctgctgggacttcgcatcgctggcgcccagctcgagcgctccgatcgtctgatgccctatgagacctggatcgaacttaccggtcggacaaataacaatcccgtcgtcattcgttccatgtcgcaaatgcgaaaagtggtcaacgccgtctgccgcaagtggggcgtgccaaacgaaatccacgttgagcttgatcgagagctcaggttgcctcagcgcgcaaaagacgagattgccaaggccaataagaagaatgagaaaaatcgtgagcgcattgccggacaaatcgctgaactgcgtggctgcacggcagatgaggtcacgggcaaacagatagagaagtaccgcctgtgggaagagcaggaatgcttcgatctttacacgggcgctaaaatcgaagtcgatcgcctaattagcgacgacacttacacgcagatcgaccacatcctgccgttctctcgcacgggagaaaactctcgcaacaacaaagtcctagtcctcgccaaaagcaatcaggacaaacgcgaacagacaccttacgaatggatgtcccacgacggcgcgccttcatgggatgcttttgagcgtcgcgttcaggaaaaccagaaactcagccgtcgcaaaaagaacttcctgctggaaaaagaccttgacaccaaggaaggcgaattcttagcacgcagcttcaccgacaccgcctatatgtcgcgagaagcatgcgcttacctcgccgactgcctactgttccccgatgatggcgcaaaggcacatgttgttcccaccactggcagagcgaccgcatggctgcgtcgcaggtgggggcttaactttggttcgaatggcgaaaaagaccgctcggacgatcgtcaccatgccaccgatgcttgtgtgattgcagcatgtagtcgaagcctcgtgattaaaaccgctcgaatcaaccaagagacacactggagcataaccagaggtatgaacgagacccaacgccgcgatgccatcatgaaggctctcgaaagtgttatgccctgggaaacctttgcgaacgaagtacgtgcggcgcacgatttcgtcgtacccacgcgctttgttccgcgtaagggaaagggcgagttgttcgagcagacggtctatcgctatgccggcgttaatgcacagggcaaagacattgctcgcaaggcgagctccgataaggacatcgtcatgggcaacgccgttgtgtcattagacgaaaagtcggtcatcaaggtgagcgaaatgctgtgtctgaggctctggcatgacccggaggccaagaaggggcagggcgcttggtacgcagacccggtctacaaggcggatattcctgcacttaaggatgggacgtatgttcccaggattgcgaaggcgcatactggccgaaaagcctggaagcccgtgcccgaaagcgctatgaaaaaaccgccgctggagatatatctgggtgatctggtacaaatcggcgattttatggggcggtttagcggctacaacatcgcaaatgcaaactggtcgtttgtcgacaggctcactaaagaagccctaggctgtcccaccgttgtcaagttggacaacaaactggctcccgccataattcgcgagtccataatcatgcactaa

SEQ ID NO: 37 - DNA, 3165 bp, synthetic construct
  source 1..3165: mol_type = other DNA
atgggaaagcctattcctaaccctctgcttggcctcgacagcacaaagagaacagctgatggcagcgagttcgagagccctaagaaaaagcgaaaagtggaaattacaatcaaccgagagatcggaaaactgggcctgcctagacacctggttctgggcatggaccccggcatcgcctcctgtggcttcgccctgatcgacaccgccaaccacgaaatcctggatctgggcgtccggctgttcgatagccctacccaccccaagaccggacagtctctggccgtgatcagaagaggcttcagaagcaccagaagaaacatcgacagaacccaggctagactgaagcactgcctgcaggtcctgaaagcctacggcttgatcccgcaggacgccaccaaagagtacctgcacaccacaaagggcgacaagcagcctctgaagctgagggtggacggcctggatagactgctcaacgaccgggagtgggctctggtgctgtacagcctgtgcaagcgtagaggctacatccctcacggcgaaggaaaccaggataagagcagcgaaggcggcaaggtgctgagcgctctcgctgccaataaggaagctatcgccgagacaagctgccggaccgtgggcgaatggctggcctggcagccccagagccggaaccggggaggaaattacgacaagtgcgtgacacacgcccaactgattgaggagacacatatcctgttcgacgcccagagatcttttggctctaagtacgccagccctgagtttgaagcggcatatatcgaggtgtgcgactgggagagaagcagaaaggatttcgaccgccggacctatgacctggtcggacactgcagctatttccccaccgagaaacgggccgccagatgcaccctgaccagcgagctggtgtccgcctacggggctctgggaaacatcaccatcatacacgaaaacggcaccagcagagccctgagcgctactgagcgggacgaatgcatcgccatcctgttttcttgtgaacccatccgaggcaacaaggattgcgccgtcaagttcggcgctctgagaaaagccctggacctgagcagcggcgattacttcaagggcgtgcctgccgccgatgaaaagaccagagaagtctacaagcctaagggctggcgggtgttgagaaataccctgaacgccgccaatcccatcttactgcagagactgagagatgatcggaacctggctgacgccgtgatggaagccgtggcctacagctctgcgctgcccgtgctgcaggagcagctgcaaggcctgcctttcagcgaggccgagatcgaggccctgtgtagactgccttattctagcaaggccctgaacggatacggcaatagaagtaaaaaggccctggatatgctgctggattgcctggaagaacctgaggtgctaaacctgacccaggccgagaatgactgcggcctgctgggcctgcggatcgccggcgcccagctggagagatctgatagactgatgccttacgaaacctggatcgagctgacaggcagaacaaacaacaatcctgtggtgatccggagcatgtctcagatgagaaaagtggtgaacgccgtgtgccggaagtggggcgtgcccaacgagatccatgtggaactggatagagagctgagactgcctcagcgggctaaagacgagatcgccaaggctaacaagaaaaacgagaagaacagagagaggatcgcaggccagattgcagaactgagaggatgtaccgccgacgaggttacaggtaaacagatcgaaaagtaccggctctgggaagagcaggagtgcttcgacctgtacaccggcgccaaaatcgaggtggacagactgatcagcgatgacacctacacacagatcgaccacatcctgcccttcagcagaaccggcgaaaacagccggaacaacaaggtgctggtgctggctaagtctaatcaagacaagagagagcagaccccttacgagtggatgagccacgacggcgcccctagctgggacgcctttgagagaagagtgcaggagaatcaaaagctgtcccggagaaagaagaacttcctgcttgagaaggacctggacaccaaagagggcgagttcctggcccggagcttcaccgacacagcttacatgtccagagaggcctgcgcctacctggccgactgcctgctgttccccgatgacggcgccaaagcccatgtggtgcctaccaccggcagagccacagcctggctgcgaagaaggtggggactgaatttcggcagcaacggcgagaaggacagaagcgacgaccggcaccacgccacagacgcctgtgtgatcgccgcctgcagcagaagcctggtgatcaagaccgctagaatcaaccaagaaacccactggagcatcacccggggcatgaacgaaacccagagaagggatgccatcatgaaggctcttgagtctgtgatgccctgggaaaccttcgccaacgaggtgcgggccgctcacgacttcgtggtgcctacaagattcgtgccaagaaaaggaaagggcgagctgtttgagcaaaccgtgtacagatacgccggagttaatgcccagggtaaagatatcgcccgtaaggccagctccgacaaggacatcgtgatgggcaacgccgtggtttccctggatgaaaagagcgtgatcaaggtgtccgaaatgctgtgcctgagactgtggcacgatcctgaggcgaagaagggccagggcgcctggtacgccgacccagtgtacaaggccgacatccctgctctgaaagatggcacctacgtgccaagaatcgccaaggcccacaccggccggaaggcctggaagcctgtgcctgagtccgccatgaagaagccccccctggaaatctacctgggggatctggtgcagatcggcgacttcatgggcagattctccggctacaacatcgccaacgccaactggtcctttgtggacagactgacaaaagaagccctgggctgtcctacagtggtgaagctggacaacaagctggcaccagccatcatccgggaatctatcatcatgcacaagagaaccgccgacggctctgagttcgagtctccaaagaagaaacggaaagtgtga

SEQ ID NO: 38 - protein, 18 aa, synthetic construct
KRTADGSEFESPKKKRKV

SEQ ID NO: 39 - protein, 7 aa, synthetic construct
PKKKRKV

SEQ ID NO: 40 - protein, 7 aa, synthetic construct
PKKKRRV

SEQ ID NO: 41 - protein, 16 aa, synthetic construct
KRPAATKKAGQAKKKK

SEQ ID NO: 42 - protein, 11 aa, synthetic construct
YGRKKRRQRRR

SEQ ID NO: 43 - protein, 9 aa, synthetic construct
RKKRRQRRR

SEQ ID NO: 44 - protein, 9 aa, synthetic construct
PAAKRVKLD

SEQ ID NO: 45 - protein, 11 aa, synthetic construct
RQRRNELKRSP

SEQ ID NO: 46 - protein, 8 aa, synthetic construct
VSRKRPRP

SEQ ID NO: 47 - protein, 8 aa, synthetic construct
PPKKARED

SEQ ID NO: 48 - protein, 8 aa, synthetic construct
PQPKKKPL

SEQ ID NO: 49 - protein, 12 aa, synthetic construct
SALIKKKKKMAP

SEQ ID NO: 50 - protein, 7 aa, synthetic construct
PKQKKRK

SEQ ID NO: 51 - protein, 10 aa, synthetic construct
RKLKKKIKKL

SEQ ID NO: 52 - protein, 10 aa, synthetic construct
REKKKFLKRR

SEQ ID NO: 53 - protein, 20 aa, synthetic construct
KRKGDEVDGVDEVAKKKSKK

SEQ ID NO: 54 - protein, 17 aa, synthetic construct
RKCLQAGMNLEARKTKK

SEQ ID NO: 55 - protein, 38 aa, synthetic construct
NQSSNFGPMKGGNFGGRSSGPYGGGGQYFAKPRNQGGY

SEQ ID NO: 56 - protein, 42 aa, synthetic construct
RMRIZFKNKGKDTAELRRRRVEVSVELRKAKKDEQILKRRNV

SEQ ID NO: 57 - protein, 14 aa, synthetic construct
GKPIPNPLLGLDST

SEQ ID NO: 58 - protein, 18 aa, synthetic construct
KRTADGSEFESPKKKRKV

SEQ ID NO: 59-72 - skipped (14 entries)

SEQ ID NO: 73 - DNA, 24 bp, synthetic construct
  source 1..24: mol_type = other DNA
gcccttcagctcgatgcggttcac

SEQ ID NO: 74 - RNA, 24 nt, synthetic construct
  source 1..24: mol_type = other RNA
gcccttcagctcgatgcggttcac

SEQ ID NO: 75 - RNA, 134 nt, synthetic construct
  source 1..134: mol_type = other RNA
gttatagcttcctttccaaatcagacatgctatagaaatattatatgtctgatttggaaaggaagtctataataatcgaagttatctttacgagtagggctctgacgtcacatataatatatgtggcgtcatcc

SEQ ID NO: 76 - RNA, 133 nt, synthetic construct; identical to SEQ ID NO: 22
  source 1..133: mol_type = other RNA

SEQ ID NO: 77 - RNA, 133 nt, synthetic construct; identical to SEQ ID NO: 23
  source 1..133: mol_type = other RNA

SEQ ID NO: 78 - RNA, 99 nt, synthetic construct; identical to SEQ ID NO: 24
  source 1..99: mol_type = other RNA

SEQ ID NO: 79 - RNA, 99 nt, synthetic construct; identical to SEQ ID NO: 25
  source 1..99: mol_type = other RNA

SEQ ID NO: 80 - DNA, 24 bp, synthetic construct
  source 1..24: mol_type = other DNA
gcccttcagctcgatgcggttcac

SEQ ID NO: 81 - DNA, 38 bp, synthetic construct
  source 1..38: mol_type = other DNA
gatgcccttcagctcgatgcggttcaccagggtgtcgc

SEQ ID NO: 82 - DNA, 28 bp, synthetic construct
  source 1..28: mol_type = other DNA
caccgcccttcagctcgatgcggttcac

SEQ ID NO: 83 - DNA, 28 bp, synthetic construct
  source 1..28: mol_type = other DNA
agacgtgaaccgcatcgagctgaagggc

SEQ ID NO: 84 - DNA, 24 bp, synthetic construct
  source 1..24: mol_type = other DNA
cctcgccggacacgctgaacttgt

SEQ ID NO: 85 - DNA, 38 bp, synthetic construct
  source 1..38: mol_type = other DNA
cgccctcgccggacacgctgaacttgtggccgtttacg

SEQ ID NO: 86 - DNA, 29 bp, synthetic construct
  source 1..29: mol_type = other DNA
caccgcctcgccggacacgctgaacttgt

SEQ ID NO: 87 - DNA, 29 bp, synthetic construct
  source 1..29: mol_type = other DNA
agacacaagttcagcgtgtccggcgaggc

SEQ ID NO: 88 - DNA, 24 bp, synthetic construct
  source 1..24: mol_type = other DNA
ggtgcgctcctggacgtagccttc

SEQ ID NO: 89 - DNA, 38 bp, synthetic construct
  source 1..38: mol_type = other DNA
gatggtgcgctcctggacgtagccttcgggcatggcgg

SEQ ID NO: 90 - DNA, 28 bp, synthetic construct
  source 1..28: mol_type = other DNA
caccggtgcgctcctggacgtagccttc

SEQ ID NO: 91 - DNA, 28 bp, synthetic construct
  source 1..28: mol_type = other DNA
gaacgaaggctacgtccaggagcgcacc

SEQ ID NO: 92 - DNA, 24 bp, synthetic construct
  source 1..24: mol_type = other DNA
gcccgaaggctacgtccaggagcg

SEQ ID NO: 93 - DNA, 38 bp, synthetic construct
  source 1..38: mol_type = other DNA
catgcccgaaggctacgtccaggagcgcaccatcttct

SEQ ID NO: 94 - DNA, 28 bp, synthetic construct
  source 1..28: mol_type = other DNA
caccgcccgaaggctacgtccaggagcg

SEQ ID NO: 95 - DNA, 28 bp, synthetic construct
  source 1..28: mol_type = other DNA
gaaccgctcctggacgtagccttcgggc

SEQ ID NO: 96 - DNA, 24 bp, synthetic construct
  source 1..24: mol_type = other DNA
tgcccctccctccctggcccaggt

SEQ ID NO: 97 - DNA, 38 bp, synthetic construct
  source 1..38: mol_type = other DNA
ctgtgcccctccctccctggcccaggtgaaggtgtggt

SEQ ID NO: 98 - DNA, 29 bp, synthetic construct
  source 1..29: mol_type = other DNA
caccgtgcccctccctccctggcccaggt

SEQ ID NO: 99 - DNA, 29 bp, synthetic construct
  source 1..29: mol_type = other DNA
agacacctgggccagggagggaggggcac

SEQ ID NO: 100 - DNA, 24 bp, synthetic construct
  source 1..24: mol_type = other DNA
cccagtggctgctctgggggcctc

SEQ ID NO: 101 - DNA, 38 bp, synthetic construct
  source 1..38: mol_type = other DNA
aggcccagtggctgctctgggggcctcctgagtttctc

SEQ ID NO: 102 - DNA, 29 bp, synthetic construct
  source 1..29: mol_type = other DNA
caccgcccagtggctgctctgggggcctc

SEQ ID NO: 103 - DNA, 29 bp, synthetic construct
  source 1..29: mol_type = other DNA
agacgaggcccccagagcagccactgggc

SEQ ID NO: 104 - DNA, 24 bp, synthetic construct
  source 1..24: mol_type = other DNA
gggcatggtttcataactaggagg

SEQ ID NO: 105 - DNA, 38 bp, synthetic construct
  source 1..38: mol_type = other DNA
aatgggcatggtttcataactaggaggtggtgtttata

SEQ ID NO: 106 - DNA, 28 bp, synthetic construct
  source 1..28: mol_type = other DNA
caccgggcatggtttcataactaggagg

SEQ ID NO: 107 - DNA, 28 bp, synthetic construct
  source 1..28: mol_type = other DNA
agaccctcctagttatgaaaccatgccc

SEQ ID NO: 108 - DNA, 24 bp, synthetic construct
  source 1..24: mol_type = other DNA
gaagcaggccaatggggaggacat

SEQ ID NO: 109 - DNA, 38 bp, synthetic construct
  source 1..38: mol_type = other DNA
cacgaagcaggccaatggggaggacatcgatgtcacct

SEQ ID NO: 110 - DNA, 28 bp, synthetic construct
  source 1..28: mol_type = other DNA
caccgaagcaggccaatggggaggacat

SEQ ID NO: 111 - DNA, 28 bp, synthetic construct
  source 1..28: mol_type = other DNA
gaacatgtcctccccattggcctgcttc

SEQ ID NO: 112 - DNA, 24 bp, synthetic construct
  source 1..24: mol_type = other DNA
gcccactgtgtcctcttcctgccc

SEQ ID NO: 113 - DNA, 38 bp, synthetic construct
  source 1..38: mol_type = other DNA
ctggcccactgtgtcctcttcctgccctgccatcccct

SEQ ID NO: 114 - DNA, 28 bp, synthetic construct
  source 1..28: mol_type = other DNA
caccgcccactgtgtcctcttcctgccc

SEQ ID NO: 115 - DNA, 28 bp, synthetic construct
  source 1..28: mol_type = other DNA
gaacgggcaggaagaggacacagtgggc

SEQ ID NO: 116 - DNA, 24 bp, synthetic construct
  source 1..24: mol_type = other DNA
cgcctgggcagccagggctggcct

SEQ ID NO: 117 - DNA, 39 bp, synthetic construct
  source 1..39: mol_type = other DNA
gctccgcctgggcagccagggctggcctcagggtgtgtt

SEQ ID NO: 118 - DNA, 29 bp, synthetic construct
  source 1..29: mol_type = other DNA
caccgcgcctgggcagccagggctggcct

SEQ ID NO: 119 - DNA, 29 bp, synthetic construct
  source 1..29: mol_type = other DNA
agacaggccagccctggctgcccaggcgc

SEQ ID NO: 120 - DNA, 24 bp, synthetic construct
  source 1..24: mol_type = other DNA
agagaattcccggaagggagacaa

SEQ ID NO: 121 - DNA, 38 bp, synthetic construct
  source 1..38: mol_type = other DNA
aagagagaattcccggaagggagacaaggcagtttctt

SEQ ID NO: 122 - DNA, 29 bp, synthetic construct
  source 1..29: mol_type = other DNA
caccgagagaattcccggaagggagacaa

SEQ ID NO: 123 - DNA, 29 bp, synthetic construct
  source 1..29: mol_type = other DNA
agacttgtctcccttccgggaattctctc

SEQ ID NO: 124 - DNA, 24 bp, synthetic construct
  source 1..24: mol_type = other DNA
cccagcaggagaccaagcagaaat

SEQ ID NO: 125 - DNA, 38 bp, synthetic construct
  source 1..38: mol_type = other DNA
aaccccagcaggagaccaagcagaaatcaccatgggag

SEQ ID NO: 126 - DNA, 29 bp, synthetic construct
  source 1..29: mol_type = other DNA
caccgcccagcaggagaccaagcagaaat

SEQ ID NO: 127 - DNA, 29 bp, synthetic construct
  source 1..29: mol_type = other DNA
gaacatttctgcttggtctcctgctgggc

SEQ ID NO: 128 - DNA, 24 bp, synthetic construct
  source 1..24: mol_type = other DNA
acaaggcagtttctttttctgtgt

SEQ ID NO: 129 - DNA, 38 bp, synthetic construct
  source 1..38: mol_type = other DNA
gagacaaggcagtttctttttctgtgtgacaataaaaa

SEQ ID NO: 130 - DNA, 29 bp, synthetic construct
  source 1..29: mol_type = other DNA
caccgacaaggcagtttctttttctgtgt

SEQ ID NO: 131 - DNA, 29 bp, synthetic construct
  source 1..29: mol_type = other DNA
gaacacacagaaaaagaaactgccttgtc

SEQ ID NO: 132 - DNA, 24 bp, synthetic construct
  source 1..24: mol_type = other DNA
gacaagtgtgatcacttgggtggt

SEQ ID NO: 133 - DNA, 38 bp, synthetic construct
  source 1..38: mol_type = other DNA
ggtgacaagtgtgatcacttgggtggtggctgtgtttg

SEQ ID NO: 134 - DNA, 28 bp, synthetic construct
  source 1..28: mol_type = other DNA
caccgacaagtgtgatcacttgggtggt

SEQ ID NO: 135 - DNA, 28 bp, synthetic construct
  source 1..28: mol_type = other DNA
agacaccacccaagtgatcacacttgtc

SEQ ID NO: 136 - DNA, 24 bp, synthetic construct
  source 1..24: mol_type = other DNA
gcaagaggctcccgagcgagcaag

SEQ ID NO: 137 - DNA, 38 bp, synthetic construct
  source 1..38: mol_type = other DNA
ccagcaagaggctcccgagcgagcaagctcagtttaca

SEQ ID NO: 138 - DNA, 28 bp, synthetic construct
  source 1..28: mol_type = other DNA
caccgcaagaggctcccgagcgagcaag

SEQ ID NO: 139 - DNA, 28 bp, synthetic construct
  source 1..28: mol_type = other DNA
agaccttgctcgctcgggagcctcttgc

SEQ ID NO: 140 - DNA, 24 bp, synthetic construct
  source 1..24: mol_type = other DNA
gcacagggctgtgaggcttatctt

SEQ ID NO: 141 - DNA, 24 bp, synthetic construct
  source 1..24: mol_type = other DNA
cttcttactgtccccttctgggct

SEQ ID NO: 142 - DNA, 38 bp, synthetic construct
  source 1..38: mol_type = other DNA
ttccttcttactgtccccttctgggctcactatgctgc

SEQ ID NO: 143 - DNA, 28 bp, synthetic construct
  source 1..28: mol_type = other DNA
cacccttcttactgtccccttctgggct

SEQ ID NO: 144 - DNA, 28 bp, synthetic construct
  source 1..28: mol_type = other DNA
gaacagcccagaaggggacagtaagaag

SEQ ID NO: 145 - DNA, 24 bp, synthetic construct
  source 1..24: mol_type = other DNA
gcatgtcaatctcccagcgtcttt

SEQ ID NO: 146 - DNA, 38 bp, synthetic construct
  source 1..38: mol_type = other DNA
aatgcatgtcaatctcccagcgtctttatccgtgttcc

SEQ ID NO: 147 - DNA, 28 bp, synthetic construct
  source 1..28: mol_type = other DNA
caccgcatgtcaatctcccagcgtcttt

SEQ ID NO: 148 - DNA, 28 bp, synthetic construct
  source 1..28: mol_type = other DNA
agacaaagacgctgggagattgacatgc

SEQ ID NO: 149 - DNA, 24 bp, synthetic construct
  source 1..24: mol_type = other DNA
ggaaggccgaagcggagcgtcccg

SEQ ID NO: 150 - DNA, 38 bp, synthetic construct
  source 1..38: mol_type = other DNA
tggggaaggccgaagcggagcgtcccgccaggtttctc

SEQ ID NO: 151 - DNA, 28 bp, synthetic construct
  source 1..28: mol_type = other DNA
caccggaaggccgaagcggagcgtcccg

SEQ ID NO: 152 - DNA, 28 bp, synthetic construct
  source 1..28: mol_type = other DNA
agaccgggacgctccgcttcggccttcc

SEQ ID NO: 153 - DNA, 38 bp, synthetic construct; identical to SEQ ID NO: 150
  source 1..38: mol_type = other DNA

SEQ ID NO: 154 - DNA, 28 bp, synthetic construct
  source 1..28: mol_type = other DNA
caccggaaggccgaagcggagcgtcccg

SEQ ID NO: 155 - DNA, 28 bp, synthetic construct
  source 1..28: mol_type = other DNA
agaccgggacgctccgcttcggccttcc

SEQ ID NO: 156 - DNA, 24 bp, synthetic construct
  source 1..24: mol_type = other DNA
gcgcgctacctgcgccacatccat

SEQ ID NO: 157 - DNA, 38 bp, synthetic construct
  source 1..38: mol_type = other DNA
tgggcgcgctacctgcgccacatccatcggcgctttgg

SEQ ID NO: 158 - DNA, 28 bp, synthetic construct
  source 1..28: mol_type = other DNA
caccgcgcgctacctgcgccacatccat

SEQ ID NO: 159 - DNA, 28 bp, synthetic construct
  source 1..28: mol_type = other DNA
agacatggatgtggcgcaggtagcgcgc

SEQ ID NO: 160 - DNA, 24 bp, synthetic construct
  source 1..24: mol_type = other DNA
gagatatatcttagagggagggct

SEQ ID NO: 161 - DNA, 38 bp, synthetic construct
  source 1..38: mol_type = other DNA
caagagatatatcttagagggagggctgagggtttgaa

SEQ ID NO: 162 - DNA, 28 bp, synthetic construct
  source 1..28: mol_type = other DNA
caccgagatatatcttagagggagggct

SEQ ID NO: 163 - DNA, 28 bp, synthetic construct
  source 1..28: mol_type = other DNA
agacagccctccctctaagatatatctc

SEQ ID NO: 164 - DNA, 24 bp, synthetic construct
  source 1..24: mol_type = other DNA
tcctgggagtagattggccaaccc

SEQ ID NO: 165 - DNA, 38 bp, synthetic construct
  source 1..38: mol_type = other DNA
tgctcctgggagtagattggccaaccctagggtgtggc

SEQ ID NO: 166 - DNA, 29 bp, synthetic construct
  source 1..29: mol_type = other DNA
caccgtcctgggagtagattggccaaccc

SEQ ID NO: 167 - DNA, 29 bp, synthetic construct
  source 1..29: mol_type = other DNA
agacgggttggccaatctactcccaggac

SEQ ID NO: 168 - DNA, 24 bp, synthetic construct
  source 1..24: mol_type = other DNA
catgaggcatttgtagggcttctc

SEQ ID NO: 169 - DNA, 38 bp, synthetic construct
  source 1..38: mol_type = other DNA
gcacatgaggcatttgtagggcttctcgcccgtgtggg

SEQ ID NO: 170 - DNA, 29 bp, synthetic construct
  source 1..29: mol_type = other DNA
caccgcatgaggcatttgtagggcttctc

SEQ ID NO: 171 - DNA, 29 bp, synthetic construct
  source 1..29: mol_type = other DNA
agacgagaagccctacaaatgcctcatgc

SEQ ID NO: 172 - DNA, 24 bp, synthetic construct
  source 1..24: mol_type = other DNA
catttcccacactcgctgcatttg

SEQ ID NO: 173 - DNA, 38 bp, synthetic construct
  source 1..38: mol_type = other DNA
gaacatttcccacactcgctgcatttgtagggtttctc

SEQ ID NO: 174 - DNA, 29 bp, synthetic construct
  source 1..29: mol_type = other DNA
caccgcatttcccacactcgctgcatttg

SEQ ID NO: 175 - DNA, 29 bp, synthetic construct
  source 1..29: mol_type = other DNA
agaccaaatgcagcgagtgtgggaaatgc

SEQ ID NO: 176 - DNA, 24 bp, synthetic construct
  source 1..24: mol_type = other DNA
ttgattcttacaacaacatgagag

SEQ ID NO: 177 - DNA, 38 bp, synthetic construct
  source 1..38: mol_type = other DNA
actttgattcttacaacaacatgagagaggggtgttgt

SEQ ID NO: 178 - DNA, 29 bp, synthetic construct
  source 1..29: mol_type = other DNA
caccgttgattcttacaacaacatgagag

SEQ ID NO: 179 - DNA, 29 bp, synthetic construct
  source 1..29: mol_type = other DNA
agacctctcatgttgttgtaagaatcaac

SEQ ID NO: 180 - DNA, 24 bp, synthetic construct
  source 1..24: mol_type = other DNA
aaagaaatactaagacatgcagag

SEQ ID NO: 181 - DNA, 38 bp, synthetic construct
  source 1..38: mol_type = other DNA
tagaaagaaatactaagacatgcagagaggtgctttgc

SEQ ID NO: 182 - DNA, 29 bp, synthetic construct
  source 1..29: mol_type = other DNA
caccgaaagaaatactaagacatgcagag

SEQ ID NO: 183 - DNA, 29 bp, synthetic construct
  source 1..29: mol_type = other DNA
agacctctgcatgtcttagtatttctttc

SEQ ID NO: 184 - DNA, 24 bp, synthetic construct
  source 1..24: mol_type = other DNA
ggggcagagagagagagtgagcga

SEQ ID NO: 185 - DNA, 38 bp, synthetic construct
  source 1..38: mol_type = other DNA
tggggggcagagagagagagtgagcgagtgagtgtgtg

SEQ ID NO: 186 - DNA, 28 bp, synthetic construct
  source 1..28: mol_type = other DNA
caccggggcagagagagagagtgagcga

SEQ ID NO: 187 - DNA, 28 bp, synthetic construct
  source 1..28: mol_type = other DNA
agactcgctcactctctctctctgcccc

SEQ ID NO: 188 - DNA, 24 bp, synthetic construct
  source 1..24: mol_type = other DNA
gggccttgtcctgattggctgggc

SEQ ID NO: 189 - DNA, 38 bp, synthetic construct
  source 1..38: mol_type = other DNA
tgcgggccttgtcctgattggctgggcacgcgtttaat

SEQ ID NO: 190 - DNA, 28 bp, synthetic construct
  source 1..28: mol_type = other DNA
caccgggccttgtcctgattggctgggc

SEQ ID NO: 191 - DNA, 28 bp, synthetic construct
  source 1..28: mol_type = other DNA
agacgcccagccaatcaggacaaggccc

SEQ ID NO: 192 - DNA, 24 bp, synthetic construct
  source 1..24: mol_type = other DNA
gctgaggtttgtgaacgcgtggag

SEQ ID NO: 193 - DNA, 38 bp, synthetic construct
  source 1..38: mol_type = other DNA
ggcgctgaggtttgtgaacgcgtggaggggcgcttggg

SEQ ID NO: 194 - DNA, 28 bp, synthetic construct
  source 1..28: mol_type = other DNA
caccgctgaggtttgtgaacgcgtggag

SEQ ID NO: 195 - DNA, 28 bp, synthetic construct
  source 1..28: mol_type = other DNA
agacctccacgcgttcacaaacctcagc

SEQ ID NO: 196 - DNA, 24 bp, synthetic construct
  source 1..24: mol_type = other DNA
gttggctgaaaaggtggtctatgt

SEQ ID NO: 197 - DNA, 38 bp, synthetic construct
  source 1..38: mol_type = other DNA
gctgttggctgaaaaggtggtctatgttggcgtctgga

SEQ ID NO: 198 - DNA, 28 bp, synthetic construct
  source 1..28: mol_type = other DNA
caccgttggctgaaaaggtggtctatgt

SEQ ID NO: 199 - DNA, 28 bp, synthetic construct
  source 1..28: mol_type = other DNA
agacacatagaccaccttttcagccaac

SEQ ID NO: 200 - DNA, 24 bp, synthetic construct
  source 1..24: mol_type = other DNA
gtcatctacacagtcaacctctac

SEQ ID NO: 201 - DNA, 38 bp, synthetic construct
  source 1..38: mol_type = other DNA
catgtcatctacacagtcaacctctacagcagtgtcct

SEQ ID NO: 202 - DNA, 28 bp, synthetic construct
  source 1..28: mol_type = other DNA
caccgtcatctacacagtcaacctctac

SEQ ID NO: 203 - DNA, 28 bp, synthetic construct
  source 1..28: mol_type = other DNA
agacgtagaggttgactgtgtagatgac

SEQ ID NO: 204 - DNA, 24 bp, synthetic construct
  source 1..24: mol_type = other DNA
tcggcggtcaggtgtcccagagcc

SEQ ID NO: 205 - DNA, 38 bp, synthetic construct
  source 1..38: mol_type = other DNA
gggtcggcggtcaggtgtcccagagccaggggtctgga

SEQ ID NO: 206 - DNA, 29 bp, synthetic construct
  source 1..29: mol_type = other DNA
caccgtcggcggtcaggtgtcccagagcc

SEQ ID NO: 207 - DNA, 29 bp, synthetic construct
  source 1..29: mol_type = other DNA
agacggctctgggacacctgaccgccgac

SEQ ID NO: 208 - DNA, 25 bp, synthetic construct
  source 1..25: mol_type = other DNA
gttcttaggtaggtggggtcggcgg

SEQ ID NO: 209 - DNA, 39 bp, synthetic construct
  source 1..39: mol_type = other DNA
atggttcttaggtaggtggggtcggcggtcaggtgtccc

SEQ ID NO: 210 - DNA, 29 bp, synthetic construct
  source 1..29: mol_type = other DNA
caccgttcttaggtaggtggggtcggcgg

SEQ ID NO: 211 - DNA, 29 bp, synthetic construct
  source 1..29: mol_type = other DNA
agacccgccgaccccacctacctaagaac

SEQ ID NO: 212 - DNA, 24 bp, synthetic construct
  source 1..24: mol_type = other DNA
tcgcctgtcaagtggcgtgacacc

SEQ ID NO: 213 - DNA, 38 bp, synthetic construct
  source 1..38: mol_type = other DNA
tactcgcctgtcaagtggcgtgacaccgggcgtgttcc

SEQ ID NO: 214 - DNA, 29 bp, synthetic construct
  source 1..29: mol_type = other DNA
caccgtcgcctgtcaagtggcgtgacacc

SEQ ID NO: 215 - DNA, 29 bp, synthetic construct
  source 1..29: mol_type = other DNA
agacggtgtcacgccacttgacaggcgac

SEQ ID NO: 216 - DNA, 24 bp, synthetic construct
  source 1..24: mol_type = other DNA
gggaggtggcagggggaggaaagc

SEQ ID NO: 217 - DNA, 38 bp, synthetic construct
  source 1..38: mol_type = other DNA
gaagggaggtggcagggggaggaaagcagaggtttggg

SEQ ID NO: 218 - DNA, 28 bp, synthetic construct
  source 1..28: mol_type = other DNA
caccgggaggtggcagggggaggaaagc

SEQ ID NO: 219 - DNA, 28 bp, synthetic construct
  source 1..28: mol_type = other DNA
agacgctttcctccccctgccacctccc

SEQ ID NO: 220 - DNA, 24 bp, synthetic construct
  source 1..24: mol_type = other DNA
gataaggccgagaccaccaatcag

SEQ ID NO: 221 - DNA, 38 bp, synthetic construct
  source 1..38: mol_type = other DNA
atggataaggccgagaccaccaatcagaggagttttag

SEQ ID NO: 222 - DNA, 28 bp, synthetic construct
  source 1..28: mol_type = other DNA
caccgataaggccgagaccaccaatcag

SEQ ID NO: 223 - DNA, 28 bp, synthetic construct
  source 1..28: mol_type = other DNA
agacctgattggtggtctcggccttatc

SEQ ID NO: 224 - DNA, 24 bp, synthetic construct
  source 1..24: mol_type = other DNA
gcctcggcgctgacgatctgggtg

SEQ ID NO: 225 - DNA, 38 bp, synthetic construct
  source 1..38: mol_type = other DNA
caggcctcggcgctgacgatctgggtgacgggtttggc

SEQ ID NO: 226 - DNA, 28 bp, synthetic construct
  source 1..28: mol_type = other DNA
caccgcctcggcgctgacgatctgggtg

SEQ ID NO: 227 - DNA, 28 bp, synthetic construct
  source 1..28: mol_type = other DNA
agaccacccagatcgtcagcgccgaggc

SEQ ID NO: 228 - DNA, 24 bp, synthetic construct
  source 1..24: mol_type = other DNA
gtcagaggaagctggtctgggcct

SEQ ID NO: 229 - DNA, 38 bp, synthetic construct
  source 1..38: mol_type = other DNA
gctgtcagaggaagctggtctgggcctgggagtctgtg

SEQ ID NO: 230 - DNA, 28 bp, synthetic construct
  source 1..28: mol_type = other DNA
caccgtcagaggaagctggtctgggcct

SEQ ID NO: 231 - DNA, 28 bp, synthetic construct
  source 1..28: mol_type = other DNA
agacaggcccagaccagcttcctctgac

SEQ ID NO: 232 - DNA, 24 bp, synthetic construct
  source 1..24: mol_type = other DNA
gaggaggtggtagctggggctggg

SEQ ID NO: 233 - DNA, 38 bp, synthetic construct
  source 1..38: mol_type = other DNA
ggggaggaggtggtagctggggctgggggcggtgtctg

SEQ ID NO: 234 - DNA, 28 bp, synthetic construct
  source 1..28: mol_type = other DNA
caccgaggaggtggtagctggggctggg

SEQ ID NO: 235 - DNA, 28 bp, synthetic construct
  source 1..28: mol_type = other DNA
agaccccagccccagctaccacctcctc

SEQ ID NO: 236 - DNA, 24 bp, synthetic construct
  source 1..24: mol_type = other DNA
ggaggtggtagctggggctggggg

SEQ ID NO: 237 - DNA, 38 bp, synthetic construct
  source 1..38: mol_type = other DNA
ggaggaggtggtagctggggctgggggcggtgtctgtc

SEQ ID NO: 238 - DNA, 28 bp, synthetic construct
  source 1..28: mol_type = other DNA
caccggaggtggtagctggggctggggg

SEQ ID NO: 239 - DNA, 28 bp, synthetic construct
  source 1..28: mol_type = other DNA
agaccccccagccccagctaccacctcc

SEQ ID NO: 240 - DNA, 24 bp, synthetic construct
  source 1..24: mol_type = other DNA
gcccattccctctttagccagagc

SEQ ID NO: 241 - DNA, 38 bp, synthetic construct
  source 1..38: mol_type = other DNA
aaagcccattccctctttagccagagccggggtgtgca

SEQ ID NO: 242 - DNA, 28 bp, synthetic construct
  source 1..28: mol_type = other DNA
caccgcccattccctctttagccagagc

SEQ ID NO: 243 - DNA, 28 bp, synthetic construct
  source 1..28: mol_type = other DNA
agacgctctggctaaagagggaatgggc

SEQ ID NO: 244 - DNA, 24 bp, synthetic construct
  source 1..24: mol_type = other DNA
gagagaggctcccatcacggggga

SEQ ID NO: 245 - DNA, 38 bp, synthetic construct
  source 1..38: mol_type = other DNA
ctggagagaggctcccatcacgggggagggagtttgct

SEQ ID NO: 246 - DNA, 28 bp, synthetic construct
  source 1..28: mol_type = other DNA
caccgagagaggctcccatcacggggga

SEQ ID NO: 247 - DNA, 28 bp, synthetic construct
  source 1..28: mol_type = other DNA
agactcccccgtgatgggagcctctctc

SEQ ID NO: 248 - DNA, 24 bp, synthetic construct
  source 1..24: mol_type = other DNA
gcagcagaaatagactaattgcat

SEQ ID NO: 249 - DNA, 38 bp, synthetic construct
  source 1..38: mol_type = other DNA
cttgcagcagaaatagactaattgcatgggcgtttccc

SEQ ID NO: 250 - DNA, 28 bp, synthetic construct
  source 1..28: mol_type = other DNA
caccgcagcagaaatagactaattgcat

SEQ ID NO: 251 - DNA, 28 bp, synthetic construct
  source 1..28: mol_type = other DNA
agacatgcaattagtctatttctgctgc

SEQ ID NO: 252 - DNA, 24 bp, synthetic construct
  source 1..24: mol_type = other DNA
aagtcaccatcacaaggaaacgct

SEQ ID NO: 253 - DNA, 38 bp, synthetic construct
  source 1..38: mol_type = other DNA
tcaaagtcaccatcacaaggaaacgcttggtgtattga

SEQ ID NO: 254 - DNA, 29 bp, synthetic construct
  source 1..29: mol_type = other DNA
caccgaagtcaccatcacaaggaaacgct

SEQ ID NO: 255 - DNA, 29 bp, synthetic construct
  source 1..29: mol_type = other DNA
agacagcgtttccttgtgatggtgacttc

SEQ ID NO: 256 - DNA, 24 bp, synthetic construct
  source 1..24: mol_type = other DNA
ccaggtcagataaattttaggaag

SEQ ID NO: 257 - DNA, 38 bp, synthetic construct
  source 1..38: mol_type = other DNA
gtcccaggtcagataaattttaggaagtgctgttttcc

SEQ ID NO: 258 - DNA, 29 bp, synthetic construct
  source 1..29: mol_type = other DNA
caccgccaggtcagataaattttaggaag

SEQ ID NO: 259 - DNA, 29 bp, synthetic construct
  source 1..29: mol_type = other DNA
agaccttcctaaaatttatctgacctggc

SEQ ID NO: 260 - DNA, 24 bp, synthetic construct
  source 1..24: mol_type = other DNA
ggcagggcctgacagcggaaaggg

SEQ ID NO: 261 - DNA, 38 bp, synthetic construct
  source 1..38: mol_type = other DNA
gtgggcagggcctgacagcggaaagggtggagctttat

SEQ ID NO: 262 - DNA, 28 bp, synthetic construct
  source 1..28: mol_type = other DNA
caccggcagggcctgacagcggaaaggg

SEQ ID NO: 263 - DNA, 28 bp, synthetic construct
  source 1..28: mol_type = other DNA
agacccctttccgctgtcaggccctgcc

SEQ ID NO: 264 - DNA, 24 bp, synthetic construct
  source 1..24: mol_type = other DNA
gacccctaatatgaagggtagagt

SEQ ID NO: 265 - DNA, 38 bp, synthetic construct
  source 1..38: mol_type = other DNA
tttgacccctaatatgaagggtagagtgagtgtgtgtg

SEQ ID NO: 266 - DNA, 28 bp, synthetic construct
  source 1..28: mol_type = other DNA
caccgacccctaatatgaagggtagagt

SEQ ID NO: 267 - DNA, 28 bp, synthetic construct
  source 1..28: mol_type = other DNA
agacactctacccttcatattaggggtc

SEQ ID NO: 268 - DNA, 24 bp, synthetic construct
  source 1..24: mol_type = other DNA
gtggctgtgcttaggtagcgtggg

SEQ ID NO: 269 - DNA, 38 bp, synthetic construct
  source 1..38: mol_type = other DNA
cttgtggctgtgcttaggtagcgtgggatgtgtgtgtt

SEQ ID NO: 270 - DNA, 28 bp, synthetic construct
  source 1..28: mol_type = other DNA
caccgtggctgtgcttaggtagcgtggg

SEQ ID NO: 271 - DNA, 28 bp, synthetic construct
  source 1..28: mol_type = other DNA
agaccccacgctacctaagcacagccac

SEQ ID NO: 272 - DNA, 24 bp, synthetic construct
  source 1..24: mol_type = other DNA
agttcttgccgtgccccttcccca

SEQ ID NO: 273 - DNA, 38 bp, synthetic construct
  source 1..38: mol_type = other DNA
accagttcttgccgtgccccttccccagggtgtgtggt

SEQ ID NO: 274 - DNA, 29 bp, synthetic construct
  source 1..29: mol_type = other DNA
caccgagttcttgccgtgccccttcccca

SEQ ID NO: 275 - DNA, 29 bp, synthetic construct
  source 1..29: mol_type = other DNA
agactggggaaggggcacggcaagaactc

SEQ ID NO: 276 - DNA, 59 bp, synthetic construct
  source 1..59: mol_type = other DNA
tcgtcggcagcgtcagatgtgtataagagacagaaaacacaccgcatacgtacgattta

SEQ ID NO: 277 - DNA, 61 bp, synthetic construct
  source 1..61: mol_type = other DNA
tcgtcggcagcgtcagatgtgtataagagacagtttaatcacaccgcatacgtacgattta

SEQ ID NO: 278 - DNA, 59 bp, synthetic construct
  source 1..59: mol_type = other DNA
gtctcgtgggctcggagatgtgtataagagacagcgttctgatttaatctgtatcaggc

SEQ ID NO: 279 - DNA, 62 bp, synthetic construct
  source 1..62: mol_type = other DNA
tcgtcggcagcgtcagatgtgtataagagacagtttttggttacgcatctgtgcggtatttc

SEQ ID NO: 280 - DNA, 58 bp, synthetic construct
  source 1..58: mol_type = other DNA
tcgtcggcagcgtcagatgtgtataagagacagcccttacgcatctgtgcggtatttc

SEQ ID NO: 281 - DNA, 59 bp, synthetic construct
  source 1..59: mol_type = other DNA
tcgtcggcagcgtcagatgtgtataagagacagtatcgatttaaataggcctgactcac

SEQ ID NO: 282 - DNA, 62 bp, synthetic construct
  source 1..62: mol_type = other DNA
tcgtcggcagcgtcagatgtgtataagagacagatttcgatttaaataggcctgactcacta

SEQ ID NO: 283 - DNA, 55 bp, synthetic construct
  source 1..55: mol_type = other DNA
gtctcgtgggctcggagatgtgtataagagacagaatcttctctcatccgccaaa

SEQ ID NO: 284 - DNA, 31 bp, synthetic construct
  source 1..31: mol_type = other DNA
cggcattcctgctgaaccgctcttccgatct

SEQ ID NO: 285 - DNA, 30 bp, synthetic construct
  source 1..30: mol_type = other DNA
gatcggaagagcggttcagcaggaatgccg

SEQ ID NO: 286 - DNA, 55 bp, synthetic construct
  source 1..55: mol_type = other DNA
tcgtcggcagcgtcagatgtgtataagagacagctgctgaaccgctcttccgatc

SEQ ID NO: 287 - DNA, 60 bp, synthetic construct
  source 1..60: mol_type = other DNA
tcgtcggcagcgtcagatgtgtataagagacagtaagactgctgaaccgctcttccgatc

SEQ ID NO: 288 - DNA, 72 bp, synthetic construct
  source 1..72: mol_type = other DNA
tcgtcggcagcgtcagatgtgtataagagacaggctagacctaatgtgatctgctgaaccgctcttccgatc

SEQ ID NO: 289 - DNA, 63 bp, synthetic construct
  source 1..63: mol_type = other DNA
gtctcgtgggctcggagatgtgtataagagacagtctgcgttctgatttaatctgtatcaggc

SEQ ID NO: 290 - DNA, 21 bp, synthetic construct
  source 1..21: mol_type = other DNA
atttcggactaccctgaggag

SEQ ID NO: 291 - DNA, 22 bp, synthetic construct
  source 1..22: mol_type = other DNA
ggaatctaccaccccaggctct

SEQ ID NO: 292 - DNA, 18 bp, synthetic construct
  source 1..18: mol_type = other DNA
ttagaaagggcaggaggc

SEQ ID NO: 293 - DNA, 20 bp, synthetic construct
  source 1..20: mol_type = other DNA
cttgtccaggagttccgctc

SEQ ID NO: 294 - DNA, 20 bp, synthetic construct
  source 1..20: mol_type = other DNA
aattgaagcggaagtctggg

SEQ ID NO: 295 - DNA, 20 bp, synthetic construct
  source 1..20: mol_type = other DNA
aacacttctctcgctatgcc

SEQ ID NO: 296 - DNA, 24 bp, synthetic construct
  source 1..24: mol_type = other DNA
atgcacagggtggaacaagatgga

SEQ ID NO: 297 - DNA, 24 bp, synthetic construct
  source 1..24: mol_type = other DNA
ctaagccatgtgcacaactctgac

SEQ ID NO: 298 - DNA, 19 bp, synthetic construct
  source 1..19: mol_type = other DNA
ggcacatcttgggactcag

SEQ ID NO: 299 - DNA, 24 bp, synthetic construct
  source 1..24: mol_type = other DNA
agcatagcgcctggcattaatagg

SEQ ID NO: 300 - DNA, 21 bp, synthetic construct
  source 1..21: mol_type = other DNA
caaagaacctctgggtccaag

SEQ ID NO: 301 - DNA, 20 bp, synthetic construct
  source 1..20: mol_type = other DNA
gcatattctggagacgcagg

SEQ ID NO: 302 - DNA, 21 bp, synthetic construct
  source 1..21: mol_type = other DNA
gactgtgggcagaggttcagc

SEQ ID NO: 303 - DNA, 24 bp, synthetic construct
  source 1..24: mol_type = other DNA
tgtatacgggacttgactcagacc

SEQ ID NO: 304 - DNA, 19 bp, synthetic construct
  source 1..19: mol_type = other DNA
atgtcctcatgccggactg

SEQ ID NO: 305 - DNA, 24 bp, synthetic construct
  source 1..24: mol_type = other DNA
tccaagagcatacgcacacattcc

SEQ ID NO: 306 - DNA, 19 bp, synthetic construct
  source 1..19: mol_type = other DNA
taggactaggctcttggag

SEQ ID NO: 307 - DNA, 26 bp, synthetic construct
  source 1..26: mol_type = other DNA
catagagtacttagtatgagcgaggc

SEQ ID NO: 308 - DNA, 20 bp, synthetic construct
  source 1..20: mol_type = other DNA
ccagtctagtgcatgccttc

SEQ ID NO: 309 - DNA, 18 bp, synthetic construct
  source 1..18: mol_type = other DNA
gttcccatcacatgtcac

SEQ ID NO: 310 - DNA, 33 bp, synthetic construct
  source 1..33: mol_type = other DNA
ggacaggatgacaataccaggcaggataaggcc

SEQ ID NO: 311 - DNA, 32 bp, synthetic construct
  source 1..32: mol_type = other DNA
agaggagttagccaagatgtgactttgaaacc

SEQ ID NO: 312 - DNA, 24 bp, synthetic construct
  source 1..24: mol_type = other DNA
acgtcgtaaagccaaggttagtcc

SEQ ID NO: 313 - DNA, 21 bp, synthetic construct
  source 1..21: mol_type = other DNA
caccctcccttcaacctgacc

SEQ ID NO: 314 - DNA, 28 bp, synthetic construct
  source 1..28: mol_type = other DNA
gtcttaatttccactcatacagtggtag

SEQ ID NO: 315 - DNA, 22 bp, synthetic construct
  source 1..22: mol_type = other DNA
cgttttgggctctgggactcag

SEQ ID NO: 316 - DNA, 22 bp, synthetic construct
  source 1..22: mol_type = other DNA
tgtgtcgtccataaacgctgcc

SEQ ID NO: 317 - DNA, 27 bp, synthetic construct
  source 1..27: mol_type = other DNA
catcttccctgaaatttcttaagaggc

SEQ ID NO: 318 - DNA, 18 bp, synthetic construct
  source 1..18: mol_type = other DNA
ctgtccctgagtcccagt

SEQ ID NO: 319 - DNA, 20 bp, synthetic construct
  source 1..20: mol_type = other DNA
ggcctagaagagcagtaagg

SEQ ID NO: 320 - DNA, 19 bp, synthetic construct
  source 1..19: mol_type = other DNA
ctgaccacgtggagctgag

SEQ ID NO: 321 - DNA, 22 bp, synthetic construct
  source 1..22: mol_type = other DNA
cttacttacccgaggtaaagcc

SEQ ID NO: 322 - DNA, 20 bp, synthetic construct
  source 1..20: mol_type = other DNA
tgcgagcagcgaaagcgaca

SEQ ID NO: 323 - DNA, 20 bp, synthetic construct
  source 1..20: mol_type = other DNA
tccaatgcacccaagacagc

SEQ ID NO: 324 - DNA, 22 bp, synthetic construct
  source 1..22: mol_type = other DNA
gcatacgtgggctccaacaggt

SEQ ID NO: 325 - DNA, 22 bp, synthetic construct
  source 1..22: mol_type = other DNA
ccgcaatgaaggggaagctcga

SEQ ID NO: 326 - DNA, 22 bp, synthetic construct
  source 1..22: mol_type = other DNA
tacagcaggactgtgtggcacg

SEQ ID NO: 327 - DNA, 23 bp, synthetic construct
  source 1..23: mol_type = other DNA
cttccatcctccatcaggtcagg

SEQ ID NO: 328 - DNA, 18 bp, synthetic construct
  source 1..18: mol_type = other DNA
tagctacgcctgtgatgg

SEQ ID NO: 329 - DNA, 25 bp, synthetic construct
  source 1..25: mol_type = other DNA
ccagagaagttgctaggatgaaagg

SEQ ID NO: 330 - DNA, 18 bp, synthetic construct
  source 1..18: mol_type = other DNA
aacaatttcagatcgcgg

SEQ ID NO: 331 - DNA, 24 bp, synthetic construct
  source 1..24: mol_type = other DNA
gtcagacgtccaaaaccagactcc

SEQ ID NO: 332 - DNA, 24 bp, synthetic construct
  source 1..24: mol_type = other DNA
tcctgggtctgagtttctgagagg

SEQ ID NO: 333 - DNA, 24 bp, synthetic construct
  source 1..24: mol_type = other DNA
acaacacagatctgcagatctccg

SEQ ID NO: 334 - DNA, 20 bp, synthetic construct
  source 1..20: mol_type = other DNA
gtcagggcgctccttccttc

SEQ ID NO: 335 - DNA, 18 bp, synthetic construct
  source 1..18: mol_type = other DNA
gtgtacagggcacctgca

SEQ ID NO: 336 - DNA, 23 bp, synthetic construct
  source 1..23: mol_type = other DNA
ctgccatccccttctgtgaatgt

SEQ ID NO: 337 - DNA, 20 bp, synthetic construct
  source 1..20: mol_type = other DNA
gaagcgattatgatctctcc

SEQ ID NO: 338 - DNA, 21 bp, synthetic construct
  source 1..21: mol_type = other DNA
atttcggactaccctgaggag

SEQ ID NO: 339 - DNA, 23 bp, synthetic construct
  source 1..23: mol_type = other DNA
ctgccatccccttctgtgaatgt

SEQ ID NO: 340 - DNA, 20 bp, synthetic construct
  source 1..20: mol_type = other DNA
gaagcgattatgatctctcc

SEQ ID NO: 341 - DNA, 18 bp, synthetic construct
  source 1..18: mol_type = other DNA
ttagaaagggcaggaggc

SEQ ID NO: 342 - DNA, 20 bp, synthetic construct
  source 1..20: mol_type = other DNA
aattgaagcggaagtctggg

SEQ ID NO: 343 - DNA, 20 bp, synthetic construct
  source 1..20: mol_type = other DNA
aacacttctctcgctatgcc

SEQ ID NO: 344 - DNA, 20 bp, synthetic construct
  source 1..20: mol_type = other DNA
aattgaagcggaagtctggg

SEQ ID NO: 345 - DNA, 18 bp, synthetic construct
  source 1..18: mol_type = other DNA
acctgttagagctactgc

SEQ ID NO: 346 - DNA, 19 bp, synthetic construct
  source 1..19: mol_type = other DNA
agaagaagaggcacagggc

SEQ ID NO: 347 - DNA, 24 bp, synthetic construct
  source 1..24: mol_type = other DNA
ctaagccatgtgcacaactctgac

SEQ ID NO: 348 - DNA, 24 bp, synthetic construct
  source 1..24: mol_type = other DNA
atgcacagggtggaacaagatgga

SEQ ID NO: 349 - DNA, 24 bp, synthetic construct
  source 1..24: mol_type = other DNA
agcatagcgcctggcattaatagg

SEQ ID NO: 350 - DNA, 19 bp, synthetic construct
  source 1..19: mol_type = other DNA
ggcacatcttgggactcag

SEQ ID NO: 351 - DNA, 21 bp, synthetic construct
  source 1..21: mol_type = other DNA
gccaggctctcttggagtgtc

SEQ ID NO: 352 - DNA, 20 bp, synthetic construct
  source 1..20: mol_type = other DNA
gcatattctggagacgcagg

SEQ ID NO: 353 - DNA, 18 bp, synthetic construct
  source 1..18: mol_type = other DNA
ctccttaaacctgtcttg

SEQ ID NO: 354 - DNA, 21 bp, synthetic construct
  source 1..21: mol_type = other DNA
gactgtgggcagaggttcagc

SEQ ID NO: 355 - DNA, 21 bp, synthetic construct
  source 1..21: mol_type = other DNA
gactgtgggcagaggttcagc

SEQ ID NO: 356 - DNA, 19 bp, synthetic construct
  source 1..19: mol_type = other DNA
atgtcctcatgccggactg

SEQ ID NO: 357 - DNA, 19 bp, synthetic construct
  source 1..19: mol_type = other DNA
taggactaggctcttggag

SEQ ID NO: 358 - DNA, 24 bp, synthetic construct
  source 1..24: mol_type = other DNA
caaccccaccactgagttattagg

SEQ ID NO: 359 - DNA, 20 bp, synthetic construct
  source 1..20: mol_type = other DNA
ccagtctagtgcatgccttc

SEQ ID NO: 360 - DNA, 18 bp, synthetic construct
  source 1..18: mol_type = other DNA
gttcccatcacatgtcac

SEQ ID NO: 361 - DNA, 33 bp, synthetic construct; identical to SEQ ID NO: 310
  source 1..33: mol_type = other DNA

SEQ ID NO: 362 - DNA, 33 bp, synthetic construct; identical to SEQ ID NO: 310
  source 1..33: mol_type = other DNA

SEQ ID NO: 363 - DNA, 21 bp, synthetic construct
  source 1..21: mol_type = other DNA
caccctcccttcaacctgacc

SEQ ID NO: 364 - DNA, 21 bp, synthetic construct
  source 1..21: mol_type = other DNA
caccctcccttcaacctgacc

SEQ ID NO: 365 - DNA, 22 bp, synthetic construct
  source 1..22: mol_type = other DNA
cgttttgggctctgggactcag

SEQ ID NO: 366 - DNA, 22 bp, synthetic construct
  source 1..22: mol_type = other DNA
tgtgtcgtccataaacgctgcc

SEQ ID NO: 367 - DNA, 20 bp, synthetic construct
  source 1..20: mol_type = other DNA
ggcctagaagagcagtaagg

SEQ ID NO: 368 - DNA, 19 bp, synthetic construct
  source 1..19: mol_type = other DNA
ctgaccacgtggagctgag

SEQ ID NO: 369 - DNA, 22 bp, synthetic construct
  source 1..22: mol_type = other DNA
cttacttacccgaggtaaagcc

SEQ ID NO: 370 - DNA, 20 bp, synthetic construct
  source 1..20: mol_type = other DNA
tgcgagcagcgaaagcgaca

SEQ ID NO: 371 - DNA, 20 bp, synthetic construct
  source 1..20: mol_type = other DNA
tgcgagcagcgaaagcgaca

SEQ ID NO: 372 - DNA, 22 bp, synthetic construct
  source 1..22: mol_type = other DNA
gcatacgtgggctccaacaggt

SEQ ID NO: 373 - DNA, 22 bp, synthetic construct
  source 1..22: mol_type = other DNA
tacagcaggactgtgtggcacg

SEQ ID NO: 374 - DNA, 18 bp, synthetic construct
  source 1..18: mol_type = other DNA
tagctacgcctgtgatgg

SEQ ID NO: 375 - DNA, 18 bp, synthetic construct
  source 1..18: mol_type = other DNA
aacaatttcagatcgcgg

SEQ ID NO: 376 - DNA, 24 bp, synthetic construct
  source 1..24: mol_type = other DNA
agagaagttggagtgaaggcagag

SEQ ID NO: 377 - DNA, 24 bp, synthetic construct
  source 1..24: mol_type = other DNA
acaacacagatctgcagatctccg

SEQ ID NO: 378 - DNA, 24 bp, synthetic construct
  source 1..24: mol_type = other DNA
tcctgggtctgagtttctgagagg

SEQ ID NO: 379 - DNA, 20 bp, synthetic construct
  source 1..20: mol_type = other DNA
gtcagggcgctccttccttc

SEQ ID NO: 380 - DNA, 18 bp, synthetic construct
  source 1..18: mol_type = other DNA
gtgtacagggcacctgca

SEQ ID NO: 381 - RNA, 56 nt, synthetic construct
  source 1..56: mol_type = other RNA
  misc_difference 1: note = a, c, u, g, unknown, or other
  misc_difference 2: note = a, c, u, g, unknown, or other
  misc_difference 3: note = a, c, u, g, unknown, or other
  misc_difference 4: note = a, c, u, g, unknown, or other
  misc_difference 5: note = a, c, u, g, unknown, or other
  misc_difference 6: note = a, c, u, g, unknown, or other
  misc_difference 7: note = a, c, u, g, unknown, or other
  misc_difference 8: note = a, c, u, g, unknown, or other
  misc_difference 9: note = a, c, u, g, unknown, or other
  misc_difference 10: note = a, c, u, g, unknown, or other
  misc_difference 11: note = a, c, u, g, unknown, or other
  misc_difference 12: note = a, c, u, g, unknown, or other
  misc_difference 13: note = a, c, u, g, unknown, or other
  misc_difference 14: note = a, c, u, g, unknown, or other
  misc_difference 15: note = a, c, u, g, unknown, or other
  misc_difference 16: note = a, c, u, g, unknown, or other
  misc_difference 17: note = a, c, u, g, unknown, or other
  misc_difference 18: note = a, c, u, g, unknown, or other
  misc_difference 19: note = a, c, u, g, unknown, or other
  misc_difference 20: note = a, c, u, g, unknown, or other
nnnnnnnnnnnnnnnnnnnngtcttgagcacgcgcccttccccaaggtgatacgct

SEQ ID NO: 382 - RNA, 56 nt, synthetic construct
  source 1..56: mol_type = other RNA
  misc_difference 1: note = a, c, u, g, unknown, or other
  misc_difference 2: note = a, c, u, g, unknown, or other
  misc_difference 3: note = a, c, u, g, unknown, or other
  misc_difference 4: note = a, c, u, g, unknown, or other
  misc_difference 5: note = a, c, u, g, unknown, or other
  misc_difference 6: note = a, c, u, g, unknown, or other
  misc_difference 7: note = a, c, u, g, unknown, or other
  misc_difference 8: note = a, c, u, g, unknown, or other
  misc_difference 9: note = a, c, u, g, unknown, or other
  misc_difference 10: note = a, c, u, g, unknown, or other
  misc_difference 11: note = a, c, u, g, unknown, or other
  misc_difference 12: note = a, c, u, g, unknown, or other
  misc_difference 13: note = a, c, u, g, unknown, or other
  misc_difference 14: note = a, c, u, g, unknown, or other
  misc_difference 15: note = a, c, u, g, unknown, or other
  misc_difference 16: note = a, c, u, g, unknown, or other
  misc_difference 17: note = a, c, u, g, unknown, or other
  misc_difference 18: note = a, c, u, g, unknown, or other
  misc_difference 19: note = a, c, u, g, unknown, or other
  misc_difference 20: note = a, c, u, g, unknown, or other
nnnnnnnnnnnnnnnnnnnngttctggtctaagttcatttcctaactgataaaatc

SEQ ID NO: 383 - RNA, 105 nt, synthetic construct; identical to SEQ ID NO: 21
  source 1..105: mol_type = other RNA

SEQ ID NO: 384 - RNA, 88 nt, synthetic construct; identical to SEQ ID NO: 14
  source 1..88: mol_type = other RNA

SEQ ID NO: 385 - RNA, 159 nt, synthetic construct
  source 1..159: mol_type = other RNA
  misc_difference 1: note = a, c, u, g, unknown, or other
  misc_difference 2: note = a, c, u, g, unknown, or other
  misc_difference 3: note = a, c, u, g, unknown, or other
  misc_difference 4: note = a, c, u, g, unknown, or other
  misc_difference 5: note = a, c, u, g, unknown, or other
  misc_difference 6: note = a, c, u, g, unknown, or other
  misc_difference 7: note = a, c, u, g, unknown, or other
  misc_difference 8: note = a, c, u, g, unknown, or other
  misc_difference 9: note = a, c, u, g, unknown, or other
  misc_difference 10: note = a, c, u, g, unknown, or other
  misc_difference 11: note = a, c, u, g, unknown, or other
  misc_difference 12: note = a, c, u, g, unknown, or other
  misc_difference 13: note = a, c, u, g, unknown, or other
  misc_difference 14: note = a, c, u, g, unknown, or other
  misc_difference 15: note = a, c, u, g, unknown, or other
  misc_difference 16: note = a, c, u, g, unknown, or other
  misc_difference 17: note = a, c, u, g, unknown, or other
  misc_difference 18: note = a, c, u, g, unknown, or other
  misc_difference 19: note = a, c, u, g, unknown, or other
  misc_difference 20: note = a, c, u, g, unknown, or other
nnnnnnnnnnnnnnnnnnnngtcttgagcacgcgcccttccccaaggtgagaaatcaccttggggaagggcgcggctccagacaaggggagccacttaagtggcttacccgtaaagtaacccccgttcaatcttcggattgggcggggcgaactttttt

SEQ ID NO: 386 - RNA, 159 nt, synthetic construct
  source 1..159: mol_type = other RNA
  misc_difference 1: note = a, c, u, g, unknown, or other
  misc_difference 2: note = a, c, u, g, unknown, or other
  misc_difference 3: note = a, c, u, g, unknown, or other
  misc_difference 4: note = a, c, u, g, unknown, or other
  misc_difference 5: note = a, c, u, g, unknown, or other
  misc_difference 6: note = a, c, u, g, unknown, or other
  misc_difference 7: note = a, c, u, g, unknown, or other
  misc_difference 8: note = a, c, u, g, unknown, or other
  misc_difference 9: note = a, c, u, g, unknown, or other
  misc_difference 10: note = a, c, u, g, unknown, or other
  misc_difference 11: note = a, c, u, g, unknown, or other
  misc_difference 12: note = a, c, u, g, unknown, or other
  misc_difference 13: note = a, c, u, g, unknown, or other
  misc_difference 14: note = a, c, u, g, unknown, or other
  misc_difference 15: note = a, c, u, g, unknown, or other
  misc_difference 16: note = a, c, u, g, unknown, or other
  misc_difference 17: note = a, c, u, g, unknown, or other
  misc_difference 18: note = a, c, u, g, unknown, or other
  misc_difference 19: note = a, c, u, g, unknown, or other
  misc_difference 20: note = a, c, u, g, unknown, or other
nnnnnnnnnnnnnnnnnnnngtcttgagcacgcgcccttccgcaaggtgagaaatcaccttgcggaagggcgcggctccagacaagcggagccacttaagtggcttacgcgtaaagtaaccgccgttcaatcttcggattgggcggcgcgaactttttt

SEQ ID NO: 387 - RNA, 125 nt, synthetic construct
  source 1..125: mol_type = other RNA
  misc_difference 1: note = a, c, u, g, unknown, or other
  misc_difference 2: note = a, c, u, g, unknown, or other
  misc_difference 3: note = a, c, u, g, unknown, or other
  misc_difference 4: note = a, c, u, g, unknown, or other
  misc_difference 5: note = a, c, u, g, unknown, or other
  misc_difference 6: note = a, c, u, g, unknown, or other
  misc_difference 7: note = a, c, u, g, unknown, or other
  misc_difference 8: note = a, c, u, g, unknown, or other
  misc_difference 9: note = a, c, u, g, unknown, or other
  misc_difference 10: note = a, c, u, g, unknown, or other
  misc_difference 11: note = a, c, u, g, unknown, or other
  misc_difference 12: note = a, c, u, g, unknown, or other
  misc_difference 13: note = a, c, u, g, unknown, or other
  misc_difference 14: note = a, c, u, g, unknown, or other
  misc_difference 15: note = a, c, u, g, unknown, or other
  misc_difference 16: note = a, c, u, g, unknown, or other
  misc_difference 17: note = a, c, u, g, unknown, or other
  misc_difference 18: note = a, c, u, g, unknown, or other
  misc_difference 19: note = a, c, u, g, unknown, or other
  misc_difference 20: note = a, c, u, g, unknown, or other
nnnnnnnnnnnnnnnnnnnngtcttgagcacgcgaaagcggctccagacaaggggagccacttaagtggcttacccgtaaagtaacccccgttcaatcttcggattgggcggggcgaactttttt

SEQ ID NO: 388 - RNA, 125 nt, synthetic construct
  source 1..125: mol_type = other RNA
  misc_difference 1: note = a, c, u, g, unknown, or other
  misc_difference 2: note = a, c, u, g, unknown, or other
  misc_difference 3: note = a, c, u, g, unknown, or other
  misc_difference 4: note = a, c, u, g, unknown, or other
  misc_difference 5: note = a, c, u, g, unknown, or other
  misc_difference 6: note = a, c, u, g, unknown, or other
  misc_difference 7: note = a, c, u, g, unknown, or other
  misc_difference 8: note = a, c, u, g, unknown, or other
  misc_difference 9: note = a, c, u, g, unknown, or other
  misc_difference 10: note = a, c, u, g, unknown, or other
  misc_difference 11: note = a, c, u, g, unknown, or other
  misc_difference 12: note = a, c, u, g, unknown, or other
  misc_difference 13: note = a, c, u, g, unknown, or other
  misc_difference 14: note = a, c, u, g, unknown, or other
  misc_difference 15: note = a, c, u, g, unknown, or other
  misc_difference 16: note = a, c, u, g, unknown, or other
  misc_difference 17: note = a, c, u, g, unknown, or other
  misc_difference 18: note = a, c, u, g, unknown, or other
  misc_difference 19: note = a, c, u, g, unknown, or other
  misc_difference 20: note = a, c, u, g, unknown, or other
nnnnnnnnnnnnnnnnnnnngtcttgagcacgcgaaagcggctccagacaagcggagccacttaagtggcttacgcgtaaagtaaccgccgttcaatcttcggattgggcggcgcgaactttttt

SEQ ID NO: 389 - RNA, 141 nt, synthetic construct
  source 1..141: mol_type = other RNA
  misc_difference 1: note = a, c, u, g, unknown, or other
  misc_difference 2: note = a, c, u, g, unknown, or other
  misc_difference 3: note = a, c, u, g, unknown, or other
  misc_difference 4: note = a, c, u, g, unknown, or other
  misc_difference 5: note = a, c, u, g, unknown, or other
  misc_difference 6: note = a, c, u, g, unknown, or other
  misc_difference 7: note = a, c, u, g, unknown, or other
  misc_difference 8: note = a, c, u, g, unknown, or other
  misc_difference 9: note = a, c, u, g, unknown, or other
  misc_difference 10: note = a, c, u, g, unknown, or other
  misc_difference 11: note = a, c, u, g, unknown, or other
  misc_difference 12: note = a, c, u, g, unknown, or other
  misc_difference 13: note = a, c, u, g, unknown, or other
  misc_difference 14: note = a, c, u, g, unknown, or other
  misc_difference 15: note = a, c, u, g, unknown, or other
  misc_difference 16: note = a, c, u, g, unknown, or other
  misc_difference 17: note = a, c, u, g, unknown, or other
  misc_difference 18: note = a, c, u, g, unknown, or other
  misc_difference 19: note = a, c, u, g, unknown, or other
  misc_difference 20: note = a, c, u, g, unknown, or other
nnnnnnnnnnnnnnnnnnnngttctggtctaagttcatttcctaactgagaaatcagttaggaaatgggctttctccactaacaagctgagagatgcacaagatgcggggtcgctatatgcgaccatttttcgtatccaaa

SEQ ID NO: 390 - RNA, 141 nt, synthetic construct
  source 1..141: mol_type = other RNA
  misc_difference 1: note = a, c, u, g, unknown, or other
  misc_difference 2: note = a, c, u, g, unknown, or other
  misc_difference 3: note = a, c, u, g, unknown, or other
  misc_difference 4: note = a, c, u, g, unknown, or other
  misc_difference 5: note = a, c, u, g, unknown, or other
  misc_difference 6: note = a, c, u, g, unknown, or other
  misc_difference 7: note = a, c, u, g, unknown, or other
  misc_difference 8: note = a, c, u, g, unknown, or other
  misc_difference 9: note = a, c, u, g, unknown, or other
  misc_difference 10: note = a, c, u, g, unknown, or other
  misc_difference 11: note = a, c, u, g, unknown, or other
  misc_difference 12: note = a, c, u, g, unknown, or other
  misc_difference 13: note = a, c, u, g, unknown, or other
  misc_difference 14: note = a, c, u, g, unknown, or other
  misc_difference 15: note = a, c, u, g, unknown, or other
  misc_difference 16: note = a, c, u, g, unknown, or other
  misc_difference 17: note = a, c, u, g, unknown, or other
  misc_difference 18: note = a, c, u, g, unknown, or other
  misc_difference 19: note = a, c, u, g, unknown, or other
  misc_difference 20: note = a, c, u, g, unknown, or other
nnnnnnnnnnnnnnnnnnnngttctggtctaagttcatttcctaactgagaaatcagttaggaaatgggctttctccactaacaagctgagagatgcacaagatgcggggtcgctatatgcgaccattattcgtatccaaa

SEQ ID NO: 391 - RNA, 109 nt, synthetic construct
  source 1..109: mol_type = other RNA
  misc_difference 1: note = a, c, u, g, unknown, or other
  misc_difference 2: note = a, c, u, g, unknown, or other
  misc_difference 3: note = a, c, u, g, unknown, or other
  misc_difference 4: note = a, c, u, g, unknown, or other
  misc_difference 5: note = a, c, u, g, unknown, or other
  misc_difference 6: note = a, c, u, g, unknown, or other
  misc_difference 7: note = a, c, u, g, unknown, or other
  misc_difference 8: note = a, c, u, g, unknown, or other
  misc_difference 9: note = a, c, u, g, unknown, or other
  misc_difference 10: note = a, c, u, g, unknown, or other
  misc_difference 11: note = a, c, u, g, unknown, or other
  misc_difference 12: note = a, c, u, g, unknown, or other
  misc_difference 13: note = a, c, u, g, unknown, or other
  misc_difference 14: note = a, c, u, g, unknown, or other
  misc_difference 15: note = a, c, u, g, unknown, or other
  misc_difference 16: note = a, c, u, g, unknown, or other
  misc_difference 17: note = a, c, u, g, unknown, or other
  misc_difference 18: note = a, c, u, g, unknown, or other
  misc_difference 19: note = a, c, u, g, unknown, or other
  misc_difference 20: note = a, c, u, g, unknown, or other
nnnnnnnnnnnnnnnnnnnngttctggtctaaggaaactttctccactaacaagctgagagatgcacaagatgcggggtcgctatatgcgaccattattcgtatccaaa

SEQ ID NO: 392 - RNA, 137 nt, synthetic construct
  source 1..137: mol_type = other RNA
  misc_difference 1: note = a, c, u, g, unknown, or other
  misc_difference 2: note = a, c, u, g, unknown, or other
  misc_difference 3: note = a, c, u, g, unknown, or other
  misc_difference 4: note = a, c, u, g, unknown, or other
  misc_difference 5: note = a, c, u, g, unknown, or other
  misc_difference 6: note = a, c, u, g, unknown, or other
  misc_difference 7: note = a, c, u, g, unknown, or other
  misc_difference 8: note = a, c, u, g, unknown, or other
  misc_difference 9: note = a, c, u, g, unknown, or other
  misc_difference 10: note = a, c, u, g, unknown, or other
  misc_difference 11: note = a, c, u, g, unknown, or other
  misc_difference 12: note = a, c, u, g, unknown, or other
  misc_difference 13: note = a, c, u, g, unknown, or other
  misc_difference 14: note = a, c, u, g, unknown, or other
  misc_difference 15: note = a, c, u, g, unknown, or other
  misc_difference 16: note = a, c, u, g, unknown, or other
  misc_difference 17: note = a, c, u, g, unknown, or other
  misc_difference 18: note = a, c, u, g, unknown, or other
  misc_difference 19: note = a, c, u, g, unknown, or other
  misc_difference 20: note = a, c, u, g, unknown, or other
nnnnnnnnnnnnnnnnnnnngttctggtctaagttcatttcctaactgagaaatcagttaggaaatgggctttctccactaacaagcgaaagcacaagatgcgggctcgctatatgcgagcattattcgtatccaaa

SEQ ID NO: 393 - RNA, 105 nt, synthetic construct
  source 1..105: mol_type = other RNA
  misc_difference 1: note = a, c, u, g, unknown, or other
  misc_difference 2: note = a, c, u, g, unknown, or other
  misc_difference 3: note = a, c, u, g, unknown, or other
  misc_difference 4: note = a, c, u, g, unknown, or other
  misc_difference 5: note = a, c, u, g, unknown, or other
  misc_difference 6: note = a, c, u, g, unknown, or other
  misc_difference 7: note = a, c, u, g, unknown, or other
  misc_difference 8: note = a, c, u, g, unknown, or other
  misc_difference 9: note = a, c, u, g, unknown, or other
  misc_difference 10: note = a, c, u, g, unknown, or other
  misc_difference 11: note = a, c, u, g, unknown, or other
  misc_difference 12: note = a, c, u, g, unknown, or other
  misc_difference 13: note = a, c, u, g, unknown, or other
  misc_difference 14: note = a, c, u, g, unknown, or other
  misc_difference 15: note = a, c, u, g, unknown, or other
  misc_difference 16: note = a, c, u, g, unknown, or other
  misc_difference 17: note = a, c, u, g, unknown, or other
  misc_difference 18: note = a, c, u, g, unknown, or other
  misc_difference 19: note = a, c, u, g, unknown, or other
  misc_difference 20: note = a, c, u, g, unknown, or other
nnnnnnnnnnnnnnnnnnnngttctggtctaaggaaactttctccactaacaagcgaaagcacaagatgcgggctcgctatatgcgagcattattcgtatccaaa

SEQ ID NO: 394 - DNA, 24 bp, synthetic construct
  source 1..24: mol_type = other DNA
cctgggcagccagggctggcctca

SEQ ID NO: 395 - DNA, 38 bp, synthetic construct
  source 1..38: mol_type = other DNA
ccgcctgggcagccagggctggcctcagggtgtgttcc

SEQ ID NO: 396 - DNA, 29 bp, synthetic construct
  source 1..29: mol_type = other DNA
caccgcctgggcagccagggctggcctca

SEQ ID NO: 397 - DNA, 29 bp, synthetic construct
  source 1..29: mol_type = other DNA
agactgaggccagccctggctgcccaggc

SEQ ID NO: 398 - DNA, 23 bp, synthetic construct
  source 1..23: mol_type = other DNA
ctgggcagccagggctggcctca

SEQ ID NO: 399 - DNA, 37 bp, synthetic construct
  source 1..37: mol_type = other DNA
cgcctgggcagccagggctggcctcagggtgtgttcc

SEQ ID NO: 400 - DNA, 28 bp, synthetic construct
  source 1..28: mol_type = other DNA
caccgctgggcagccagggctggcctca

SEQ ID NO: 401 - DNA, 28 bp, synthetic construct
  source 1..28: mol_type = other DNA
agactgaggccagccctggctgcccagc

SEQ ID NO: 402 - DNA, 22 bp, synthetic construct
  source 1..22: mol_type = other DNA
tgggcagccagggctggcctca

SEQ ID NO: 403 - DNA, 36 bp, synthetic construct
  source 1..36: mol_type = other DNA
gcctgggcagccagggctggcctcagggtgtgttcc

SEQ ID NO: 404 - DNA, 27 bp, synthetic construct
  source 1..27: mol_type = other DNA
caccgtgggcagccagggctggcctca

SEQ ID NO: 405 - DNA, 27 bp, synthetic construct
  source 1..27: mol_type = other DNA
agactgaggccagccctggctgcccac

SEQ ID NO: 406 - DNA, 24 bp, synthetic construct
  source 1..24: mol_type = other DNA
tctcctcaggagtcaggtgcacca

SEQ ID NO: 407 - DNA, 38 bp, synthetic construct
  source 1..38: mol_type = other DNA
acttctcctcaggagtcaggtgcaccatggtgtctgtt

SEQ ID NO: 408 - DNA, 29 bp, synthetic construct
  source 1..29: mol_type = other DNA
caccgtctcctcaggagtcaggtgcacca

SEQ ID NO: 409 - DNA, 29 bp, synthetic construct
  source 1..29: mol_type = other DNA
agactggtgcacctgactcctgaggagac

SEQ ID NO: 410 - DNA, 23 bp, synthetic construct
  source 1..23: mol_type = other DNA
ctcctcaggagtcaggtgcacca

SEQ ID NO: 411 - DNA, 37 bp, synthetic construct
  source 1..37: mol_type = other DNA
cttctcctcaggagtcaggtgcaccatggtgtctgtt

SEQ ID NO: 412 - DNA, 28 bp, synthetic construct
  source 1..28: mol_type = other DNA
caccgctcctcaggagtcaggtgcacca

SEQ ID NO: 413 - DNA, 28 bp, synthetic construct
  source 1..28: mol_type = other DNA
agactggtgcacctgactcctgaggagc

SEQ ID NO: 414 - DNA, 22 bp, synthetic construct
  source 1..22: mol_type = other DNA
tcctcaggagtcaggtgcacca

SEQ ID NO: 415 - DNA, 36 bp, synthetic construct
  source 1..36: mol_type = other DNA
ttctcctcaggagtcaggtgcaccatggtgtctgtt

SEQ ID NO: 416 - DNA, 27 bp, synthetic construct
  source 1..27: mol_type = other DNA
caccgtcctcaggagtcaggtgcacca

SEQ ID NO: 417 - DNA, 27 bp, synthetic construct
  source 1..27: mol_type = other DNA
agactggtgcacctgactcctgaggac

SEQ ID NO: 418 - DNA, 24 bp, synthetic construct
  source 1..24: mol_type = other DNA
agagctcttggtacctgaagttat

SEQ ID NO: 419 - DNA, 38 bp, synthetic construct
  source 1..38: mol_type = other DNA
gatagagctcttggtacctgaagttataggggtttagt

SEQ ID NO: 420 - DNA, 29 bp, synthetic construct
  source 1..29: mol_type = other DNA
caccgagagctcttggtacctgaagttat

SEQ ID NO: 421 - DNA, 29 bp, synthetic construct
  source 1..29: mol_type = other DNA
agacataacttcaggtaccaagagctctc

SEQ ID NO: 422 - DNA, 24 bp, synthetic construct
  source 1..24: mol_type = other DNA
gaggataccaggacttcttttgtc

SEQ ID NO: 423 - DNA, 38 bp, synthetic construct
  source 1..38: mol_type = other DNA
atagaggataccaggacttcttttgtcagccgtttttt

SEQ ID NO: 424 - DNA, 28 bp, synthetic construct
  source 1..28: mol_type = other DNA
caccgaggataccaggacttcttttgtc

SEQ ID NO: 425 - DNA, 28 bp, synthetic construct
  source 1..28: mol_type = other DNA
agacgacaaaagaagtcctggtatcctc

SEQ ID NO: 426 - DNA, 24 bp, synthetic construct
  source 1..24: mol_type = other DNA
gaaatgacccatggcgtctggact

SEQ ID NO: 427 - DNA, 38 bp, synthetic construct
  source 1..38: mol_type = other DNA
tgtgaaatgacccatggcgtctggactaggagcttatt

SEQ ID NO: 428 - DNA, 28 bp, synthetic construct
  source 1..28: mol_type = other DNA
caccgaaatgacccatggcgtctggact

SEQ ID NO: 429 - DNA, 28 bp, synthetic construct
  source 1..28: mol_type = other DNA
agacagtccagacgccatgggtcatttc

SEQ ID NO: 430 - DNA, 24 bp, synthetic construct
  source 1..24: mol_type = other DNA
gcttgcattgtatgtctggctatt

SEQ ID NO: 431 - DNA, 38 bp, synthetic construct
  source 1..38: mol_type = other DNA
aatgcttgcattgtatgtctggctattctgtgttttta

SEQ ID NO: 432 - DNA, 28 bp, synthetic construct
  source 1..28: mol_type = other DNA
caccgcttgcattgtatgtctggctatt

SEQ ID NO: 433 - DNA, 28 bp, synthetic construct
  source 1..28: mol_type = other DNA
agacaatagccagacatacaatgcaagc

SEQ ID NO: 434 - DNA, 24 bp, synthetic construct
  source 1..24: mol_type = other DNA
atcattatgctgaggatttggaaa

SEQ ID NO: 435 - DNA, 38 bp, synthetic construct
  source 1..38: mol_type = other DNA
ctaatcattatgctgaggatttggaaagggtgtttatt

SEQ ID NO: 436 - DNA, 29 bp, synthetic construct
  source 1..29: mol_type = other DNA
caccgatcattatgctgaggatttggaaa

SEQ ID NO: 437 - DNA, 29 bp, synthetic construct
  source 1..29: mol_type = other DNA
agactttccaaatcctcagcataatgatc

SEQ ID NO: 438 - DNA, 24 bp, synthetic construct
  source 1..24: mol_type = other DNA
gcacctaatctcctagaggactta

SEQ ID NO: 439 - DNA, 38 bp, synthetic construct
  source 1..38: mol_type = other DNA
gcagcacctaatctcctagaggacttagcccgtgtcac

SEQ ID NO: 440 - DNA, 28 bp, synthetic construct
  source 1..28: mol_type = other DNA
caccgcacctaatctcctagaggactta

SEQ ID NO: 441 - DNA, 28 bp, synthetic construct
  source 1..28: mol_type = other DNA
agactaagtcctctaggagattaggtgc

SEQ ID NO: 442 - DNA, 24 bp, synthetic construct
  source 1..24: mol_type = other DNA
gaggcaggaggatcactagaggcc

SEQ ID NO: 443 - DNA, 38 bp, synthetic construct
  source 1..38: mol_type = other DNA
gctgaggcaggaggatcactagaggccaggagtttgag

SEQ ID NO: 444 - DNA, 28 bp, synthetic construct
  source 1..28: mol_type = other DNA
caccgaggcaggaggatcactagaggcc

SEQ ID NO: 445 - DNA, 28 bp, synthetic construct
  source 1..28: mol_type = other DNA
agacggcctctagtgatcctcctgcctc

SEQ ID NO: 446 - DNA, 24 bp, synthetic construct
  source 1..24: mol_type = other DNA
gttaggaactttattggctggaac

SEQ ID NO: 447 - DNA, 38 bp, synthetic construct
  source 1..38: mol_type = other DNA
gtagttaggaactttattggctggaactggagtttcct

SEQ ID NO: 448 - DNA, 28 bp, synthetic construct
  source 1..28: mol_type = other DNA
caccgttaggaactttattggctggaac

SEQ ID NO: 449 - DNA, 28 bp, synthetic construct
  source 1..28: mol_type = other DNA
agacgttccagccaataaagttcctaac

SEQ ID NO: 450 - DNA, 23 bp, synthetic construct
  source 1..23: mol_type = other DNA
gttgaggggtctccttgtggtgg

SEQ ID NO: 451 - DNA, 37 bp, synthetic construct
  source 1..37: mol_type = other DNA
gtggttgaggggtctccttgtggtggaggagtctcct

SEQ ID NO: 452 - DNA, 27 bp, synthetic construct
  source 1..27: mol_type = other DNA
caccgttgaggggtctccttgtggtgg

SEQ ID NO: 453 - DNA, 27 bp, synthetic construct
  source 1..27: mol_type = other DNA
agacccaccacaaggagacccctcaac

SEQ ID NO: 454 - DNA, 24 bp, synthetic construct
  source 1..24: mol_type = other DNA
gaaaggttttactattcaacaatt

SEQ ID NO: 455 - DNA, 38 bp, synthetic construct
  source 1..38: mol_type = other DNA
gcagaaaggttttactattcaacaattaggagtgtcca

SEQ ID NO: 456 - DNA, 28 bp, synthetic construct
  source 1..28: mol_type = other DNA
caccgaaaggttttactattcaacaatt

SEQ ID NO: 457 - DNA, 28 bp, synthetic construct
  source 1..28: mol_type = other DNA
agacaattgttgaatagtaaaacctttc

SEQ ID NO: 458 - DNA, 23 bp, synthetic construct
  source 1..23: mol_type = other DNA
ggaatgaaataatttgtatgcca

SEQ ID NO: 459 - DNA, 37 bp, synthetic construct
  source 1..37: mol_type = other DNA
atgggaatgaaataatttgtatgccatgccgtgtgga

SEQ ID NO: 460 - DNA, 27 bp, synthetic construct
  source 1..27: mol_type = other DNA
caccggaatgaaataatttgtatgcca

SEQ ID NO: 461 - DNA, 27 bp, synthetic construct
  source 1..27: mol_type = other DNA
agactggcatacaaattatttcattcc

SEQ ID NO: 462 - DNA, 23 bp, synthetic construct
  source 1..23: mol_type = other DNA
gccctgtaaaggaaactggaaca

SEQ ID NO: 463 - DNA, 37 bp, synthetic construct
  source 1..37: mol_type = other DNA
ctggccctgtaaaggaaactggaacacaaagcataga

SEQ ID NO: 464 - DNA, 27 bp, synthetic construct
  source 1..27: mol_type = other DNA
caccgccctgtaaaggaaactggaaca

SEQ ID NO: 465 - DNA, 27 bp, synthetic construct
  source 1..27: mol_type = other DNA
agactgttccagtttcctttacagggc

SEQ ID NO: 466 - DNA, 24 bp, synthetic construct
  source 1..24: mol_type = other DNA
gagtattccatgtcctattgtgta

SEQ ID NO: 467 - DNA, 38 bp, synthetic construct
  source 1..38: mol_type = other DNA
agtgagtattccatgtcctattgtgtagattgtgtttt

SEQ ID NO: 468 - DNA, 28 bp, synthetic construct
  source 1..28: mol_type = other DNA
caccgagtattccatgtcctattgtgta

SEQ ID NO: 469 - DNA, 28 bp, synthetic construct
  source 1..28: mol_type = other DNA
agactacacaataggacatggaatactc

SEQ ID NO: 470 - DNA, 23 bp, synthetic construct
  source 1..23: mol_type = other DNA
tggaaagtgagtattccatgtcc

SEQ ID NO: 471 - DNA, 37 bp, synthetic construct
  source 1..37: mol_type = other DNA
ccttggaaagtgagtattccatgtcctattgtgtaga

SEQ ID NO: 472 - DNA, 28 bp, synthetic construct
  source 1..28: mol_type = other DNA
caccgtggaaagtgagtattccatgtcc

SEQ ID NO: 473 - DNA, 28 bp, synthetic construct
  source 1..28: mol_type = other DNA
agacggacatggaatactcactttccac

SEQ ID NO: 474 - DNA, 22 bp, synthetic construct
  source 1..22: mol_type = other DNA
gagtgaaggcagagaggggtta

SEQ ID NO: 475 - DNA, 37 bp, synthetic construct
  source 1..37: mol_type = other DNA
ttggagtgaaggcagagaggggttaaggtagcatata

SEQ ID NO: 476 - DNA, 27 bp, synthetic construct
  source 1..27: mol_type = other DNA
caccgagtgaaggcagagaggggttaa

SEQ ID NO: 477 - DNA, 27 bp, synthetic construct
  source 1..27: mol_type = other DNA
agacttaacccctctctgccttcactc

SEQ ID NO: 478 - DNA, 19 bp, synthetic construct
  source 1..19: mol_type = other DNA
tggtttcataactaggagg

SEQ ID NO: 479 - DNA, 28 bp, synthetic construct
  source 1..28: mol_type = other DNA
gcatggtttcataactaggaggtggtgt

SEQ ID NO: 480 - DNA, 24 bp, synthetic construct
  source 1..24: mol_type = other DNA
caccgtggtttcataactaggagg

SEQ ID NO: 481 - DNA, 24 bp, synthetic construct
  source 1..24: mol_type = other DNA
aaaccctcctagttatgaaaccac

SEQ ID NO: 482 - DNA, 20 bp, synthetic construct
  source 1..20: mol_type = other DNA
ggcagccagggctggcctca

SEQ ID NO: 483 - DNA, 29 bp, synthetic construct
  source 1..29: mol_type = other DNA
ctgggcagccagggctggcctcagggtgt

SEQ ID NO: 484 - DNA, 24 bp, synthetic construct
  source 1..24: mol_type = other DNA
caccggcagccagggctggcctca

SEQ ID NO: 485 - DNA, 24 bp, synthetic construct
  source 1..24: mol_type = other DNA
aaactgaggccagccctggctgcc

SEQ ID NO: 486 - DNA, 20 bp, synthetic construct
  source 1..20: mol_type = other DNA
gctacctgcgccacatccat

SEQ ID NO: 487 - DNA, 29 bp, synthetic construct
  source 1..29: mol_type = other DNA
cgcgctacctgcgccacatccatcggcgc

SEQ ID NO: 488 - DNA, 24 bp, synthetic construct
  source 1..24: mol_type = other DNA
caccgctacctgcgccacatccat

SEQ ID NO: 489 - DNA, 24 bp, synthetic construct
  source 1..24: mol_type = other DNA
aaacatggatgtggcgcaggtagc

SEQ ID NO: 490 - DNA, 19 bp, synthetic construct
  source 1..19: mol_type = other DNA
tcaggagtcaggtgcacca

SEQ ID NO: 491 - DNA, 28 bp, synthetic construct
  source 1..28: mol_type = other DNA
tcctcaggagtcaggtgcaccatggtgt

SEQ ID NO: 492 - DNA, 24 bp, synthetic construct
  source 1..24: mol_type = other DNA
caccgtcaggagtcaggtgcacca

SEQ ID NO: 493 - DNA, 24 bp, synthetic construct
  source 1..24: mol_type = other DNA
aaactggtgcacctgactcctgac

SEQ ID NO: 494 - DNA, 19 bp, synthetic construct
  source 1..19: mol_type = other DNA
ctccaccatgtgggttctc

SEQ ID NO: 495 - DNA, 28 bp, synthetic construct
  source 1..28: mol_type = other DNA
cttctccaccatgtgggttctccggtgt

SEQ ID NO: 496 - DNA, 24 bp, synthetic construct
  source 1..24: mol_type = other DNA
caccgctccaccatgtgggttctc

SEQ ID NO: 497 - DNA, 24 bp, synthetic construct
  source 1..24: mol_type = other DNA
aaacgagaacccacatggtggagc

SEQ ID NO: 498 - DNA, 19 bp, synthetic construct
  source 1..19: mol_type = other DNA
tcttacaacaacatgagag

SEQ ID NO: 499 - DNA, 28 bp, synthetic construct
  source 1..28: mol_type = other DNA
gattcttacaacaacatgagagaggggt

SEQ ID NO: 500 - DNA, 24 bp, synthetic construct
  source 1..24: mol_type = other DNA
caccgtcttacaacaacatgagag

SEQ ID NO: 501 - DNA, 24 bp, synthetic construct
  source 1..24: mol_type = other DNA
aaacctctcatgttgttgtaagac

SEQ ID NO: 502 - DNA, 19 bp, synthetic construct
  source 1..19: mol_type = other DNA
aatactaagacatgcagag

SEQ ID NO: 503 - DNA, 28 bp, synthetic construct
  source 1..28: mol_type = other DNA
agaaatactaagacatgcagagaggtgc

SEQ ID NO: 504 - DNA, 24 bp, synthetic construct
  source 1..24: mol_type = other DNA
caccgaatactaagacatgcagag

SEQ ID NO: 505 - DNA, 24 bp, synthetic construct
  source 1..24: mol_type = other DNA
aaacctctgcatgtcttagtattc

SEQ ID NO: 506 - DNA, 19 bp, synthetic construct
  source 1..19: mol_type = other DNA
ggtttgtgaacgcgtggag

SEQ ID NO: 507 - DNA, 28 bp, synthetic construct
  source 1..28: mol_type = other DNA
tgaggtttgtgaacgcgtggaggggcgc

SEQ ID NO: 508 - DNA, 24 bp, synthetic construct
  source 1..24: mol_type = other DNA
caccgggtttgtgaacgcgtggag

SEQ ID NO: 509 - DNA, 24 bp, synthetic construct
  source 1..24: mol_type = other DNA
aaacctccacgcgttcacaaaccc

SEQ ID NO: 510 - DNA, 20 bp, synthetic construct
  source 1..20: mol_type = other DNA
gctgaaaaggtggtctatgt

SEQ ID NO: 511 - DNA, 29 bp, synthetic construct
  source 1..29: mol_type = other DNA
ttggctgaaaaggtggtctatgttggcgt

SEQ ID NO: 512 - DNA, 24 bp, synthetic construct
  source 1..24: mol_type = other DNA
caccgctgaaaaggtggtctatgt

SEQ ID NO: 513 - DNA, 24 bp, synthetic construct
  source 1..24: mol_type = other DNA
aaacacatagaccaccttttcagc

SEQ ID NO: 514 - DNA, 19 bp, synthetic construct
  source 1..19: mol_type = other DNA
ggtcaggtgtcccagagcc

SEQ ID NO: 515 - DNA, 28 bp, synthetic construct
  source 1..28: mol_type = other DNA
ggcggtcaggtgtcccagagccaggggt

SEQ ID NO: 516 - DNA, 24 bp, synthetic construct
  source 1..24: mol_type = other DNA
caccgggtcaggtgtcccagagcc

SEQ ID NO: 517 - DNA, 24 bp, synthetic construct
  source 1..24: mol_type = other DNA
aaacggctctgggacacctgaccc

SEQ ID NO: 518 - DNA, 19 bp, synthetic construct
  source 1..19: mol_type = other DNA
tgtcaagtggcgtgacacc

SEQ ID NO: 519 - DNA, 28 bp, synthetic construct
  source 1..28: mol_type = other DNA
gcctgtcaagtggcgtgacaccgggcgt

SEQ ID NO: 520 - DNA, 24 bp, synthetic construct
  source 1..24: mol_type = other DNA
caccgtgtcaagtggcgtgacacc

SEQ ID NO: 521 - DNA, 24 bp, synthetic construct
  source 1..24: mol_type = other DNA
aaacggtgtcacgccacttgacac

SEQ ID NO: 522 - DNA, 19 bp, synthetic construct
  source 1..19: mol_type = other DNA
ggccgagaccaccaatcag

SEQ ID NO: 523 - DNA, 28 bp, synthetic construct
  source 1..28: mol_type = other DNA
taaggccgagaccaccaatcagaggagt

SEQ ID NO: 524 - DNA, 24 bp, synthetic construct
  source 1..24: mol_type = other DNA
caccgggccgagaccaccaatcag

SEQ ID NO: 525 - DNA, 24 bp, synthetic construct
  source 1..24: mol_type = other DNA
aaacctgattggtggtctcggccc

SEQ ID NO: 526 - DNA, 20 bp, synthetic construct
  source 1..20: mol_type = other DNA
gaggaagctggtctgggcct

SEQ ID NO: 527 - DNA, 29 bp, synthetic construct
  source 1..29: mol_type = other DNA
tcagaggaagctggtctgggcctgggagt

SEQ ID NO: 528 - DNA, 24 bp, synthetic construct
  source 1..24: mol_type = other DNA
caccgaggaagctggtctgggcct

SEQ ID NO: 529 - DNA, 24 bp, synthetic construct
  source 1..24: mol_type = other DNA
aaacaggcccagaccagcttcctc

SEQ ID NO: 530 - DNA, 20 bp, synthetic construct
  source 1..20: mol_type = other DNA
gtggtagctggggctggggg

SEQ ID NO: 531 - DNA, 29 bp, synthetic construct
  source 1..29: mol_type = other DNA
gaggtggtagctggggctgggggcggtgt

SEQ ID NO: 532 - DNA, 24 bp, synthetic construct
  source 1..24: mol_type = other DNA
caccgtggtagctggggctggggg

SEQ ID NO: 533 - DNA, 24 bp, synthetic construct
  source 1..24: mol_type = other DNA
aaaccccccagccccagctaccac

SEQ ID NO: 534 - DNA, 19 bp, synthetic construct
  source 1..19: mol_type = other DNA
ttccctctttagccagagc

SEQ ID NO: 535 - DNA, 28 bp, synthetic construct
  source 1..28: mol_type = other DNA
ccattccctctttagccagagccggggt

SEQ ID NO: 536 - DNA, 24 bp, synthetic construct
  source 1..24: mol_type = other DNA
caccgttccctctttagccagagc

SEQ ID NO: 537 - DNA, 24 bp, synthetic construct
  source 1..24: mol_type = other DNA
aaacgctctggctaaagagggaac

SEQ ID NO: 538 - DNA, 20 bp, synthetic construct
  source 1..20: mol_type = other DNA
gaggctcccatcacggggga

SEQ ID NO: 539 - DNA, 29 bp, synthetic construct
  source 1..29: mol_type = other DNA
agagaggctcccatcacgggggagggagt

SEQ ID NO: 540 - DNA, 24 bp, synthetic construct
  source 1..24: mol_type = other DNA
caccgaggctcccatcacggggga

SEQ ID NO: 541 - DNA, 24 bp, synthetic construct
  source 1..24: mol_type = other DNA
aaactcccccgtgatgggagcctc

SEQ ID NO: 542 - DNA, 19 bp, synthetic construct
  source 1..19: mol_type = other DNA
agaaatagactaattgcat

SEQ ID NO: 543 - DNA, 28 bp, synthetic construct
  source 1..28: mol_type = other DNA
agcagaaatagactaattgcatgggcgt

SEQ ID NO: 544 - DNA, 24 bp, synthetic construct
  source 1..24: mol_type = other DNA
caccgagaaatagactaattgcat

SEQ ID NO: 545 - DNA, 24 bp, synthetic construct
  source 1..24: mol_type = other DNA
aaacatgcaattagtctatttctc

SEQ ID NO: 546 - DNA, 19 bp, synthetic construct
  source 1..19: mol_type = other DNA
accatcacaaggaaacgct

SEQ ID NO: 547 - DNA, 28 bp, synthetic construct
  source 1..28: mol_type = other DNA
gtcaccatcacaaggaaacgcttggtgt

SEQ ID NO: 548 - DNA, 24 bp, synthetic construct
  source 1..24: mol_type = other DNA
caccgaccatcacaaggaaacgct

SEQ ID NO: 549 - DNA, 24 bp, synthetic construct
  source 1..24: mol_type = other DNA
aaacagcgtttccttgtgatggtc

SEQ ID NO: 550 - DNA, 20 bp, synthetic construct
  source 1..20: mol_type = other DNA
gggcctgacagcggaaaggg

SEQ ID NO: 551 - DNA, 29 bp, synthetic construct
  source 1..29: mol_type = other DNA
gcagggcctgacagcggaaagggtggagc

SEQ ID NO: 552 - DNA, 24 bp, synthetic construct
  source 1..24: mol_type = other DNA
caccgggcctgacagcggaaaggg

SEQ ID NO: 553 - DNA, 24 bp, synthetic construct
  source 1..24: mol_type = other DNA
aaacccctttccgctgtcaggccc

SEQ ID NO: 554 - DNA, 19 bp, synthetic construct
  source 1..19: mol_type = other DNA
ttgccgtgccccttcccca

SEQ ID NO: 555 - DNA, 28 bp, synthetic construct
  source 1..28: mol_type = other DNA
ttcttgccgtgccccttccccagggtgt

SEQ ID NO: 556 - DNA, 24 bp, synthetic construct
  source 1..24: mol_type = other DNA
caccgttgccgtgccccttcccca

SEQ ID NO: 557 - DNA, 24 bp, synthetic construct
  source 1..24: mol_type = other DNA
aaactggggaaggggcacggcaac

SEQ ID NO: 558 - DNA, 19 bp, synthetic construct
  source 1..19: mol_type = other DNA
tcttggtacctgaagttat

SEQ ID NO: 559 - DNA, 28 bp, synthetic construct
  source 1..28: mol_type = other DNA
agctcttggtacctgaagttataggggt

SEQ ID NO: 560 - DNA, 24 bp, synthetic construct
  source 1..24: mol_type = other DNA
caccgtcttggtacctgaagttat

SEQ ID NO: 561 - DNA, 24 bp, synthetic construct
  source 1..24: mol_type = other DNA
aaacataacttcaggtaccaagac

SEQ ID NO: 562 - DNA, 19 bp, synthetic construct
  source 1..19: mol_type = other DNA
gacccatggcgtctggact

SEQ ID NO: 563 - DNA, 28 bp, synthetic construct
  source 1..28: mol_type = other DNA
aatgacccatggcgtctggactaggagc

SEQ ID NO: 564 - DNA, 24 bp, synthetic construct
  source 1..24: mol_type = other DNA
caccggacccatggcgtctggact

SEQ ID NO: 565 - DNA, 24 bp, synthetic construct
  source 1..24: mol_type = other DNA
aaacagtccagacgccatgggtcc

SEQ ID NO: 566 - DNA, 19 bp, synthetic construct
  source 1..19: mol_type = other DNA
tatgctgaggatttggaaa

SEQ ID NO: 567 - DNA, 28 bp, synthetic construct
  source 1..28: mol_type = other DNA
cattatgctgaggatttggaaagggtgt

SEQ ID NO: 568 - DNA, 24 bp, synthetic construct
  source 1..24: mol_type = other DNA
caccgtatgctgaggatttggaaa

SEQ ID NO: 569 - DNA, 24 bp, synthetic construct
  source 1..24: mol_type = other DNA
aaactttccaaatcctcagcatac

SEQ ID NO: 570 - DNA, 19 bp, synthetic construct
  source 1..19: mol_type = other DNA
aggaggatcactagaggcc

SEQ ID NO: 571 - DNA, 28 bp, synthetic construct
  source 1..28: mol_type = other DNA
ggcaggaggatcactagaggccaggagt

SEQ ID NO: 572 - DNA, 24 bp, synthetic construct
  source 1..24: mol_type = other DNA
caccgaggaggatcactagaggcc

SEQ ID NO: 573 - DNA, 24 bp, synthetic construct
  source 1..24: mol_type = other DNA
aaacggcctctagtgatcctcctc

SEQ ID NO: 574 - DNA, 19 bp, synthetic construct
  source 1..19: mol_type = other DNA
ttatcacaggctccaggaa

SEQ ID NO: 575 - DNA, 27 bp, synthetic construct
  source 1..27: mol_type = other DNA
cttttatcacaggctccaggaagggtt

SEQ ID NO: 576 - DNA, 24 bp, synthetic construct
  source 1..24: mol_type = other DNA
caccgttatcacaggctccaggaa

SEQ ID NO: 577 - DNA, 24 bp, synthetic construct
  source 1..24: mol_type = other DNA
aaacttcctggagcctgtgataac

SEQ ID NO: 578 - DNA, 20 bp, synthetic construct
  source 1..20: mol_type = other DNA
gaacacaaagcatagactgc

SEQ ID NO: 579 - DNA, 28 bp, synthetic construct
  source 1..28: mol_type = other DNA
ctggaacacaaagcatagactgcggggc

SEQ ID NO: 580 - DNA, 24 bp, synthetic construct
  source 1..24: mol_type = other DNA
caccgaacacaaagcatagactgc

SEQ ID NO: 581 - DNA, 24 bp, synthetic construct
  source 1..24: mol_type = other DNA
aaacgcagtctatgctttgtgttc

SEQ ID NO: 582 - DNA, 20 bp, synthetic construct
  source 1..20: mol_type = other DNA
gtgagtattccatgtcctat

SEQ ID NO: 583 - DNA, 28 bp, synthetic construct
  source 1..28: mol_type = other DNA
aaagtgagtattccatgtcctattgtgt

SEQ ID NO: 584 - DNA, 24 bp, synthetic construct
  source 1..24: mol_type = other DNA
caccgtgagtattccatgtcctat

SEQ ID NO: 585 - DNA, 24 bp, synthetic construct
  source 1..24: mol_type = other DNA
aaacataggacatggaatactcac

SEQ ID NO: 586 - DNA, 19 bp, synthetic construct
  source 1..19: mol_type = other DNA
gaaggcagagaggggttaa

SEQ ID NO: 587 - DNA, 27 bp, synthetic construct
  source 1..27: mol_type = other DNA
agtgaaggcagagaggggttaaggtag

SEQ ID NO: 588 - DNA, 24 bp, synthetic construct
  source 1..24: mol_type = other DNA
caccggaaggcagagaggggttaa

SEQ ID NO: 589 - DNA, 24 bp, synthetic construct
  source 1..24: mol_type = other DNA
aaacttaacccctctctgccttcc

SEQ ID NO: 590 - DNA, 22 bp, synthetic construct
  source 1..22: mol_type = other DNA
gaactttattggctggaactgg

SEQ ID NO: 591 - DNA, 35 bp, synthetic construct
  source 1..35: mol_type = other DNA
taggaactttattggctggaactggagtttcctct

SEQ ID NO: 592 - DNA, 26 bp, synthetic construct
  source 1..26: mol_type = other DNA
caccgaactttattggctggaactgg

SEQ ID NO: 593 - DNA, 26 bp, synthetic construct
  source 1..26: mol_type = other DNA
caacccagttccagccaataaagttc

SEQ ID NO: 594 - DNA, 23 bp, synthetic construct
  source 1..23: mol_type = other DNA
gctacttgtgaaacatcttgaac

SEQ ID NO: 595 - DNA, 35 bp, synthetic construct
  source 1..35: mol_type = other DNA
tatgctacttgtgaaacatcttgaacaacacccct

SEQ ID NO: 596 - DNA, 27 bp, synthetic construct
  source 1..27: mol_type = other DNA
caccgctacttgtgaaacatcttgaac

SEQ ID NO: 597 - DNA, 27 bp, synthetic construct
  source 1..27: mol_type = other DNA
caacgttcaagatgtttcacaagtagc

SEQ ID NO: 598 - DNA, 23 bp, synthetic construct
  source 1..23: mol_type = other DNA
gaggggtctccttgtggtggagg

SEQ ID NO: 599 - DNA, 36 bp, synthetic construct
  source 1..36: mol_type = other DNA
gttgaggggtctccttgtggtggaggagtctccttc

SEQ ID NO: 600 - DNA, 27 bp, synthetic construct
  source 1..27: mol_type = other DNA
caccgaggggtctccttgtggtggagg

SEQ ID NO: 601 - DNA, 27 bp, synthetic construct
  source 1..27: mol_type = other DNA
caaccctccaccacaaggagacccctc

SEQ ID NO: 602 - DNA, 23 bp, synthetic construct
  source 1..23: mol_type = other DNA
gttactcgcctgtcaagtggcgt

SEQ ID NO: 603 - DNA, 35 bp, synthetic construct
  source 1..35: mol_type = other DNA
tctgttactcgcctgtcaagtggcgtgacaccggg

SEQ ID NO: 604 - DNA, 27 bp, synthetic construct
  source 1..27: mol_type = other DNA
caccgttactcgcctgtcaagtggcgt

SEQ ID NO: 605 - DNA, 27 bp, synthetic construct
  source 1..27: mol_type = other DNA
caacacgccacttgacaggcgagtaac

SEQ ID NO: 606 - DNA, 22 bp, synthetic construct
  source 1..22: mol_type = other DNA
gttttactattcaacaattagg

SEQ ID NO: 607 - DNA, 35 bp, synthetic construct
  source 1..35: mol_type = other DNA
aaggttttactattcaacaattaggagtgtccaag

SEQ ID NO: 608 - DNA, 26 bp, synthetic construct
  source 1..26: mol_type = other DNA
caccgttttactattcaacaattagg

SEQ ID NO: 609 - DNA, 26 bp, synthetic construct
  source 1..26: mol_type = other DNA
caaccctaattgttgaatagtaaaac

SEQ ID NO: 610 - DNA, 23 bp, synthetic construct
  source 1..23: mol_type = other DNA
gagtcaatgcagatagagctctt

SEQ ID NO: 611 - DNA, 35 bp, synthetic construct
  source 1..35: mol_type = other DNA
cctgagtcaatgcagatagagctcttggtacctga

SEQ ID NO: 612 - DNA, 27 bp, synthetic construct
  source 1..27: mol_type = other DNA
caccgagtcaatgcagatagagctctt

SEQ ID NO: 613 - DNA, 27 bp, synthetic construct
  source 1..27: mol_type = other DNA
caacaagagctctatctgcattgactc

SEQ ID NO: 614 - DNA, 23 bp, synthetic construct
  source 1..23: mol_type = other DNA
ggcaccagttcttgccgtgcccc

SEQ ID NO: 615 - DNA, 35 bp, synthetic construct
  source 1..35: mol_type = other DNA
cctggcaccagttcttgccgtgccccttccccagg

SEQ ID NO: 616 - DNA, 27 bp, synthetic construct
  source 1..27: mol_type = other DNA
caccggcaccagttcttgccgtgcccc

SEQ ID NO: 617 - DNA, 27 bp, synthetic construct
  source 1..27: mol_type = other DNA
caacggggcacggcaagaactggtgcc

SEQ ID NO: 618 - DNA, 22 bp, synthetic construct
  source 1..22: mol_type = other DNA
gggctgtcagaggaagctggtc

SEQ ID NO: 619 - DNA, 34 bp, synthetic construct
  source 1..34: mol_type = other DNA
gctgggctgtcagaggaagctggtctgggcctgg

SEQ ID NO: 620 - DNA, 26 bp, synthetic construct
  source 1..26: mol_type = other DNA
caccgggctgtcagaggaagctggtc

SEQ ID NO: 621 - DNA, 26 bp, synthetic construct
  source 1..26: mol_type = other DNA
caacgaccagcttcctctgacagccc

SEQ ID NO: 622 - DNA, 22 bp, synthetic construct
  source 1..22: mol_type = other DNA
ggtggcaatggataaggccgag

SEQ ID NO: 623 - DNA, 34 bp, synthetic construct
  source 1..34: mol_type = other DNA
tttggtggcaatggataaggccgagaccaccaat

SEQ ID NO: 624 - DNA, 26 bp, synthetic construct
  source 1..26: mol_type = other DNA
caccggtggcaatggataaggccgag

SEQ ID NO: 625 - DNA, 26 bp, synthetic construct
  source 1..26: mol_type = other DNA
caacctcggccttatccattgccacc

SEQ ID NO: 626 - DNA, 20 bp, synthetic construct
  source 1..20: mol_type = other DNA
ggaactttattggctggaac

SEQ ID NO: 627 - DNA, 32 bp, synthetic construct
  source 1..32: mol_type = other DNA
ttaggaactttattggctggaactggagtttc

SEQ ID NO: 628 - DNA, 24 bp, synthetic construct
  source 1..24: mol_type = other DNA
caccggaactttattggctggaac

SEQ ID NO: 629 - DNA, 24 bp, synthetic construct
  source 1..24: mol_type = other DNA
aaacgttccagccaataaagttcc

SEQ ID NO: 630 - DNA, 22 bp, synthetic construct
  source 1..22: mol_type = other DNA
gattcttacaacaacatgagag

SEQ ID NO: 631 - DNA, 34 bp, synthetic construct
  source 1..34: mol_type = other DNA
tttgattcttacaacaacatgagagaggggtgtt

SEQ ID NO: 632 - DNA, 26 bp, synthetic construct
  source 1..26: mol_type = other DNA
caccgattcttacaacaacatgagag

SEQ ID NO: 633 - DNA, 26 bp, synthetic construct
  source 1..26: mol_type = other DNA
aaacctctcatgttgttgtaagaatc

SEQ ID NO: 634 - DNA, 20 bp, synthetic construct
  source 1..20: mol_type = other DNA
gaggggtctccttgtggtgg

SEQ ID NO: 635 - DNA, 32 bp, synthetic construct
  source 1..32: mol_type = other DNA
gttgaggggtctccttgtggtggaggagtctc

SEQ ID NO: 636 - DNA, 24 bp, synthetic construct
  source 1..24: mol_type = other DNA
caccgaggggtctccttgtggtgg

SEQ ID NO: 637 - DNA, 24 bp, synthetic construct
  source 1..24: mol_type = other DNA
aaacccaccacaaggagacccctc

SEQ ID NO: 638 - DNA, 20 bp, synthetic construct
  source 1..20: mol_type = other DNA
gtgacaccgggcgtgttccc

SEQ ID NO: 639 - DNA, 32 bp, synthetic construct
  source 1..32: mol_type = other DNA
ggcgtgacaccgggcgtgttccccagagtgac

SEQ ID NO: 640 - DNA, 24 bp, synthetic construct
  source 1..24: mol_type = other DNA
caccgtgacaccgggcgtgttccc

SEQ ID NO: 641 - DNA, 24 bp, synthetic construct
  source 1..24: mol_type = other DNA
aaacgggaacacgcccggtgtcac

SEQ ID NO: 642 - DNA, 20 bp, synthetic construct
  source 1..20: mol_type = other DNA
ggttttactattcaacaatt

SEQ ID NO: 643 - DNA, 32 bp, synthetic construct
  source 1..32: mol_type = other DNA
aaaggttttactattcaacaattaggagtgtc

SEQ ID NO: 644 - DNA, 24 bp, synthetic construct
  source 1..24: mol_type = other DNA
caccggttttactattcaacaatt

SEQ ID NO: 645 - DNA, 24 bp, synthetic construct
  source 1..24: mol_type = other DNA
aaacaattgttgaatagtaaaacc

SEQ ID NO: 646 - DNA, 21 bp, synthetic construct
  source 1..21: mol_type = other DNA
gctcttggtacctgaagttat

SEQ ID NO: 647 - DNA, 33 bp, synthetic construct
  source 1..33: mol_type = other DNA
agagctcttggtacctgaagttataggggttta

SEQ ID NO: 648 - DNA, 25 bp, synthetic construct
  source 1..25: mol_type = other DNA
caccgctcttggtacctgaagttat

SEQ ID NO: 649 - DNA, 25 bp, synthetic construct
  source 1..25: mol_type = other DNA
aaacataacttcaggtaccaagagc

SEQ ID NO: 650 - DNA, 21 bp, synthetic construct
  source 1..21: mol_type = other DNA
gttcttgccgtgccccttccc

SEQ ID NO: 651 - DNA, 33 bp, synthetic construct
  source 1..33: mol_type = other DNA
ggtgttcttgccgtgccccttccccagggtgtg

SEQ ID NO: 652 - DNA, 25 bp, synthetic construct
  source 1..25: mol_type = other DNA
caccgttcttgccgtgccccttccc

SEQ ID NO: 653 - DNA, 25 bp, synthetic construct
  source 1..25: mol_type = other DNA
aaacgggaaggggcacggcaagaac

SEQ ID NO: 654 - DNA, 20 bp, synthetic construct
  source 1..20: mol_type = other DNA
gaggaagctggtctgggcct

SEQ ID NO: 655 - DNA, 32 bp, synthetic construct
  source 1..32: mol_type = other DNA
tcagaggaagctggtctgggcctgggagtctg

SEQ ID NO: 656 - DNA, 24 bp, synthetic construct
  source 1..24: mol_type = other DNA
caccgaggaagctggtctgggcct

SEQ ID NO: 657 - DNA, 24 bp, synthetic construct
  source 1..24: mol_type = other DNA
aaacaggcccagaccagcttcctc

SEQ ID NO: 658 - DNA, 20 bp, synthetic construct
  source 1..20: mol_type = other DNA
aggccgagaccaccaatcag

SEQ ID NO: 659 - DNA, 32 bp, synthetic construct
  source 1..32: mol_type = other DNA
ataaggccgagaccaccaatcagaggagtttt

SEQ ID NO: 660 - DNA, 25 bp, synthetic construct
  source 1..25: mol_type = other DNA
caccgaggccgagaccaccaatcag

SEQ ID NO: 661 - DNA, 25 bp, synthetic construct
  source 1..25: mol_type = other DNA
aaacctgattggtggtctcggcctc

SEQ ID NO: 662 - DNA, 23 bp, synthetic construct
  source 1..23: mol_type = other DNA
tctacgtgcccttctccaatgcg

SEQ ID NO: 663 - DNA, 37 bp, synthetic construct
  source 1..37: mol_type = other DNA
acttctacgtgcccttctccaatgcgacgggtgtggt

SEQ ID NO: 664 - DNA, 28 bp, synthetic construct
  source 1..28: mol_type = other DNA
caccgtctacgtgcccttctccaatgcg

SEQ ID NO: 665 - DNA, 28 bp, synthetic construct
  source 1..28: mol_type = other DNA
agaccgcattggagaagggcacgtagac

SEQ ID NO: 666 - DNA, 24 bp, synthetic construct
  source 1..24: mol_type = other DNA
ctcgaaggggctgcgtaccacacc

SEQ ID NO: 667 - DNA, 38 bp, synthetic construct
  source 1..38: mol_type = other DNA
gtactcgaaggggctgcgtaccacacccgtcgcattgg

SEQ ID NO: 668 - DNA, 29 bp, synthetic construct
  source 1..29: mol_type = other DNA
caccgctcgaaggggctgcgtaccacacc

SEQ ID NO: 669 - DNA, 29 bp, synthetic construct
  source 1..29: mol_type = other DNA
agacggtgtggtacgcagccccttcgagc

SEQ ID NO: 670 - DNA, 24 bp, synthetic construct
  source 1..24: mol_type = other DNA
ggtagtactgtgggtactcgaagg

SEQ ID NO: 671 - DNA, 38 bp, synthetic construct
  source 1..38: mol_type = other DNA
ccaggtagtactgtgggtactcgaaggggctgcgtacc

SEQ ID NO: 672 - DNA, 28 bp, synthetic construct
  source 1..28: mol_type = other DNA
caccggtagtactgtgggtactcgaagg

SEQ ID NO: 673 - DNA, 28 bp, synthetic construct
  source 1..28: mol_type = other DNA
agacccttcgagtacccacagtactacc

SEQ ID NO: 674 - DNA, 24 bp, synthetic construct
  source 1..24: mol_type = other DNA
acgatcagcagaaacatgtaggcg

SEQ ID NO: 675 - DNA, 38 bp, synthetic construct
  source 1..38: mol_type = other DNA
agcacgatcagcagaaacatgtaggcggccagcatgga

SEQ ID NO: 676 - DNA, 29 bp, synthetic construct
  source 1..29: mol_type = other DNA
caccgacgatcagcagaaacatgtaggcg

SEQ ID NO: 677 - DNA, 29 bp, synthetic construct
  source 1..29: mol_type = other DNA
agaccgcctacatgtttctgctgatcgtc

SEQ ID NO: 678 - DNA, 24 bp, synthetic construct
  source 1..24: mol_type = other DNA
ggcagttctccatgctggccgcct

SEQ ID NO: 679 - DNA, 38 bp, synthetic construct
  source 1..38: mol_type = other DNA
catggcagttctccatgctggccgcctacatgtttctg

SEQ ID NO: 680 - DNA, 28 bp, synthetic construct
  source 1..28: mol_type = other DNA
caccggcagttctccatgctggccgcct

SEQ ID NO: 681 - DNA, 28 bp, synthetic construct
  source 1..28: mol_type = other DNA
agacaggcggccagcatggagaactgcc

SEQ ID NO: 682 - DNA, 24 bp, synthetic construct
  source 1..24: mol_type = other DNA
gcctacatgtttctgctgatcgtg

SEQ ID NO: 683 - DNA, 38 bp, synthetic construct
  source 1..38: mol_type = other DNA
gccgcctacatgtttctgctgatcgtgctgggcttccc

SEQ ID NO: 684 - DNA, 28 bp, synthetic construct
  source 1..28: mol_type = other DNA
caccgcctacatgtttctgctgatcgtg

SEQ ID NO: 685 - DNA, 28 bp, synthetic construct
  source 1..28: mol_type = other DNA
agaccacgatcagcagaaacatgtaggc

SEQ ID NO: 686 - DNA, 23 bp, synthetic construct
  source 1..23: mol_type = other DNA
gcttcttgtgctggacggtgacg

SEQ ID NO: 687 - DNA, 37 bp, synthetic construct
  source 1..37: mol_type = other DNA
gcagcttcttgtgctggacggtgacgtagagcgtgag

SEQ ID NO: 688 - DNA, 27 bp, synthetic construct
  source 1..27: mol_type = other DNA
caccgcttcttgtgctggacggtgacg

SEQ ID NO: 689 - DNA, 27 bp, synthetic construct
  source 1..27: mol_type = other DNA
agaccgtcaccgtccagcacaagaagc

SEQ ID NO: 690 - DNA, 23 bp, synthetic construct
  source 1..23: mol_type = other DNA
gcaggatgtagttgagaggcgtg

SEQ ID NO: 691 - DNA, 37 bp, synthetic construct
  source 1..37: mol_type = other DNA
tgagcaggatgtagttgagaggcgtgcgcagcttctt

SEQ ID NO: 692 - DNA, 27 bp, synthetic construct
  source 1..27: mol_type = other DNA
caccgcaggatgtagttgagaggcgtg

SEQ ID NO: 693 - DNA, 27 bp, synthetic construct
  source 1..27: mol_type = other DNA
agaccacgcctctcaactacatcctgc

SEQ ID NO: 694 - DNA, 24 bp, synthetic construct
  source 1..24: mol_type = other DNA
gctaggttgagcaggatgtagttg

SEQ ID NO: 695 - DNA, 38 bp, synthetic construct
  source 1..38: mol_type = other DNA
acggctaggttgagcaggatgtagttgagaggcgtgcg

SEQ ID NO: 696 - DNA, 28 bp, synthetic construct
  source 1..28: mol_type = other DNA
caccgctaggttgagcaggatgtagttg

SEQ ID NO: 697 - DNA, 28 bp, synthetic construct
  source 1..28: mol_type = other DNA
agaccaactacatcctgctcaacctagc

SEQ ID NO: 698 - DNA, 24 bp, synthetic construct
  source 1..24: mol_type = other DNA
gtggctgacctcttcatggtccta

SEQ ID NO: 699 - DNA, 38 bp, synthetic construct
  source 1..38: mol_type = other DNA
gccgtggctgacctcttcatggtcctaggtggcttcac

SEQ ID NO: 700 - DNA, 28 bp, synthetic construct
  source 1..28: mol_type = other DNA
caccgtggctgacctcttcatggtccta

SEQ ID NO: 701 - DNA, 28 bp, synthetic construct
  source 1..28: mol_type = other DNA
agactaggaccatgaagaggtcagccac

SEQ ID NO: 702 - DNA, 24 bp, synthetic construct
  source 1..24: mol_type = other DNA
gcttcaccagcaccctctacacct

SEQ ID NO: 703 - DNA, 38 bp, synthetic construct
  source 1..38: mol_type = other DNA
gtggcttcaccagcaccctctacacctctctgcatgga

SEQ ID NO: 704 - DNA, 28 bp, synthetic construct
  source 1..28: mol_type = other DNA
caccgcttcaccagcaccctctacacct

SEQ ID NO: 705 - DNA, 28 bp, synthetic construct
  source 1..28: mol_type = other DNA
agacaggtgtagagggtgctggtgaagc

SEQ ID NO: 706 - DNA, 24 bp, synthetic construct
  source 1..24: mol_type = other DNA
aattgcatcctgtgggcccgaaga

SEQ ID NO: 707 - DNA, 38 bp, synthetic construct
  source 1..38: mol_type = other DNA
ccaaattgcatcctgtgggcccgaagacgaagtatcca

SEQ ID NO: 708 - DNA, 29 bp, synthetic construct
  source 1..29: mol_type = other DNA
caccgaattgcatcctgtgggcccgaaga

SEQ ID NO: 709 - DNA, 29 bp, synthetic construct
  source 1..29: mol_type = other DNA
agactcttcgggcccacaggatgcaattc

SEQ ID NO: 710 - DNA, 24 bp, synthetic construct
  source 1..24: mol_type = other DNA
ttggagggcttctttgccaccctg

SEQ ID NO: 711 - DNA, 38 bp, synthetic construct
  source 1..38: mol_type = other DNA
aatttggagggcttctttgccaccctgggcggtatgag

SEQ ID NO: 712 - DNA, 29 bp, synthetic construct
  source 1..29: mol_type = other DNA
caccgttggagggcttctttgccaccctg

SEQ ID NO: 713 - DNA, 29 bp, synthetic construct
  source 1..29: mol_type = other DNA
agaccagggtggcaaagaagccctccaac

SEQ ID NO: 714 - DNA, 24 bp, synthetic construct
  source 1..24: mol_type = other DNA
cccgaagacgaagtatccatgcag

SEQ ID NO: 715 - DNA, 38 bp, synthetic construct
  source 1..38: mol_type = other DNA
gggcccgaagacgaagtatccatgcagagaggtgtaga

SEQ ID NO: 716 - DNA, 29 bp, synthetic construct
  source 1..29: mol_type = other DNA
caccgcccgaagacgaagtatccatgcag

SEQ ID NO: 717 - DNA, 29 bp, synthetic construct
  source 1..29: mol_type = other DNA
agacctgcatggatacttcgtcttcgggc

SEQ ID NO: 718 - DNA, 24 bp, synthetic construct
  source 1..24: mol_type = other DNA
ccagggtggcaaagaagccctcca

SEQ ID NO: 719 - DNA, 38 bp, synthetic construct
  source 1..38: mol_type = other DNA
cgcccagggtggcaaagaagccctccaaattgcatcct

SEQ ID NO: 720 - DNA, 29 bp, synthetic construct
  source 1..29: mol_type = other DNA
caccgccagggtggcaaagaagccctcca

SEQ ID NO: 721 - DNA, 29 bp, synthetic construct
  source 1..29: mol_type = other DNA
agactggagggcttctttgccaccctggc

SEQ ID NO: 722 - DNA, 24 bp, synthetic construct
  source 1..24: mol_type = other DNA
ttcgggcccacaggatgcaatttg

SEQ ID NO: 723 - DNA, 38 bp, synthetic construct
  source 1..38: mol_type = other DNA
gtcttcgggcccacaggatgcaatttggagggcttctt

SEQ ID NO: 724 - DNA, 29 bp, synthetic construct
  source 1..29: mol_type = other DNA
caccgttcgggcccacaggatgcaatttg

SEQ ID NO: 725 - DNA, 29 bp, synthetic construct
  source 1..29: mol_type = other DNA
agaccaaattgcatcctgtgggcccgaac

SEQ ID NO: 726 - DNA, 24 bp, synthetic construct
  source 1..24: mol_type = other DNA
cctctacacctctctgcatggata

SEQ ID NO: 727 - DNA, 38 bp, synthetic construct
  source 1..38: mol_type = other DNA
caccctctacacctctctgcatggatacttcgtcttcg

SEQ ID NO: 728 - DNA, 29 bp, synthetic construct
  source 1..29: mol_type = other DNA
caccgcctctacacctctctgcatggata

SEQ ID NO: 729 - DNA, 29 bp, synthetic construct
  source 1..29: mol_type = other DNA
agactatccatgcagagaggtgtagaggc

SEQ ID NO: 730 - DNA, 20 bp, synthetic construct
  source 1..20: mol_type = other DNA
gggcacgggcagcttgccgg

SEQ ID NO: 731 - DNA, 29 bp, synthetic construct
  source 1..29: mol_type = other DNA
ccagggcacgggcagcttgccggtggtgc

SEQ ID NO: 732 - DNA, 24 bp, synthetic construct
  source 1..24: mol_type = other DNA
caccgggcacgggcagcttgccgg

SEQ ID NO: 733 - DNA, 24 bp, synthetic construct
  source 1..24: mol_type = other DNA
gaacccggcaagctgcccgtgccc

SEQ ID NO: 734 - DNA, 24 bp, synthetic construct
  source 1..24: mol_type = other DNA
gcccatcctggtcgagctggacgg

SEQ ID NO: 735 - DNA, 38 bp, synthetic construct
  source 1..38: mol_type = other DNA
ggtgcccatcctggtcgagctggacggcgacgtaacga

SEQ ID NO: 736 - DNA, 28 bp, synthetic construct
  source 1..28: mol_type = other DNA
caccgcccatcctggtcgagctggacgg

SEQ ID NO: 737 - DNA, 28 bp, synthetic construct
  source 1..28: mol_type = other DNA
taacccgtccagctcgaccaggatgggc

SEQ ID NO: 738 - DNA, 23 bp, synthetic construct
  source 1..23: mol_type = other DNA
gacggcaactacaagacccgcgc

SEQ ID NO: 739 - DNA, 37 bp, synthetic construct
  source 1..37: mol_type = other DNA
gacgacggcaactacaagacccgcgccgaggtgaagt

SEQ ID NO: 740 - DNA, 27 bp, synthetic construct
  source 1..27: mol_type = other DNA
caccgacggcaactacaagacccgcgc

SEQ ID NO: 741 - DNA, 27 bp, synthetic construct
  source 1..27: mol_type = other DNA
agacgcgcgggtcttgtagttgccgtc

SEQ ID NO: 742 - DNA, 23 bp, synthetic construct
  source 1..23: mol_type = other DNA
atgtctgttactcgcctgtcaag

SEQ ID NO: 743 - DNA, 37 bp, synthetic construct
  source 1..37: mol_type = other DNA
tccatgtctgttactcgcctgtcaagtggcgtgacac

SEQ ID NO: 744 - DNA, 28 bp, synthetic construct
  source 1..28: mol_type = other DNA
caccgatgtctgttactcgcctgtcaag

SEQ ID NO: 745 - DNA, 28 bp, synthetic construct
  source 1..28: mol_type = other DNA
agaccttgacaggcgagtaacagacatc

SEQ ID NO: 746 - DNA, 23 bp, synthetic construct
  source 1..23: mol_type = other DNA
aaagctgtgggaaatcgggtcgc

SEQ ID NO: 747 - DNA, 37 bp, synthetic construct
  source 1..37: mol_type = other DNA
tgaaaagctgtgggaaatcgggtcgctggaggaaggg

SEQ ID NO: 748 - DNA, 28 bp, synthetic construct
  source 1..28: mol_type = other DNA
caccgaaagctgtgggaaatcgggtcgc

SEQ ID NO: 749 - DNA, 28 bp, synthetic construct
  source 1..28: mol_type = other DNA
agacgcgacccgatttcccacagctttc

SEQ ID NO: 750 - DNA, 24 bp, synthetic construct
  source 1..24: mol_type = other DNA
gcccggtgtcacgccacttgacag

SEQ ID NO: 751 - DNA, 38 bp, synthetic construct
  source 1..38: mol_type = other DNA
cacgcccggtgtcacgccacttgacaggcgagtaacag

SEQ ID NO: 752 - DNA, 28 bp, synthetic construct
  source 1..28: mol_type = other DNA
caccgcccggtgtcacgccacttgacag

SEQ ID NO: 753 - DNA, 28 bp, synthetic construct
  source 1..28: mol_type = other DNA
taacctgtcaagtggcgtgacaccgggc

SEQ ID NO: 754 - DNA, 24 bp, synthetic construct
  source 1..24: mol_type = other DNA
gagccaaattcaccgagcaggagt

SEQ ID NO: 755 - DNA, 38 bp, synthetic construct
  source 1..38: mol_type = other DNA
gctgagccaaattcaccgagcaggagtgagggaaacgg

SEQ ID NO: 756 - DNA, 28 bp, synthetic construct
  source 1..28: mol_type = other DNA
caccgagccaaattcaccgagcaggagt

SEQ ID NO: 757 - DNA, 28 bp, synthetic construct
  source 1..28: mol_type = other DNA
taacactcctgctcggtgaatttggctc

SEQ ID NO: 758 - DNA, 24 bp, synthetic construct
  source 1..24: mol_type = other DNA
gggaaagacccagcatccgtgggt

SEQ ID NO: 759 - DNA, 38 bp, synthetic construct
  source 1..38: mol_type = other DNA
agtgggaaagacccagcatccgtgggtcgctgaaaagc

SEQ ID NO: 760 - DNA, 28 bp, synthetic construct
  source 1..28: mol_type = other DNA
caccgggaaagacccagcatccgtgggt

SEQ ID NO: 761 - DNA, 28 bp, synthetic construct
  source 1..28: mol_type = other DNA
taacacccacggatgctgggtctttccc

SEQ ID NO: 762 - DNA, 22 bp, synthetic construct
  source 1..22: mol_type = other DNA
gaaggatagagggtgggagagg

SEQ ID NO: 763 - DNA, 19 bp, synthetic construct
  source 1..19: mol_type = other DNA
tggagtgcaatggcgtgac

SEQ ID NO: 764 - DNA, 22 bp, synthetic construct
  source 1..22: mol_type = other DNA
ctaaggggtctgacacagactg

SEQ ID NO: 765 - DNA, 22 bp, synthetic construct
  source 1..22: mol_type = other DNA
gtggctacaagacatttcctcc

SEQ ID NO: 766 - DNA, 23 bp, synthetic construct
  source 1..23: mol_type = other DNA
gcctgtgagattgacaagaacag

SEQ ID NO: 767 - DNA, 20 bp, synthetic construct
  source 1..20: mol_type = other DNA
tactgcgctgaaactgtggc

SEQ ID NO: 768 - DNA, 25 bp, synthetic construct
  source 1..25: mol_type = other DNA
acagttactaatatcatcttacacc

SEQ ID NO: 769 - DNA, 18 bp, synthetic construct
  source 1..18: mol_type = other DNA
ggctgaaaggagagaact

SEQ ID NO: 770 - DNA, 22 bp, synthetic construct
  source 1..22: mol_type = other DNA
cagcagctgttctgagtacttg

SEQ ID NO: 771 - DNA, 21 bp, synthetic construct
  source 1..21: mol_type = other DNA
cccttgacccagaaattccac

SEQ ID NO: 772 - DNA, 22 bp, synthetic construct
  source 1..22: mol_type = other DNA
ctggtttggattagatcagagg

SEQ ID NO: 773 - DNA, 19 bp, synthetic construct
  source 1..19: mol_type = other DNA
gttccaagtgcaattcatg

SEQ ID NO: 774 - DNA, 20 bp, synthetic construct
  source 1..20: mol_type = other DNA
gcagtactgcaagcatcctg

SEQ ID NO: 775 - DNA, 21 bp, synthetic construct
  source 1..21: mol_type = other DNA
gctccaagatggccaactagc

SEQ ID NO: 776 - DNA, 21 bp, synthetic construct
  source 1..21: mol_type = other DNA
tccacatccctccctttcatg

SEQ ID NO: 777 - DNA, 21 bp, synthetic construct
  source 1..21: mol_type = other DNA
ccagagtagaaggcagctagc

SEQ ID NO: 778 - DNA, 22 bp, synthetic construct
  source 1..22: mol_type = other DNA
cagtgatagagatctccctatc

SEQ ID NO: 779 - DNA, 20 bp, synthetic construct
  source 1..20: mol_type = other DNA
gagatagatgcgggcttcca

SEQ ID NO: 780 - DNA, 21 bp, synthetic construct
  source 1..21: mol_type = other DNA
ggacttgggagttatctgtag

SEQ ID NO: 781 - DNA, 20 bp, synthetic construct
  source 1..20: mol_type = other DNA
gaggcaagtcagttgggaac

SEQ ID NO: 782 - DNA, 26 bp, synthetic construct
  source 1..26: mol_type = other DNA
cactgccattctaccaacaatagagg

SEQ ID NO: 783 - DNA, 28 bp, synthetic construct
  source 1..28: mol_type = other DNA
ctaaaacatccaaccttgatagaacacc

SEQ ID NO: 784 - DNA, 22 bp, synthetic construct
  source 1..22: mol_type = other DNA
tgagttttgccacattggccag

SEQ ID NO: 785 - DNA, 22 bp, synthetic construct
  source 1..22: mol_type = other DNA
agagcatgcacccttaacctca

SEQ ID NO: 786 - protein, 1055 aa, synthetic construct
MGKPIPNPLLGLDSTKRTADGSEFESPKKKRKVKKKIFGFDLGIASIGWAVIDHSDENFDPETGEIIEGKVVGCGVRCFPVAENPKDGSSLAAPRREKRLLRRITRRKARRMLGIKRLFVAKGLAASTAELETLYAAQTGGDVWNLRAEALRRPLSKEELLRVLTHLAKHRGFKSYRKAAEEADKESGRILTAIAENRKETAGFQTLAQMIVERAKHSDDHKMRNYTSQEGENKGVAVYVNSIPREEIEKETKLIFEYQKQFGLFTEDLYRDFCKIASRYREAGSVGHMVGRCRFEPEQPRAPKEAPSAELFVALSKINNLKVTVDGERRFLNGEERKALLELLKNTKEVKYLTIKNKLFKGREVFFDDVNYAQKTKKGKSGEEKAVNPEDAKFYAMKGWHKLKAAFSPEQWKEVGSNLPLLDLGMTAVVCEKNDAGIERFLSEKGIPEDYREVFKKLTGSEFINLSLKALYKLNPYLAEGLKYNQACEKAGYDFREDGIKLAEEKGLLLPPIADDKLTTVPVVNRAVAQFRKVYNAMVRTYGAPDQINLEIGRDLKKSRDERNQIMRRQKENEAERKEAEDWLEKEGLAANGKNMLKYRLYRQQNGKCIYSGKAIDLRRLDENGYCDVDHIIPYSRSLDDGQNNKVLCLAEENRKKGSQTPYEYLEPLGRWEEFETVVNTTPSINRYKRNNLLNKDYKEKENDLEFRERNANDNSYIARYVKRYLEDAIDFSASSCTIGNRVQVRTGSLTDYLRHQWGLIKDRDASDRHHAQDAVVVACATQGMVQKLSKLSAIFENKDDFRRKKAEELGHEEAEAWYKYVKQQIREPWSGFRAEVLASLEKVFVSRPPRKNATGEIHQETIRTVNPKRKKYNEKEILSGIKIRGGLAKNGLMLRTDVFVKKNKKGKDEFYLVPVYLSDMGKELPNKAMVPGKKENEWIELDETCQFKFSFYMDDLIKIKKKENEIFGYFRGTNRATASVSVTTHDRSHTFEGIGVKTQDGIEKYQVDPLGRIAKVKKEIRLPLTMMKKNRHKKEEKRTADGSEFESPKKKRKV

SEQ ID NO: 787 - protein, 1054 aa, synthetic construct
MGKPIPNPLLGLDSTKRTADGSEFESPKKKRKVEITINREIGKLGLPRHLVLGMDPGIASCGFALIDTANHEILDLGVRLFDSPTHPKTGQSLAVIRRGFRSTRRNIDRTQARLKHCLQVLKAYGLIPQDATKEYLHTTKGDKQPLKLRVDGLDRLLNDREWALVLYSLCKRRGYIPHGEGNQDKSSEGGKVLSALAANKEAIAETSCRTVGEWLAWQPQSRNRGGNYDKCVTHAQLIEETHILFDAQRSFGSKYASPEFEAAYIEVCDWERSRKDFDRRTYDLVGHCSYFPTEKRAARCTLTSELVSAYGALGNITIIHENGTSRALSATERDECIAILFSCEPIRGNKDCAVKFGALRKALDLSSGDYFKGVPAADEKTREVYKPKGWRVLRNTLNAANPILLQRLRDDRNLADAVMEAVAYSSALPVLQEQLQGLPFSEAEIEALCRLPYSSKALNGYGNRSKKALDMLLDCLEEPEVLNLTQAENDCGLLGLRIAGAQLERSDRLMPYETWIELTGRTNNNPVVIRSMSQMRKVVNAVCRKWGVPNEIHVELDRELRLPQRAKDEIAKANKKNEKNRERIAGQIAELRGCTADEVTGKQIEKYRLWEEQECFDLYTGAKIEVDRLISDDTYTQIDHILPFSRTGENSRNNKVLVLAKSNQDKREQTPYEWMSHDGAPSWDAFERRVQENQKLSRRKKNFLLEKDLDTKEGEFLARSFTDTAYMSREACAYLADCLLFPDDGAKAHVVPTTGRATAWLRRRWGLNFGSNGEKDRSDDRHHATDACVIAACSRSLVIKTARINQETHWSITRGMNETQRRDAIMKALESVMPWETFANEVRAAHDFVVPTRFVPRKGKGELFEQTVYRYAGVNAQGKDIARKASSDKDIVMGNAVVSLDEKSVIKVSEMLCLRLWHDPEAKKGQGAWYADPVYKADIPALKDGTYVPRIAKAHTGRKAWKPVPESAMKKPPLEIYLGDLVQIGDFMGRFSGYNIANANWSFVDRLTKEALGCPTVVKLDNKLAPAIIRESIIMHKRTADGSEFESPKKKRKV

SEQ ID NO: 788 - RNA, 36 nt, synthetic construct
  source 1..36: mol_type = other RNA
gttatagcttcctttccaaatcagacatgctataat

SEQ ID NO: 789 - RNA, 100 nt, synthetic construct
  source 1..100: mol_type = other RNA
ttattttatgtctgatttggaaaggaagtctataataatcgaagttttctttacgagtagggctctgacgtctcatataatatatgaggcgtcatccttt

SEQ ID NO: 790 - RNA, 36 nt, synthetic construct; identical to SEQ ID NO: 20
  source 1..36: mol_type = other RNA

SEQ ID NO: 791 - RNA, 105 nt, synthetic construct; identical to SEQ ID NO: 21
  source 1..105: mol_type = other RNA

SEQ ID NO: 792 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 793 - protein, 1241 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSEITINREIGKLGLPRHLVLGMAPGIASCGFALIDTANREILDLGVRLFDSPTHPKTGQSLAVIRRGFRSTRRNIDRTQARLKHCLQILKAYGLIPQDATKEYFHTTKGDKQPLKLRVDGLDRLLNDREWALVLYSLCKRRGYIPHGEGNQDKSSEGGKVLSALAANKEAIAETSCRTVGEWLAQQPQSRNRGGNYDKCVTHAQLIEETHILFDAQRSFGSKYASPEFEAAYIEVCDWERSRKDFDRRTYDLVGHCSYFPTEKRAARCTLTSELVSAYGALGNITIIHDDGTSRALSATERDECIAILFSCEPIRGNKDCAVKFGALRKALDLSSGDYFKGVPAADEKTREVYKPKGWRVLRNTLNAANPILLQRLRDDRNLADAVMEAVAYSSALPVLQEQLQGLPLSEAEIEALCRLPYSSKALNGYGNRSKKALDMLLDCLEEPEVLNLTQAENDCGLLGLRIAGTQLERSDRLMPYETWIERTGRTNNNPVVIRAMSQMRKVVNAICRKWGVPNEIHVELDRELRLPQRAKDEIAKANKKNEKNRERIAGQIAELRGCTADEVTGKQIEKYRLWEEQECFDLYTGAKIEVDRLISDDTYTQIDHILPFSRTGENSRNNKVLVLAKSNQDKREQTPYEWMSHDGAPSWDAFERRVQENQKLSRRKKNFLLEKDLDTKEGEFLARSFTDTAYMSREVCAYLADCLLFPDDGAKAHVVPTTGRATAWLRRRWGLNFGSNGEKDRSDDRHHATDACVIAACSRSLVIKTARINQETHWSITRGMNETQRRDAIMKALESVMPWETFANEVRAAHDFVVPTRFVPRKGKGELFEQTVYRYAGVNAQGKDIARKASSDKDIVMGNAVVSADEKSVIKVSEMLCLRLWHDPEAKKGQGAWYADPVYKADIPALKDGTYVPRIAKQKYGRKVWKAVPNSALTQKPLEIYLGDLIKVGDKLGRYNGYNIATANWSFVDALTKKEIAFPSVGMLSNELQPIIIRESILDNSGGSKRTADGSEFEPKKKRKV

SEQ ID NO: 794 - DNA, 24 bp, synthetic construct
  source 1..24: mol_type = other DNA
ggcttctccaccatgtgggttctc

SEQ ID NO: 795 - DNA, 38 bp, synthetic construct
  source 1..38: mol_type = other DNA
tagggcttctccaccatgtgggttctccggtgtgtggc

SEQ ID NO: 796 - DNA, 28 bp, synthetic construct
  source 1..28: mol_type = other DNA
caccggcttctccaccatgtgggttctc

SEQ ID NO: 797 - DNA, 28 bp, synthetic construct
  source 1..28: mol_type = other DNA
agacgagaacccacatggtggagaagcc

SEQ ID NO: 798 - DNA, 24 bp, synthetic construct
  source 1..24: mol_type = other DNA
ggggcagagagagagagtgagcga

SEQ ID NO: 799 - DNA, 38 bp, synthetic construct; identical to SEQ ID NO: 185
  source 1..38: mol_type = other DNA

SEQ ID NO: 800 - DNA, 28 bp, synthetic construct
  source 1..28: mol_type = other DNA
caccggggcagagagagagagtgagcga

SEQ ID NO: 801 - DNA, 28 bp, synthetic construct
  source 1..28: mol_type = other DNA
agactcgctcactctctctctctgcccc

SEQ ID NO: 802 - DNA, 24 bp, synthetic construct
  source 1..24: mol_type = other DNA
aaagaaatactaagacatgcagag

SEQ ID NO: 803 - DNA, 38 bp, synthetic construct; identical to SEQ ID NO: 181
  source 1..38: mol_type = other DNA

SEQ ID NO: 804 - DNA, 29 bp, synthetic construct
  source 1..29: mol_type = other DNA
caccgaaagaaatactaagacatgcagag

SEQ ID NO: 805 - DNA, 29 bp, synthetic construct
  source 1..29: mol_type = other DNA
agacctctgcatgtcttagtatttctttc

SEQ ID NO: 806 - DNA, 24 bp, synthetic construct
  source 1..24: mol_type = other DNA
gtcatctacacagtcaacctctac

SEQ ID NO: 807 - DNA, 38 bp, synthetic construct; identical to SEQ ID NO: 201
  source 1..38: mol_type = other DNA

SEQ ID NO: 808 - DNA, 28 bp, synthetic construct
  source 1..28: mol_type = other DNA
caccgtcatctacacagtcaacctctac

SEQ ID NO: 809 - DNA, 28 bp, synthetic construct
  source 1..28: mol_type = other DNA
agacgtagaggttgactgtgtagatgac

SEQ ID NO: 810 - DNA, 24 bp, synthetic construct
  source 1..24: mol_type = other DNA
gttggctgaaaaggtggtctatgt

SEQ ID NO: 811 - DNA, 38 bp, synthetic construct; identical to SEQ ID NO: 197
  source 1..38: mol_type = other DNA

SEQ ID NO: 812 - DNA, 28 bp, synthetic construct
  source 1..28: mol_type = other DNA
caccgttggctgaaaaggtggtctatgt

SEQ ID NO: 813 - DNA, 28 bp, synthetic construct
  source 1..28: mol_type = other DNA
agacacatagaccaccttttcagccaac

SEQ ID NO: 814 - DNA, 25 bp, synthetic construct
  source 1..25: mol_type = other DNA
gttcttaggtaggtggggtcggcgg

SEQ ID NO: 815 - DNA, 39 bp, synthetic construct; identical to SEQ ID NO: 209
  source 1..39: mol_type = other DNA

SEQ ID NO: 816 - DNA, 29 bp, synthetic construct
  source 1..29: mol_type = other DNA
caccgttcttaggtaggtggggtcggcgg

SEQ ID NO: 817 - DNA, 29 bp, synthetic construct
  source 1..29: mol_type = other DNA
agacccgccgaccccacctacctaagaac

SEQ ID NO: 818 - DNA, 24 bp, synthetic construct
  source 1..24: mol_type = other DNA
tcggcggtcaggtgtcccagagcc

SEQ ID NO: 819 - DNA, 38 bp, synthetic construct; identical to SEQ ID NO: 205
  source 1..38: mol_type = other DNA

SEQ ID NO: 820 - DNA, 29 bp, synthetic construct
  source 1..29: mol_type = other DNA
caccgtcggcggtcaggtgtcccagagcc

SEQ ID NO: 821 - DNA, 29 bp, synthetic construct
  source 1..29: mol_type = other DNA
agacggctctgggacacctgaccgccgac

SEQ ID NO: 822 - RNA, 83 nt, synthetic construct
  source 1..83: mol_type = other RNA
gtcttgagcacgcgaaagcggctccagacaagcggagccacttaagtggcttacgcgtaaagtaaccgccgaaaggcgcgaac

SEQ ID NO: 823 - RNA, 89 nt, synthetic construct
  source 1..89: mol_type = other RNA
gtcttgagcacgcgaaagcggctccagacaagcggagccacttaagtggcttacgcgtaaagtaaccgccgaaaggcgcgaactttttt

SEQ ID NO: 824 - RNA, 106 nt, synthetic construct
  source 1..106: mol_type = other RNA
atcaccttggggaagggcgcggctccagacaaggggagccacttaagtggcttacccgtaaagtaacccccgttcaatcttcggattgggcggggcgaactttttt

SEQ ID NO: 825 - RNA, 55 nt, synthetic construct
  source 1..55: mol_type = other RNA
cgtagtgtagctagcgtatagctagtcttgagcacgcgcccttccccaaggtgat

SEQ ID NO: 826 - RNA, 137 nt, synthetic construct
  source 1..137: mol_type = other RNA
gttatagcttcctttccaaatcagacatgctatagaaatattatatgtctgatttggaaaggaagtctataataatcgaagttatctttacgagtagggctctgacgtcacatataatatatgtggcgtcatccttt

SEQ ID NO: 827 - DNA, 38 bp, synthetic construct
  source 1..38: mol_type = other DNA
gaggcacagggctgtgaggcttatcttcaccatcatga

SEQ ID NO: 828 - DNA, 28 bp, synthetic construct
  source 1..28: mol_type = other DNA
caccgcacagggctgtgaggcttatctt

SEQ ID NO: 829 - DNA, 28 bp, synthetic construct
  source 1..28: mol_type = other DNA
gaacaagataagcctcacagccctgtgc

SEQ ID NO: 830 - DNA, 24 bp, synthetic construct
  source 1..24: mol_type = other DNA
ggaaggccgaagcggagcgtcccg

(19) United States
(12) Patent Application Publication
(10) Pub. No.: US 2025/0197854 A1
(43) Pub. Date: Jun. 19, 2025

(54) TYPE II CAS PROTEINS AND APPLICATIONS THEREOF

(71) Applicant: Alia Therapeutics Srl, Milano (IT)

(72) Inventors: Antonio Casini, Trento (IT); Anna Cereseto, Trento (IT); Nicola Segata, Trento (IT); Michele Demozzi, Trento (IT); Eleonora Pedrazzoli, Moglia (IT); Matteo Ciciani, Schio (IT); Elisabetta Visentin, San Biagio Callalta (IT); Laura Pezzè, Trento (IT)

(73) Assignee: Alia Therapeutics Srl, Milano (IT)

(21) Appl. No.: 18/722,217

(22) PCT Filed: Dec. 21, 2022
(86) PCT No.: PCT/EP2022/087314

§ 371 Date: Jun. 20, 2024

Related U.S. Application Data
(60) Provisional application No. 63/430,886, filed on Dec. 7, 2022.
(60) Provisional application No. 63/407,256, filed on Sep. 16, 2022.
(60) Provisional application No. 63/292,147, filed on Dec. 21, 2021.

(51) Int. Cl. C12N 15/113; A61K 48/00; C12N 9/22; C12N 9/78; C12N 15/62

(52) U.S. Cl. CPC C12N 15/113; A61K 48/005; C12N 9/22; C12N 9/78; C12N 15/625; C12Y 305/04004; C12N 2310/20

(57) ABSTRACT

Type II Cas proteins, for example Type II Cas proteins referred to as AIK Type II Cas proteins, BNK Type II Cas proteins, HPLH Type II Cas proteins, and ANAB Type II Cas proteins; gRNAs for Type II Cas proteins; systems comprising Type II Cas proteins and gRNAs; nucleic acids encoding the Type II Cas proteins, gRNAs and systems; particles comprising the foregoing; pharmaceutical compositions of the foregoing; and uses of the foregoing, for example to alter the genomic DNA of a cell.

### 1. CROSS-REFERENCE TO RELATED APPLICATIONS

This application claims the priority benefit of U.S. provisional application No. 63/292,147, filed Dec. 21, 2021, 63/407,256, filed Sep. 16, 2022, and 63/430,886, filed Dec. 7, 2022, the contents of each which are incorporated herein in their entireties by reference thereto.

### 2. SEQUENCE LISTING

The instant application contains a Sequence Listing XML which has been submitted electronically and is hereby incorporated by reference in its entirety. Said Sequence Listing XML, created on Dec. 15, 2022, is named ALA-006WO_SL.xml and is 862,131 bytes in size.

### 3. BACKGROUND

CRISPR-Cas genome editing with Type II Cas proteins and associated guide RNAs (gRNAs) is a powerful tool with the potential to treat a variety of genetic diseases. Adeno-associated viral vectors (AAVs) are commonly used to deliver Cas proteins, for example Streptococcus pyogenes Cas9 (SpCas9), and their guide RNAs (gRNAs). However, packaging a large Cas protein such as SpCas9 together with a guide RNA into a single AAV vector can be challenging due to the limited packaging capacity of AAVs. Thus, there is a need for Type II Cas nucleases with smaller sizes that can be packaged together with a gRNA in a single AAV. In addition, the discovery of novel nucleases with new PAM specificities can broaden the range of targetable sites in the cell genome, making genome editing more flexible and efficient.

### 4. SUMMARY

This disclosure is based, in part, on the discovery of a Type II Cas protein from an unclassified Proteobacterium (referred to herein as “wild-type BNK Type II Cas”), a Type II Cas protein from the genus Collinsella (referred to herein as “wild-type AIK Type II Cas”), a Type II Cas protein from Alphaproteobacterium (referred to herein as “wild-type HPLH Type II Cas”), and a Type II Cas protein from Collinsella aerofaciens (referred to herein as “wild-type ANAB Type II Cas”. Wild-type BNK, AIK, HPLH, and ANAB Type II Cas proteins are each approximately 1000 amino acids in length, significantly shorter than SpCas9.

In one aspect, the disclosure provides Type II Cas proteins whose amino acid sequence comprises an amino acid sequence that is at least 50% identical (e.g., at least 55%, at least 60%, at least 65%, at least 70%, at least 75%, at least 85%, at least 90%, at least 95% identical, or more) to SEQ ID NO:1 (such proteins referred to herein as “BNK Type II Cas proteins”). Exemplary BNK Type II Cas protein sequences are set forth in SEQ ID NO:1, SEQ ID NO:2, and SEQ ID NO:3.

In another aspect, the disclosure provides Type II Cas proteins whose amino acid sequence comprises an amino acid sequence that is at least 50% identical (e.g., at least 55%, at least 60%, at least 65%, at least 70%, at least 75%, at least 85%, at least 90%, at least 95% identical, or more) identical to SEQ ID NO:7 (such proteins referred to herein as “AIK Type II Cas proteins”). Exemplary AIK Type II Cas protein sequences are set forth in SEQ ID NO:7, SEQ ID NO:8, and SEQ ID NO:9.

In another aspect, the disclosure provides Type II Cas proteins whose amino acid sequence comprises an amino acid sequence that is at least 50% identical (e.g., at least 55%, at least 60%, at least 65%, at least 70%, at least 75%, at least 85%, at least 90%, at least 95% identical, or more) identical to SEQ ID NO:30 (such proteins referred to herein as “HPLH Type II Cas proteins”). Exemplary HPLH Type II Cas protein sequences are set forth in SEQ ID NO:30, SEQ ID NO:31, and SEQ ID NO:786.

In another aspect, the disclosure provides Type II Cas proteins whose amino acid sequence comprises an amino acid sequence that is at least 50% identical (e.g., at least 55%, at least 60%, at least 65%, at least 70%, at least 75%, at least 85%, at least 90%, at least 95% identical, or more) identical to SEQ ID NO:34 (such proteins referred to herein as “ANAB Type II Cas proteins”). Exemplary ANAB Type II Cas protein sequences are set forth in SEQ ID NO:34, SEQ ID NO:35, and SEQ ID NO:787.

In another aspect, the disclosure provides Type II Cas proteins comprising an amino acid sequence having at least 50% (e.g., at least 55%, at least 60%, at least 65%, at least 70%, at least 75%, at least 85%, at least 90%, at least 95%, or more) sequence identity to a RuvC-I domain, RuvC-II domain, RuvC-III domain, BH domain, REC domain, HNH domain, WED domain, or PID domain of a BNK Type II Cas protein, AIK Type II Cas protein, HPLH Type II Cas protein, or ANAB Type II Cas protein. In some embodiments, a Type II Cas protein of the disclosure is a chimeric Type II Cas protein, for example, comprising one or more domains from a BNK Type II, AIK Type II, HPLH Type II, and/or ANAB Type II Cas protein and one or more domains from a different Type II Cas protein such as SpCas9.

In some embodiments, the Type II Cas proteins of the disclosure are in the form of a fusion protein, for example, comprising a BNK Type II Cas protein, AIK Type II Cas protein, HPLH Type II Cas protein, or ANAB Type II Cas protein sequence fused to one or more additional amino acid sequences, for example, one or more nuclear localization signals and/or one or more tags. Other exemplary fusion partners can enable base editing (e.g., where the fusion partner is nucleoside deaminase) or prime editing (e.g., where the fusion partner is a reverse transcriptase).

Exemplary features of Type II Cas proteins of the disclosure are described in Section 6.2 and specific embodiments 1 to 194 and 449 to 450, infra.

In further aspects, the disclosure provides guide (gRNA) molecules, for example single guide RNAs (sgRNAs). In various embodiments, the disclosure provides gRNAs that can be used with the BNK Type II Cas proteins of the disclosure, gRNAs that can be used with the AIK Type II Cas proteins of the disclosure, gRNAs that can be used with the HPLH Type II Cas proteins of the disclosure, and gRNAs that can be used with the ANAB Type II Cas proteins of the disclosure. Exemplary features of the gRNAs of the disclosure are described in Section 6.3 and specific embodiments 195 to 298, infra.

In further aspects, the disclosure provides systems comprising a Type II Cas protein of the disclosure and one or more gRNAs, e.g., sgRNAs. For example, a system can comprise a ribonucleoprotein (RNP) comprising a Type II Cas protein complexed with a gRNA, e.g., an sgRNA or separate crRNA and tracrRNA. Exemplary features of systems are described in Section 6.4 and specific embodiments 299 to 399, infra.

In another aspect, the disclosure provides nucleic acids and pluralities of nucleic acids encoding a Type II Cas protein of the disclosure and, optionally, a guide RNA, for example a sgRNA. In some embodiments, the nucleic acids comprise a Type II Cas protein of the disclosure operably linked to a heterologous promoter, e.g., a mammalian promoter, for example a human promoter.

In another aspect, the disclosure provides nucleic acids encoding a gRNA, for example a sgRNA, of the disclosure and, optionally, a Type II Cas protein, for example a BNK Type II Cas protein, an AIK Type II Cas protein, an HPLH Type II Cas protein, or an ANAB Type II Cas protein.

Exemplary features of nucleic and pluralities of nucleic acids of the disclosure are described in Section 6.5 and specific embodiments 400 to 448, infra.

In further aspects, the disclosure provides particles comprising the Type II Cas proteins, gRNAs, nucleic acids, and systems of the disclosure. Exemplary features of particles of the disclosure are described in Section 6.6 and specific embodiments 452 to 467, infra.

In another aspect, the disclosure provides cells and populations of cells containing or contacted with a Type II Cas protein, gRNA, nucleic acid, plurality of nucleic acids, system, or particle of the disclosure. Exemplary features of such cells and cell populations are described in Section 6.6 and specific embodiments 469 to 476 and 500, infra.

In another aspect, the disclosure provides pharmaceutical compositions comprising a Type II Cas protein, gRNA, nucleic acid, plurality of nucleic acids, system, particle, cell, or population of cells together with one or more excipients. Exemplary features of pharmaceutical compositions are described in Section 6.7 and specific embodiment 468, infra.

In another aspect, the disclosure provides methods of altering cells (e.g., editing the genome of a cell) using the Type II Cas proteins, gRNAs, nucleic acids, systems, particles, and pharmaceutical compositions of the disclosure. Cells altered according to the methods of the disclosure can be used, for example, to treat subjects having a disease or disorder, e.g., genetic disease or disorder. Features of exemplary methods of altering cells are described in Section 6.8 and specific embodiments 477 to 499, infra.

### 5. BRIEF DESCRIPTION OF THE FIGURES

FIGS. 1A-1C show exemplary AIK Type II Cas and BNK Type II Cas sgRNA scaffolds. FIGS. 1A-1B show schematic representations of the hairpin structure generated for visualization after in silico folding using RNA folding form v2.3 (www.unafold.org) of exemplary sgRNA scaffolds (not including the spacer sequence) designed from crRNAs and tracrRNAs identified for AIK Type II Cas (sgRNA_V1, FIG. 1A) and BNK Type II Cas (sgRNA_V2, FIG. 1B). FIG. 1C shows an exemplary trimmed version of the BNK sgRNA (sgRNA_V3). The illustrated exemplary BNK Type II Cas sgRNAs contain an U>A substitution to interrupt a polyU stretch which may affect the efficiency of PolIII-mediated transcription of the guide. FIGS. 1A-1C disclose SEQ ID NOS 26, 16, and 17, respectively, in order of appearance.

FIGS. 2A-2F illustrate BNK Type II Cas and AIK Type II Cas PAM specificities. FIG. 2A: PAM sequence logo for BNK Type II Cas resulting from the bacterial PAM depletion assay. FIG. 2B: PAM enrichment heatmaps calculated for BNK Type II Cas from the same bacterial PAM depletion assay showing the nucleotide preferences at positions 2,3 and 5,6 of the PAM. FIG. 2C: PAM sequence logo for BNK Type II Cas resulting from the in vitro PAM discovery assay. FIG. 2D: PAM enrichment heatmaps calculated for BNK Type II Cas from the same in vitro PAM discovery assay showing the nucleotide preferences at positions 2,3 and 5,6 of the PAM. FIG. 2E: PAM sequence logo for AIK Type II Cas obtained using an in vitro PAM discovery assay. FIG. 2F: PAM enrichment heatmap for AIK Type II Cas showing the nucleotide preferences at position 5, 6, 7 and 8 of the PAM.

FIG. 3 shows activity of AIK Type II Cas and BNK Type II Cas against an EGFP reporter in mammalian cells.

FIGS. 4A-4B show activity of AIK Type II Cas and BNK Type II Cas against endogenous genomic loci in mammalian cells. FIG. 4A: activity of BNK Type II Cas evaluated on a panel of endogenous genomic loci (CCR5, EMX1, Fas) by transient transfection in HEK293T cells. Two guides were evaluated for each target. For targeting the EMX1 locus the BNK_sgRNA_V2 scaffold was used while for the other loci the BNK_sgRNA_V3 scaffold was evaluated. FIG. 4B: indel formation promoted by AIK Type II Cas on a panel of endogenous genomic loci by transient transfection in HEK293T cells. For the majority of the target loci multiple guide RNAs were evaluated for activity, as indicated on the graph.

FIGS. 5A-5B show exemplary BNK Type II Cas (FIG. 5A) and AIK Type II Cas (FIG. 5B) 3′ sgRNA scaffolds and exemplary modifications that can be made to produce trimmed scaffolds. FIG. 5A discloses base sequence and exemplary modified sequences as SEQ ID NOS 15-19. FIG. 5B discloses base sequence and exemplary modified sequences as SEQ ID NOS 26-29.

FIGS. 6A-6B illustrate features of AIK Type II Cas locus and crRNA and tracrRNA. FIG. 6A is a schematic representation of the AIK Type II Cas CRISPR locus. FIG. 6B is a schematic representation of a natural AIK Type II Cas crRNA and tracRNA with its secondary structure. The scheme shows the repeat:antirepeat base pairing region favoring the interaction between the two RNAs. FIG. 6B discloses SEQ ID NOS 824-825, respectively, in order of appearance.

FIG. 7 is a schematic representation of the secondary structure of an HPLH Type II Cas sgRNA generated for visualization after in silico folding using RNA folding form v2.3 (www.unafold.org). The sgRNA was obtained by direct fusion of HPLH crRNA and tracrRNA through a GAAA tetraloop (Table 4C) with additional modifications to improve folding and expression, as highlighted (U:A base flip and T>A base substitution) (SEQ ID NO: 826). The sequence does not include a spacer.

FIGS. 8A-8D illustrate HPLH and ANAB Type II Cas PAM specificities. FIG. A: PAM sequence logo for ANAB Type II Cas resulting from an in vitro PAM discovery assay. FIG. 8B: PAM enrichment heatmaps calculated for ANAB Type II Cas from the same in vitro PAM discovery assay showing the nucleotide preferences at positions 5,6 and 7,8 of the PAM. FIG. 8C: PAM sequence logo for HPLH Type II Cas resulting from the in vitro PAM discovery assay. FIG. 8D: PAM enrichment heatmaps calculated for HPLH Type II Cas from the same in vitro PAM discovery assay showing the nucleotide preferences at positions 5,6 and 7,8 of the PAM.

FIG. 9 shows the activity of AIK, ANAB and HPLH nucleases in human cells. The activity of the three Type II Cas proteins was evaluated through an EGFP disruption assay in U2OS reporter cells by transient transfection. SpCas9 activity is reported as a benchmark. Data are reported as mean±SEM for n≥3 independent studies.

FIGS. 10A-10B illustrate AIK Type II Cas PAM guide RNA preferences. FIG. 10A: An optimal sgRNA spacer length for AIK Type II Cas was assessed by targeting HBB and FAS genes by transient transfection in HEK293T cells using spacers ranging from 22 to 24 bp. Each spacer contained an appended extra 5′ G for efficient transcription from the U6 promoter. FIG. 10B: Side-by-side comparison of alternative AIK Type II Cas sgRNA scaffolds. AIK full scaffold (sgRNAv1), obtained by direct repeat and antirepeat fusion through a GAAA tetraloop, was compared with three alternative sgRNA designs (Table 4B): one containing base substitutions aimed at increasing the stability of its secondary structure (sgRNAv2), a trimmed version characterized by a shorter repeat-antirepeat loop (sgRNAv3), and a stabilized version of the trimmed scaffold (sgRNAv4). The editing activity was evaluated on two endogenous genomic loci (B2M and DNMT1). In all panels editing was evaluated via TIDE analysis and, data reported as mean±SEM for n≥3 independent studies.

FIGS. 11A-11C show in-depth characterization of AIK Type II Cas activity in a human cell line. FIG. 11A: Editing activity of AIK Type II Cas evaluated by transient transfection of HEK293T cells on a panel of 26 endogenous genomic loci. FIG. 11B: Side-by-side comparison of the editing activity of AIK Type II Cas and SpCas9 on a panel of 24 genomic loci in HEK293T cells using overlapping spacers. FIG. 11C: Violin plot summarizing the indel percentages reported in FIG. 11B. In all panels, editing was evaluated via TIDE analysis, and data reported as mean±SEM for n≥3 independent studies.

FIGS. 12A-12B show in-depth characterization of ANAB and HPLP Type II Cas activity in a human cell line. FIG. 12A: Editing activity of ANAB Type II Cas on the DNMT1 and HEKsite1 endogenous genomic loci measured after transient transfection of HEK293T cells. FIG. 12B: Editing activity of HPLH Type II Cas on the DNMT1 (guides g1 and g2) and HEKsite1 endogenous genomic loci measured after transient transfection of HEK293T cells. In FIG. 12B, data are reported as mean±SEM for n=3 independent studies.

FIGS. 13A-13B display a comparison of AIK Type II Cas with small Cas9 orthologs. FIG. 13A: Side-by-side evaluation of the editing activity on nine matched genomic targets after transient transfection of HEK293T cells with AIK Type II Cas, Nme2Cas9 and SaCas9. Nme2Cas9 was evaluated only in six out of nine sites. The sites which were not evaluated are marked as “na” on the graph. FIG. 13B: Violin plot summarizing the editing data presented in FIG. 13A. In all panels editing was evaluated via TIDE analysis, and data reported as mean±SEM for n=3 independent studies.

FIGS. 14A-14B illustrate the genome-wide specificity of AIK Type II Cas. FIG. 14A: Total number of genome wide off-target sites detected by GUIDE-seq in HEK293T cells for AIK Type II Cas and the benchmark nuclease SpCas9 on a panel of matched genomic targets. FIG. 14B: Distribution of the GUIDE-seq reads among the on-target site and the detected off-targets for AIK Type II Cas and SpCas9 on each of the loci evaluated in FIG. 14A.

FIG. 15 shows an AIK Type II Cas base editing heatmap. A-to-G conversions promoted on a panel of representative genomic loci by the ABE8e-AIK adenine base editor. The position of each modified adenine along the spacer sequence, counting from the PAM-proximal side, is indicated on the heatmap. Cells not containing any indicated base editing percentage correspond to positions where a non-modifiable non-A nucleotide is present on the target sequence. The heatmap reports the mean for n=3 independent studies.

FIGS. 16A-16G display ABE8e-AIK and ABE8e-NG base editing on non-overlapping sites. FIG. 16A-D show the base editing efficiency of the ABE8e-AIK adenine base editor on a panel of genomic loci, while FIG. 16E-G demonstrate the efficacy of the benchmark ABE8e-NG on neighboring non-overlapping sites. For each target the position of each A nucleotide is indicated (counting from the PAM-proximal side) with the relative percentage of A-to-G conversion in order to define the editing window of the two base editors. The data relative to ABE8e-AIK are also summarized in the heatmap of FIG. 15. The data are reported as mean±SEM for n=3 independent studies.

FIGS. 17A-17D show side-by-side comparisons of the base editing efficacy and of the base editing window of ABE8e-AIK and ABE8e-NG base editors on overlapping genomic sites obtained by transient transfection of HEK293T cells. The position of the target A nucleotides is counted starting from the PAM-proximal side of the spacer. The data are reported as mean±SEM for n=3 independent studies.

FIGS. 18A-18B show AIK TYPE II Cas RHO gene targeting. FIG. 18A: Evaluation of the editing efficacy of a panel of AIK Type II Cas guide RNAs targeting the first exon of human RHO obtained by transient transfection of HEK293 RHO-EGFP cells. FIG. 18B: Evaluation of the downregulation of RHO-EGFP expression induced by the AIK guides presented in FIG. 18A in the same conditions. The data are reported as mean±SEM for n=3 independent studies.

FIGS. 19A-19D illustrate the delivery of AIK Type II Cas and ABE8e-AIK using all-in-one AAV vectors. FIG. 19A: Schematic representation of the all-in-one AAV vectors used to deliver AIK Type II Cas and the ABE8e-AIK adenine base editor. FIG. 19B: Indel formation in the RHO gene after transduction of HEK293 RHO-EGFP cells with all-in-one AAV vectors expressing AIK and the two best sgRNA identified to target RHO exon 1 among the ones presented in FIG. 18. FIG. 19C: Downregulation of RHO-EGFP expression as measured by FACS analysis after transduction of HEK293 RHO-EGFP cells with all-in-one AIK-expressing AAV vectors as described in FIG. 19B. FIG. 19D: Base editing efficacy of ABE8e-AIK on the HEKsite2 locus when delivered using an all-in-one AAV vector in HEK293T cells. The position of the editable A nucleotides along the spacer sequence is reported on the graph counting from the PAM-proximal side. The data are reported as mean±SEM for n=2 independent studies.

FIG. 20 shows an exemplary AIK Type II Cas sgRNA scaffold (AIK Type II Cas sgRNA_v5) (SEQ ID NO:823). The scaffold is based on the AIK Type II Cas sgRNA_v4 scaffold and includes an additionally trimmed stem-loop (substitution with a GAAA tetraloop).

FIG. 21 shows a side-by-side comparison of indel formation by AIK Type II Cas and guide RNAs having the AIK Type II Cas sgRNA_v1, AIK Type II Cas sgRNA_v4, or AIK Type II Cas sgRNA_v5 scaffold.

### 6. DETAILED DESCRIPTION

In one aspect, the disclosure provides Type II Cas proteins (e.g., BNK Type II Cas proteins, AIK Type II Cas proteins, HPLH Type II Cas proteins, and ANAB Type II Cas proteins). Type II Cas proteins of the disclosure can be in the form of fusion proteins. Unless required otherwise by context, disclosures relating to Type II Cas proteins encompass Type II Cas proteins which are not fusion proteins and Type II Cas proteins which are in the form of fusion proteins (e.g., Type II Cas protein comprising one or more nuclear localization signals and/or one or more tags).

In some embodiments, a Type II Cas protein of the disclosure comprises an amino acid sequence having at least 50% (e.g., at least 55%, at least 60%, at least 65%, at least 70%, at least 75%, at least 85%, at least 90%, at least 95%, or more) sequence identity to a RuvC-I domain, RuvC-II domain, RuvC-III domain, BH domain, REC domain, HNH domain, WED domain, or PID domain of a BNK Type II Cas protein, AIK Type II Cas protein, HPLH Type II Cas protein, or ANAB Type II Cas protein. In some embodiments, a Type II Cas protein of the disclosure is a chimeric Type II Cas protein, for example, comprising one or more domains from a BNK Type II and/or AIK Type II Cas protein; or comprising one or more domains from a BNK Type II, AIK Type II, HPLH Type II, and/or ANAB Type II Cas protein and one or more domains from a different Type II Cas protein such as SpCas9.

Exemplary features of Type II Cas proteins of the disclosure are described in Section 6.2.

In another aspect, the disclosure provides guide (gRNA) molecules, for example single guide RNAs (sgRNAs). Exemplary features of the gRNAs of the disclosure are described in Section 6.3.

In further aspects, the disclosure provides systems comprising a Type II Cas protein of the disclosure and one or more gRNAs, e.g., sgRNAs. Exemplary features of systems are described in Section 6.4.

In further aspects, the disclosure provides nucleic acids and pluralities of nucleic acids encoding a Type II Cas protein of the disclosure and, optionally, a guide RNA, for example a sgRNA, and provides nucleic acids encoding a gRNA, for example a sgRNA, of the disclosure and, optionally, a Type II Cas protein. Exemplary features of nucleic and pluralities of nucleic acids of the disclosure are described in Section 6.5.

In further aspects, the disclosure provides particles comprising the Type II Cas proteins, gRNAs, nucleic acids, and systems of the disclosure. Exemplary features of particles of the disclosure are described in Section 6.6.

In another aspect, the disclosure provides cells and populations of cells containing or contacted with a Type II Cas protein, gRNA, nucleic acid, plurality of nucleic acids, system, or particle of the disclosure. Exemplary features of such cells and cell populations are described in Section 6.6.

In another aspect, the disclosure provides pharmaceutical compositions comprising a Type II Cas protein, gRNA, nucleic acid, plurality of nucleic acids, system, particle, cell, or population of cells together with one or more excipients. Exemplary features of pharmaceutical compositions are described in Section 6.7.

In another aspect, the disclosure provides methods of altering cells (e.g., editing the genome of a cell) using the Type II Cas proteins, gRNAs, nucleic acids, systems, particles, and pharmaceutical compositions of the disclosure. Features of exemplary methods of altering cells are described in Section 6.8.

Those skilled in the relevant art will recognize and appreciate that many changes can be made to the various embodiments described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### 6.1. Definitions

Unless defined otherwise, all technical and scientific terms used herein have the same meaning as commonly understood to one of ordinary skill in the art to which this invention belongs. The following definitions are provided for the full understanding of terms used in this specification.

As used in the specification and claims, the singular form “a,” “an,” and “the” include plural references unless the context clearly dictates otherwise. For example, the term “an agent” includes a plurality of agents, including mixtures thereof.

Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

A Type II Cas protein refers to a wild-type or engineered Type II Cas protein. Engineered Type II Cas proteins can also be referred to as Type II Cas variants. For the avoidance of doubt, any disclosure pertaining to a “Type II Cas” or “Type II Cas protein” pertains to wild-type Type II Cas proteins and Type II Cas variants, unless the context dictates otherwise. A Type II Cas protein can have nuclease activity or be catalytically inactive (e.g., as in a dCas).

As used herein, the percentage identity between two nucleotide sequences or between two amino acid sequences is calculated by multiplying the number of matches between a pair of aligned sequences by 100, and dividing by the length of the aligned region. Identity scoring only counts perfect matches and does not consider the degree of similarity of amino acids to one another, nor does it consider substitutions or deletions as matches. Alignment for purposes of determining percent sequence identity can be achieved in various ways that are within the skill in the art, for instance, by manual alignment or using publicly available computer software such as BLAST, BLAST-2, ALIGN, ALIGN-2 or Megalign (DNASTAR) software. Those skilled in the art can determine appropriate parameters for achieving maximum alignment.

Guide RNA molecule (gRNA) refers to an RNA capable of forming a complex with a Type II Cas protein and which can direct the Type II Cas protein to a target DNA. gRNAs typically comprise a spacer of 15 to 30 nucleotides in length in length. gRNAs of the disclosure are in some embodiments single guide RNAs (sgRNAs), which typically comprise a spacer at the 5′ end of the molecule and a 3′ sgRNA scaffold. Various non-limiting examples of 3′ sgRNA scaffolds are described in Section 6.3.

An sgRNA can in some embodiments comprise no uracil base at the 3′ end of the sgRNA sequence. Alternatively, a sgRNA can comprise one or more uracil bases at the 3′ end of the sgRNA sequence. For example, a sgRNA can comprise 1 uracil (U) at the 3′ end of the sgRNA sequence, 2 uracil (UU) at the 3′ end of the sgRNA sequence, 3 uracil (UUU) at the 3′ end of the sgRNA sequence, 4 uracil (UUUU) at the 3′ end of the sgRNA sequence, 5 uracil (UUUUU) at the 3′ end of the sgRNA sequence, 6 uracil (UUUUUU) at the 3′ end of the sgRNA sequence, 7 uracil (UUUUUUU) at the 3′ end of the sgRNA sequence, or 8 uracil (UUUUUUUU) at the 3′ end of the sgRNA sequence. Different length stretches of uracil can be appended at the 3′ end of a sgRNA as terminators. Thus, for example, the 3′ sgRNA scaffolds set forth in Section 6.3 can be modified by adding or removing one or more uracils at the end of the sequence.

Peptide, protein, and polypeptide are used interchangeably to refer to a natural or synthetic molecule comprising two or more amino acids linked by the carboxyl group of one amino acid to the alpha amino group of another. The amino acids may be natural or synthetic, and can contain chemical modifications such as disulfide bridges, substitution of radioisotopes, phosphorylation, substrate chelation (e.g., chelation of iron or copper atoms), glycosylation, acetylation, formylation, amidation, biotinylation, and a wide range of other modifications. A polypeptide may be attached to other molecules, for instance molecules required for function. Examples of molecules which may be attached to a polypeptide include, without limitation, cofactors, polynucleotides, lipids, metal ions, phosphate, etc. Non-limiting examples of polypeptides include peptide fragments, denatured/unstructured polypeptides, polypeptides having quaternary or aggregated structures, etc. There is expressly no requirement that a polypeptide must contain an intended function; a polypeptide can be functional, non-functional, function for unexpected/unintended purposes, or have unknown function. A polypeptide is comprised of approximately twenty, standard naturally occurring amino acids, although natural and synthetic amino acids which are not members of the standard twenty amino acids may also be used. The standard twenty amino acids include alanine (Ala, A), arginine (Arg, R), asparagine (Asn, N), aspartic acid (Asp, D), cysteine (Cys, C), glutamine (Gln, Q), glutamic acid (Glu, E), glycine (Gly, G), histidine, (His, H), isoleucine (lie, 1), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V). The terms “polypeptide sequence” or “amino acid sequence” are an alphabetical representation of a polypeptide molecule.

Polynucleotide and oligonucleotide are used interchangeably and refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, primers and gRNAs. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. A polynucleotide is composed of a specific sequence of four nucleotide bases: adenine (A); cytosine (C); guanine (G); thymine (T); and uracil (U) for thymine (T) when the polynucleotide is RNA. Thus, the term “nucleotide sequence” is the alphabetical representation of a polynucleotide molecule. The letters used in polynucleotide sequences described herein correspond to IUPAC notation. For example, the letter “N” in a nucleotide sequence represents a nucleotide which can be A, T, C, or G in a DNA sequence, or A, U, C, or G in a RNA sequence; the letter “R” in a nucleotide sequence represents a nucleotide which can be A or G; and the letter “V” in a nucleotide sequence represents a nucleotide which can be “A, C, or G.

Protospacer adjacent motif (PAM) refers to a DNA sequence downstream (e.g., immediately downstream) of a target sequence on the non-target strand recognized by a Type II Cas protein. A PAM sequence is located 3′ of the target sequence on the non-target strand.

Spacer refers to a region of a gRNA molecule which is partially or fully complementary to a target sequence found in the + or − strand of genomic DNA. When complexed with a Type II Cas protein, the gRNA directs the Type II Cas to the target sequence in the genomic DNA. A spacer of a Type II Cas gRNA is typically 15 to 30 nucleotides in length (e.g., 20-25 nucleotides). The nucleotide sequence of a spacer can be, but is not necessarily, fully complementary to the target sequence. For example, a spacer can contain one or more mismatches with a target sequence, e.g., the spacer can comprise one, two, or three mismatches with the target sequence.

### 6.2. Type II Cas Proteins

### 6.2.1. BNK Type II Cas Proteins

In one aspect, the disclosure provides BNK Type II Cas proteins. The BNK Type II Cas proteins typically comprise an amino acid sequence that is at least 50%, at least 55%, at least 60%, at least 65%, at least 70%, at least 75%, at least 85%, at least 90%, or at least 95% identical to SEQ ID NO:1. In some embodiments, the BNK Type II Cas proteins comprise an amino acid sequence that is at least 95%, at least 96%, at least 97%, at least 98%, or at least 99% identical to SEQ ID NO:1. In some embodiments, a BNK Type II Cas protein comprises an amino acid sequence that is identical to SEQ ID NO:1.

Exemplary BNK Type II Cas protein sequences and nucleotide sequences encoding exemplary BNK Type II Cas proteins are set forth in Table 1A.

TABLE 1A
BNK Type II Cas Sequences

| | | SEQ ID |
|---|---|---|
| Name | Sequence | NO |
| BNK Type II | KMQDSVSKMKYRLGIDLGTTSLGWAMLRLDEQNEP | 1 |
| Cas coding | YAVIRAGVRIFNNGRDPKTEASLAVARRLARQQRR | |
| sequence | TRDRKIRRKERLIGELVDMGFFPKDPVKRRQLASL | |
| (aa) (without | DPFKLRTEALDRALSPEEFARAIFHLARRRGFKSN | |
| N-terminal | RKTDSGDTESSKMKEAIKRTLNELQNKGFRTVGEW | |
| methionine) | LNMRHQQRLGTRSRIKNVPTGSGKQTTAYDFYLNR | |
| | FMIEYEFDRIWEKQSQMNPGLFTNERKAILKDIIF | |
| | YQRPLRPVEPGRCTFMPDNPRAPLALPQQQDFRIY | |
| | QEVNNLRKIDPTSLLEVNLTLPERDRIVELLQRKP | |
| | ALTFDAVRKALCFNGTFNLEGENRSELKGNLTNCA | |
| | LAKKKLFGESWYSFDAHKRFEIVEHLLQEESEENL | |
| | VSWLQKECNLSEEYAKNVASVRLPAGYGALCQEAL | |
| | DLILPYLKAEVITYDKAVQKAGMNHSELTLAQETG | |
| | EILPELPYYGQYLKRHVGFGTGKPEDSAEKRYGKI | |
| | PNPTVHIALNQLRTVVNALIRRYGKPTQIVIELAR | |
| | ELKQNKKAKDQYRIEMNHNQNRNERIRADISMILG | |
| | INPENVKRKDIEKQILWEELNLKDATARCCPYSGK | |
| | QISAEMLFTDEVEIDHILPFSRTLDDSKNNKVVCI | |
| | REANRIKGNRTPWEARKDFEKRGWSVEAMTARAQA | |
| | MPKAKRFRFAEDGYKVWLKDFDGFEARALTDTQYM | |
| | SRVAREYLQLICPGQTWSVPGQLTGMLRRFLGLND | |
| | ILGVNGEKNRDDHRHHAVDACVIALTDRSMLQRIS | |
| | TASARAENKHLTRLLESFPAPWATFYEHVTRAVKS | |
| | ICVSHKPEHAYQGAMNEQTAYGLRPDGYVKYRQNG | |
| | KVEHKKLNVIPQVSVKGTWRHGLNSDGSLKAYKGL | |
| | KGGSNFCIEIVMGEGGRWEGDVITTYEAYQIVRAK | |
| | GEAALYGSVSRSGKPLVMRLMQKDIVEMTLADGRC | |
| | KMLLYIITQNKQMFFYRIENAGGGREDVSKRPGSL | |
| | QKALAKKIIVSPIGDFRKEKL | |
| BNK Type II | MKMQDSVSKMKYRLGIDLGTTSLGWAMLRLDEQNE | 2 |
| Cas coding | PYAVIRAGVRIFNNGRDPKTEASLAVARRLARQQR | |
| sequence | RTRDRKIRRKERLIGELVDMGFFPKDPVKRRQLAS | |
| (aa) | LDPFKLRTEALDRALSPEEFARAIFHLARRRGFKS | |
| | NRKTDSGDTESSKMKEAIKRTLNELQNKGFRTVGE | |
| | WLNMRHQQRLGTRSRIKNVPTGSGKQTTAYDFYLN | |
| | RFMIEYEFDRIWEKQSQMNPGLFTNERKAILKDII | |
| | FYQRPLRPVEPGRCTFMPDNPRAPLALPQQQDFRI | |
| | YQEVNNLRKIDPTSLLEVNLTLPERDRIVELLQRK | |
| | PALTFDAVRKALCFNGTFNLEGENRSELKGNLTNC | |
| | ALAKKKLFGESWYSFDAHKRFEIVEHLLQEESEEN | |
| | LVSWLQKECNLSEEYAKNVASVRLPAGYGALCQEA | |
| | LDLILPYLKAEVITYDKAVQKAGMNHSELTLAQET | |
| | GEILPELPYYGQYLKRHVGFGTGKPEDSAEKRYGK | |
| | IPNPTVHIALNQLRTVVNALIRRYGKPTQIVIELA | |
| | RELKQNKKAKDQYRIEMNHNQNRNERIRADISMIL | |
| | GINPENVKRKDIEKQILWEELNLKDATARCCPYSG | |
| | KQISAEMLFTDEVEIDHILPFSRTLDDSKNNKVVC | |
| | IREANRIKGNRTPWEARKDFEKRGWSVEAMTARAQ | |
| | AMPKAKRFRFAEDGYKVWLKDFDGFEARALTDTQY | |
| | MSRVAREYLQLICPGQTWSVPGQLTGMLRRFLGLN | |
| | DILGVNGEKNRDDHRHHAVDACVIALTDRSMLQRI | |
| | STASARAENKHLTRLLESFPAPWATFYEHVTRAVK | |
| | SICVSHKPEHAYQGAMNEQTAYGLRPDGYVKYRQN | |
| | GKVEHKKLNVIPQVSVKGTWRHGLNSDGSLKAYKG | |
| | LKGGSNFCIEIVMGEGGRWEGDVITTYEAYQIVRA | |
| | KGEAALYGSVSRSGKPLVMRLMQKDIVEMTLADGR | |
| | CKMLLYIITQNKQMFFYRIENAGGGREDVSKRPGS | |
| | LQKALAKKIIVSPIGDFRKEKL | |
| BNK Type II | MGKPIPNPLLGLDSTKRTADGSEFESPKKKRKVKM | 3 |
| Cas | QDSVSKMKYRLGIDLGTTSLGWAMLRLDEQNEPYA | |
| mammalian | VIRAGVRIFNNGRDPKTEASLAVARRLARQQRRTR | |
| expression | DRKIRRKERLIGELVDMGFFPKDPVKRRQLASLDP | |
| construct | FKLRTEALDRALSPEEFARAIFHLARRRGFKSNRK | |
| (includes N- | TDSGDTESSKMKEAIKRTLNELQNKGFRTVGEWLN | |
| terminal SV5 | MRHQQRLGTRSRIKNVPTGSGKQTTAYDFYLNRFM | |
| tag and NLS | IEYEFDRIWEKQSQMNPGLFTNERKAILKDIIFYQ | |
| and C- | RPLRPVEPGRCTFMPDNPRAPLALPQQQDFRIYQE | |
| terminal NLS) | VNNLRKIDPTSLLEVNLTLPERDRIVELLQRKPAL | |
| (aa) | TFDAVRKALCFNGTFNLEGENRSELKGNLTNCALA | |
| | KKKLFGESWYSFDAHKRFEIVEHLLQEESEENLVS | |
| | WLQKECNLSEEYAKNVASVRLPAGYGALCQEALDL | |
| | ILPYLKAEVITYDKAVQKAGMNHSELTLAQETGEI | |
| | LPELPYYGQYLKRHVGFGTGKPEDSAEKRYGKIPN | |
| | PTVHIALNQLRTVVNALIRRYGKPTQIVIELAREL | |
| | KQNKKAKDQYRIEMNHNQNRNERIRADISMILGIN | |
| | PENVKRKDIEKQILWEELNLKDATARCCPYSGKQI | |
| | SAEMLFTDEVEIDHILPFSRTLDDSKNNKVVCIRE | |
| | ANRIKGNRTPWEARKDFEKRGWSVEAMTARAQAMP | |
| | KAKRFRFAEDGYKVWLKDFDGFEARALTDTQYMSR | |
| | VAREYLQLICPGQTWSVPGQLTGMLRRFLGLNDIL | |
| | GVNGEKNRDDHRHHAVDACVIALTDRSMLQRISTA | |
| | SARAENKHLTRLLESFPAPWATFYEHVTRAVKSIC | |
| | VSHKPEHAYQGAMNEQTAYGLRPDGYVKYRQNGKV | |
| | EHKKLNVIPQVSVKGTWRHGLNSDGSLKAYKGLKG | |
| | GSNFCIEIVMGEGGRWEGDVITTYEAYQIVRAKGE | |
| | AALYGSVSRSGKPLVMRLMQKDIVEMTLADGRCKM | |
| | LLYIITQNKQMFFYRIENAGGGREDVSKRPGSLQK | |
| | ALAKKIIVSPIGDFRKEKLKRTADGSEFESPKKKR | |
| | KV | |
| BNK Type II | ATGAAAATGCAAGACTCTGTCTCAAAAATGAAATA | 4 |
| Cas coding | CAGACTCGGAATCGACCTTGGAACCACTTCCTTGG | |
| sequence (nt) | GCTGGGCTATGTTGCGGCTTGACGAACAAAACGAA | |
| (not codon | CCTTACGCCGTAATTCGAGCCGGGGTTCGTATCTT | |
| optimized) | TAATAACGGTCGAGACCCGAAAACGGAAGCCTCTT | |
| | TGGCCGTAGCGCGCCGTCTTGCACGTCAACAAAGA | |
| | AGGACTCGTGACAGAAAAATCAGGCGTAAAGAACG | |
| | CCTTATCGGGGAGCTGGTGGACATGGGGTTCTTCC | |
| | CGAAAGATCCCGTTAAACGGCGTCAACTCGCCTCG | |
| | TTAGATCCTTTTAAACTTCGAACTGAAGCCTTAGA | |
| | TAGGGCTCTTTCTCCGGAAGAATTTGCCAGAGCGA | |
| | TTTTCCATTTAGCCAGACGACGCGGCTTCAAAAGC | |
| | AATCGGAAAACAGATTCCGGCGATACCGAATCGAG | |
| | CAAGATGAAAGAGGCTATCAAACGCACTTTAAATG | |
| | AACTACAAAACAAGGGCTTTCGGACCGTTGGTGAG | |
| | TGGCTCAATATGCGCCATCAACAACGCCTCGGCAC | |
| | GCGTTCGCGCATTAAAAATGTCCCCACCGGTTCCG | |
| | GTAAGCAAACCACCGCATACGACTTCTACTTAAAT | |
| | CGATTCATGATTGAGTATGAATTTGATCGTATTTG | |
| | GGAAAAGCAGTCCCAAATGAATCCCGGCCTTTTCA | |
| | CCAATGAACGCAAGGCCATCTTAAAAGACATTATC | |
| | TTTTACCAAAGACCCCTTCGTCCTGTGGAACCCGG | |
| | ACGTTGCACCTTTATGCCGGACAATCCACGAGCAC | |
| | CGCTAGCTCTTCCTCAACAACAAGACTTTCGTATT | |
| | TATCAAGAAGTTAACAACTTGAGGAAAATTGACCC | |
| | AACCTCCCTGCTTGAAGTTAACCTCACGCTGCCCG | |
| | AAAGAGATCGAATCGTCGAATTGCTTCAACGAAAA | |
| | CCTGCTCTGACCTTTGATGCCGTTAGAAAGGCGCT | |
| | TTGCTTTAACGGAACATTTAATTTAGAAGGAGAAA | |
| | ATCGTTCCGAGTTAAAAGGCAATCTCACAAACTGT | |
| | GCGCTCGCTAAGAAAAAACTATTTGGAGAAAGTTG | |
| | GTATTCCTTCGATGCCCATAAACGATTTGAAATCG | |
| | TTGAACATCTTTTGCAAGAGGAATCCGAAGAAAAC | |
| | CTCGTTTCCTGGTTGCAAAAAGAATGCAATCTTTC | |
| | TGAAGAGTATGCAAAGAATGTCGCTTCCGTGCGAC | |
| | TCCCTGCAGGATACGGAGCCTTGTGTCAAGAGGCC | |
| | TTAGATTTAATCCTTCCATATCTGAAAGCTGAAGT | |
| | CATCACATACGACAAAGCGGTCCAAAAAGCCGGGA | |
| | TGAACCACAGCGAACTGACATTAGCACAAGAAACC | |
| | GGTGAAATCCTTCCAGAGCTTCCTTACTACGGTCA | |
| | GTATCTCAAACGTCACGTGGGTTTCGGGACCGGGA | |
| | AACCCGAGGATTCCGCAGAGAAGCGTTACGGAAAA | |
| | ATTCCCAACCCGACGGTACACATCGCCCTCAATCA | |
| | ATTGAGGACCGTTGTCAATGCTTTAATCCGCAGAT | |
| | ATGGAAAACCGACCCAGATTGTTATCGAGCTTGCT | |
| | CGAGAACTCAAACAAAACAAAAAGGCAAAAGATCA | |
| | ATATCGAATCGAGATGAATCACAACCAGAATCGGA | |
| | ATGAACGGATTCGCGCGGATATTTCGATGATTCTC | |
| | GGAATCAATCCAGAAAATGTGAAACGGAAAGATAT | |
| | TGAAAAACAGATTTTATGGGAAGAACTTAATCTGA | |
| | AGGACGCAACGGCTCGTTGTTGTCCATATAGCGGA | |
| | AAGCAAATCAGCGCAGAAATGCTTTTTACCGATGA | |
| | AGTTGAAATCGATCATATTCTCCCGTTTTCCAGGA | |
| | CTTTGGACGATTCAAAGAATAATAAAGTCGTTTGT | |
| | ATCCGAGAAGCCAACCGCATTAAAGGGAATCGAAC | |
| | CCCTTGGGAAGCACGAAAGGATTTTGAGAAGCGAG | |
| | GATGGTCGGTAGAAGCGATGACGGCACGCGCCCAA | |
| | GCAATGCCGAAAGCCAAGCGATTTCGCTTCGCCGA | |
| | GGACGGATATAAAGTTTGGTTAAAAGATTTCGATG | |
| | GCTTCGAGGCACGCGCGTTGACAGACACACAATAC | |
| | ATGAGTCGAGTCGCTCGCGAATATCTTCAGTTAAT | |
| | TTGCCCCGGTCAAACCTGGTCGGTTCCCGGACAAC | |
| | TCACCGGAATGTTAAGAAGATTTTTAGGGCTAAAT | |
| | GACATTTTGGGGGTTAATGGTGAGAAAAACCGCGA | |
| | TGACCACCGCCACCATGCCGTTGATGCCTGCGTGA | |
| | TTGCCCTCACGGATCGTTCCATGTTACAAAGGATT | |
| | TCGACGGCAAGTGCGAGGGCTGAAAATAAACATCT | |
| | TACTCGTCTGTTGGAATCTTTCCCGGCTCCATGGG | |
| | CTACGTTCTATGAGCATGTTACCCGCGCCGTGAAA | |
| | TCGATTTGTGTCAGTCACAAACCGGAGCACGCCTA | |
| | TCAAGGGGCCATGAACGAACAAACAGCCTACGGCT | |
| | TAAGACCGGACGGATATGTCAAATACAGACAAAAC | |
| | GGAAAAGTTGAACATAAGAAGTTAAATGTTATCCC | |
| | TCAGGTATCGGTCAAGGGAACCTGGAGACATGGTC | |
| | TTAACTCTGACGGATCATTAAAAGCGTACAAAGGA | |
| | CTAAAGGGAGGAAGCAATTTTTGTATTGAAATTGT | |
| | AATGGGAGAGGGCGGTCGCTGGGAAGGCGACGTTA | |
| | TTACAACATACGAGGCCTACCAAATCGTACGGGCG | |
| | AAAGGAGAAGCTGCGCTTTATGGGAGTGTGAGTCG | |
| | TTCCGGAAAACCGCTTGTAATGCGCTTGATGCAAA | |
| | AGGATATCGTTGAAATGACTCTTGCGGATGGCCGA | |
| | TGCAAAATGCTTCTTTACATAATCACCCAAAACAA | |
| | ACAAATGTTCTTTTACCGCATCGAAAATGCCGGCG | |
| | GTGGAAGAGAAGATGTTTCCAAGAGACCAGGATCC | |
| | TTACAAAAGGCACTTGCGAAAAAAATCATAGTCTC | |
| | TCCGATAGGGGATTTCCGTAAGGAAAAATTATGA | |
| BNK Type II | ATGAAGATGCAAGACAGCGTCAGCAAGATGAAATA | 5 |
| Cas coding | TAGACTCGGCATCGATCTCGGAACAACATCTCTGG | |
| sequence (nt) | GATGGGCCATGCTGAGACTGGACGAGCAGAACGAA | |
| (human | CCCTACGCCGTGATTAGGGCTGGAGTGAGAATTTT | |
| codon- | TAACAACGGAAGGGACCCCAAGACCGAAGCCTCTC | |
| optimized) | TGGCTGTGGCTAGGAGACTGGCCAGACAACAGAGA | |
| | AGGACAAGAGATAGAAAAATTAGAAGGAAGGAAAG | |
| | ACTCATCGGCGAGCTGGTCGACATGGGCTTCTTCC | |
| | CTAAAGACCCCGTGAAGAGGAGACAGCTGGCTTCT | |
| | CTGGACCCCTTCAAGCTCAGAACCGAGGCCCTCGA | |
| | TAGAGCTCTGAGCCCCGAGGAGTTCGCTAGAGCCA | |
| | TCTTCCATCTGGCTAGAAGGAGAGGCTTCAAGAGC | |
| | AATAGAAAGACAGACAGCGGCGACACCGAGAGCAG | |
| | CAAAATGAAGGAAGCCATTAAAAGGACACTGAACG | |
| | AGCTCCAAAACAAGGGATTTAGAACCGTGGGCGAG | |
| | TGGCTCAACATGAGACATCAGCAAAGGCTCGGCAC | |
| | AAGATCTAGAATCAAAAACGTGCCCACCGGATCCG | |
| | GAAAGCAGACCACAGCCTACGACTTCTATCTGAAT | |
| | AGATTCATGATTGAGTACGAGTTTGATAGAATCTG | |
| | GGAGAAACAGAGCCAGATGAACCCCGGACTGTTCA | |
| | CAAATGAAAGGAAAGCTATTCTGAAAGATATCATT | |
| | TTCTACCAAAGACCTCTCAGACCCGTGGAGCCCGG | |
| | AAGATGCACCTTCATGCCCGACAACCCCAGAGCCC | |
| | CTCTGGCTCTCCCCCAACAGCAAGACTTTAGAATC | |
| | TATCAAGAGGTGAATAATCTGAGAAAAATCGACCC | |
| | CACCTCTCTGCTGGAAGTCAATCTGACACTCCCCG | |
| | AAAGAGATAGAATCGTGGAGCTGCTGCAGAGAAAG | |
| | CCCGCTCTGACCTTCGACGCCGTCAGAAAGGCCCT | |
| | CTGCTTCAATGGCACCTTCAACCTCGAGGGAGAGA | |
| | ATAGAAGCGAACTCAAGGGCAACCTCACCAATTGC | |
| | GCCCTCGCTAAGAAGAAGCTCTTTGGCGAGAGCTG | |
| | GTATAGCTTCGACGCCCACAAGAGGTTCGAAATCG | |
| | TGGAACATCTGCTGCAAGAGGAGAGCGAAGAGAAT | |
| | CTGGTGAGCTGGCTGCAGAAAGAGTGCAATCTCAG | |
| | CGAGGAGTACGCCAAGAATGTCGCTAGCGTGAGAC | |
| | TGCCCGCCGGATACGGCGCTCTCTGCCAAGAAGCC | |
| | CTCGATCTCATCCTCCCCTACCTCAAGGCCGAGGT | |
| | GATCACCTACGATAAAGCCGTGCAGAAAGCCGGCA | |
| | TGAACCACTCCGAGCTCACACTGGCCCAAGAAACC | |
| | GGCGAAATCCTCCCCGAGCTGCCCTATTATGGCCA | |
| | ATACCTCAAGAGGCACGTCGGCTTTGGAACCGGCA | |
| | AGCCCGAAGATAGCGCTGAGAAGAGATATGGCAAG | |
| | ATCCCCAATCCCACAGTCCATATTGCTCTGAACCA | |
| | GCTGAGAACAGTGGTGAATGCCCTCATCAGAAGGT | |
| | ATGGAAAGCCCACACAAATCGTGATCGAACTCGCT | |
| | AGGGAACTGAAGCAGAACAAGAAGGCCAAGGATCA | |
| | GTATAGGATCGAAATGAATCACAATCAGAACAGAA | |
| | ACGAGAGGATTAGAGCCGACATCAGCATGATTCTG | |
| | GGCATCAATCCCGAGAACGTGAAGAGGAAGGACAT | |
| | CGAGAAGCAAATTCTGTGGGAGGAGCTGAATCTGA | |
| | AAGACGCCACCGCTAGATGCTGCCCTTACAGCGGA | |
| | AAACAGATTTCCGCCGAAATGCTCTTCACAGACGA | |
| | AGTGGAGATCGACCACATTCTGCCCTTCAGCAGAA | |
| | CACTGGACGACAGCAAGAATAACAAGGTGGTGTGC | |
| | ATTAGGGAGGCCAACAGAATCAAGGGCAACAGAAC | |
| | CCCTTGGGAGGCCAGAAAAGACTTCGAGAAAAGGG | |
| | GATGGAGCGTGGAGGCTATGACAGCTAGGGCCCAA | |
| | GCCATGCCCAAGGCCAAGAGATTCAGATTCGCCGA | |
| | GGATGGCTACAAGGTGTGGCTGAAGGACTTTGATG | |
| | GATTCGAAGCCAGAGCTCTGACCGACACCCAGTAC | |
| | ATGTCTAGAGTCGCCAGAGAGTATCTGCAACTGAT | |
| | CTGCCCCGGCCAGACATGGTCCGTGCCCGGCCAGC | |
| | TGACCGGCATGCTGAGAAGATTTCTGGGACTGAAC | |
| | GACATCCTCGGAGTCAACGGCGAGAAGAATAGAGA | |
| | TGACCACAGACATCACGCCGTGGACGCTTGCGTGA | |
| | TTGCTCTCACAGATAGAAGCATGCTGCAAAGAATC | |
| | TCCACCGCCAGCGCTAGAGCTGAGAACAAGCACCT | |
| | CACAAGACTGCTGGAGTCCTTCCCCGCCCCTTGGG | |
| | CCACCTTCTATGAACACGTGACCAGAGCCGTGAAG | |
| | AGCATCTGCGTGTCCCATAAACCCGAGCACGCCTA | |
| | CCAAGGCGCTATGAACGAGCAGACAGCCTACGGCC | |
| | TCAGACCCGACGGATATGTGAAGTATAGGCAGAAC | |
| | GGCAAGGTCGAACACAAGAAGCTGAACGTGATCCC | |
| | CCAAGTGTCCGTCAAAGGAACATGGAGGCATGGAC | |
| | TGAATTCCGACGGCTCTCTGAAAGCTTACAAGGGA | |
| | CTGAAAGGCGGATCCAACTTCTGCATCGAGATCGT | |
| | GATGGGCGAGGGAGGAAGATGGGAGGGAGATGTGA | |
| | TCACCACCTACGAGGCCTACCAGATTGTGAGAGCC | |
| | AAAGGAGAGGCTGCTCTCTACGGCTCCGTCTCTAG | |
| | AAGCGGAAAGCCCCTCGTCATGAGGCTCATGCAGA | |
| | AGGATATCGTCGAGATGACACTGGCCGACGGCAGA | |
| | TGCAAGATGCTGCTGTACATCATCACCCAGAATAA | |
| | ACAGATGTTCTTTTATAGAATTGAGAACGCCGGCG | |
| | GAGGAAGAGAAGATGTCAGCAAAAGACCCGGCAGC | |
| | CTCCAGAAAGCTCTGGCCAAGAAGATTATCGTGAG | |
| | CCCCATCGGCGACTTTAGAAAGGAGAAGCTGTGA | |
| BNK Type II | ATGGGAAAACCTATCCCTAACCCTCTGCTGGGACT | 6 |
| Cas | CGATAGCACAAAGAGAACCGCCGATGGAAGCGAGT | |
| mammalian | TCGAGTCCCCTAAGAAGAAGAGGAAAGTCAAGATG | |
| expression | CAAGACAGCGTCAGCAAGATGAAATATAGACTCGG | |
| construct | CATCGATCTCGGAACAACATCTCTGGGATGGGCCA | |
| (includes N- | TGCTGAGACTGGACGAGCAGAACGAACCCTACGCC | |
| terminal SV5 | GTGATTAGGGCTGGAGTGAGAATTTTTAACAACGG | |
| tag and NLS | AAGGGACCCCAAGACCGAAGCCTCTCTGGCTGTGG | |
| and C- | CTAGGAGACTGGCCAGACAACAGAGAAGGACAAGA | |
| terminal NLS) | GATAGAAAAATTAGAAGGAAGGAAAGACTCATCGG | |
| (nt) | CGAGCTGGTCGACATGGGCTTCTTCCCTAAAGACC | |
| | CCGTGAAGAGGAGACAGCTGGCTTCTCTGGACCCC | |
| | TTCAAGCTCAGAACCGAGGCCCTCGATAGAGCTCT | |
| | GAGCCCCGAGGAGTTCGCTAGAGCCATCTTCCATC | |
| | TGGCTAGAAGGAGAGGCTTCAAGAGCAATAGAAAG | |
| | ACAGACAGCGGCGACACCGAGAGCAGCAAAATGAA | |
| | GGAAGCCATTAAAAGGACACTGAACGAGCTCCAAA | |
| | ACAAGGGATTTAGAACCGTGGGCGAGTGGCTCAAC | |
| | ATGAGACATCAGCAAAGGCTCGGCACAAGATCTAG | |
| | AATCAAAAACGTGCCCACCGGATCCGGAAAGCAGA | |
| | CCACAGCCTACGACTTCTATCTGAATAGATTCATG | |
| | ATTGAGTACGAGTTTGATAGAATCTGGGAGAAACA | |
| | GAGCCAGATGAACCCCGGACTGTTCACAAATGAAA | |
| | GGAAAGCTATTCTGAAAGATATCATTTTCTACCAA | |
| | AGACCTCTCAGACCCGTGGAGCCCGGAAGATGCAC | |
| | CTTCATGCCCGACAACCCCAGAGCCCCTCTGGCTC | |
| | TCCCCCAACAGCAAGACTTTAGAATCTATCAAGAG | |
| | GTGAATAATCTGAGAAAAATCGACCCCACCTCTCT | |
| | GCTGGAAGTCAATCTGACACTCCCCGAAAGAGATA | |
| | GAATCGTGGAGCTGCTGCAGAGAAAGCCCGCTCTG | |
| | ACCTTCGACGCCGTCAGAAAGGCCCTCTGCTTCAA | |
| | TGGCACCTTCAACCTCGAGGGAGAGAATAGAAGCG | |
| | AACTCAAGGGCAACCTCACCAATTGCGCCCTCGCT | |
| | AAGAAGAAGCTCTTTGGCGAGAGCTGGTATAGCTT | |
| | CGACGCCCACAAGAGGTTCGAAATCGTGGAACATC | |
| | TGCTGCAAGAGGAGAGCGAAGAGAATCTGGTGAGC | |
| | TGGCTGCAGAAAGAGTGCAATCTCAGCGAGGAGTA | |
| | CGCCAAGAATGTCGCTAGCGTGAGACTGCCCGCCG | |
| | GATACGGCGCTCTCTGCCAAGAAGCCCTCGATCTC | |
| | ATCCTCCCCTACCTCAAGGCCGAGGTGATCACCTA | |
| | CGATAAAGCCGTGCAGAAAGCCGGCATGAACCACT | |
| | CCGAGCTCACACTGGCCCAAGAAACCGGCGAAATC | |
| | CTCCCCGAGCTGCCCTATTATGGCCAATACCTCAA | |
| | GAGGCACGTCGGCTTTGGAACCGGCAAGCCCGAAG | |
| | ATAGCGCTGAGAAGAGATATGGCAAGATCCCCAAT | |
| | CCCACAGTCCATATTGCTCTGAACCAGCTGAGAAC | |
| | AGTGGTGAATGCCCTCATCAGAAGGTATGGAAAGC | |
| | CCACACAAATCGTGATCGAACTCGCTAGGGAACTG | |
| | AAGCAGAACAAGAAGGCCAAGGATCAGTATAGGAT | |
| | CGAAATGAATCACAATCAGAACAGAAACGAGAGGA | |
| | TTAGAGCCGACATCAGCATGATTCTGGGCATCAAT | |
| | CCCGAGAACGTGAAGAGGAAGGACATCGAGAAGCA | |
| | AATTCTGTGGGAGGAGCTGAATCTGAAAGACGCCA | |
| | CCGCTAGATGCTGCCCTTACAGCGGAAAACAGATT | |
| | TCCGCCGAAATGCTCTTCACAGACGAAGTGGAGAT | |
| | CGACCACATTCTGCCCTTCAGCAGAACACTGGACG | |
| | ACAGCAAGAATAACAAGGTGGTGTGCATTAGGGAG | |
| | GCCAACAGAATCAAGGGCAACAGAACCCCTTGGGA | |
| | GGCCAGAAAAGACTTCGAGAAAAGGGGATGGAGCG | |
| | TGGAGGCTATGACAGCTAGGGCCCAAGCCATGCCC | |
| | AAGGCCAAGAGATTCAGATTCGCCGAGGATGGCTA | |
| | CAAGGTGTGGCTGAAGGACTTTGATGGATTCGAAG | |
| | CCAGAGCTCTGACCGACACCCAGTACATGTCTAGA | |
| | GTCGCCAGAGAGTATCTGCAACTGATCTGCCCCGG | |
| | CCAGACATGGTCCGTGCCCGGCCAGCTGACCGGCA | |
| | TGCTGAGAAGATTTCTGGGACTGAACGACATCCTC | |
| | GGAGTCAACGGCGAGAAGAATAGAGATGACCACAG | |
| | ACATCACGCCGTGGACGCTTGCGTGATTGCTCTCA | |
| | CAGATAGAAGCATGCTGCAAAGAATCTCCACCGCC | |
| | AGCGCTAGAGCTGAGAACAAGCACCTCACAAGACT | |
| | GCTGGAGTCCTTCCCCGCCCCTTGGGCCACCTTCT | |
| | ATGAACACGTGACCAGAGCCGTGAAGAGCATCTGC | |
| | GTGTCCCATAAACCCGAGCACGCCTACCAAGGCGC | |
| | TATGAACGAGCAGACAGCCTACGGCCTCAGACCCG | |
| | ACGGATATGTGAAGTATAGGCAGAACGGCAAGGTC | |
| | GAACACAAGAAGCTGAACGTGATCCCCCAAGTGTC | |
| | CGTCAAAGGAACATGGAGGCATGGACTGAATTCCG | |
| | ACGGCTCTCTGAAAGCTTACAAGGGACTGAAAGGC | |
| | GGATCCAACTTCTGCATCGAGATCGTGATGGGCGA | |
| | GGGAGGAAGATGGGAGGGAGATGTGATCACCACCT | |
| | ACGAGGCCTACCAGATTGTGAGAGCCAAAGGAGAG | |
| | GCTGCTCTCTACGGCTCCGTCTCTAGAAGCGGAAA | |
| | GCCCCTCGTCATGAGGCTCATGCAGAAGGATATCG | |
| | TCGAGATGACACTGGCCGACGGCAGATGCAAGATG | |
| | CTGCTGTACATCATCACCCAGAATAAACAGATGTT | |
| | CTTTTATAGAATTGAGAACGCCGGCGGAGGAAGAG | |
| | AAGATGTCAGCAAAAGACCCGGCAGCCTCCAGAAA | |
| | GCTCTGGCCAAGAAGATTATCGTGAGCCCCATCGG | |
| | CGACTTTAGAAAGGAGAAGCTGAAGAGAACCGCTG | |
| | ACGGCAGCGAATTCGAAAGCCCCAAAAAGAAGAGA | |
| | AAGGTGTGA | |

In some embodiments a BNK Type II Cas protein comprises an amino acid sequence of SEQ ID NO:1, SEQ ID NO:2, or SEQ ID NO:3. In some embodiments, a BNK Type II Cas protein has nickase activity, for example resulting from one or more amino acid substitutions relative to the sequence of SEQ ID NO:1, SEQ ID NO:2, or SEQ ID NO:3. In some embodiments, the one or more amino acid substitutions providing nickase activity is a D23A substitution, wherein the position of the D23A substitution is defined with respect to the amino acid numbering of SEQ ID NO:8. The corresponding position in SEQ ID NO:1, SEQ ID NO:2, or SEQ ID NO:3 can be determined, for example, by performing a sequence alignment of SEQ ID NO:1, SEQ ID NO:2, or SEQ ID NO:3 with SEQ ID NO:8 (e.g., by BLAST).

### 6.2.2. AIK Type II Cas Proteins

In one aspect, the disclosure provides AIK Type II Cas proteins. The AIK Type II Cas proteins typically comprise an amino acid sequence that is at least 50%, at least 55%, at least 60%, at least 65%, at least 70%, at least 75%, at least 85%, at least 90%, or at least 95% identical to SEQ ID NO:7. In some embodiments, the AIK Type II Cas proteins comprise an amino acid sequence that is at least 95%, at least 96%, at least 97%, at least 98%, or at least 99% identical to SEQ ID NO:7. In some embodiments, an AIK Type II Cas protein comprises an amino acid sequence that is identical to SEQ ID NO:7.

Exemplary AIK Type II Cas protein sequences and nucleotide sequences encoding exemplary AIK Type II Cas proteins are set forth in Table 1B.

TABLE 1B
AIK Type II Cas Sequences

| | | SEQ ID |
|---|---|---|
| Name | Sequence | NO: |
| AIK Type II | EITINREIGKLGLPRHLVLGMDPGIASCGFALIDT | 7 |
| Cas coding | ANREILDLGVRLFDSPTHPKTGQSLAVIRRGFRST | |
| sequence | RRNIDRTQARLKHCLQILKAYGLIPQDATKEYFHT | |
| (aa) (without | TKGDKQPLKLRVDGLDRLLNDREWALVLYSLCKRR | |
| N-terminal | GYIPHGEGNQDKSSEGGKVLSALAANKEAIAETSC | |
| methionine) | RTVGEWLAQQPQSRNRGGNYDKCVTHAQLIEETHI | |
| | LFDAQRSFGSKYASPEFEAAYIEVCDWERSRKDFD | |
| | RRTYDLVGHCSYFPTEKRAARCTLTSELVSAYGAL | |
| | GNITIIHDDGTSRALSATERDECIAILFSCEPIRG | |
| | NKDCAVKFGALRKALDLSSGDYFKGVPAADEKTRE | |
| | VYKPKGWRVLRNTLNAANPILLQRLRDDRNLADAV | |
| | MEAVAYSSALPVLQEQLQGLPLSEAEIEALCRLPY | |
| | SSKALNGYGNRSKKALDMLLDCLEEPEVLNLTQAE | |
| | NDCGLLGLRIAGTQLERSDRLMPYETWIERTGRTN | |
| | NNPVVIRAMSQMRKVVNAICRKWGVPNEIHVELDR | |
| | ELRLPQRAKDEIAKANKKNEKNRERIAGQIAELRG | |
| | CTADEVTGKQIEKYRLWEEQECFDLYTGAKIEVDR | |
| | LISDDTYTQIDHILPFSRTGENSRNNKVLVLAKSN | |
| | QDKREQTPYEWMSHDGAPSWDAFERRVQENQKLSR | |
| | RKKNFLLEKDLDTKEGEFLARSFTDTAYMSREVCA | |
| | YLADCLLFPDDGAKAHVVPTTGRATAWLRRRWGLN | |
| | FGSNGEKDRSDDRHHATDACVIAACSRSLVIKTAR | |
| | INQETHWSITRGMNETQRRDAIMKALESVMPWETF | |
| | ANEVRAAHDFVVPTRFVPRKGKGELFEQTVYRYAG | |
| | VNAQGKDIARKASSDKDIVMGNAVVSADEKSVIKV | |
| | SEMLCLRLWHDPEAKKGQGAWYADPVYKADIPALK | |
| | DGTYVPRIAKQKYGRKVWKAVPNSALTQKPLEIYL | |
| | GDLIKVGDKLGRYNGYNIATANWSFVDALTKKEIA | |
| | FPSVGMLSNELQPIIIRESILDN | |
| AIK Type II | MEITINREIGKLGLPRHLVLGMDPGIASCGFALID | 8 |
| Cas coding | TANREILDLGVRLFDSPTHPKTGQSLAVIRRGFRS | |
| sequence | TRRNIDRTQARLKHCLQILKAYGLIPQDATKEYFH | |
| (aa) | TTKGDKQPLKLRVDGLDRLLNDREWALVLYSLCKR | |
| | RGYIPHGEGNQDKSSEGGKVLSALAANKEAIAETS | |
| | CRTVGEWLAQQPQSRNRGGNYDKCVTHAQLIEETH | |
| | ILFDAQRSFGSKYASPEFEAAYIEVCDWERSRKDF | |
| | DRRTYDLVGHCSYFPTEKRAARCTLTSELVSAYGA | |
| | LGNITIIHDDGTSRALSATERDECIAILFSCEPIR | |
| | GNKDCAVKFGALRKALDLSSGDYFKGVPAADEKTR | |
| | EVYKPKGWRVLRNTLNAANPILLQRLRDDRNLADA | |
| | VMEAVAYSSALPVLQEQLQGLPLSEAEIEALCRLP | |
| | YSSKALNGYGNRSKKALDMLLDCLEEPEVLNLTQA | |
| | ENDCGLLGLRIAGTQLERSDRLMPYETWIERTGRT | |
| | NNNPVVIRAMSQMRKVVNAICRKWGVPNEIHVELD | |
| | RELRLPQRAKDEIAKANKKNEKNRERIAGQIAELR | |
| | GCTADEVTGKQIEKYRLWEEQECFDLYTGAKIEVD | |
| | RLISDDTYTQIDHILPFSRTGENSRNNKVLVLAKS | |
| | NQDKREQTPYEWMSHDGAPSWDAFERRVQENQKLS | |
| | RRKKNFLLEKDLDTKEGEFLARSFTDTAYMSREVC | |
| | AYLADCLLFPDDGAKAHVVPTTGRATAWLRRRWGL | |
| | NFGSNGEKDRSDDRHHATDACVIAACSRSLVIKTA | |
| | RINQETHWSITRGMNETQRRDAIMKALESVMPWET | |
| | FANEVRAAHDFVVPTRFVPRKGKGELFEQTVYRYA | |
| | GVNAQGKDIARKASSDKDIVMGNAVVSADEKSVIK | |
| | VSEMLCLRLWHDPEAKKGQGAWYADPVYKADIPAL | |
| | KDGTYVPRIAKQKYGRKVWKAVPNSALTQKPLEIY | |
| | LGDLIKVGDKLGRYNGYNIATANWSFVDALTKKEI | |
| | AFPSVGMLSNELQPIIIRESILDN | |
| AIK Type II | MGKPIPNPLLGLDSTKRTADGSEFESPKKKRKVEI | 9 |
| Cas | TINREIGKLGLPRHLVLGMDPGIASCGFALIDTAN | |
| mammalian | REILDLGVRLFDSPTHPKTGQSLAVIRRGFRSTRR | |
| expression | NIDRTQARLKHCLQILKAYGLIPQDATKEYFHTTK | |
| construct | GDKQPLKLRVDGLDRLLNDREWALVLYSLCKRRGY | |
| (includes N- | IPHGEGNQDKSSEGGKVLSALAANKEAIAETSCRT | |
| terminal SV5 | VGEWLAQQPQSRNRGGNYDKCVTHAQLIEETHILF | |
| tag and NLS | DAQRSFGSKYASPEFEAAYIEVCDWERSRKDFDRR | |
| and C- | TYDLVGHCSYFPTEKRAARCTLTSELVSAYGALGN | |
| terminal NLS) | ITIIHDDGTSRALSATERDECIAILFSCEPIRGNK | |
| (aa) | DCAVKFGALRKALDLSSGDYFKGVPAADEKTREVY | |
| | KPKGWRVLRNTLNAANPILLQRLRDDRNLADAVME | |
| | AVAYSSALPVLQEQLQGLPLSEAEIEALCRLPYSS | |
| | KALNGYGNRSKKALDMLLDCLEEPEVLNLTQAEND | |
| | CGLLGLRIAGTQLERSDRLMPYETWIERTGRTNNN | |
| | PVVIRAMSQMRKVVNAICRKWGVPNEIHVELDREL | |
| | RLPQRAKDEIAKANKKNEKNRERIAGQIAELRGCT | |
| | ADEVTGKQIEKYRLWEEQECFDLYTGAKIEVDRLI | |
| | SDDTYTQIDHILPFSRTGENSRNNKVLVLAKSNQD | |
| | KREQTPYEWMSHDGAPSWDAFERRVQENQKLSRRK | |
| | KNFLLEKDLDTKEGEFLARSFTDTAYMSREVCAYL | |
| | ADCLLFPDDGAKAHVVPTTGRATAWLRRRWGLNFG | |
| | SNGEKDRSDDRHHATDACVIAACSRSLVIKTARIN | |
| | QETHWSITRGMNETQRRDAIMKALESVMPWETFAN | |
| | EVRAAHDFVVPTRFVPRKGKGELFEQTVYRYAGVN | |
| | AQGKDIARKASSDKDIVMGNAVVSADEKSVIKVSE | |
| | MLCLRLWHDPEAKKGQGAWYADPVYKADIPALKDG | |
| | TYVPRIAKQKYGRKVWKAVPNSALTQKPLEIYLGD | |
| | LIKVGDKLGRYNGYNIATANWSFVDALTKKEIAFP | |
| | SVGMLSNELQPIIIRESILDNKRTADGSEFESPKK | |
| | KRKV | |
| AIK Type II | ATGGAGATCACCATCAATCGCGAAATTGGGAAGCT | 10 |
| Cas coding | CGGACTTCCCAGGCATCTTGTGCTTGGCATGGATC | |
| sequence (nt) | CAGGAATTGCAAGCTGCGGATTCGCACTTATCGAC | |
| not codon | ACAGCCAATCGTGAAATCCTGGATTTGGGCGTCAG | |
| optimized | ATTATTTGACTCTCCAACTCATCCTAAAACGGGCC | |
| | AAAGTCTTGCGGTTATTCGCAGGGGCTTCCGCTCT | |
| | ACCCGTCGAAACATTGACCGTACCCAGGCGCGCTT | |
| | GAAGCACTGTCTCCAAATCCTCAAGGCTTATGGCC | |
| | TCATCCCCCAAGACGCCACCAAAGAGTACTTCCAC | |
| | ACCACAAAAGGCGACAAGCAGCCGCTCAAGCTTCG | |
| | CGTTGATGGGCTTGACCGCCTGCTCAACGATCGCG | |
| | AGTGGGCGCTAGTCCTATACTCCCTCTGCAAGCGC | |
| | CGTGGATACATCCCCCACGGAGAAGGCAATCAGGA | |
| | TAAATCAAGCGAAGGCGGCAAGGTTCTATCTGCCC | |
| | TTGCGGCCAACAAGGAAGCAATTGCGGAGACCTCG | |
| | TGCCGCACCGTTGGCGAATGGCTCGCTCAGCAGCC | |
| | TCAAAGTCGCAATCGTGGCGGCAATTACGACAAGT | |
| | GTGTAACGCACGCCCAGCTTATCGAAGAAACTCAT | |
| | ATCCTATTTGATGCTCAACGCTCCTTTGGCTCCAA | |
| | ATACGCTTCGCCGGAATTTGAGGCCGCATATATCG | |
| | AGGTTTGCGATTGGGAGCGTTCGCGCAAAGACTTC | |
| | GACCGCCGCACGTACGACCTCGTTGGACACTGCTC | |
| | ATACTTCCCAACAGAAAAACGAGCCGCACGCTGCA | |
| | CGCTTACGAGCGAACTTGTTTCAGCCTATGGCGCA | |
| | CTCGGCAACATCACCATCATCCACGATGACGGGAC | |
| | CTCTCGCGCCTTGAGCGCAACGGAGCGCGATGAAT | |
| | GCATTGCAATCCTGTTCTCGTGCGAGCCAATTCGA | |
| | GGCAACAAAGATTGTGCTGTTAAATTCGGCGCCCT | |
| | CAGAAAAGCGCTCGACCTTAGTTCAGGCGATTACT | |
| | TCAAAGGGGTTCCAGCTGCCGACGAAAAAACGCGC | |
| | GAGGTGTACAAGCCCAAGGGATGGCGCGTGCTCCG | |
| | CAATACCCTCAATGCAGCCAACCCCATTCTCCTGC | |
| | AGCGTTTACGCGATGACCGCAATCTCGCCGATGCC | |
| | GTTATGGAGGCGGTAGCATATTCCTCGGCCCTTCC | |
| | CGTACTCCAAGAGCAGCTTCAGGGGTTACCGCTCT | |
| | CGGAAGCGGAGATCGAGGCGCTTTGTAGGCTTCCC | |
| | TATTCATCCAAAGCTCTTAACGGCTATGGCAACCG | |
| | TTCCAAAAAAGCACTCGACATGCTGCTCGATTGCC | |
| | TCGAGGAGCCTGAGGTCCTCAACCTTACGCAGGCC | |
| | GAAAATGACTGCGGTCTGCTGGGACTGCGCATCGC | |
| | TGGCACCCAGCTCGAGCGCTCCGATCGCCTGATGC | |
| | CCTATGAGACCTGGATCGAACGTACCGGTCGAACA | |
| | AATAACAATCCCGTCGTCATTCGCGCCATGTCGCA | |
| | AATGCGAAAAGTGGTCAACGCCATCTGCCGCAAGT | |
| | GGGGCGTGCCAAACGAAATCCACGTTGAGCTTGAT | |
| | CGAGAGCTCAGGTTGCCTCAGCGCGCAAAAGACGA | |
| | GATTGCCAAGGCCAATAAGAAGAACGAGAAAAATC | |
| | GCGAGCGCATTGCCGGGCAAATCGCTGAGCTGCGT | |
| | GGCTGCACGGCAGATGAGGTCACGGGCAAACAGAT | |
| | AGAGAAGTACCGCCTGTGGGAAGAGCAGGAATGCT | |
| | TCGACCTTTACACGGGCGCTAAAATCGAAGTCGAT | |
| | CGCCTAATTAGCGACGACACCTACACGCAGATTGA | |
| | CCACATCCTGCCGTTCTCTCGCACGGGAGAAAACT | |
| | CGCGCAACAATAAAGTCCTGGTCCTCGCCAAAAGC | |
| | AATCAGGATAAACGCGAACAGACACCTTACGAATG | |
| | GATGTCCCACGACGGTGCGCCTTCATGGGATGCTT | |
| | TTGAGCGTCGCGTTCAGGAAAACCAGAAACTCAGC | |
| | CGTCGCAAAAAGAACTTCCTGCTGGAAAAAGACCT | |
| | TGATACCAAGGAAGGCGAATTCTTAGCACGCAGCT | |
| | TCACCGACACCGCCTATATGTCGCGAGAAGTATGC | |
| | GCTTACCTCGCCGACTGCCTGCTGTTCCCCGATGA | |
| | TGGCGCAAAGGCACATGTTGTTCCTACCACTGGCA | |
| | GAGCGACCGCATGGCTGCGTCGCAGGTGGGGGCTT | |
| | AACTTTGGTTCGAATGGCGAAAAAGACCGCTCGGA | |
| | CGATCGTCACCATGCCACCGATGCTTGTGTGATTG | |
| | CAGCATGTAGTCGAAGCCTCGTGATTAAAACCGCT | |
| | CGAATCAACCAAGAGACACACTGGAGCATAACCAG | |
| | AGGTATGAACGAGACCCAACGCCGCGATGCCATCA | |
| | TGAAGGCTCTCGAAAGTGTTATGCCCTGGGAAACC | |
| | TTTGCGAACGAAGTACGCGCGGCGCACGATTTCGT | |
| | CGTACCCACCCGCTTTGTTCCGCGTAAGGGAAAGG | |
| | GCGAGTTGTTCGAGCAGACGGTCTATCGCTATGCC | |
| | GGCGTTAATGCACAGGGCAAAGACATTGCTCGCAA | |
| | GGCGAGCTCCGATAAGGACATCGTCATGGGCAACG | |
| | CCGTTGTGTCGGCAGACGAGAAGTCGGTCATCAAG | |
| | GTGAGCGAAATGCTGTGTCTGAGGCTCTGGCATGA | |
| | CCCGGAGGCCAAGAAGGGGCAGGGCGCTTGGTACG | |
| | CAGACCCCGTCTATAAGGCGGATATTCCTGCACTT | |
| | AAGGATGGGACGTATGTGCCCAGGATTGCGAAGCA | |
| | AAAATATGGGCGGAAGGTCTGGAAAGCCGTTCCCA | |
| | ATAGCGCTTTAACTCAAAAACCACTCGAAATATAT | |
| | CTGGGTGACCTCATTAAAGTCGGGGATAAGCTCGG | |
| | TCGCTACAATGGCTACAACATTGCAACGGCCAATT | |
| | GGTCCTTTGTTGACGCTCTCACGAAAAAGGAGATT | |
| | GCATTCCCCTCGGTTGGAATGCTCTCAAACGAATT | |
| | GCAACCGATAATTATTCGCGAGAGCATTCTCGATA | |
| | ATTAA | |
| AIK Type II | ATGGAGATCACAATTAATAGGGAAATTGGCAAGCT | 11 |
| Cas coding | GGGACTCCCTAGACATCTGGTGCTGGGCATGGATC | |
| sequence (nt) | CCGGCATTGCCAGCTGCGGATTCGCTCTGATCGAC | |
| codon | ACAGCCAATAGAGAAATTCTGGATCTGGGCGTGAG | |
| optimized | GCTGTTCGATTCCCCTACCCATCCTAAGACCGGCC | |
| | AGTCTCTGGCTGTCATCAGAAGGGGCTTCAGATCC | |
| | ACAAGAAGGAATATCGACAGAACCCAAGCTAGACT | |
| | CAAGCACTGCCTCCAGATCCTCAAAGCCTATGGCC | |
| | TCATTCCCCAAGACGCCACCAAAGAGTACTTCCAC | |
| | ACCACCAAGGGAGATAAGCAGCCTCTGAAACTGAG | |
| | AGTGGACGGACTGGATAGACTGCTGAACGATAGAG | |
| | AATGGGCTCTGGTGCTGTACAGCCTCTGTAAGAGA | |
| | AGGGGCTACATCCCTCACGGCGAGGGAAATCAAGA | |
| | CAAGTCCAGCGAAGGAGGCAAAGTGCTGAGCGCCC | |
| | TCGCCGCCAACAAGGAAGCTATCGCCGAGACAAGC | |
| | TGTAGAACCGTGGGCGAATGGCTCGCTCAACAGCC | |
| | TCAGAGCAGAAATAGAGGCGGAAACTATGACAAGT | |
| | GCGTCACCCATGCTCAGCTCATTGAGGAAACCCAT | |
| | ATCCTCTTCGACGCTCAGAGAAGCTTTGGCAGCAA | |
| | GTACGCCAGCCCCGAGTTCGAAGCCGCCTACATTG | |
| | AAGTGTGTGACTGGGAAAGGTCTAGAAAGGATTTT | |
| | GATAGAAGGACATACGATCTGGTCGGCCACTGCAG | |
| | CTACTTCCCTACCGAAAAGAGAGCCGCTAGATGCA | |
| | CACTGACCAGCGAGCTGGTCAGCGCCTACGGAGCT | |
| | CTCGGCAACATCACCATCATCCACGACGACGGAAC | |
| | CAGCAGAGCTCTGAGCGCCACCGAAAGGGATGAGT | |
| | GCATCGCCATCCTCTTTAGCTGCGAGCCCATTAGA | |
| | GGCAATAAGGATTGTGCCGTGAAGTTCGGAGCTCT | |
| | GAGAAAGGCTCTGGACCTCTCCTCCGGAGATTACT | |
| | TCAAGGGAGTCCCCGCCGCCGACGAAAAGACCAGA | |
| | GAGGTCTACAAGCCCAAGGGCTGGAGAGTGCTGAG | |
| | AAACACCCTCAACGCCGCCAACCCTATTCTGCTCC | |
| | AGAGACTGAGAGATGACAGAAACCTCGCTGACGCT | |
| | GTGATGGAAGCTGTCGCTTACAGCAGCGCTCTGCC | |
| | CGTGCTCCAAGAGCAGCTGCAAGGACTGCCTCTCT | |
| | CCGAGGCTGAGATCGAGGCTCTGTGCAGACTGCCT | |
| | TACAGCTCCAAAGCTCTGAACGGCTACGGCAATAG | |
| | AAGCAAAAAAGCTCTGGACATGCTGCTCGATTGTC | |
| | TGGAGGAACCCGAAGTGCTGAACCTCACCCAAGCC | |
| | GAAAACGATTGTGGACTGCTCGGACTGAGAATCGC | |
| | CGGAACCCAGCTGGAGAGATCCGATAGACTCATGC | |
| | CTTATGAAACATGGATCGAGAGAACCGGAAGAACC | |
| | AATAACAACCCCGTGGTCATCAGAGCCATGAGCCA | |
| | AATGAGAAAGGTGGTCAACGCCATCTGCAGAAAGT | |
| | GGGGCGTGCCCAACGAAATTCATGTGGAGCTGGAT | |
| | AGAGAGCTGAGACTGCCCCAAAGGGCTAAGGACGA | |
| | GATCGCCAAGGCTAATAAGAAGAATGAAAAGAACA | |
| | GAGAGAGAATCGCCGGCCAGATTGCTGAACTGAGA | |
| | GGCTGTACAGCTGACGAGGTGACCGGCAAGCAGAT | |
| | TGAGAAGTATAGACTCTGGGAGGAGCAAGAGTGCT | |
| | TTGATCTGTACACCGGAGCCAAGATCGAGGTGGAT | |
| | AGGCTCATCAGCGATGATACATACACCCAGATCGA | |
| | CCACATTCTGCCTTTTAGCAGAACCGGCGAGAACT | |
| | CTAGAAACAACAAAGTGCTGGTGCTCGCTAAGTCC | |
| | AATCAAGACAAGAGGGAGCAGACCCCCTATGAATG | |
| | GATGAGCCATGATGGCGCTCCCAGCTGGGACGCCT | |
| | TCGAAAGGAGGGTGCAAGAGAACCAGAAGCTGTCT | |
| | AGAAGGAAGAAGAACTTTCTGCTCGAGAAGGATCT | |
| | GGACACCAAGGAGGGAGAGTTTCTCGCTAGAAGCT | |
| | TCACCGACACAGCCTATATGTCTAGAGAGGTGTGT | |
| | GCCTATCTGGCCGACTGTCTGCTCTTCCCCGACGA | |
| | TGGAGCTAAGGCCCATGTGGTGCCTACAACCGGAA | |
| | GAGCCACCGCTTGGCTCAGAAGGAGATGGGGACTG | |
| | AACTTCGGCTCCAATGGCGAGAAAGATAGATCCGA | |
| | CGACAGACATCACGCTACAGATGCTTGCGTGATCG | |
| | CTGCTTGCTCCAGATCTCTGGTGATTAAGACCGCT | |
| | AGAATCAACCAAGAGACACATTGGAGCATCACAAG | |
| | AGGAATGAACGAAACCCAGAGGAGGGATGCCATCA | |
| | TGAAGGCTCTGGAGTCCGTCATGCCTTGGGAAACC | |
| | TTCGCCAACGAGGTGAGAGCTGCCCACGATTTTGT | |
| | CGTGCCTACCAGATTCGTGCCCAGAAAAGGCAAAG | |
| | GCGAGCTCTTCGAGCAGACCGTGTATAGATACGCT | |
| | GGCGTGAACGCCCAAGGCAAGGATATCGCTAGAAA | |
| | GGCCTCCTCCGATAAAGACATCGTCATGGGAAACG | |
| | CCGTGGTGAGCGCCGATGAAAAGAGCGTGATCAAA | |
| | GTGAGCGAGATGCTGTGTCTGAGACTGTGGCACGA | |
| | TCCCGAGGCCAAGAAGGGCCAAGGCGCTTGGTATG | |
| | CTGACCCCGTGTACAAGGCTGATATCCCCGCTCTG | |
| | AAAGATGGCACATACGTCCCTAGAATCGCCAAACA | |
| | GAAATATGGAAGAAAGGTCTGGAAGGCCGTGCCCA | |
| | ATAGCGCTCTGACCCAAAAACCTCTGGAGATCTAC | |
| | CTCGGAGATCTGATTAAGGTCGGCGATAAGCTGGG | |
| | CAGATACAACGGCTACAACATCGCCACCGCCAATT | |
| | GGTCCTTTGTCGACGCTCTGACCAAGAAGGAAATT | |
| | GCTTTCCCTAGCGTCGGCATGCTGAGCAATGAACT | |
| | CCAACCTATCATCATCAGAGAAAGCATTCTGGACA | |
| | ACTGA | |
| AIK Type II | ATGGGAAAGCCTATTCCCAACCCTCTGCTGGGACT | 12 |
| Cas | GGACTCCACAAAAAGAACAGCCGACGGCAGCGAGT | |
| mammalian | TCGAAAGCCCCAAGAAGAAGAGAAAAGTGGAGATC | |
| expression | ACAATTAATAGGGAAATTGGCAAGCTGGGACTCCC | |
| construct | TAGACATCTGGTGCTGGGCATGGATCCCGGCATTG | |
| (includes N- | CCAGCTGCGGATTCGCTCTGATCGACACAGCCAAT | |
| terminal SV5 | AGAGAAATTCTGGATCTGGGCGTGAGGCTGTTCGA | |
| tag and NLS | TTCCCCTACCCATCCTAAGACCGGCCAGTCTCTGG | |
| and C- | CTGTCATCAGAAGGGGCTTCAGATCCACAAGAAGG | |
| terminal NLS) | AATATCGACAGAACCCAAGCTAGACTCAAGCACTG | |
| (nt) | CCTCCAGATCCTCAAAGCCTATGGCCTCATTCCCC | |
| | AAGACGCCACCAAAGAGTACTTCCACACCACCAAG | |
| | GGAGATAAGCAGCCTCTGAAACTGAGAGTGGACGG | |
| | ACTGGATAGACTGCTGAACGATAGAGAATGGGCTC | |
| | TGGTGCTGTACAGCCTCTGTAAGAGAAGGGGCTAC | |
| | ATCCCTCACGGCGAGGGAAATCAAGACAAGTCCAG | |
| | CGAAGGAGGCAAAGTGCTGAGCGCCCTCGCCGCCA | |
| | ACAAGGAAGCTATCGCCGAGACAAGCTGTAGAACC | |
| | GTGGGCGAATGGCTCGCTCAACAGCCTCAGAGCAG | |
| | AAATAGAGGCGGAAACTATGACAAGTGCGTCACCC | |
| | ATGCTCAGCTCATTGAGGAAACCCATATCCTCTTC | |
| | GACGCTCAGAGAAGCTTTGGCAGCAAGTACGCCAG | |
| | CCCCGAGTTCGAAGCCGCCTACATTGAAGTGTGTG | |
| | ACTGGGAAAGGTCTAGAAAGGATTTTGATAGAAGG | |
| | ACATACGATCTGGTCGGCCACTGCAGCTACTTCCC | |
| | TACCGAAAAGAGAGCCGCTAGATGCACACTGACCA | |
| | GCGAGCTGGTCAGCGCCTACGGAGCTCTCGGCAAC | |
| | ATCACCATCATCCACGACGACGGAACCAGCAGAGC | |
| | TCTGAGCGCCACCGAAAGGGATGAGTGCATCGCCA | |
| | TCCTCTTTAGCTGCGAGCCCATTAGAGGCAATAAG | |
| | GATTGTGCCGTGAAGTTCGGAGCTCTGAGAAAGGC | |
| | TCTGGACCTCTCCTCCGGAGATTACTTCAAGGGAG | |
| | TCCCCGCCGCCGACGAAAAGACCAGAGAGGTCTAC | |
| | AAGCCCAAGGGCTGGAGAGTGCTGAGAAACACCCT | |
| | CAACGCCGCCAACCCTATTCTGCTCCAGAGACTGA | |
| | GAGATGACAGAAACCTCGCTGACGCTGTGATGGAA | |
| | GCTGTCGCTTACAGCAGCGCTCTGCCCGTGCTCCA | |
| | AGAGCAGCTGCAAGGACTGCCTCTCTCCGAGGCTG | |
| | AGATCGAGGCTCTGTGCAGACTGCCTTACAGCTCC | |
| | AAAGCTCTGAACGGCTACGGCAATAGAAGCAAAAA | |
| | AGCTCTGGACATGCTGCTCGATTGTCTGGAGGAAC | |
| | CCGAAGTGCTGAACCTCACCCAAGCCGAAAACGAT | |
| | TGTGGACTGCTCGGACTGAGAATCGCCGGAACCCA | |
| | GCTGGAGAGATCCGATAGACTCATGCCTTATGAAA | |
| | CATGGATCGAGAGAACCGGAAGAACCAATAACAAC | |
| | CCCGTGGTCATCAGAGCCATGAGCCAAATGAGAAA | |
| | GGTGGTCAACGCCATCTGCAGAAAGTGGGGCGTGC | |
| | CCAACGAAATTCATGTGGAGCTGGATAGAGAGCTG | |
| | AGACTGCCCCAAAGGGCTAAGGACGAGATCGCCAA | |
| | GGCTAATAAGAAGAATGAAAAGAACAGAGAGAGAA | |
| | TCGCCGGCCAGATTGCTGAACTGAGAGGCTGTACA | |
| | GCTGACGAGGTGACCGGCAAGCAGATTGAGAAGTA | |
| | TAGACTCTGGGAGGAGCAAGAGTGCTTTGATCTGT | |
| | ACACCGGAGCCAAGATCGAGGTGGATAGGCTCATC | |
| | AGCGATGATACATACACCCAGATCGACCACATTCT | |
| | GCCTTTTAGCAGAACCGGCGAGAACTCTAGAAACA | |
| | ACAAAGTGCTGGTGCTCGCTAAGTCCAATCAAGAC | |
| | AAGAGGGAGCAGACCCCCTATGAATGGATGAGCCA | |
| | TGATGGCGCTCCCAGCTGGGACGCCTTCGAAAGGA | |
| | GGGTGCAAGAGAACCAGAAGCTGTCTAGAAGGAAG | |
| | AAGAACTTTCTGCTCGAGAAGGATCTGGACACCAA | |
| | GGAGGGAGAGTTTCTCGCTAGAAGCTTCACCGACA | |
| | CAGCCTATATGTCTAGAGAGGTGTGTGCCTATCTG | |
| | GCCGACTGTCTGCTCTTCCCCGACGATGGAGCTAA | |
| | GGCCCATGTGGTGCCTACAACCGGAAGAGCCACCG | |
| | CTTGGCTCAGAAGGAGATGGGGACTGAACTTCGGC | |
| | TCCAATGGCGAGAAAGATAGATCCGACGACAGACA | |
| | TCACGCTACAGATGCTTGCGTGATCGCTGCTTGCT | |
| | CCAGATCTCTGGTGATTAAGACCGCTAGAATCAAC | |
| | CAAGAGACACATTGGAGCATCACAAGAGGAATGAA | |
| | CGAAACCCAGAGGAGGGATGCCATCATGAAGGCTC | |
| | TGGAGTCCGTCATGCCTTGGGAAACCTTCGCCAAC | |
| | GAGGTGAGAGCTGCCCACGATTTTGTCGTGCCTAC | |
| | CAGATTCGTGCCCAGAAAAGGCAAAGGCGAGCTCT | |
| | TCGAGCAGACCGTGTATAGATACGCTGGCGTGAAC | |
| | GCCCAAGGCAAGGATATCGCTAGAAAGGCCTCCTC | |
| | CGATAAAGACATCGTCATGGGAAACGCCGTGGTGA | |
| | GCGCCGATGAAAAGAGCGTGATCAAAGTGAGCGAG | |
| | ATGCTGTGTCTGAGACTGTGGCACGATCCCGAGGC | |
| | CAAGAAGGGCCAAGGCGCTTGGTATGCTGACCCCG | |
| | TGTACAAGGCTGATATCCCCGCTCTGAAAGATGGC | |
| | ACATACGTCCCTAGAATCGCCAAACAGAAATATGG | |
| | AAGAAAGGTCTGGAAGGCCGTGCCCAATAGCGCTC | |
| | TGACCCAAAAACCTCTGGAGATCTACCTCGGAGAT | |
| | CTGATTAAGGTCGGCGATAAGCTGGGCAGATACAA | |
| | CGGCTACAACATCGCCACCGCCAATTGGTCCTTTG | |
| | TCGACGCTCTGACCAAGAAGGAAATTGCTTTCCCT | |
| | AGCGTCGGCATGCTGAGCAATGAACTCCAACCTAT | |
| | CATCATCAGAGAAAGCATTCTGGACAACAAGAGGA | |
| | CAGCTGACGGAAGCGAGTTCGAGAGCCCCAAGAAA | |
| | AAGAGAAAAGTCTGA | |

In some embodiments an AIK Type II Cas protein comprises an amino acid sequence of SEQ ID NO:7, SEQ ID NO:8, or SEQ ID NO:9 In some embodiments, an AIK Type II Cas protein has nickase activity, for example resulting from one or more amino acid substitutions relative to the sequence of SEQ ID NO:7, SEQ ID NO:8, or SEQ ID NO:9 In some embodiments, the one or more amino acid substitutions providing nickase activity is a D23A substitution, wherein the position of the D23A substitution is defined with respect to the amino acid numbering of SEQ ID NO:8.

### 6.2.3. HPLH Type II Cas Proteins

In one aspect, the disclosure provides HPLH Type II Cas proteins. The HPLH Type II Cas proteins typically comprise an amino acid sequence that is at least 50%, at least 55%, at least 60%, at least 65%, at least 70%, at least 75%, at least 85%, at least 90%, or at least 95% identical to SEQ ID NO:30. In some embodiments, the HPLH Type II Cas proteins comprise an amino acid sequence that is at least 95%, at least 96%, at least 97%, at least 98%, or at least 99% identical to SEQ ID NO:30. In some embodiments, an HPLH Type II Cas protein comprises an amino acid sequence that is identical to SEQ ID NO:30.

Exemplary HPLH Type II Cas protein sequences and nucleotide sequences encoding exemplary HPLH Type II Cas proteins are set forth in Table 10.

TABLE 1C
HPLH Type II Cas Sequences

| | | SEQ ID |
|---|---|---|
| Name | Sequence | NO: |
| HPLH Type II | KKKIFGFDLGIASIGWAVIDHSDENFDPETGEIIE | 30 |
| Cas coding | GKVVGCGVRCFPVAENPKDGSSLAAPRREKRLLRR | |
| sequence | ITRRKARRMLGIKRLFVAKGLAASTAELETLYAAQ | |
| (aa) (without | TGGDVWNLRAEALRRPLSKEELLRVLTHLAKHRGF | |
| N-terminal | KSYRKAAEEADKESGRILTAIAENRKETAGFQTLA | |
| methionine) | QMIVERAKHSDDHKMRNYTSQEGENKGVAVYVNSI | |
| | PREEIEKETKLIFEYQKQFGLFTEDLYRDFCKIAS | |
| | RYREAGSVGHMVGRCRFEPEQPRAPKEAPSAELFV | |
| | ALSKINNLKVTVDGERRFLNGEERKALLELLKNTK | |
| | EVKYLTIKNKLFKGREVFFDDVNYAQKTKKGKSGE | |
| | EKAVNPEDAKFYAMKGWHKLKAAFSPEQWKEVGSN | |
| | LPLLDLGMTAVVCEKNDAGIERFLSEKGIPEDYRE | |
| | VFKKLTGSEFINLSLKALYKLNPYLAEGLKYNQAC | |
| | EKAGYDFREDGIKLAEEKGLLLPPIADDKLTTVPV | |
| | VNRAVAQFRKVYNAMVRTYGAPDQINLEIGRDLKK | |
| | SRDERNQIMRRQKENEAERKEAEDWLEKEGLAANG | |
| | KNMLKYRLYRQQNGKCIYSGKAIDLRRLDENGYCD | |
| | VDHIIPYSRSLDDGQNNKVLCLAEENRKKGSQTPY | |
| | EYLEPLGRWEEFETWVNTTPSINRYKRNNLLNKDY | |
| | KEKENDLEFRERNANDNSYIARYVKRYLEDAIDFS | |
| | ASSCTIGNRVQVRTGSLTDYLRHQWGLIKDRDASD | |
| | RHHAQDAVWVACATQGMVQKLSKLSAIFENKDDFR | |
| | RKKAEELGHEEAEAWYKYVKQQIREPWSGFRAEVL | |
| | ASLEKVFVSRPPRKNATGEIHQETIRTVNPKRKKY | |
| | NEKEILSGIKIRGGLAKNGLMLRTDVFVKKNKKGK | |
| | DEFYLVPVYLSDMGKELPNKAMVPGKKENEWIELD | |
| | ETCQFKFSFYMDDLIKIKKKENEIFGYFRGTNRAT | |
| | ASVSVTTHDRSHTFEGIGVKTQDGIEKYQVDPLGR | |
| | IAKVKKEIRLPLTMMKKNRHKKEE | |
| HPLH Type II | MKKKIFGFDLGIASIGWAVIDHSDENFDPETGEII | 31 |
| Cas coding | EGKVVGCGVRCFPVAENPKDGSSLAAPRREKRLLR | |
| sequence | RITRRKARRMLGIKRLFVAKGLAASTAELETLYAA | |
| (aa) | QTGGDVWNLRAEALRRPLSKEELLRVLTHLAKHRG | |
| | FKSYRKAAEEADKESGRILTAIAENRKETAGFQTL | |
| | AQMIVERAKHSDDHKMRNYTSQEGENKGVAVYVNS | |
| | IPREEIEKETKLIFEYQKQFGLFTEDLYRDFCKIA | |
| | SRYREAGSVGHMVGRCRFEPEQPRAPKEAPSAELF | |
| | VALSKINNLKVTVDGERRFLNGEERKALLELLKNT | |
| | KEVKYLTIKNKLFKGREVFFDDVNYAQKTKKGKSG | |
| | EEKAVNPEDAKFYAMKGWHKLKAAFSPEQWKEVGS | |
| | NLPLLDLGMTAVVCEKNDAGIERFLSEKGIPEDYR | |
| | EVFKKLTGSEFINLSLKALYKLNPYLAEGLKYNQA | |
| | CEKAGYDFREDGIKLAEEKGLLLPPIADDKLTTVP | |
| | VVNRAVAQFRKVYNAMVRTYGAPDQINLEIGRDLK | |
| | KSRDERNQIMRRQKENEAERKEAEDWLEKEGLAAN | |
| | GKNMLKYRLYRQQNGKCIYSGKAIDLRRLDENGYC | |
| | DVDHIIPYSRSLDDGQNNKVLCLAEENRKKGSQTP | |
| | YEYLEPLGRWEEFETVVNTTPSINRYKRNNLLNKD | |
| | YKEKENDLEFRERNANDNSYIARYVKRYLEDAIDF | |
| | SASSCTIGNRVQVRTGSLTDYLRHQWGLIKDRDAS | |
| | DRHHAQDAWVVACATQGMVQKLSKLSAIFENKDDF | |
| | RRKKAEELGHEEAEAWYKYVKQQIREPWSGFRAEV | |
| | LASLEKVFVSRPPRKNATGEIHQETIRTVNPKRKK | |
| | YNEKEILSGIKIRGGLAKNGLMLRTDVFVKKNKKG | |
| | KDEFYLVPVYLSDMGKELPNKAMVPGKKENEWIEL | |
| | DETCQFKFSFYMDDLIKIKKKENEIFGYFRGTNRA | |
| | TASVSVTTHDRSHTFEGIGVKTQDGIEKYQVDPLG | |
| | RIAKVKKEIRLPLTMMKKNRHKKEE | |
| HPLH Type II | MGKPIPNPLLGLDSTKRTADGSEFESPKKKRKVKK | 786 |
| Cas | KIFGFDLGIASIGWAVIDHSDENFDPETGEIIEGK | |
| mammalian | VVGCGVRCFPVAENPKDGSSLAAPRREKRLLRRIT | |
| expression | RRKARRMLGIKRLFVAKGLAASTAELETLYAAQTG | |
| construct | GDVWNLRAEALRRPLSKEELLRVLTHLAKHRGFKS | |
| (includes N- | YRKAAEEADKESGRILTAIAENRKETAGFQTLAQM | |
| terminal SV5 | IVERAKHSDDHKMRNYTSQEGENKGVAVYVNSIPR | |
| tag and NLS | EEIEKETKLIFEYQKQFGLFTEDLYRDFCKIASRY | |
| and C- | REAGSVGHMVGRCRFEPEQPRAPKEAPSAELFVAL | |
| terminal NLS) | SKINNLKVTVDGERRFLNGEERKALLELLKNTKEV | |
| (aa) | KYLTIKNKLFKGREVFFDDVNYAQKTKKGKSGEEK | |
| | AVNPEDAKFYAMKGWHKLKAAFSPEQWKEVGSNLP | |
| | LLDLGMTAVVCEKNDAGIERFLSEKGIPEDYREVF | |
| | KKLTGSEFINLSLKALYKLNPYLAEGLKYNQACEK | |
| | AGYDFREDGIKLAEEKGLLLPPIADDKLTTVPVVN | |
| | RAVAQFRKVYNAMVRTYGAPDQINLEIGRDLKKSR | |
| | DERNQIMRRQKENEAERKEAEDWLEKEGLAANGKN | |
| | MLKYRLYRQQNGKCIYSGKAIDLRRLDENGYCDVD | |
| | HIIPYSRSLDDGQNNKVLCLAEENRKKGSQTPYEY | |
| | LEPLGRWEEFETVVNTTPSINRYKRNNLLNKDYKE | |
| | KENDLEFRERNANDNSYIARYVKRYLEDAIDFSAS | |
| | SCTIGNRVQVRTGSLTDYLRHQWGLIKDRDASDRH | |
| | HAQDAVVVACATQGMVQKLSKLSAIFENKDDFRRK | |
| | KAEELGHEEAEAWYKYVKQQIREPWSGFRAEVLAS | |
| | LEKVFVSRPPRKNATGEIHQETIRTVNPKRKKYNE | |
| | KEILSGIKIRGGLAKNGLMLRTDVFVKKNKKGKDE | |
| | FYLVPVYLSDMGKELPNKAMVPGKKENEWIELDET | |
| | CQFKFSFYMDDLIKIKKKENEIFGYFRGTNRATAS | |
| | VSVTTHDRSHTFEGIGVKTQDGIEKYQVDPLGRIA | |
| | KVKKEIRLPLTMMKKNRHKKEEKRTADGSEFESPK | |
| | KKRKV | |
| HPLH Type II | ATGAAAAAGAAAATTTTTGGTTTTGATTTGGGGAT | 32 |
| Cas coding | TGCTTCGATCGGTTGGGCGGTTATTGATCATAGTG | |
| sequence (nt) | ATGAAAATTTCGATCCGGAAACAGGAGAGATTATT | |
| not codon | GAAGGAAAAGTCGTTGGCTGCGGAGTGCGCTGTTT | |
| optimized | TCCGGTAGCGGAAAACCCGAAGGACGGTTCCTCGC | |
| | TGGCGGCGCCCCGGCGGGAAAAACGCTTGTTGCGC | |
| | CGGATCACCCGCCGAAAGGCCAGGAGAATGCTCGG | |
| | TATTAAGCGTCTGTTTGTCGCCAAGGGACTGGCTG | |
| | CTTCAACGGCGGAATTGGAAACGCTTTATGCCGCA | |
| | CAAACAGGCGGCGACGTTTGGAATCTGCGGGCAGA | |
| | GGCCTTGCGGCGTCCGCTGTCAAAAGAGGAACTGC | |
| | TGCGAGTTTTGACTCATTTGGCAAAACATCGCGGT | |
| | TTCAAGTCTTACCGTAAGGCTGCTGAAGAGGCGGA | |
| | CAAGGAAAGCGGCCGCATCTTGACTGCGATTGCGG | |
| | AAAACCGGAAGGAAACGGCCGGTTTTCAAACGCTG | |
| | GCGCAGATGATCGTTGAACGGGCCAAACATTCCGA | |
| | CGATCATAAAATGCGCAACTATACGTCGCAGGAAG | |
| | GCGAAAACAAGGGCGTAGCCGTTTATGTCAATTCC | |
| | ATTCCGCGGGAAGAAATTGAAAAGGAAACAAAACT | |
| | GATTTTTGAGTATCAGAAGCAATTCGGTTTGTTTA | |
| | CCGAGGACTTATACCGCGATTTTTGCAAGATTGCC | |
| | TCCCGTTACCGGGAAGCGGGGAGTGTCGGGCACAT | |
| | GGTTGGCAGATGCCGGTTTGAACCGGAACAGCCGC | |
| | GGGCGCCGAAAGAAGCCCCTTCGGCGGAGCTCTTC | |
| | GTTGCTTTAAGCAAAATTAATAACCTGAAAGTGAC | |
| | CGTTGACGGCGAACGCCGTTTCTTAAACGGAGAAG | |
| | AGCGGAAGGCTTTGCTTGAATTGCTGAAAAACACC | |
| | AAGGAAGTCAAATATTTGACTATCAAAAACAAATT | |
| | GTTTAAAGGCCGGGAAGTCTTTTTTGACGACGTTA | |
| | ACTATGCGCAAAAAACAAAAAAAGGAAAAAGCGGA | |
| | GAAGAAAAAGCGGTAAATCCCGAAGACGCAAAGTT | |
| | TTACGCCATGAAAGGCTGGCATAAGCTGAAAGCCG | |
| | CTTTTTCACCGGAGCAGTGGAAAGAGGCGGTTCGA | |
| | ATTTGCCGTTGCTGGATCTCGGCATGACCGCGGTC | |
| | GTCTGCGAAAAAAACGACGCCGGAATAGAGCGCTT | |
| | TTTAAGCGAAAAAGGAATACCGGAGGATTATCGGG | |
| | AAGTTTTTAAAAAGCTGACCGGCAGCGAGTTTATT | |
| | AATCTGTCACTGAAAGCCCTTTATAAGCTCAATCC | |
| | TTATCTGGCGGAAGGTTTGAAGTATAACCAAGCCT | |
| | GCGAAAAGGCCGGATACGACTTCCGCGAGGACGGC | |
| | ATCAAACTGGCGGAGGAAAAAGGTTTGTTGCTGCC | |
| | GCCGATTGCAGATGACAAACTGACGACGGTGCCGG | |
| | TCGTTAACCGTGCGGTTGCCCAGTTCCGCAAAGTA | |
| | TATAACGCCATGGTTCGGACATATGGTGCGCCGGA | |
| | TCAGATAAATCTGGAAATCGGCCGGGATTTGAGAA | |
| | AAGCCGTGACGAGCGCAATCAGATCATGCGGCGGC | |
| | AAAAGGAAAACGAGGCCGAACGGAAAGAAGCCGAG | |
| | GACTGGTTGGAAAAGGAAGGACTTGCCGCAAACGG | |
| | TAAAAATATGTTGAAATACCGTTTGTACCGGCAGC | |
| | AAAACGGCAAATGCATCTATTCCGGAAAAGCGATT | |
| | GACCTCCGCCGCCTGGACGAAAACGGTTATTGCGA | |
| | CGTTGACCACATCATCCCTTATTCCCGTTCGCTTG | |
| | ACGACGGTCAGAACAACAAAGTGCTTTGTCTGGCC | |
| | GAAGAAAACCGCAAGAAAGGAAGCCAAACTCCTTA | |
| | TGAATATCTGGAGCCGCTCGGACGATGGGAAGAGT | |
| | TTGAAACCGTTGTTAACACCACGCCGTCGATTAAC | |
| | CGTTACAAAAGAAACAACCTGTTGAACAAAGATTA | |
| | TAAAGAAAAAGAAAACGATTTGGAATTTCGCGAAA | |
| | GAAACGCCAATGACAACTCCTATATTGCCCGCTAT | |
| | GTCAAACGGTATTTGGAAGATGCCATTGATTTTTC | |
| | CGCCAGTTCCTGCACAATCGGAAACCGGGTTCAGG | |
| | TGCGCACCGGTTCGTTAACCGATTACCTCCGCCAT | |
| | CAGTGGGGGCTGATAAAAGATCGTGACGCAAGCGA | |
| | CAGGCATCATGCTCAGGACGCGGTTGTCGTTGCCT | |
| | GCGCCACGCAGGGAATGGTGCAGAAACTGTCAAAA | |
| | CTTTCCGCGATTTTTGAAAACAAGGACGATTTCCG | |
| | CAGAAAGAAAGCGGAAGAACTCGGGCACGAGGAGG | |
| | CCGAAGCCTGGTACAAATACGTCAAACAGCAAATT | |
| | CGGGAACCCTGGAGCGGTTTTCGGGCTGAAGTACT | |
| | GGCCAGCCTGGAAAAGGTTTTCGTTTCCCGTCCGC | |
| | CGCGCAAAAACGCAACCGAGAGATTCACCAGGAAA | |
| | CGATTCGCACGGTTAATCCGAAACGTAAAAAATAT | |
| | AATGAAAAGGAAATTCTGTCCGGCATCAAAATCCG | |
| | CGGCGGGCTGGCCAAAAACGGCCTGATGCTGCGAA | |
| | CGGACGTTTTTGTAAAAAAGAACAAAAAGGGAAAA | |
| | GACGAATTTTACCTGGTGCCGGTTTATCTTTCCGA | |
| | TATGGGAAAAGAGCTGCCGAACAAGGCGATGGTTC | |
| | CGGGTAAAAAAGAAAACGAATGGATTGAACTGGAT | |
| | GAAACCTGTCAGTTTAAATTCAGCTTTTATATGGA | |
| | CGATTTGATAAAAATCAAAAAAAAGGAAAATGAGA | |
| | TTTTCGGCTATTTCAGAGGAACAAACAGGGCGACG | |
| | GCGTCAGTATCCGTTACCACCCATGACCGCAGTCA | |
| | TACTTTTGAAGGCATCGGCGTCAAAACTCAGGACG | |
| | GTATCGAAAAATATCAGGTGGATCCGCTGGGACGT | |
| | ATTGCCAAAGTCAAAAAAGAAATCCGGCTCCCGCT | |
| | GACGATGATGAAAAAGAACCGGCATAAAAAGGAGG | |
| | AGTGA | |
| HPLH Type II | ATGGGCAAGCCCATCCCTAATCCTCTGCTGGGACT | 33 |
| Cas coding | GGACAGCACCAAAAGAACCGCTGACGGATCCGAGT | |
| sequence (nt) | TCGAGAGCCCCAAGAAAAAGAGGAAGGTCAAGAAA | |
| codon | AAGATTTTTGGCTTCGATCTCGGAATTGCTAGCAT | |
| optimized | CGGATGGGCCGTGATTGACCACTCCGACGAGAACT | |
| (including | TCGACCCCGAAACCGGCGAGATTATCGAGGGCAAG | |
| V5-tag and N- | GTGGTCGGCTGCGGAGTGAGATGTTTCCCCGTGGC | |
| and C- | CGAGAATCCCAAGGACGGAAGCTCCCTCGCTGCCC | |
| terminal NLS) | CTAGGAGGGAGAAGAGGCTGCTGAGAAGGATCACC | |
| | AGAAGAAAGGCCAGAAGGATGCTGGGCATCAAAAG | |
| | GCTGTTCGTGGCCAAAGGACTGGCCGCTAGCACAG | |
| | CTGAGCTGGAGACACTCTACGCCGCTCAGACCGGC | |
| | GGAGATGTGTGGAATCTGAGGGCTGAGGCCCTCAG | |
| | AAGGCCTCTGAGCAAGGAGGAACTGCTCAGAGTGC | |
| | TCACCCATCTGGCCAAGCATAGAGGATTTAAGAGC | |
| | TATAGAAAAGCCGCCGAGGAAGCCGACAAAGAGTC | |
| | CGGAAGAATCCTCACCGCTATCGCCGAGAATAGGA | |
| | AGGAAACCGCCGGCTTTCAAACACTGGCCCAGATG | |
| | ATTGTGGAAAGAGCCAAGCACAGCGATGACCACAA | |
| | GATGAGGAATTACACCTCCCAAGAGGGCGAGAACA | |
| | AAGGCGTGGCCGTGTACGTCAACTCCATTCCTAGA | |
| | GAGGAGATCGAAAAGGAAACCAAACTGATTTTCGA | |
| | ATACCAGAAGCAGTTCGGACTGTTCACCGAAGATC | |
| | TGTATAGAGACTTCTGCAAGATCGCCAGCAGATAT | |
| | AGAGAGGCTGGCTCCGTGGGACACATGGTCGGAAG | |
| | GTGCAGATTTGAGCCCGAGCAACCCAGAGCTCCCA | |
| | AGGAGGCCCCTTCCGCCGAACTGTTCGTGGCTCTG | |
| | TCCAAGATCAACAACCTCAAAGTGACAGTGGATGG | |
| | CGAGAGAAGATTTCTGAACGGCGAGGAGAGAAAAG | |
| | CCCTCCTCGAGCTGCTCAAAAACACCAAGGAAGTC | |
| | AAGTACCTCACCATTAAGAATAAGCTCTTCAAGGG | |
| | CAGAGAGGTCTTCTTCGATGACGTGAACTACGCCC | |
| | AGAAAACCAAAAAAGGCAAGAGCGGCGAGGAAAAG | |
| | GCTGTGAACCCCGAGGACGCCAAGTTTTACGCTAT | |
| | GAAGGGATGGCACAAGCTCAAGGCTGCCTTTTCCC | |
| | CCGAACAGTGGAAAGAGGTGGGCAGCAATCTGCCC | |
| | CTCCTCGATCTGGGAATGACAGCCGTGGTCTGCGA | |
| | GAAGAACGACGCTGGCATCGAGAGATTTCTGTCCG | |
| | AAAAGGGCATTCCCGAAGACTATAGAGAGGTGTTT | |
| | AAAAAACTGACCGGCTCCGAGTTCATCAACCTCTC | |
| | TCTGAAGGCTCTCTATAAGCTGAACCCCTATCTGG | |
| | CCGAGGGACTGAAATACAACCAAGCTTGCGAAAAA | |
| | GCCGGCTACGACTTTAGAGAGGACGGCATTAAGCT | |
| | GGCCGAAGAAAAAGGACTGCTGCTGCCCCCCATTG | |
| | CCGATGATAAACTGACCACCGTGCCCGTGGTCAAC | |
| | AGAGCCGTGGCCCAGTTCAGAAAAGTGTATAACGC | |
| | TATGGTGAGAACATACGGAGCCCCCGACCAAATCA | |
| | ATCTGGAAATTGGAAGAGATCTGAAGAAGTCTAGA | |
| | GATGAGAGGAACCAAATCATGAGGAGGCAAAAAGA | |
| | GAACGAGGCCGAGAGGAAGGAAGCCGAGGATTGGC | |
| | TCGAAAAGGAGGGACTGGCTGCCAACGGAAAAAAC | |
| | ATGCTCAAGTACAGACTGTATAGGCAGCAGAACGG | |
| | CAAGTGCATCTACAGCGGCAAAGCTATCGATCTGA | |
| | GAAGGCTGGACGAAAATGGATACTGCGACGTGGAT | |
| | CACATCATCCCTTACTCCAGATCTCTGGACGACGG | |
| | ACAAAACAACAAAGTGCTGTGTCTCGCCGAGGAAA | |
| | ATAGAAAGAAGGGCAGCCAAACCCCTTACGAGTAT | |
| | CTGGAGCCTCTGGGCAGATGGGAGGAATTCGAAAC | |
| | CGTGGTGAACACCACACCCTCCATCAATAGATATA | |
| | AGAGAAATAATCTGCTCAATAAAGATTATAAGGAA | |
| | AAGGAGAACGACCTCGAGTTTAGGGAGAGGAACGC | |
| | CAACGACAACAGCTACATCGCTAGATACGTGAAGA | |
| | GGTATCTGGAGGACGCCATCGACTTTAGCGCTTCC | |
| | AGCTGCACCATCGGCAATAGAGTGCAAGTGAGAAC | |
| | CGGCAGCCTCACCGACTATCTGAGACACCAATGGG | |
| | GACTCATTAAGGATAGAGACGCTAGCGACAGACAC | |
| | CATGCCCAAGACGCTGTGGTCGTCGCTTGCGCCAC | |
| | CCAAGGAATGGTGCAGAAGCTCTCCAAACTCAGCG | |
| | CTATCTTTGAAAATAAAGATGATTTTAGAAGAAAG | |
| | AAGGCCGAGGAACTGGGACACGAAGAGGCTGAGGC | |
| | TTGGTACAAGTACGTGAAGCAGCAGATTAGAGAAC | |
| | CTTGGTCCGGATTTAGGGCCGAGGTGCTGGCCTCT | |
| | CTGGAGAAGGTGTTCGTCTCTAGACCTCCCAGAAA | |
| | GAACGCTACCGGAGAGATCCACCAAGAAACCATTA | |
| | GGACCGTCAACCCCAAGAGAAAAAAATACAACGAG | |
| | AAAGAGATCCTCTCCGGCATCAAGATTAGAGGAGG | |
| | CCTCGCCAAGAACGGCCTCATGCTGAGAACCGATG | |
| | TCTTTGTGAAGAAAAATAAAAAGGGCAAGGACGAA | |
| | TTCTACCTCGTCCCCGTGTATCTGTCCGACATGGG | |
| | CAAAGAGCTGCCTAATAAGGCTATGGTGCCCGGCA | |
| | AGAAGGAAAACGAGTGGATCGAACTGGACGAGACA | |
| | TGCCAATTCAAATTTTCCTTCTACATGGATGACCT | |
| | CATCAAGATTAAGAAAAAGGAAAATGAGATCTTCG | |
| | GCTATTTTAGAGGAACAAACAGAGCCACCGCTAGC | |
| | GTCTCCGTGACCACCCACGATAGAAGCCACACATT | |
| | TGAGGGCATCGGAGTGAAGACCCAAGACGGAATTG | |
| | AGAAGTACCAAGTGGACCCTCTCGGAAGAATCGCC | |
| | AAGGTGAAAAAGGAGATTAGACTGCCTCTGACCAT | |
| | GATGAAAAAGAACAGACATAAGAAGGAGGAGAAGA | |
| | GAACAGCTGATGGCAGCGAGTTCGAATCCCCTAAG | |
| | AAGAAGAGGAAGGTGTGA | |

In some embodiments an HPLH Type II Cas protein comprises an amino acid sequence of SEQ ID NO:30, SEQ ID NO:31, or SEQ ID NO:786. In some embodiments, an HPLH Type II Cas protein has nickase activity, for example resulting from one or more amino acid substitutions relative to the sequence of SEQ ID NO:30, SEQ ID NO:31, or SEQ ID NO:786. In some embodiments, the one or more amino acid substitutions providing nickase activity is a D23A substitution, wherein the position of the D23A substitution is defined with respect to the amino acid numbering of SEQ ID NO:8. The corresponding position in SEQ ID NO:30, SEQ ID NO:31, or SEQ ID NO:786 can be determined, for example, by performing a sequence alignment of SEQ ID NO:30, SEQ ID NO:31, or SEQ ID NO:786 with SEQ ID NO:8 (e.g., by BLAST).

### 6.2.4. ANAB Type II Cas Proteins

In one aspect, the disclosure provides ANAB Type II Cas proteins. The ANAB Type II Cas proteins typically comprise an amino acid sequence that is at least 50%, at least 55%, at least 60%, at least 65%, at least 70%, at least 75%, at least 85%, at least 90%, or at least 95% identical to SEQ ID NO:34. In some embodiments, the ANAB Type II Cas proteins comprise an amino acid sequence that is at least 95%, at least 96%, at least 97%, at least 98%, or at least 99% identical to SEQ ID NO:34. In some embodiments, an ANAB Type II Cas protein comprises an amino acid sequence that is identical to SEQ ID NO:34.

Exemplary ANAB Type II Cas protein sequences and nucleotide sequences encoding exemplary ANAB proteins are set forth in Table 1 D.

TABLE 1D
ANAB Type II Cas Sequences

| | | SEQ ID |
|---|---|---|
| Name | Sequence | NO: |
| ANAB Type II | EITINREIGKLGLPRHLVLGMDPGIASCGFALIDT | 34 |
| Cas coding | ANHEILDLGVRLFDSPTHPKTGQSLAVIRRGFRST | |
| sequence | RRNIDRTQARLKHCLQVLKAYGLIPQDATKEYLHT | |
| (aa) (without | TKGDKQPLKLRVDGLDRLLNDREWALVLYSLCKRR | |
| N-terminal | GYIPHGEGNQDKSSEGGKVLSALAANKEAIAETSC | |
| methionine) | RTVGEWLAWQPQSRNRGGNYDKCVTHAQLIEETHI | |
| | LFDAQRSFGSKYASPEFEAAYIEVCDWERSRKDFD | |
| | RRTYDLVGHCSYFPTEKRAARCTLTSELVSAYGAL | |
| | GNITIIHENGTSRALSATERDECIAILFSCEPIRG | |
| | NKDCAVKFGALRKALDLSSGDYFKGVPAADEKTRE | |
| | VYKPKGWRVLRNTLNAANPILLQRLRDDRNLADAV | |
| | MEAVAYSSALPVLQEQLQGLPFSEAEIEALCRLPY | |
| | SSKALNGYGNRSKKALDMLLDCLEEPEVLNLTQAE | |
| | NDCGLLGLRIAGAQLERSDRLMPYETWIELTGRTN | |
| | NNPVVIRSMSQMRKVVNAVCRKWGVPNEIHVELDR | |
| | ELRLPQRAKDEIAKANKKNEKNRERIAGQIAELRG | |
| | CTADEVTGKQIEKYRLWEEQECFDLYTGAKIEVDR | |
| | LISDDTYTQIDHILPFSRTGENSRNNKVLVLAKSN | |
| | QDKREQTPYEWMSHDGAPSWDAFERRVQENQKLSR | |
| | RKKNFLLEKDLDTKEGEFLARSFTDTAYMSREACA | |
| | YLADCLLFPDDGAKAHVVPTTGRATAWLRRRWGLN | |
| | FGSNGEKDRSDDRHHATDACVIAACSRSLVIKTAR | |
| | INQETHWSITRGMNETQRRDAIMKALESVMPWETF | |
| | ANEVRAAHDFVVPTRFVPRKGKGELFEQTVYRYAG | |
| | VNAQGKDIARKASSDKDIVMGNAVVSLDEKSVIKV | |
| | SEMLCLRLWHDPEAKKGQGAWYADPVYKADIPALK | |
| | DGTYVPRIAKAHTGRKAWKPVPESAMKKPPLEIYL | |
| | GDLVQIGDFMGRFSGYNIANANWSFVDRLTKEALG | |
| | CPTVVKLDNKLAPAIIRESIIMH | |
| ANAB Type II | MEITINREIGKLGLPRHLVLGMDPGIASCGFALID | 35 |
| Cas coding | TANHEILDLGVRLFDSPTHPKTGQSLAVIRRGFRS | |
| sequence | TRRNIDRTQARLKHCLQVLKAYGLIPQDATKEYLH | |
| (aa) | TTKGDKQPLKLRVDGLDRLLNDREWALVLYSLCKR | |
| | RGYIPHGEGNQDKSSEGGKVLSALAANKEAIAETS | |
| | CRTVGEWLAWQPQSRNRGGNYDKCVTHAQLIEETH | |
| | ILFDAQRSFGSKYASPEFEAAYIEVCDWERSRKDF | |
| | DRRTYDLVGHCSYFPTEKRAARCTLTSELVSAYGA | |
| | LGNITIIHENGTSRALSATERDECIAILFSCEPIR | |
| | GNKDCAVKFGALRKALDLSSGDYFKGVPAADEKTR | |
| | EVYKPKGWRVLRNTLNAANPILLQRLRDDRNLADA | |
| | VMEAVAYSSALPVLQEQLQGLPFSEAEIEALCRLP | |
| | YSSKALNGYGNRSKKALDMLLDCLEEPEVLNLTQA | |
| | ENDCGLLGLRIAGAQLERSDRLMPYETWIELTGRT | |
| | NNNPVVIRSMSQMRKVVNAVCRKWGVPNEIHVELD | |
| | RELRLPQRAKDEIAKANKKNEKNRERIAGQIAELR | |
| | GCTADEVTGKQIEKYRLWEEQECFDLYTGAKIEVD | |
| | RLISDDTYTQIDHILPFSRTGENSRNNKVLVLAKS | |
| | NQDKREQTPYEWMSHDGAPSWDAFERRVQENQKLS | |
| | RRKKNFLLEKDLDTKEGEFLARSFTDTAYMSREAC | |
| | AYLADCLLFPDDGAKAHVVPTTGRATAWLRRRWGL | |
| | NFGSNGEKDRSDDRHHATDACVIAACSRSLVIKTA | |
| | RINQETHWSITRGMNETQRRDAIMKALESVMPWET | |
| | FANEVRAAHDFVVPTRFVPRKGKGELFEQTVYRYA | |
| | GVNAQGKDIARKASSDKDIVMGNAVVSLDEKSVIK | |
| | VSEMLCLRLWHDPEAKKGQGAWYADPVYKADIPAL | |
| | KDGTYVPRIAKAHTGRKAWKPVPESAMKKPPLEIY | |
| | LGDLVQIGDFMGRFSGYNIANANWSFVDRLTKEAL | |
| | GCPTVVKLDNKLAPAIIRESIIMH | |
| ANAB Type II | MGKPIPNPLLGLDSTKRTADGSEFESPKKKRKVEI | 787 |
| Cas | TINREIGKLGLPRHLVLGMDPGIASCGFALIDTAN | |
| mammalian | HEILDLGVRLFDSPTHPKTGQSLAVIRRGFRSTRR | |
| expression | NIDRTQARLKHCLQVLKAYGLIPQDATKEYLHTTK | |
| construct | GDKQPLKLRVDGLDRLLNDREWALVLYSLCKRRGY | |
| (includes N- | IPHGEGNQDKSSEGGKVLSALAANKEAIAETSCRT | |
| terminal SV5 | VGEWLAWQPQSRNRGGNYDKCVTHAQLIEETHILF | |
| tag and NLS | DAQRSFGSKYASPEFEAAYIEVCDWERSRKDFDRR | |
| and C- | TYDLVGHCSYFPTEKRAARCTLTSELVSAYGALGN | |
| terminal NLS) | ITIIHENGTSRALSATERDECIAILFSCEPIRGNK | |
| (aa) | DCAVKFGALRKALDLSSGDYFKGVPAADEKTREVY | |
| | KPKGWRVLRNTLNAANPILLQRLRDDRNLADAVME | |
| | AVAYSSALPVLQEQLQGLPFSEAEIEALCRLPYSS | |
| | KALNGYGNRSKKALDMLLDCLEEPEVLNLTQAEND | |
| | CGLLGLRIAGAQLERSDRLMPYETWIELTGRTNNN | |
| | PVVIRSMSQMRKVVNAVCRKWGVPNEIHVELDREL | |
| | RLPQRAKDEIAKANKKNEKNRERIAGQIAELRGCT | |
| | ADEVTGKQIEKYRLWEEQECFDLYTGAKIEVDRLI | |
| | SDDTYTQIDHILPFSRTGENSRNNKVLVLAKSNQD | |
| | KREQTPYEWMSHDGAPSWDAFERRVQENQKLSRRK | |
| | KNFLLEKDLDTKEGEFLARSFTDTAYMSREACAYL | |
| | ADCLLFPDDGAKAHVVPTTGRATAWLRRRWGLNFG | |
| | SNGEKDRSDDRHHATDACVIAACSRSLVIKTARIN | |
| | QETHWSITRGMNETQRRDAIMKALESVMPWETFAN | |
| | EVRAAHDFVVPTRFVPRKGKGELFEQTVYRYAGVN | |
| | AQGKDIARKASSDKDIVMGNAVVSLDEKSVIKVSE | |
| | MLCLRLWHDPEAKKGQGAWYADPVYKADIPALKDG | |
| | TYVPRIAKAHTGRKAWKPVPESAMKKPPLEIYLGD | |
| | LVQIGDFMGRFSGYNIANANWSFVDRLTKEALGCP | |
| | TVVKLDNKLAPAIIRESIIMHKRTADGSEFESPKK | |
| | KRKV | |
| ANAB Type II | ATGGAGATCACCATCAATCGCGAAATTGGGAAGCT | 36 |
| Cas coding | CGGACTTCCCAGGCATCTTGTGCTTGGCATGGATC | |
| sequence (nt) | CAGGAATTGCAAGCTGCGGATTCGCACTTATCGAC | |
| not codon | ACGGCCAATCATGAAATCCTGGATTTGGGCGTCAG | |
| optimized | ATTATTTGACTCTCCAACTCATCCTAAAACGGGAC | |
| | AAAGCCTTGCGGTTATTCGCAGGGGATTCCGCTCT | |
| | ACCCGTCGAAACATTGACCGTACCCAGGCGCGCTT | |
| | GAAGCACTGTCTCCAAGTCCTCAAGGCTTATGGCC | |
| | TCATCCCCCAAGACGCCACCAAAGAGTACCTCCAC | |
| | ACCACAAAAGGCGACAAGCAGCCGCTCAAGCTTCG | |
| | TGTTGATGGCCTTGACCGCCTGCTCAACGATCGCG | |
| | AGTGGGCACTAGTCCTATACTCCCTCTGCAAGCGC | |
| | CGTGGATACATCCCCCACGGAGAAGGCAATCAGGA | |
| | TAAATCAAGCGAAGGCGGCAAGGTTCTATCCGCCC | |
| | TTGCGGCCAACAAGGAGGCAATTGCGGAGACCTCG | |
| | TGCCGCACCGTTGGCGAATGGCTCGCTTGGCAACC | |
| | TCAAAGTCGCAATCGTGGCGGCAATTACGACAAGT | |
| | GTGTAACGCACGCCCAGCTTATCGAAGAAACTCAT | |
| | ATCCTATTTGATGCTCAACGCTCCTTTGGCTCCAA | |
| | ATACGCTTCGCCGGAATTTGAGGCCGCATATATCG | |
| | AGGTTTGCGATTGGGAGCGTTCGCGCAAAGACTTC | |
| | GACCGCCGCACGTACGACCTCGTTGGCCACTGCTC | |
| | ATACTTCCCAACAGAAAAACGAGCCGCACGCTGCA | |
| | CGCTTACGAGCGAACTTGTTTCAGCCTATGGAGCA | |
| | CTCGGCAACATCACCATCATCCACGAAAACGGGAC | |
| | CTCTCGCGCCCTGAGCGCAACGGAGCGTGATGAGT | |
| | GCATTGCAATCCTGTTCTCGTGCGAACCAATTCGA | |
| | GGCAACAAAGATTGTGCTGTTAAATTCGGCGCCCT | |
| | CAGAAAAGCGCTCGACCTTAGTTCCGGCGATTACT | |
| | TTAAGGGAGTTCCAGCCGCCGACGAAAAAACGCGA | |
| | GAGGTGTACAAGCCCAAGGGATGGCGCGTGCTCCG | |
| | CAATACCCTCAATGCGGCCAACCCCATTCTCCTGC | |
| | AGCGTTTGCGCGATGACCGCAATCTCGCCGATGCC | |
| | GTTATGGAGGCGGTGGCATATTCCTCGGCCCTTCC | |
| | CGTACTCCAAGAGCAGCTTCAGGGGTTGCCGTTCT | |
| | CGGAAGCGGAGATCGAGGCGCTTTGTAGGCTTCCC | |
| | TATTCATCCAAAGCTCTTAACGGCTATGGCAACCG | |
| | TTCCAAAAAAGCACTCGACATGCTGCTCGATTGCC | |
| | TCGAGGAGCCCGAGGTCCTCAACCTTACACAGGCC | |
| | GAAAATGACTGCGGCCTGCTGGGACTTCGCATCGC | |
| | TGGCGCCCAGCTCGAGCGCTCCGATCGTCTGATGC | |
| | CCTATGAGACCTGGATCGAACTTACCGGTCGGACA | |
| | AATAACAATCCCGTCGTCATTCGTTCCATGTCGCA | |
| | AATGCGAAAAGTGGTCAACGCCGTCTGCCGCAAGT | |
| | GGGGCGTGCCAAACGAAATCCACGTTGAGCTTGAT | |
| | CGAGAGCTCAGGTTGCCTCAGCGCGCAAAAGACGA | |
| | GATTGCCAAGGCCAATAAGAAGAATGAGAAAAATC | |
| | GTGAGCGCATTGCCGGACAAATCGCTGAACTGCGT | |
| | GGCTGCACGGCAGATGAGGTCACGGGCAAACAGAT | |
| | AGAGAAGTACCGCCTGTGGGAAGAGCAGGAATGCT | |
| | TCGATCTTTACACGGGCGCTAAAATCGAAGTCGAT | |
| | CGCCTAATTAGCGACGACACTTACACGCAGATCGA | |
| | CCACATCCTGCCGTTCTCTCGCACGGGAGAAAACT | |
| | CTCGCAACAACAAAGTCCTAGTCCTCGCCAAAAGC | |
| | AATCAGGACAAACGCGAACAGACACCTTACGAATG | |
| | GATGTCCCACGACGGCGCGCCTTCATGGGATGCTT | |
| | TTGAGCGTCGCGTTCAGGAAAACCAGAAACTCAGC | |
| | CGTCGCAAAAAGAACTTCCTGCTGGAAAAAGACCT | |
| | TGACACCAAGGAAGGCGAATTCTTAGCACGCAGCT | |
| | TCACCGACACCGCCTATATGTCGCGAGAAGCATGC | |
| | GCTTACCTCGCCGACTGCCTACTGTTCCCCGATGA | |
| | TGGCGCAAAGGCACATGTTGTTCCCACCACTGGCA | |
| | GAGCGACCGCATGGCTGCGTCGCAGGTGGGGGCTT | |
| | AACTTTGGTTCGAATGGCGAAAAAGACCGCTCGGA | |
| | CGATCGTCACCATGCCACCGATGCTTGTGTGATTG | |
| | CAGCATGTAGTCGAAGCCTCGTGATTAAAACCGCT | |
| | CGAATCAACCAAGAGACACACTGGAGCATAACCAG | |
| | AGGTATGAACGAGACCCAACGCCGCGATGCCATCA | |
| | TGAAGGCTCTCGAAAGTGTTATGCCCTGGGAAACC | |
| | TTTGCGAACGAAGTACGTGCGGCGCACGATTTCGT | |
| | CGTACCCACGCGCTTTGTTCCGCGTAAGGGAAAGG | |
| | GCGAGTTGTTCGAGCAGACGGTCTATCGCTATGCC | |
| | GGCGTTAATGCACAGGGCAAAGACATTGCTCGCAA | |
| | GGCGAGCTCCGATAAGGACATCGTCATGGGCAACG | |
| | CCGTTGTGTCATTAGACGAAAAGTCGGTCATCAAG | |
| | GTGAGCGAAATGCTGTGTCTGAGGCTCTGGCATGA | |
| | CCCGGAGGCCAAGAAGGGGCAGGGCGCTTGGTACG | |
| | CAGACCCGGTCTACAAGGCGGATATTCCTGCACTT | |
| | AAGGATGGGACGTATGTTCCCAGGATTGCGAAGGC | |
| | GCATACTGGCCGAAAAGCCTGGAAGCCCGTGCCCG | |
| | AAAGCGCTATGAAAAAACCGCCGCTGGAGATATAT | |
| | CTGGGTGATCTGGTACAAATCGGCGATTTTATGGG | |
| | GCGGTTTAGCGGCTACAACATCGCAAATGCAAACT | |
| | GGTCGTTTGTCGACAGGCTCACTAAAGAAGCCCTA | |
| | GGCTGTCCCACCGTTGTCAAGTTGGACAACAAACT | |
| | GGCTCCCGCCATAATTCGCGAGTCCATAATCATGC | |
| | ACTAA | |
| ANAB Type II | ATGGGAAAGCCTATTCCTAACCCTCTGCTTGGCCT | 37 |
| Cas coding | CGACAGCACAAAGAGAACAGCTGATGGCAGCGAGT | |
| sequence (nt) | TCGAGAGCCCTAAGAAAAAGCGAAAAGTGGAAATT | |
| codon | ACAATCAACCGAGAGATCGGAAAACTGGGCCTGCC | |
| optimized | TAGACACCTGGTTCTGGGCATGGACCCCGGCATCG | |
| (including | CCTCCTGTGGCTTCGCCCTGATCGACACCGCCAAC | |
| V5-tag and N- | CACGAAATCCTGGATCTGGGCGTCCGGCTGTTCGA | |
| and C- | TAGCCCTACCCACCCCAAGACCGGACAGTCTCTGG | |
| terminal NLS) | CCGTGATCAGAAGAGGCTTCAGAAGCACCAGAAGA | |
| | AACATCGACAGAACCCAGGCTAGACTGAAGCACTG | |
| | CCTGCAGGTCCTGAAAGCCTACGGCTTGATCCCGC | |
| | AGGACGCCACCAAAGAGTACCTGCACACCACAAAG | |
| | GGCGACAAGCAGCCTCTGAAGCTGAGGGTGGACGG | |
| | CCTGGATAGACTGCTCAACGACCGGGAGTGGGCTC | |
| | TGGTGCTGTACAGCCTGTGCAAGCGTAGAGGCTAC | |
| | ATCCCTCACGGCGAAGGAAACCAGGATAAGAGCAG | |
| | CGAAGGCGGCAAGGTGCTGAGCGCTCTCGCTGCCA | |
| | ATAAGGAAGCTATCGCCGAGACAAGCTGCCGGACC | |
| | GTGGGCGAATGGCTGGCCTGGCAGCCCCAGAGCCG | |
| | GAACCGGGGAGGAAATTACGACAAGTGCGTGACAC | |
| | ACGCCCAACTGATTGAGGAGACACATATCCTGTTC | |
| | GACGCCCAGAGATCTTTTGGCTCTAAGTACGCCAG | |
| | CCCTGAGTTTGAAGCGGCATATATCGAGGTGTGCG | |
| | ACTGGGAGAGAAGCAGAAAGGATTTCGACCGCCGG | |
| | ACCTATGACCTGGTCGGACACTGCAGCTATTTCCC | |
| | CACCGAGAAACGGGCCGCCAGATGCACCCTGACCA | |
| | GCGAGCTGGTGTCCGCCTACGGGGCTCTGGGAAAC | |
| | ATCACCATCATACACGAAAACGGCACCAGCAGAGC | |
| | CCTGAGCGCTACTGAGCGGGACGAATGCATCGCCA | |
| | TCCTGTTTTCTTGTGAACCCATCCGAGGCAACAAG | |
| | GATTGCGCCGTCAAGTTCGGCGCTCTGAGAAAAGC | |
| | CCTGGACCTGAGCAGCGGCGATTACTTCAAGGGCG | |
| | TGCCTGCCGCCGATGAAAAGACCAGAGAAGTCTAC | |
| | AAGCCTAAGGGCTGGCGGGTGTTGAGAAATACCCT | |
| | GAACGCCGCCAATCCCATCTTACTGCAGAGACTGA | |
| | GAGATGATCGGAACCTGGCTGACGCCGTGATGGAA | |
| | GCCGTGGCCTACAGCTCTGCGCTGCCCGTGCTGCA | |
| | GGAGCAGCTGCAAGGCCTGCCTTTCAGCGAGGCCG | |
| | AGATCGAGGCCCTGTGTAGACTGCCTTATTCTAGC | |
| | AAGGCCCTGAACGGATACGGCAATAGAAGTAAAAA | |
| | GGCCCTGGATATGCTGCTGGATTGCCTGGAAGAAC | |
| | CTGAGGTGCTAAACCTGACCCAGGCCGAGAATGAC | |
| | TGCGGCCTGCTGGGCCTGCGGATCGCCGGCGCCCA | |
| | GCTGGAGAGATCTGATAGACTGATGCCTTACGAAA | |
| | CCTGGATCGAGCTGACAGGCAGAACAAACAACAAT | |
| | CCTGTGGTGATCCGGAGCATGTCTCAGATGAGAAA | |
| | AGTGGTGAACGCCGTGTGCCGGAAGTGGGGCGTGC | |
| | CCAACGAGATCCATGTGGAACTGGATAGAGAGCTG | |
| | AGACTGCCTCAGCGGGCTAAAGACGAGATCGCCAA | |
| | GGCTAACAAGAAAAACGAGAAGAACAGAGAGAGGA | |
| | TCGCAGGCCAGATTGCAGAACTGAGAGGATGTACC | |
| | GCCGACGAGGTTACAGGTAAACAGATCGAAAAGTA | |
| | CCGGCTCTGGGAAGAGCAGGAGTGCTTCGACCTGT | |
| | ACACCGGCGCCAAAATCGAGGTGGACAGACTGATC | |
| | AGCGATGACACCTACACACAGATCGACCACATCCT | |
| | GCCCTTCAGCAGAACCGGCGAAAACAGCCGGAACA | |
| | ACAAGGTGCTGGTGCTGGCTAAGTCTAATCAAGAC | |
| | AAGAGAGAGCAGACCCCTTACGAGTGGATGAGCCA | |
| | CGACGGCGCCCCTAGCTGGGACGCCTTTGAGAGAA | |
| | GAGTGCAGGAGAATCAAAAGCTGTCCCGGAGAAAG | |
| | AAGAACTTCCTGCTTGAGAAGGACCTGGACACCAA | |
| | AGAGGGCGAGTTCCTGGCCCGGAGCTTCACCGACA | |
| | CAGCTTACATGTCCAGAGAGGCCTGCGCCTACCTG | |
| | GCCGACTGCCTGCTGTTCCCCGATGACGGCGCCAA | |
| | AGCCCATGTGGTGCCTACCACCGGCAGAGCCACAG | |
| | CCTGGCTGCGAAGAAGGTGGGGACTGAATTTCGGC | |
| | AGCAACGGCGAGAAGGACAGAAGCGACGACCGGCA | |
| | CCACGCCACAGACGCCTGTGTGATCGCCGCCTGCA | |
| | GCAGAAGCCTGGTGATCAAGACCGCTAGAATCAAC | |
| | CAAGAAACCCACTGGAGCATCACCCGGGGCATGAA | |
| | CGAAACCCAGAGAAGGGATGCCATCATGAAGGCTC | |
| | TTGAGTCTGTGATGCCCTGGGAAACCTTCGCCAAC | |
| | GAGGTGCGGGCCGCTCACGACTTCGTGGTGCCTAC | |
| | AAGATTCGTGCCAAGAAAAGGAAAGGGCGAGCTGT | |
| | TTGAGCAAACCGTGTACAGATACGCCGGAGTTAAT | |
| | GCCCAGGGTAAAGATATCGCCCGTAAGGCCAGCTC | |
| | CGACAAGGACATCGTGATGGGCAACGCCGTGGTTT | |
| | CCCTGGATGAAAAGAGCGTGATCAAGGTGTCCGAA | |
| | ATGCTGTGCCTGAGACTGTGGCACGATCCTGAGGC | |
| | GAAGAAGGGCCAGGGCGCCTGGTACGCCGACCCAG | |
| | TGTACAAGGCCGACATCCCTGCTCTGAAAGATGGC | |
| | ACCTACGTGCCAAGAATCGCCAAGGCCCACACCGG | |
| | CCGGAAGGCCTGGAAGCCTGTGCCTGAGTCCGCCA | |
| | TGAAGAAGCCCCCCCTGGAAATCTACCTGGGGGAT | |
| | CTGGTGCAGATCGGCGACTTCATGGGCAGATTCTC | |
| | CGGCTACAACATCGCCAACGCCAACTGGTCCTTTG | |
| | TGGACAGACTGACAAAAGAAGCCCTGGGCTGTCCT | |
| | ACAGTGGTGAAGCTGGACAACAAGCTGGCACCAGC | |
| | CATCATCCGGGAATCTATCATCATGCACAAGAGAA | |
| | CCGCCGACGGCTCTGAGTTCGAGTCTCCAAAGAAG | |
| | AAACGGAAAGTGTGA | |

In some embodiments an ANAB Type II Cas protein comprises an amino acid sequence of SEQ ID NO:34, SEQ ID NO:35, or SEQ ID NO:787. In some embodiments, an ANAB Type II Cas protein has nickase activity, for example resulting from one or more amino acid substitutions relative to the sequence of SEQ ID NO:34, SEQ ID NO:35, or SEQ ID NO:787. In some embodiments, the one or more amino acid substitutions providing nickase activity is a D23A substitution, wherein the position of the D23A substitution is defined with respect to the amino acid numbering of SEQ ID NO:8. The corresponding position in SEQ ID NO:34, SEQ ID NO:35, or SEQ ID NO:787 can be determined, for example, by performing a sequence alignment of SEQ ID NO:34, SEQ ID NO:35, or SEQ ID NO:787 with SEQ ID NO:8 (e.g., by BLAST).

### 6.2.5. Fusion and Chimeric Proteins

The disclosure provides Type II Cas proteins (e.g., a BNK Type II Cas protein as described in Section 6.2.1, an AIK Type II Cas protein as described in Section 6.2.2, an HPLH Type II Cas protein as described in Section 6.2.3, or an ANAB Type II Cas protein as described in Section 6.2.4) which are in the form of fusion proteins comprising a Type II Cas protein sequence fused with one or more additional amino acid sequences, such as one or more nuclear localization signals and/or one or more non-native tags. Fusion proteins can also comprise an amino acid sequence of, for example, a nucleoside deaminase, a reverse transcriptase, a transcriptional activator, a transcriptional repressor, a histone-modifying protein, an integrase, or a recombinase.

In some embodiments, a fusion protein of the disclosure comprises a means for localizing the Type II Cas protein to the nucleus, for example a nuclear localization signal.

Non-limiting examples of nuclear localization signals include KRTADGSEFESPKKKRKV (SEQ ID NO:38), PKKKRKV (SEQ ID NO:39), PKKKRRV (SEQ ID NO:40), KRPAATKKAGQAKKKK (SEQ ID NO:41), YGRKKRRQRRR (SEQ ID NO:42), RKKRRQRRR (SEQ ID NO:43), PAAKRVKLD (SEQ ID NO:44), RQRRNELKRSP (SEQ ID NO:45), VSRKRPRP (SEQ ID NO:46), PPKKARED (SEQ ID NO:47), PQPKKKPL (SEQ ID NO:48), SALIKKKKKMAP (SEQ ID NO:49), PKQKKRK (SEQ ID NO:50), RKLKKKIKKL (SEQ ID NO:51), REKKKFLKRR (SEQ ID NO:52), KRKGDEVDGVDEVAKKKSKK (SEQ ID NO:53), RKCLQAGMNLEARKTKK (SEQ ID NO:54),

(SEQ ID NO: 55)
NQSSNFGPMKGGNFGGRSSGPYGGGGQYFAKPRNQGGY,
and
(SEQ ID NO: 56)
RMRIZFKNKGKDTAELRRRRVEVSVELRKAKKDEQILKRRNV.

Exemplary fusion partners include protein tags (e.g., V5-tag (e.g., having the sequence GKPIPNPLLGLDST (SEQ ID NO:57), FLAG-tag, myc-tag, HA-tag, GST-tag, polyHis-tag, MBP-tag), protein domains, transcription modulators, enzymes acting on small molecule substrates, DNA, RNA and protein modification enzymes (e.g., adenosine deaminase, cytidine deaminase, guanosyl transferase, DNA methyltransferase, RNA methyltransferases, DNA demethylases, RNA demethylases, dioxygenases, polyadenylate polymerases, pseudouridine synthases, acetyltransferases, deacetylase, ubiquitin-ligases, deubiquitinases, kinases, phosphatases, NEDD8-ligases, de-NEDDylases, SUMO-ligases, deSUMOylases, histone deacetylases, reverse transcriptases, histone acetyltransferases histone methyltransferases, histone demethylases), protein DNA binding domains, RNA binding proteins, polypeptide sequences with specific biological functions (e.g., nuclear localization signals, mitochondrial localization signals, plastid localization signals, subcellular localization signals, destabilizing signals, Geminin destruction box motifs), and biological tethering domains (e.g., MS2, Csy4 and lambda N protein). Various Type II Cas fusion proteins are described in Ribeiro et al., 2018, In. J. Genomics, Article ID:1652567; Jayavaradhan, et al., 2019, Nat Commun 10:2866; Xiao et al., 2019, The CRISPR Journal, 2(1):51-63; Mali et al., 2013, Nat Methods. 10(10):957-63; U.S. Pat. Nos. 9,322,037, and 9,388,430. In some embodiments, a fusion partner is an adenosine deaminase. An exemplary adenosine deaminase is the tRNA adenosine deaminase (TadA) moiety contained in the adenine base editor ABE8e (Richter, 2020, Nature Biotechnology 38:883-891). The TadA moiety of ABE8e comprises the following amino acid sequence:

(SEQ ID NO: 792)
SEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRI
GRVVFGVRNSKRGAAGSLMNVLNYPGMNHRVEITEGILADECAALLCDF
YRMPRQVFNAQKKAQSSIN

In some embodiments, an adenosine deaminase fusion partner comprises an amino acid sequence having at least 50%, at least 60%, at least 70%, at least 80%, at least 90%, at least 95%, at least 97%, at least 98%, at least 99% amino acid sequence identity with SEQ ID NO:792.

Type II Cas proteins of the disclosure in the form of a fusion protein comprising an adenosine deaminase can be used as an adenine base editor to change an “A” to a “G” in DNA. Type II Cas proteins of the disclosure in the form of a fusion protein comprising a cytidine deaminase can be used as a cytosine base editor to change a “C” to a “T” in DNA.

In some embodiments, a fusion protein of the disclosure comprises a means for deaminating adenosine, for example an adenosine deaminase, e.g., a TadA variant. In some embodiments, a fusion protein of the disclosure comprises a means for deaminating cytidine, for example a cytodine deaminase, e.g., cytidine deaminase 1 (CDA1) or an apolipoprotein B mRNA-editing complex (APOBEC) family deaminase (Cheng et al., 2019, Nat Commun. 10(1):3612; Gehrke et al., 2018, Nat Biotechnol. 36(10):977-982).

In some embodiments, a fusion protein of the disclosure comprises a means for synthesizing DNA from a single-stranded template, for example a reverse transcriptase. Type II Cas proteins of the disclosure in the form of a fusion protein comprising a reverse transcriptase (RT) can be used as a prime editor to carry out precise base editing without double-stranded DNA breaks.

In some embodiments, a fusion protein of the disclosure is a prime editor, e.g., a Type II Cas protein fused to a suitable RT (e.g., Moloney murine leukemia virus (M-MLV) RT or other RT enzyme). Such fusion proteins can be used in conjunction with a prime editing guide RNA (pegRNA) that both specifies the target site and encodes the desired edit (Anzalone et al., 2019, Nature, 576(7785):149-157).

In some embodiments, a fusion protein of the disclosure comprises one or more nuclear localization signals positioned N-terminal and/or C-terminal to a Type II Cas protein sequence (e.g., a BNK Type II Cas protein having a sequence of SEQ ID NO:1, an AIK Type II Cas protein having a sequence of SEQ ID NO:7, an HPLH Type II Cas protein having a sequence of SEQ ID NO:30, or an ANAB Type II Cas protein having a sequence of SEQ ID NO: 34). In some embodiments, a fusion protein of the disclosure comprises an N-terminal and a C-terminal nuclear localization signal, for example each having the sequence KRTADGSEFESPKKKRKV (SEQ ID NO:58).

The disclosure provides chimeric Type II Cas proteins comprising one or more domains of a BNK Type II Cas protein and one or more domains of one or more different proteins (e.g., one or more different Type II Cas proteins), chimeric Type II Cas proteins comprising one or more domains of an AIK Type II Cas protein and one or more domains of one or more different proteins (e.g., one or more different Type II Cas proteins), chimeric Type II Cas proteins comprising one or more domains of an HPLH Type II Cas protein and one or more domains of one or more different proteins (e.g., one or more different Type II Cas proteins), and chimeric Type II Cas proteins comprising one or more domains of an ANAB Type II Cas protein and one or more domains of one or more different proteins (e.g., one or more different Type II Cas proteins).

The domain structures of wild-type AIK, BNK, HPLH, and ANAB Type II Cas proteins were inferred by multiple alignment with the amino acid sequences of Type II Cas proteins for which the crystal structure is known and for which it is thus possible to define the boundaries of each functional domain. The domains identified in Type II Cas proteins are: the RuvC catalytic domain (discontinuous, represented by RuvC-I, RuvC-II, and RuvC-III domains), bridge helix (BH), recognition (REC) domain, HNH catalytic domain, wedge (WED) domain, and PAM-interacting domain (PID).

Table 2 below reports the amino acid positions corresponding to the boundaries between different functional domains in wild-type BNK (SEQ ID NO:2), AIK (SEQ ID NO:8), HPLH (SEQ ID NO:31, and ANAB (SEQ ID NO:35) Type II Cas proteins.

TABLE 2
Amino Acid Positions of AIK, BNK, HPLH, and ANAB Domains

| | Wild-type | Wild-type | Wild-type | Wild-type |
|---|---|---|---|---|
| | AIK | BNK | HPLH | ANAB |
| Domain | Type II Cas | Type II Cas | Type II Cas | Type II Cas |
| RuvC-I | 1-61 | 1-59 | 1-59 | 1-61 |
| BH | 62-94 | 60-92 | 60-92 | 62-94 |
| REC | 95-468 | 93-453 | 93-472 | 95-468 |
| RuvC-II | 469-527 | 454-527 | 473-523 | 469-527 |
| HNH | 528-689 | 528-695 | 524-680 | 528-689 |
| RuvC-III | 690-835 | 696-825 | 681-830 | 690-835 |
| WED | 836-877 | 826-879 | 831-860 | 836-877 |
| PID | 878-1004 | 880-1002 | 861-1005 | 878-1004 |

A chimeric Type II Cas protein can comprise one of more of the following domains (e.g., one or more, two or more, three or more, four or more, five or more, six or more, seven or more) from a BNK Type II Cas protein, AIK Type II Cas protein, HPLH Type II Cas protein, and/or ANAB Type II Cas protein, and one or more domains from one or more other proteins, for example SaCas9, SpCas9 or a Type II Cas protein described in US 2020/0332273, US 2019/0169648, or 2015/0247150 (the contents of each of which are incorporated herein by reference in their entirety): RuvC-I, BH, REC, RuvC-II, HNH, RuvC-III, WED, PID. For example, the PID domain can be swapped between different Type II Cas proteins to change the PAM specificity of the resulting chimeric protein (which is given by the donor PID domain). Swapping of other domains or portions of them is also within the scope of the disclosure (e.g., through protein shuffling).

In some embodiments, a Type II Cas protein of the disclosure comprises one, two, three, four, five, six, seven, or eight of a RuvC-I domain, a BH domain, a REC domain, a RuvC-II domain, a HNH domain, a RuvC-III domain, a WED domain, and a PID domain arranged in the N-terminal to C-terminal direction. In some embodiments, all domains are from a BNK Type II Cas protein (e.g., a BNK Type II Cas protein whose amino acid sequence comprises SEQ ID NO:1, 2, or 3) from an AIK Type II Cas protein (e.g., an AIK Type II Cas protein whose amino acid sequence comprises SEQ ID NO:7, 8, or 9), from an HPLH Type II Cas protein whose amino acid sequence comprises SEQ ID NO:30, 31, or 786, or from an ANAB Type II Cas protein whose amino acid sequence comprises SEQ ID NO:34, 35 or 787. In other embodiments, one or more domains (e.g., one domain), e.g., a PID domain, is from another Type II Cas protein.

In addition, one or more amino acid substitutions can be introduced in one or more domains to modify the properties of the resulting nuclease in terms of editing activity, targeting specificity or PAM recognition specificity. For example, one or more amino acid substitutions can be introduced to provide nickase activity. An exemplary amino acid substitution to provide nickase activity is the D23A substitution, wherein the position of the D23A substitution is defined with respect to the amino acid numbering of SEQ ID NO:8.

### 6.3. Guide RNAs

The disclosure provides gRNA molecules that can be used with Type II Cas proteins of the disclosure to edit genomic DNA, for example mammalian DNA, e.g., human DNA. gRNAs of the disclosure typically comprise a spacer of 15 to 30 nucleotides in length. The spacer can be positioned 5′ of a crRNA scaffold to form a full crRNA. The crRNA can be used with a tracrRNA to effect cleavage of a target genomic sequence.

An exemplary crRNA scaffold sequence that can be used for BNK Type II Cas gRNAs comprises GUUCUGGUCUAAGUUCAUUUCCUAACUGAUAAAAUC (SEQ ID NO:13) and an exemplary tracrRNA sequence that can be used for BNK Type II Cas gRNAs comprises UCAGUUAGGAAAUGGGCUUUCUCCACUAACAAGCUGAGAGAUGCACAAGAUGCGGGGUCGCUAU AUGCGACCAUUUUUCGUAUCCAAA (SEQ ID NO:14).

An exemplary crRNA scaffold sequence that can be used for AIK Type II Cas gRNAs comprises GUCUUGAGCACGCGCCCUUCCCCAAGGUGAUACGCU (SEQ ID NO:20) and an exemplary tracrRNA sequence that can be used for AIK Type II Cas gRNAs comprises UCACCUUGGGGAAGGGCGCGGCUCCAGACAAGGGGAGCCACUUAAGUGGCUUACCCGUAAAGUA ACCCCCGUUCAAUCUUCGGAUUGGGCGGGGCGAACUUUUUU (SEQ ID NO:21).

An exemplary crRNA scaffold sequence that can be used for HPLH Type II Cas gRNAs comprises GUUAUAGCUUCCUUUCCAAAUCAGACAUGCUAUAAU (SEQ ID NO:788) and an exemplary tracrRNA sequence that can be used for HPLH Type II Cas gRNAs comprises UUAUUUUAUGUCUGAUUUGGAAAGGAAGUCUAUAAUAAUCGAAGUUUUCUUUACGAGUAGGGCU CUGACGUCUCAUAUAAUAUAUGAGGCGUCAUCCUUU (SEQ ID NO:789).

An exemplary crRNA scaffold sequence that can be used for ANAB Type II Cas gRNAs comprises GUCUUGAGCACGCGCCCUUCCCCAAGGUGAUACGCU (SEQ ID NO:790) and an exemplary tracrRNA sequence that can be used for ANAB Type II Cas gRNAs comprises UCACCUUGGGGAAGGGCGCGGCUCCAGACAAGGGGAGCCACUUAAGUGGCUUACCCGUAAAGUA ACCCCCGUUCAAUCUUCGGAUUGGGCGGGGCGAACUUUUUU (SEQ ID NO:791).

gRNAs of the disclosure are in some embodiments single guide RNAs (sgRNAs), which typically comprise the spacer at the 5′ end of the molecule and a 3′ sgRNA scaffold. Alternatively, gRNAs can comprise separate crRNA and tracrRNA molecules.

Further features of exemplary gRNA spacer sequences are described in Section 6.3.1 and further features of exemplary 3′ sgRNA scaffolds are described in Section 6.3.2.

### 6.3.1. Spacers

The spacer sequence is partially or fully complementary to a target sequence found in a genomic DNA sequence, for example a human genomic DNA sequence. For example, a spacer sequence can be partially or fully complementary to a nucleotide sequence in a gene having a disease causing mutation. A spacer that is partially complementary to a target sequence can have, for example, one, two, or three mismatches with the target sequence.

gRNAs of the disclosure can comprise a spacer that is 15 to 30 nucleotides in length (e.g., 15 to 25, 16 to 24, 17 to 23, 18 to 22, 19 to 21, 18 to 30, 20 to 28, 22 to 26, or 23 to 25 nucleotides in length). In some embodiments, a spacer is 15 nucleotides in length. In other embodiments, a spacer is 16 nucleotides in length. In other embodiments, a spacer is 17 nucleotides in length. In other embodiments, a spacer is 18 nucleotides in length. In other embodiments, a spacer is 19 nucleotides in length. In other embodiments, a spacer is 20 nucleotides in length. In other embodiments, a spacer is 21 nucleotides in length. In other embodiments, a spacer is 22 nucleotides in length. In other embodiments, a spacer is 23 nucleotides in length. In other embodiments, a spacer is 24 nucleotides in length. In other embodiments, a spacer is 25 nucleotides in length. In other embodiments, a spacer is 26 nucleotides in length. In other embodiments, a spacer is 27 nucleotides in length. In other embodiments, a spacer is 28 nucleotides in length. In other embodiments, a spacer is 29 nucleotides in length. In other embodiments, a spacer is 30 nucleotides in length.

Type II Cas endonucleases require a specific sequence, called a protospacer adjacent motif (PAM) that is downstream (e.g., directly downstream) of the target sequence on the non-target strand. Thus, spacer sequences for targeting a gene of interest can be identified by scanning the gene for PAM sequences recognized by the Type II Cas protein. Exemplary PAM sequences for BNK Type II Cas proteins are shown in Table 3A. Exemplary PAM sequences for AIK Type II Cas proteins are shown in Table 3B. Exemplary PAM sequences for HPLH Type II Cas proteins are shown in Table 3C. Exemplary PAM sequences for ANAB Type II Cas proteins are shown in Table 3D.

TABLE 3A
Exemplary BNK PAM Sequences
Sequence

NRVNRT
NRCNAT
N = A, T, C, or G
R = A or G
V = A, C, or G

TABLE 3B
Exemplary AIK PAM Sequences
Sequence

N₄RHNT
N₄RYNT
N₄GYNT
N₄GTNT
N₄GTTT
N₄GTGT
N₄GCTT
N = A, T, C, or G
R = A or G
H = A, C, or T
Y = C or T

TABLE 3C
Exemplary HPLH PAM Sequences
Sequence

N₄GWAN
N₄GWAA
N₄GNAA
N = A, T, C, or G
W = T or A

TABLE 3D
Exemplary ANAB PAM Sequences
Sequence

N₄RNKA
N₄GHKA
N = A, T, C, or G
R = A or G
K = G or T
H = A, C, or T

Examples 1 and 2 describes exemplary sequences that can be used to target CCR5, EMX1, Fas, FANCF, HBB, ZSCAN2, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, BCR, ATM, HBG1, HPRT, IL2RG, NF1, USH2A, RHO, BcLenh, and CTFR genomic sequences. In some embodiments, a gRNA of the disclosure comprises a spacer sequence targeting one of the foregoing. For example, the gRNA can comprise a spacer corresponding to one of the protospacer sequences disclosed in Table 5 or Table 12 (e.g., a spacer sequence corresponding to the protospacer sequence GCCCTTCAGCTCGATGCGGTTCAC (SEQ ID NO:73) is GCCCUUCAGCUCGAUGCGGUUCAC (SEQ ID NO:74)).

6.3.2. sgRNA Molecules

gRNAs of the disclosure can be single-guide RNA (sgRNA) molecules. A sgRNA can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins.

The sgRNA can comprise a variable length spacer sequence (e.g., 15 to 30 nucleotides) at the 5′ end of the sgRNA sequence and a 3′ sgRNA segment.

Type II Cas gRNAs typically comprise a repeat-antirepeat duplex and/or one or more stem-loops generated by the gRNA's secondary structure. The length of the repeat-antirepeat duplex and/or one or more stem-loops can be modified in order to modulate (e.g., increase) the editing efficacy of a Type II Cas nuclease, and/or to reduce the size of a guide RNA for easier vectorization in situations in which the cargo size of the vector is limiting (e.g., AAV vectors).

For example, the repeat-antirepeat duplex (which in a sgRNA is fused through a synthetic linker to become an additional stem loop in the structure) can be trimmed at different lengths without generally having detrimental effects on nuclease function and in some cases even producing increased enzymatic activity. If bulges are present within this duplex they generally should be retained in the final guide RNA sequence.

Further optimization of the structure can be obtained by introducing targeted base changes into the stems of the gRNA to increase their stability and folding. Such base changes will preferably correspond to the introduction of G:C couples, which are known to generate the strongest Watson-Crick pairing. For the sake of clarity, these substitutions can consist in the introduction of a G or a C in a specific position of a stem together with a complementary substitution in another position of the gRNA sequence which is predicted to base pair with the former, for example according to available bioinformatic tools for RNA folding such as UNAfold or RNAfold.

Stem-loop trimming can also be exploited to stabilize desired secondary structures by removing portions of the guide RNA producing unwanted secondary structures through annealing with other regions of the RNA molecule.

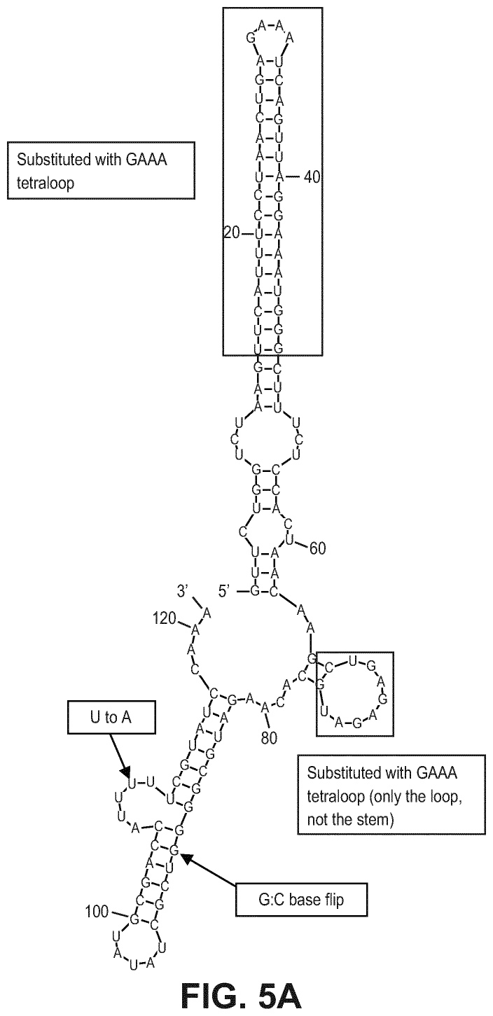

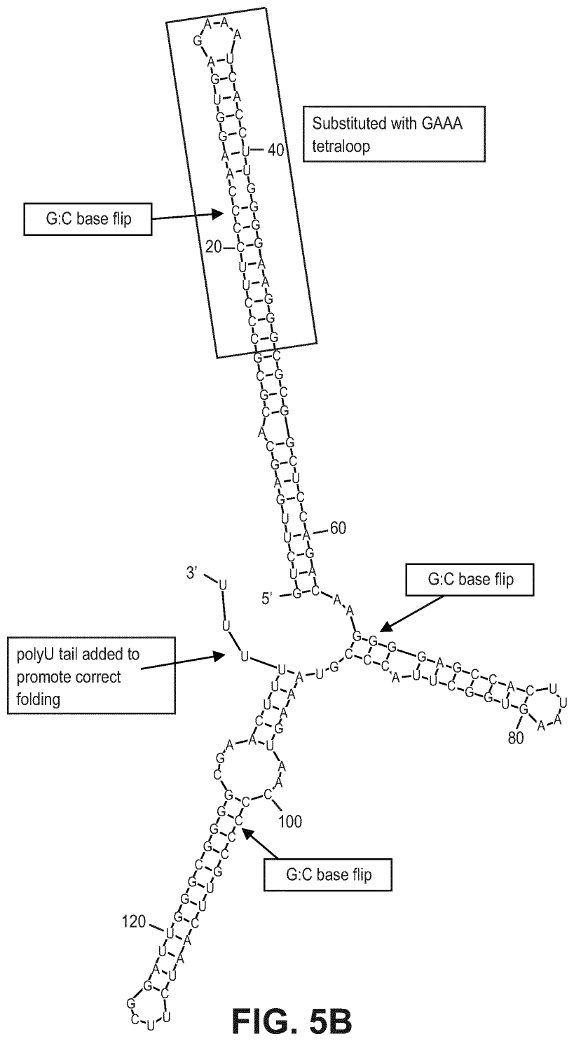

Examples of modifications to that can be made to exemplary BNK and AIK Type II Cas gRNA 3′ scaffolds to make trimmed scaffolds are illustrated in FIG. 5A and FIG. 5B, respectively. For example, referring to FIG. 5A, bases 14-49 (which includes the GAAA tetraloop) can be substituted with a GAAA tetraloop, and the second loop can be substituted with a tetraloop (GAAA) to make a trimmed scaffold. Referring to FIG. 5B, bases 15-50 of (which includes the GAAA tetraloop) can be substituted with a GAAA tetraloop to make a trimmed scaffold.

Further exemplary 3′ sgRNA scaffold sequences for BNK Type II Cas sgRNAs are shown in Table 4A. Further exemplary 3′ sgRNA scaffold sequences for AIK Type II Cas sgRNAs are shown in Table 4B. Exemplary 3′ sgRNA scaffold sequences for HPLH Type II Cas sgRNAs are shown in Table 4C. Exemplary 3′ sgRNA scaffold sequences for ANAB Type II Cas sgRNAs are shown in Table 4D.

TABLE 4A
Sequences of sgRNA Scaffolds for BNK Type II Cas

| | | SEQ ID |
|---|---|---|
| Name | Sequence | NO: |
| BNK Type II | GUUCUGGUCUAAGUUCAUUUCCUAACUGAGAAAUCAGUUAGGAAAUGG | 15 |
| Cas sgRNA_v1 | GCUUUCUCCACUAACAAGCUGAGAGAUGCACAAGAUGCGGGGUCGCUA | |
| scaffold | UAUGCGACCAUUUUUCGUAUCCAAA | |
| BNK Type II | GUUCUGGUCUAAGUUCAUUUCCUAACUGAGAAAUCAGUUAGGAAAUGG | 16 |
| Cas sgRNA_v2 | GCUUUCUCCACUAACAAGCUGAGAGAUGCACAAGAUGCGGGGUCGCUA | |
| scaffold | UAUGCGACCAUUAUUCGUAUCCAAA | |
| BNK Type II | GUUCUGGUCUAAGGAAACUUUCUCCACUAACAAGCUGAGAGAUGCACA | 17 |
| Cas sgRNA_v3 | AGAUGCGGGGUCGCUAUAUGCGACCAUUAUUCGUAUCCAAA | |
| scaffold | | |
| BNK Type II | GUUCUGGUCUAAGUUCAUUUCCUAACUGAGAAAUCAGUUAGGAAAUGG | 18 |
| Cas sgRNA_v4 | GCUUUCUCCACUAACAAGCGAAAGCACAAGAUGCGGGCUCGCUAUAUG | |
| scaffold | CGAGCAUUAUUCGUAUCCAAA | |
| BNK Type II | GUUCUGGUCUAAGGAAACUUUCUCCACUAACAAGCGAAAGCACAAGAU | 19 |
| Cas sgRNA_v5 | GCGGGCUCGCUAUAUGCGAGCAUUAUUCGUAUCCAAA | |
| scaffold | | |

TABLE 4B
Sequences of sgRNA Scaffolds for AIK Type II Cas

| | | SEQ ID |
|---|---|---|
| Name | Sequence | NO: |
| AIK Type II Cas | GUCUUGAGCACGCGCCCUUCCCCAAGGUGAGAAAUCACCUUGGGGAA | 22 |
| sgRNA_v1 | GGGCGCGGCUCCAGACAAGGGGAGCCACUUAAGUGGCUUACCCGUAA | |
| scaffold | AGUAACCCCCGUUCAAUCUUCGGAUUGGGCGGGGCGAAC | |
| AIK Type II Cas | GUCUUGAGCACGCGCCCUUCCGCAAGGUGAGAAAUCACCUUGCGGAA | 23 |
| sgRNA_v2 | GGGCGCGGCUCCAGACAAGCGGAGCCACUUAAGUGGCUUACGCGUAA | |
| scaffold | AGUAACCGCCGUUCAAUCUUCGGAUUGGGCGGCGCGAAC | |
| AIK Type II Cas | GUCUUGAGCACGCGAAAGCGGCUCCAGACAAGGGGAGCCACUUAAGU | 24 |
| sgRNA_v3 | GGCUUACCCGUAAAGUAACCCCCGUUCAAUCUUCGGAUUGGGGGGGG | |
| scaffold | CGAAC | |
| AIK Type II Cas | GUCUUGAGCACGCGAAAGCGGCUCCAGACAAGCGGAGCCACUUAAGU | 25 |
| sgRNA_v4 | GGCUUACGCGUAAAGUAACCGCCGUUCAAUCUUCGGAUUGGGCGGCG | |
| scaffold | CGAAC | |
| AIK Type II Cas | GUCUUGAGCACGCGAAAGCGGCUCCAGACAAGCGGAGCCACUUAAGU | 822 |
| sgRNA_v5 | GGCUUACGCGUAAAGUAACCGCCGAAAGGCGCGAAC | |
| scaffold | | |

TABLE 4C
Sequences of sgRNA Scaffolds for HPLH Type II Cas

| | | SEQ ID |
|---|---|---|
| Name | Sequence | NO: |
| HPLH Type II | GUUAUAGCUUCCUUUCCAAAUCAGACAUGCUAUAGAAAUAUUAUAUGU | 75 |
| Cas sgRNA_v1 | CUGAUUUGGAAAGGAAGUCUAUAAUAAUCGAAGUUAUCUUUACGAGUA | |
| scaffold | GGGCUCUGACGUCACAUAUAAUAUAUGUGGCGUCAUCC | |

TABLE 4D
Sequences of sgRNA Scaffolds for ANAB Type II Cas

| | | SEQ ID |
|---|---|---|
| Name | Sequence | NO: |
| ANAB Type II | GUCUUGAGCACGCGCCCUUCCCCAAGGUGAGAAAUCACCUUGGGGAA | 76 |
| Cas sgRNA_v1 | GGGCGCGGCUCCAGACAAGGGGAGCCACUUAAGUGGCUUACCCGUAA | |
| scaffold | AGUAACCCCCGUUCAAUCUUCGGAUUGGGGGGGGCGAAC | |
| ANAB Type II | GUCUUGAGCACGCGCCCUUCCGCAAGGUGAGAAAUCACCUUGCGGAA | 177 |
| Cas sgRNA_v2 | GGGCGCGGCUCCAGACAAGCGGAGCCACUUAAGUGGCUUACGCGUAA | |
| scaffold | AGUAACCGCCGUUCAAUCUUCGGAUUGGGCGGCGCGAAC | |
| ANAB Type II | GUCUUGAGCACGCGAAAGCGGCUCCAGACAAGGGGAGCCACUUAAGU | 78 |
| Cas sgRNA_v3 | GGCUUACCCGUAAAGUAACCCCCGUUCAAUCUUCGGAUUGGGCGGGG | |
| scaffold | CGAAC | |
| ANAB Type II | GUCUUGAGCACGCGAAAGCGGCUCCAGACAAGCGGAGCCACUUAAGU | 79 |
| Cas sgRNA_v4 | GGCUUACGCGUAAAGUAACCGCCGUUCAAUCUUCGGAUUGGGCGGCG | |
| scaffold | CGAAC | |
| ANAB Type II | GUCUUGAGCACGCGAAAGCGGCUCCAGACAAGCGGAGCCACUUAAGU | 822 |
| Cas sgRNA_v5 | GGCUUACGCGUAAAGUAACCGCCGAAAGGCGCGAAC | |
| scaffold | | |

The sgRNA (e.g., for use with BNK Type II Cas proteins, AIK Type II Cas proteins, HPLH Type II Cas proteins, or ANAB Type II Cas proteins) can comprise no uracil base at the 3′ end of the sgRNA sequence. Typically, however, the sgRNA comprises one or more uracil bases at the 3′ end of the sgRNA sequence, for example to promote correct sgRNA folding. For example, the sgRNA can comprise 1 uracil (U) at the 3′ end of the sgRNA sequence. The sgRNA can comprise 2 uracil (UU) at the 3′ end of the sgRNA sequence. The sgRNA can comprise 3 uracil (UUU) at the 3′ end of the sgRNA sequence. The sgRNA can comprise 4 uracil (UUUU) at the 3′ end of the sgRNA sequence. The sgRNA can comprise 5 uracil (UUUUU) at the 3′ end of the sgRNA sequence. The sgRNA can comprise 6 uracil (UUUUUU) at the 3′ end of the sgRNA sequence. The sgRNA can comprise 7 uracil (UUUUUUU) at the 3′ end of the sgRNA sequence. The sgRNA can comprise 8 uracil (UUUUUUUU) at the 3′ end of the sgRNA sequence. Different length stretches of uracil can be appended at the 3′end of a sgRNA as terminators. Thus, for example, the 3′ sgRNA sequences set forth in Table 4A, Table 4B, Table 4C, and Table 4D can be modified by adding (or removing) one or more uracils at the end of the sequence.

In some embodiments, a sgRNA scaffold for use with an AIK Type II Cas protein comprises the sequence GUCUUGAGCACGCGCCCUUCCCCAAGGUGAGAAAUCACCUUGGGGAAGGGCGCGGCUCCAGACA AGGGGAGCCACUUAAGUGGCUUACCCGUAAAGUAACCCCCGUUCAAUCUUCGGAUUGGGCGGGG CGAACUUUUUU (SEQ ID NO:26).

In some embodiments, a sgRNA scaffold for use with an AIK Type II Cas protein comprises the sequence GUCUUGAGCACGCGCCCUUCCGCAAGGUGAGAAAUCACCUUGCGGAAGGGCGCGGCUCCAGACA AGCGGAGCCACUUAAGUGGCUUACGCGUAAAGUAACCGCCGUUCAAUCUUCGGAUUGGGCGGCG CGAACUUUUUU (SEQ ID NO:27).

In some embodiments, a sgRNA scaffold for use with an AIK Type II Cas protein comprises the sequence GUCUUGAGCACGCGAAAGCGGCUCCAGACAAGGGGAGCCACUUAAGUGGCUUACCCGUAAAGUA ACCCCCGUUCAAUCUUCGGAUUGGGCGGGGCGAACUUUUUU (SEQ ID NO:28).

In some embodiments, a sgRNA scaffold for use with an AIK Type II Cas protein comprises the sequence GUCUUGAGCACGCGAAAGCGGCUCCAGACAAGCGGAGCCACUUAAGUGGCUUACGCGUAAAGUA ACCGCCGUUCAAUCUUCGGAUUGGGCGGCGCGAACUUUUUU (SEQ ID NO:29).

In some embodiments, a sgRNA scaffold for use with an AIK Type II Cas protein comprises the sequence GUCUUGAGCACGCGAAAGCGGCUCCAGACAAGCGGAGCCACUUAAGUGGCUUACGCGUAAAGUA ACCGCCGAAAGGCGCGAACUUUUUU (SEQ ID NO:823).

6.3.3. Modified gRNA Molecules

Guide RNAs can be readily synthesized by chemical means, enabling a number of modifications to be readily incorporated, as described in the art. The disclosed gRNA (e.g., sgRNA) molecules can be unmodified or can contain any one or more of an array of chemical modifications.

While chemical synthetic procedures are continually expanding, purifications of such RNAs by procedures such as high-performance liquid chromatography (HPLC, which avoids the use of gels such as PAGE) tends to become more challenging as polynucleotide lengths increase significantly beyond a hundred or so nucleotides. One approach that can be used for generating chemically modified RNAs of greater length is to produce two or more molecules that are ligated together. Much longer RNAs, such as those encoding a Type II Cas endonuclease, are more readily generated enzymatically. While fewer types of modifications are available for use in enzymatically produced RNAs, there are still modifications that can be used to, for instance, enhance stability, reduce the likelihood or degree of innate immune response, and/or enhance other attributes, as described herein and in the art.

By way of illustration of various types of modifications, especially those used frequently with smaller chemically synthesized RNAs, modifications can comprise one or more nucleotides modified at the 2′ position of the sugar, for instance a 2′-O-alkyl, 2′-O-alkyl-O-alkyl, or 2′-fluoro-modified nucleotide. In some examples, RNA modifications can comprise 2′-fluoro, 2′-amino or 2′-O-methyl modifications on the ribose of pyrimidines, abasic residues, or an inverted base at the 3′ end of the RNA. Such modifications can be routinely incorporated into oligonucleotides and these oligonucleotides have been shown to have a higher Tm (thus, higher target binding affinity) than 2′-deoxyoligonucleotides against a given target.

A number of nucleotide and nucleoside modifications have been shown to make the oligonucleotide into which they are incorporated more resistant to nuclease digestion than the native oligonucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Some oligonucleotides are oligonucleotides with phosphorothioate backbones and those with heteroatom backbones, particularly CH₂—NH—O—CH₂, CH, ˜N(CH₃)—O—CH₂ (known as a methylene(methylimino) or MMI backbone), CH₂—O—N(CH₃)—CH₂, CH₂ —N(CH₃)—N(CH₃)—CH₂ and O—N(CH₃)— CH₂ —CH₂ backbones, wherein the native phosphodiester backbone is represented as O— P— O— CH,); amide backbones (see De Mesmaeker et al. 1995, Ace. Chem. Res., 28:366-374); morpholino backbone structures (see U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., 1991, Science 254:1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Morpholino-based oligomeric compounds are described in Braasch and David Corey, 2002, Biochemistry, 41(14):4503-4510; Genesis, Volume 30, Issue 3, (2001); Heasman, 2002, Dev. Biol., 243: 209-214; Nasevicius et al., 2000, Nat. Genet., 26:216-220; Lacerra et al., 2000, Proc. Natl. Acad. Sci., 97: 9591-9596; and U.S. Pat. No. 5,034,506.

Cyclohexenyl nucleic acid oligonucleotide mimetics are described in Wang et al., 2000, J. Am. Chem. Soc., 122: 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

One or more substituted sugar moieties can also be included, e.g., one of the following at the 2′ position: OH, SH, SCH₃, F, OCN, OCH₃, OCH₃ O(CH₂)n CH₃, O(CH₂)n NH₂, or O(CH₂)n CH₃, where n is from 1 to about 10; C₁ to C₁₀ lower alkyl, alkoxyalkoxy, substituted lower alkyl, alkaryl or aralkyl; Cl; Br; CN; CF₃; OCF₃; O-, S-, or bi-alkyl; O-, S-, or N-alkenyl; SOCH₃; SO₂ CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. In some aspects, a modification includes 2′-methoxyethoxy (2′-O—CH₂CH₂OCH₃, also known as 2′-O-(2-methoxyethyl)) (Martin et al., 1995, Helv. Chim. Acta, 78, 486). Other modifications include 2′-methoxy (2′-O—CH₃), 2′-propoxy (2′-OCH₂ CH₂CH₃) and 2′-fluoro (2′-F). Similar modifications can also be made at other positions on the oligonucleotide, particularly the 3′ position of the sugar on the 3′ terminal nucleotide and the 5′ position of 5′ terminal nucleotide. Oligonucleotides can also have sugar mimetics, such as cyclobutyls in place of the pentofuranosyl group.

In some examples, both a sugar and an internucleoside linkage (in the backbone) of the nucleotide units can be replaced with novel groups. The base units can be maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide can be replaced with an amide containing backbone, for example, an aminoethylglycine backbone. The nucleobases can be retained and bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262. Further teaching of PNA compounds can be found in Nielsen et al., 1991, Science, 254: 1497-1500.

RNAs such as guide RNAs can also include, additionally or alternatively, nucleobase (often referred to in the art simply as “base”) modifications or substitutions. As used herein, “unmodified” or “natural” nucleobases include adenine (A), guanine (G), thymine (T), cytosine (C), and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxy cytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino) adenine, 2-(imidazolylalkyl)adenine, 2-(aminoalklyamino) adenine or other heterosubstituted alkyladenines, 2-thiouracil, 2-thiothymine, 5-bromouracil, 5-hydroxymethyluracil, 8-azaguanine, 7-deazaguanine, N₆ (6-aminohexyl) adenine, and 2,6-diaminopurine. Komberg, A., DNA Replication, W. H. Freeman & Co., San Francisco, pp. 75-77 (1980); Gebeyehu et al., Nucl. Acids Res. 15:4513 (1997). A “universal” base known in the art, e.g., inosine, can also be included. 5-Me-C substitutions have been shown to increase nucleic acid duplex stability by about 0.6-1.2° C. (Sanghvi, Y. S., in Crooke, S. T. and Lebleu, B., eds., Antisense Research and Applications, CRC Press, Boca Raton, 1993, pp. 276-278) and are aspects of base substitutions.

Modified nucleobases can comprise other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine, and 3-deazaguanine and 3-deazaadenine.

Further, nucleobases can comprise those disclosed in U.S. Pat. No. 3,687,808, those disclosed in ‘The Concise Encyclopedia of Polymer Science and Engineering’, 858-859, Kroschwitz, J. I., ed. John Wiley & Sons, 1990, those disclosed by Englisch et al., Angewandle Chemie, International Edition’, 1991, 30, p. 613, and those disclosed by Sanghvi, Y. S., Chapter 15, Antisense Research and Applications’, 289-302, Crooke, S. T. and Lebleu, B. ea., CRC Press, 1993. Certain of these nucleobases can be useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by about 0.6-1.2° C. (Sanghvi, Y. S., Crooke, S. T. and Lebleu, B., eds, ‘Antisense Research and Applications’, CRC Press, Boca Raton, 1993, 276-278) and are aspects of base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,681,941; 5,750,692; 5,763,588; 5,830,653; 6,005,096; and U.S. Patent Application Publication 2003/0158403.

Thus, a modified gRNA can include, for example, one or more non-natural sugars, internucleotide linkages and/or bases. It is not necessary for all positions in a given gRNA to be uniformly modified, and in fact more than one of the aforementioned modifications can be incorporated in a single oligonucleotide, or even in a single nucleoside within an oligonucleotide.

The guide RNAs and/or mRNA (or DNA) encoding an endonuclease can be chemically linked to one or more moieties or conjugates that enhance the activity, cellular distribution, or cellular uptake of the oligonucleotide. Such moieties comprise, but are not limited to, lipid moieties such as a cholesterol moiety (Letsinger et al. 1989, Proc. Natl. Acad. Sci. USA, 86: 6553-6556); cholic acid (Manoharan et al, 1994, Bioorg. Med. Chem. Let., 4: 1053-1060); a thioether, e.g., hexyl-S-tritylthiol (Manoharan et al, 1992, Ann. N. Y. Acad. Sci., 660: 306-309; Manoharan et al., 1993, Bioorg. Med. Chem. Let., 3: 2765-2770); a thiocholesterol (Oberhauser et al., 1992, Nucl. Acids Res., 20: 533-538); an aliphatic chain, e.g., dodecandiol or undecyl residues (Kabanov et al, 1990, FEBS Lett., 259: 327-330; Svinarchuk et al, 1993, Biochimie, 75: 49-54); a phospholipid, e.g., di-hexadecyl-rac-glycerol or triethylammonium 1,2-di-O-hexadecyl-rac-glycero-3-H-phosphonate (Manoharan et al., 1995, Tetrahedron Lett., 36: 3651-3654; and Shea et al, 1990, Nucl. Acids Res., 18: 3777-3783); a polyamine or a polyethylene glycol chain (Mancharan et al, 1995, Nucleosides & Nucleotides, 14: 969-973); adamantane acetic acid (Manoharan et al, 1995, Tetrahedron Lett., 36: 3651-3654); a palmityl moiety (Mishra et al., 1995, Biochim. Biophys. Acta, 1264: 229-237); or an octadecylamine or hexylamino-carbonyl-t oxycholesterol moiety (Crooke et al, 1996, J. Pharmacol. Exp. Ther., 277: 923-937). See also U.S. Pat. Nos. 4,828,979; 4,948,882; 5,218,105; 5,525,465; 5,541,313; 5,545,730; 5,552,538; 5,578,717; 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241; 5,391,723; 5,416,203; 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Sugars and other moieties can be used to target proteins and complexes comprising nucleotides, such as cationic polysomes and liposomes, to particular sites. For example, hepatic cell directed transfer can be mediated via asialoglycoprotein receptors (ASGPRs); see, e.g., Hu, et al., 2014, Protein Pept Lett. 21(10):1025-30. Other systems known in the art and regularly developed can be used to target biomolecules of use in the present case and/or complexes thereof to particular target cells of interest.

Targeting moieties or conjugates can include conjugate groups covalently bound to functional groups, such as primary or secondary hydroxyl groups. Conjugate groups of the present disclosure include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this present disclosure, include groups that improve uptake, enhance resistance to degradation, and/or strengthen sequence-specific hybridization with the target nucleic acid. Groups that enhance the pharmacokinetic properties, in the context of this disclosure, include groups that improve uptake, distribution, metabolism or excretion of the compounds of the present disclosure. Representative conjugate groups are disclosed in International Patent Application Publication WO1993007883, and U.S. Pat. No. 6,287,860. Conjugate moieties include, but are not limited to, lipid moieties such as a cholesterol moiety, cholic acid, a thioether, e.g., hexyl-5-trityl thiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241; 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

A large variety of modifications have been developed and applied to enhance RNA stability, reduce innate immune responses, and/or achieve other benefits that can be useful in connection with the introduction of polynucleotides into human cells, as described herein; see, e.g., the reviews by Whitehead K A et al., 2011, Annual Review of Chemical and Biomolecular Engineering, 2: 77-96; Gaglione and Messere, 2010, Mini Rev Med Chem, 10(7):578-95; Chernolovskaya et al, 2010, Curr Opin Mol Ther., 12(2): 158-67; Deleavey et al., 2009, Curr Protoc Nucleic Acid Chem Chapter 16:Unit 16.3; Behlke, 2008, Oligonucleotides 18(4):305-19; Fucini et al, 2012, Nucleic Acid Ther 22(3): 205-210; Bremsen et al, 2012, Front Genet 3: 154.

### 6.4. Systems

The disclosure provides systems comprising a Type II Cas protein of the disclosure (e.g., as described in Section 6.2) and a means for targeting the Type II Cas protein to a target genomic sequence. The means for targeting the Type II Cas protein to a target genomic sequence can be a guide RNA (gRNA) (e.g., as described in Section 6.3).

The disclosure also provides systems comprising a Type II Cas protein of the disclosure (e.g., as described in Section 6.2) and a gRNA (e.g., as described in Section 6.3). The systems can comprise a ribonucleoprotein particle (RNP) in which a Type II Cas protein is complexed with a gRNA, for example a sgRNA or separate crRNA and tracrRNA. Systems of the disclosure can in some embodiments further comprise genomic DNA complexed with the Type II Cas protein and the gRNA. Accordingly, the disclosure provides systems comprising a Type II Cas protein, a genomic DNA, and gRNA, all complexed with one another.

The systems of the disclosure can exist within a cell (whether the cell is in vivo, ex vivo, or in vitro) or outside a cell (e.g., in a particle our outside of a particle).

### 6.5. Nucleic Acids

The disclosure provides nucleic acids (e.g., DNA or RNA) encoding Type II Cas proteins (e.g., BNK Type II Cas proteins, AIK Type II Cas proteins, HPLH Type II Cas proteins, and ANAB Type II Cas proteins), nucleic acids encoding gRNAs of the disclosure, nucleic acids encoding both Type II Cas proteins and gRNAs, and pluralities of nucleic acids, for example comprising a nucleic acid encoding a Type II Cas protein and a gRNA.

A nucleic acid encoding a Type II Cas protein and/or gRNA can be, for example, a plasmid or a viral genome (e.g., a lentivirus, retrovirus, adenovirus, or adeno-associated virus genome). Plasmids can be, for example, plasmids for producing virus particles, e.g., lentivirus particles, or plasmids for propagating the Type II Cas and gRNA coding sequences in bacterial (e.g., E. coli) or eukaryotic (e.g., yeast) cells.

A nucleic acid encoding a Type II Cas protein can, in some embodiments, further encode a gRNA. Alternatively, a gRNA can be encoded by a separate nucleic acid (e.g., DNA or mRNA).

Nucleic acids encoding a Type II Cas protein can be codon optimized, e.g., where at least one non-common codon or less-common codon has been replaced by a codon that is common in a host cell. For example, a codon optimized nucleic acid can direct the synthesis of an optimized messenger mRNA, e.g., optimized for expression in a mammalian expression system. As an example, if the intended target nucleic acid is within a human cell, a human codon-optimized polynucleotide encoding Type II Cas can be used for producing a Type II Cas polypeptide. Exemplary codon-optimized sequences are shown in Table 1A, Table 1B, Table 1C, and Table 1D.

Nucleic acids of the disclosure, e.g., plasmids and viral vectors, can comprise one or more regulatory elements such as promoters, enhancers, and other expression control elements (e.g., transcription termination signals, such as polyadenylation signals and poly-U sequences). Such regulatory elements are described, for example, in Goeddel, 1990, GENE EXPRESSION TECHNOLOGY: METHODS IN ENZYMOLOGY 185, Academic Press, San Diego, Calif. Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest or in particular cell types. Regulatory elements may also direct expression in a temporal-dependent manner, such as in a cell-cycle dependent or developmental stage-dependent manner, which may or may not also be tissue or cell-type specific. In some embodiments, a nucleic acid of the disclosure comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof, e.g., to express a Type II Cas protein and a gRNA separately. Examples of pol III promoters include, but are not limited to, U6 and H1 promoters. Examples of pol II promoters include, but are not limited to, the retroviral Rous Sarcoma virus (RSV) LTR promoter (optionally with the RSV enhancer), the cytomegalovirus (CMV) promoter (optionally with the CMV enhancer) (see, e.g., Boshart et al, 1985, Cell 41:521-530), the SV40 promoter, the dihydrofolate reductase promoter, the β-actin promoter, the phosphoglycerol kinase (PGK) promoter, and EF1α promoters (for example, full length EF1α promoter and the EFS promoter, which is a short, intron-less form of the full EF1α promoter). Exemplary enhancer elements include WPRE; CMV enhancers; the R-U5′ segment in LTR of HTLV-I; SV40 enhancer; and the intron sequence between exons 2 and 3 of rabbit 3-globin. It will be appreciated by those skilled in the art that the design of an expression vector can depend on such factors as the choice of the host cell, the level of expression desired, etc.

The term “vector” refers to a polynucleotide molecule capable of transporting another nucleic acid to which it has been linked. One type of polynucleotide vector includes a “plasmid”, which refers to a circular double-stranded DNA loop into which additional nucleic acid segments are or can be ligated. Another type of polynucleotide vector is a viral vector; wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

In some examples, vectors can be capable of directing the expression of nucleic acids to which they are operably linked. Such vectors can be referred to herein as “recombinant expression vectors”, or more simply “expression vectors”, which serve equivalent functions.

The term “operably linked” means that the nucleotide sequence of interest is linked to regulatory sequence(s) in a manner that allows for expression of the nucleotide sequence. The term “regulatory sequence” is intended to include, for example, promoters, enhancers and other expression control elements (e.g., polyadenylation signals). Such regulatory sequences are well known in the art and are described, for example, in Goeddel; Gene Expression Technology: Methods in Enzymology 185, Academic Press, San Diego, CA (1990). Regulatory sequences include those that direct constitutive expression of a nucleotide sequence in many types of host cells, and those that direct expression of the nucleotide sequence only in certain host cells (e.g., tissue-specific regulatory sequences). It will be appreciated by those skilled in the art that the design of the expression vector can depend on such factors as the choice of the target cell, the level of expression desired, and the like.

Vectors can include, but are not limited to, viral vectors based on vaccinia virus, poliovirus, adenovirus, adeno-associated virus (e.g., AAV2, AAV5, AAV7m8, AAV8, AAV9, AAVrh8r, AAVrh10), SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXTI, pSG5, pSVK3, pBPV, pMSG, and pSVLSV40 (Pharmacia). Additional vectors contemplated for eukaryotic target cells include, but are not limited to, the vectors pCTx-1, pCTx-2, and pCTx-3. Other vectors can be used so long as they are compatible with the host cell.

In some examples, a vector can comprise one or more transcription and/or translation control elements. Depending on the host/vector system utilized, any of a number of suitable transcription and translation control elements, including constitutive and inducible promoters, transcription enhancer elements, transcription terminators, etc. can be used in the expression vector. The vector can be a self-inactivating vector that either inactivates the viral sequences or the components of the CRISPR machinery or other elements.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-I promoters (for example, the full EF1α promoter and the EFS promoter), a hybrid construct comprising the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I.

An expression vector can also contain a ribosome binding site for translation initiation and a transcription terminator. The expression vector can also comprise appropriate sequences for amplifying expression. The expression vector can also include nucleotide sequences encoding non-native tags (e.g., histidine tag, hemagglutinin tag, green fluorescent protein, etc.) that are fused to the site-directed polypeptide, thus resulting in a fusion protein.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter). In some cases, the promoter can be a spatially restricted and/or temporally restricted promoter (e.g., a tissue specific promoter, for example a human RHO promoter or human rhodopsin kinase promoter (hGRK), a cell type specific promoter, etc.).

### 6.6. Particles and Cells

The disclosure further provides particles comprising a Type II Cas protein of the disclosure (e.g., a BNK Type II Cas protein, an AIK Type II Cas protein, an HPLH Type II Cas protein, or an ANAB Type II Cas protein), particles comprising a gRNA of the disclosure, particles comprising a system of the disclosure, and particles comprising a nucleic acid or plurality of nucleic acids of the disclosure. The particles can in some embodiments comprise or further comprise a gRNA, or a nucleic acid encoding the gRNA (e.g., DNA or mRNA). For example, the particles can comprise a RNP of the disclosure. Exemplary particles include lipid nanoparticles, vesicles, viral-like particles (VLPs) and gold nanoparticles. See, e.g., WO 2020/012335, the contents of which are incorporated herein by reference in their entireties, which describes vesicles that can be used to deliver gRNA molecules and Type II Cas proteins to cells (e.g., complexed together as a RNP).

The disclosure provides particles (e.g., virus particles) comprising a nucleic acid encoding a Type II Cas protein of the disclosure. The particles can further comprise a nucleic acid encoding a gRNA. Alternatively, a nucleic acid encoding a Type II Cas protein can further encode a gRNA.

The disclosure further provides pluralities of particles (e.g., pluralities of virus particles). Such pluralities can include a particle encoding a Type II Cas protein and a different particle encoding a gRNA. For example, a plurality of particles can comprise a virus particle (e.g., a AAV2, AAV5, AAV7m8, AAV8, AAV9, AAVrh8r, or AAVrh10 virus particle) encoding a Type II Cas protein and a second virus particle (e.g., a AAV2, AAV5, AAV7m8, AAV8, AAV9, AAVrh8r, or AAVrh10 virus particle) encoding a gRNA. Alternatively, a plurality of particles can comprise a plurality of virus particles where each particle encodes a Type II Cas protein and a gRNA.

The disclosure further provides cells and populations of cells (e.g., ex vivo cells and populations of cells) that can comprise a Type II Cas protein (e.g., introduced to the cell as a RNP) or a nucleic acid encoding the Type II Cas protein (e.g., DNA or mRNA) (optionally also encoding a gRNA). The disclosure further provides cells and populations of cells comprising a gRNA of the disclosure (optionally complexed with a Type II Cas protein) or a nucleic acid encoding the gRNA (e.g., DNA or mRNA) (optionally also encoding a Type II Cas protein). The cells and populations of cells can be, for example, human cells such as a stem cell, e.g., a hematopoietic stem cell (HSC), a pluripotent stem cell, an induced pluripotent stem cell (iPS), or an embryonic stem cell. Methods for introducing proteins and nucleic acids to cells are known in the art. For example, a RNP can be produced by mixing a Type II Cas protein and one or more guide RNAs in an appropriate buffer. An RNP can be introduced to a cell, for example, via electroporation and other methods known in the art.

The cell populations of the disclosure can be cells in which gene editing by the systems of the disclosure has taken place, or cells in which the components of a system of the disclosure have been introduced or expressed but gene editing has not taken place, or a combination thereof. A cell population can comprise, for example, a population in which at least 1%, at least 5%, at least 10%, at least 15%, at least 20%, at least 30%, at least 40%, at least 50%, at least 60%, or at least 70% of the cells have undergone gene editing by a system of the disclosure.

### 6.7. Pharmaceutical Compositions

Also disclosed herein are pharmaceutical formulations and medicaments comprising a Type II Cas protein, gRNA, nucleic acid or plurality of nucleic acids, system, particle, or plurality of particles of the disclosure together with a pharmaceutically acceptable excipient.

Suitable excipients include, but are not limited to, salts, diluents, (e.g., Tris-HCl, acetate, phosphate), preservatives (e.g., Thimerosal, benzyl alcohol, parabens), binders, fillers, solubilizers, disintegrants, sorbents, solvents, pH modifying agents, antioxidants, antinfective agents, suspending agents, wetting agents, viscosity modifiers, tonicity agents, stabilizing agents, and other components and combinations thereof. Suitable pharmaceutically acceptable excipients can be selected from materials which are generally recognized as safe (GRAS), and may be administered to an individual without causing undesirable biological side effects or unwanted interactions. Suitable excipients and their formulations are described in Remington's Pharmaceutical Sciences, 16th ed. 1980, Mack Publishing Co. In addition, such compositions can be complexed with polyethylene glycol (PEG), metal ions, or incorporated into polymeric compounds such as polyacetic acid, polyglycolic acid, hydrogels, etc., or incorporated into liposomes, microemulsions, micelles, unilamellar or multilamellar vesicles, erythrocyte ghosts or spheroblasts. Suitable dosage forms for administration, e.g., parenteral administration, include solutions, suspensions, and emulsions.

The components of the pharmaceutical formulation can be dissolved or suspended in a suitable solvent such as, for example, water, Ringer's solution, phosphate buffered saline (PBS), or isotonic sodium chloride. The formulation may also be a sterile solution, suspension, or emulsion in a nontoxic, parenterally acceptable diluent or solvent such as 1,3-butanediol.

In some cases, formulations can include one or more tonicity agents to adjust the isotonic range of the formulation. Suitable tonicity agents are well known in the art and include glycerin, mannitol, sorbitol, sodium chloride, and other electrolytes. In some cases, the formulations can be buffered with an effective amount of buffer necessary to maintain a pH suitable for parenteral administration. Suitable buffers are well known by those skilled in the art and some examples of useful buffers are acetate, borate, carbonate, citrate, and phosphate buffers.

In some embodiments, the formulation can be distributed or packaged in a liquid form, or alternatively, as a solid, obtained, for example by lyophilization of a suitable liquid formulation, which can be reconstituted with an appropriate carrier or diluent prior to administration. In some embodiments, the formulations can comprise a guide RNA and a Type II Cas protein in a pharmaceutically effective amount sufficient to edit a gene in a cell. The pharmaceutical compositions can be formulated for medical and/or veterinary use.

### 6.8. Methods of Altering a Cell

The disclosure further provides methods of using the Type II Cas proteins, gRNAs, nucleic acids (including pluralities of nucleic acids), systems, and particles (including pluralities of particles) of the disclosure for altering cells.

In one aspect, a method of altering a cell comprises contacting a eukaryotic cell (e.g., a human cell) with a nucleic acid, particle, system or pharmaceutical composition described herein.

Contacting a cell with a disclosed nucleic acid, particle, system or pharmaceutical composition can be achieved by any method known in the art and can be performed in vivo, ex vivo, or in vitro. In some embodiments, the methods can include obtaining one or more cells from a subject prior to contacting the cell(s) with a herein disclosed nucleic acid, particle, system or pharmaceutical composition. In some embodiments, the methods can further comprise returning or implanting the contacted cell or a progeny thereof to the subject.

Type II Cas and gRNA, as well as nucleic acids encoding Type II Cas and gRNAs can be delivered to a cell by any means known in the art, for example, by viral or non-viral delivery vehicles, electroporation or lipid nanoparticles.

A polynucleotide encoding Type II Cas and a gRNA, can be delivered to a cell (ex vivo or in vivo) by a lipid nanoparticle (LNP). LNPs can have, for example, a diameter of less than 1000 nm, 500 nm, 250 nm, 200 nm, 150 nm, 100 nm, 75 nm, 50 nm, or 25 nm. Alternatively, a nanoparticle can range in size from 1-1000 nm, 1-500 nm, 1-250 nm, 25-200 nm, 25-100 nm, 35-75 nm, or 25-60 nm. LNPs can be made from cationic, anionic, neutral lipids, and combinations thereof. Neutral lipids, such as the fusogenic phospholipid DOPE or the membrane component cholesterol, can be included in LNPs as ‘helper lipids’ to enhance transfection activity and nanoparticle stability.

LNPs can also be comprised of hydrophobic lipids, hydrophilic lipids, or both hydrophobic and hydrophilic lipids. Lipids and combinations of lipids that are known in the art can be used to produce a LNP. Examples of lipids used to produce LNPs are: DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids are: 98N12-5, C12-200, DLin-KC2-DMA (KC2), DLin-MC3-DMA (MC3), XTC, MD1, and 7C1. Examples of neutral lipids are: DPSC, DPPC, POPC, DOPE, and SM. Examples of PEG-modified lipids are: PEG-DMG, PEG-CerCl4, and PEG-CerC20. Lipids can be combined in any number of molar ratios to produce a LNP. In addition, the polynucleotide(s) can be combined with lipid(s) in a wide range of molar ratios to produce a LNP.

Type II Cas and/or gRNAs can be delivered to a cell via an adeno-associated viral vector (e.g., of an AAV2, AAV5, AAV7m8, AAV8, AAV9, AAVrh8r, or AAVrh10 serotype), or by another viral vector. Other viral vectors include, but are not limited to lentivirus, adenovirus, alphavirus, enterovirus, pestivirus, baculovirus, herpesvirus, Epstein Barr virus, papovavirus, poxvirus, vaccinia virus, and herpes simplex virus. In some embodiments, a Type II Cas mRNA is formulated in a lipid nanoparticle, while a sgRNA is delivered to a cell in an AAV or other viral vector. In some embodiments, one or more AAV vectors (e.g., one or more AAV2, AAV5, AAV7m8, AAV8, AAV9, AAVrh8r, or AAVrh10 serotype) are used to deliver both a sgRNA and a Type II Cas. In some embodiments, a Type II Cas and a sgRNA are delivered using separate vectors. In other embodiments, a Type II Cas and a sgRNA are delivered using a single vector. BNK Type II Cas and AIK Type II Cas, with their relatively small size, can be delivered with a gRNA (e.g., sgRNA) using a single AAV vector.

Compositions and methods for delivering Type II Cas and gRNAs to a cell and/or subject are further described in PCT Patent Application Publications WO 2019/102381, WO 2020/012335, and WO 2020/053224, each of which is incorporated by reference herein in its entirety.

DNA cleavage can result in a single-strand break (SSB) or double-strand break (DSB) at particular locations within the DNA molecule. Such breaks can be and regularly are repaired by natural, endogenous cellular processes, such as homology-dependent repair (HDR) and non-homologous end-joining (NHEJ). These repair processes can edit the targeted polynucleotide by introducing a mutation, thereby resulting in a polynucleotide having a sequence which differs from the polynucleotide's sequence prior to cleavage by a Type II Cas.

NHEJ and HDR DNA repair processes consist of a family of alternative pathways. Non-homologous end-joining (NHEJ) refers to the natural, cellular process in which a double-stranded DNA-break is repaired by the direct joining of two non-homologous DNA segments. See, e.g. Cahill et al., 2006, Front. Biosci. 11:1958-1976. DNA repair by non-homologous end-joining is error-prone and frequently results in the untemplated addition or deletion of DNA sequences at the site of repair. Thus, NHEJ repair mechanisms can introduce mutations into the coding sequence which can disrupt gene function. NHEJ directly joins the DNA ends resulting from a double-strand break, sometimes with a modification of the polynucleotide sequence such as a loss of or addition of nucleotides in the polynucleotide sequence. The modification of the polynucleotide sequence can disrupt (or perhaps enhance) gene expression.

Homology-dependent repair (HDR) utilizes a homologous sequence, or donor sequence, as a template for inserting a defined DNA sequence at the break point. The homologous sequence can be in the endogenous genome, such as a sister chromatid. Alternatively, the donor can be an exogenous nucleic acid, such as a plasmid, a single-strand oligonucleotide, a double-stranded oligonucleotide, a duplex oligonucleotide or a virus, that has regions of high homology with the nuclease-cleaved locus, but which can also contain additional sequence or sequence changes including deletions that can be incorporated into the cleaved target locus.

A third repair mechanism includes microhomology-mediated end joining (MMEJ), also referred to as “Alternative NHEJ (ANHEJ)”, in which the genetic outcome is similar to NHEJ in that small deletions and insertions can occur at the cleavage site. MMEJ can make use of homologous sequences of a few base pairs flanking the DNA break site to drive a more favored DNA end joining repair outcome. In some instances, it may be possible to predict likely repair outcomes based on analysis of potential microhomologies at the site of the DNA break.

Modifications of a cleaved polynucleotide by HDR, NHEJ, and/or ANHEJ can result in, for example, mutations, deletions, alterations, integrations, gene correction, gene replacement, gene tagging, transgene insertion, nucleotide deletion, gene disruption, translocations and/or gene mutation. The aforementioned process outcomes are examples of editing a polynucleotide.

Advantages of ex vivo cell therapy approaches include the ability to conduct a comprehensive analysis of the therapeutic prior to administration. Nuclease-based therapeutics can have some level of off-target effects. Performing gene correction ex vivo allows a method user to characterize the corrected cell population prior to implantation, including identifying any undesirable off-target effects. Where undesirable effects are observed, a method user may opt not to implant the cells or cell progeny, may further edit the cells, or may select new cells for editing and analysis. Other advantages include ease of genetic correction in iPSCs compared to other primary cell sources. iPSCs are prolific, making it easy to obtain the large number of cells that will be required for a cell-based therapy. Furthermore, iPSCs are an ideal cell type for performing clonal isolations. This allows screening for the correct genomic correction, without risking a decrease in viability.

Although certain cells present an attractive target for ex vivo treatment and therapy, increased efficacy in delivery may permit direct in vivo delivery to such cells. Ideally the targeting and editing is directed to the relevant cells. Cleavage in other cells can also be prevented by the use of promoters only active in certain cell types and/or developmental stages.

Additional promoters are inducible, and therefore can be temporally controlled if the nuclease is delivered as a plasmid. The amount of time that delivered protein and RNA remain in the cell can also be adjusted using treatments or domains added to change the half-life. In vivo treatment would eliminate a number of treatment steps, but a lower rate of delivery can require higher rates of editing. In vivo treatment can eliminate problems and losses from ex vivo treatment and engraftment.

An advantage of in vivo gene therapy can be the ease of therapeutic production and administration. The same therapeutic approach and therapy has the potential to be used to treat more than one patient, for example a number of patients who share the same or similar genotype or allele. In contrast, ex vivo cell therapy typically requires using a subject's own cells, which are isolated, manipulated and returned to the same patient.

Progenitor cells (also referred to as stem cells herein) are capable of both proliferation and giving rise to more progenitor cells, which in turn have the ability to generate a large number of cells that can in turn give rise to differentiated or differentiable daughter cells. The daughter cells themselves can be induced to proliferate and produce progeny that subsequently differentiate into one or more mature cell types, while also retaining one or more cells with parental developmental potential. The term “stem cell” refers then to a cell with the capacity or potential, under particular circumstances, to differentiate to a more specialized or differentiated phenotype, and which retains the capacity, under certain circumstances, to proliferate without substantially differentiating. In one aspect, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell can derive from a multipotent cell that itself is derived from a multipotent cell, and so on. While each of these multipotent cells can be considered stem cells, the range of cell types that each can give rise to can vary considerably. Some differentiated cells also have the capacity to give rise to cells of greater developmental potential. Such capacity can be natural or can be induced artificially upon treatment with various factors. In many biological instances, stem cells can also be “multipotent” because they can produce progeny of more than one distinct cell type, but this is not required.

Human cells described herein can be induced pluripotent stem cells (iPSCs). An advantage of using iPSCs in the methods of the disclosure is that the cells can be derived from the same subject to which the progenitor cells are to be administered. That is, a somatic cell can be obtained from a subject, reprogrammed to an induced pluripotent stem cell, and then differentiated into a progenitor cell to be administered to the subject (e.g., an autologous cell). Because progenitors are essentially derived from an autologous source, the risk of engraftment rejection or allergic response can be reduced compared to the use of cells from another subject or group of subjects. In addition, the use of iPSCs negates the need for cells obtained from an embryonic source. Thus, in one aspect, the stem cells used in the disclosed methods are not embryonic stem cells.

Methods are known in the art that can be used to generate pluripotent stem cells from somatic cells. Pluripotent stem cells generated by such methods can be used in the method of the disclosure.

Reprogramming methodologies for generating pluripotent cells using defined combinations of transcription factors have been described. Mouse somatic cells can be converted to ES cell-like cells with expanded developmental potential by the direct transduction of Oct4, Sox2, Klf4, and c-Myc; see, e.g., Takahashi and Yamanaka, 2006, Cell 126(4): 663-76. iPSCs resemble ES cells, as they restore the pluripotency-associated transcriptional circuitry and much of the epigenetic landscape. In addition, mouse iPSCs satisfy all the standard assays for pluripotency: specifically, in vitro differentiation into cell types of the three germ layers, teratoma formation, contribution to chimeras, germline transmission (see, e.g., Maherali and Hochedlinger, 2008, Cell Stem Cell. 3(6):595-605), and tetraploid complementation.

Human iPSCs can be obtained using similar transduction methods, and the transcription factor trio, OCT4, SOX2, and NANOG, has been established as the core set of transcription factors that govern pluripotency; see, e.g., 2014, Budniatzky and Gepstein, Stem Cells Transl Med. 3(4):448-57; Barrett et al, 2014, Stem Cells Trans Med 3: 1-6 sctm.2014-0121; Focosi et al, 2014, Blood Cancer Journal 4: e211. The production of iPSCs can be achieved by the introduction of nucleic acid sequences encoding stem cell-associated genes into an adult, somatic cell, historically using viral vectors.

iPSCs can be generated or derived from terminally differentiated somatic cells, as well as from adult stem cells, or somatic stem cells. That is, a non-pluripotent progenitor cell can be rendered pluripotent or multipotent by reprogramming. In such instances, it may not be necessary to include as many reprogramming factors as required to reprogram a terminally differentiated cell. Further, reprogramming can be induced by the non-viral introduction of reprogramming factors, e.g., by introducing the proteins themselves, or by introducing nucleic acids that encode the reprogramming factors, or by introducing messenger RNAs that upon translation produce the reprogramming factors (see e.g., Warren et al., 2010, Cell Stem Cell, 7(5):618-30. Reprogramming can be achieved by introducing a combination of nucleic acids encoding stem cell-associated genes, including, for example, Oct-4 (also known as Oct-3/4 or Pouf51), SoxI, Sox2, Sox3, Sox 15, Sox 18, NANOG, Klfl, Klf2, Klf4, Klf5, NR5A2, c-Myc, 1-Myc, n-Myc, Rem2, Tert, and LIN28. Reprogramming using the methods and compositions described herein can further comprise introducing one or more of Oct-3/4, a member of the Sox family, a member of the Klf family, and a member of the Myc family to a somatic cell. The methods and compositions described herein can further comprise introducing one or more of each of Oct-4, Sox2, Nanog, c-MYC and Klf4 for reprogramming. As noted above, the exact method used for reprogramming is not necessarily critical to the methods and compositions described herein. However, where cells differentiated from the reprogrammed cells are to be used in, e.g., human therapy, in one aspect the reprogramming is not affected by a method that alters the genome. Thus, in such examples, reprogramming can be achieved, e.g., without the use of viral or plasmid vectors.

Efficiency of reprogramming (the number of reprogrammed cells) derived from a population of starting cells can be enhanced by the addition of various agents, e.g., small molecules, as shown by Shi et al., 2008, Cell-Stem Cell 2:525-528; Huangfu et al., 2008, Nature Biotechnology 26(7):795-797; and Marson et al., 2008, Cell-Stem Cell 3: 132-135. Thus, an agent or combination of agents that enhance the efficiency or rate of induced pluripotent stem cell production can be used in the production of patient-specific or disease-specific iPSCs. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HD AC) inhibitors, valproic acid, 5′-azacytidine, dexamethasone, suberoylanilide, hydroxamic acid (SAHA), vitamin C, and trichostatin (TSA), among others. Other non-limiting examples of reprogramming enhancing agents include: Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-IH,3H-benzo[de]isoquinolin-2-yl)-N-hydroxybutanamide), Phenylbutyrate (e.g., sodium phenylbutyrate) and Valproic Acid ((VP A) and other short chain fatty acids), Scriptaid, Suramin Sodium, Trichostatin A (TSA), APHA Compound 8, Apicidin, Sodium Butyrate, pi valoyloxy methyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-C1-UCHA (e.g., 6-(3-chlorophenylureido)caproic hydroxamic acid), AOE (2-amino-8-oxo-9, 10-epoxy decanoic acid), CHAP31 and CHAP 50. Other reprogramming enhancing agents include, for example, dominant negative forms of the HDACs (e.g, catalytically inactive forms), siRNA inhibitors of the HDACs, and antibodies that specifically bind to the HDACs. Such inhibitors are available, e.g., from BIOMOL International, Fukasawa, Merck Biosciences, Novartis, Gloucester Pharmaceuticals, Titan Pharmaceuticals, MethylGene, and Sigma Aldrich.

To confirm the induction of pluripotent stem cells, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, FbxI5, EcatI, EsgI, Eras, Gdfi, Fgf4, Cripto, Daxl, Zpf296, Slc2a3, Rexl, Utfl, and Natl. In one case, for example, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. Detection can involve not only RT-PCR, but also detection of protein markers. Intracellular markers can be best identified via RT-PCR, or protein detection methods such as immunocytochemistry, while cell surface markers are readily identified, e.g., by immunocytochemistry.

Pluripotency of isolated cells can be confirmed by tests evaluating the ability of the iPSCs to differentiate into cells of each of the three germ layers. As one example, teratoma formation in nude mice can be used to evaluate the pluripotent character of the isolated clones. The cells can be introduced into nude mice and histology and/or immunohistochemistry can be performed on a tumor arising from the cells. The growth of a tumor comprising cells from all three germ layers, for example, further indicates that the cells are pluripotent stem cells.

Patient-specific iPS cells or cell line can be created. There are many established methods in the art for creating patient specific iPS cells, e.g., as described in Takahashi and Yamanaka 2006; Takahashi, Tanabe et al. 2007. For example, the creating step can comprise: a) isolating a somatic cell, such as a skin cell or fibroblast, from the patient; and b) introducing a set of pluripotency-associated genes into the somatic cell in order to induce the cell to become a pluripotent stem cell. The set of pluripotency-associated genes can be one or more of the genes selected from the group consisting of OCT4, SOX1, SOX2, SOX3, SOX15, SOX18, NANOG, KLF1, KLF2, KLF4, KLF5, c-MYC, n-MYC, REM2, TERT and LIN28.

In some aspects, a biopsy or aspirate of a subject's bone marrow can be performed. A biopsy or aspirate is a sample of tissue or fluid taken from the body. There are many different kinds of biopsies or aspirates. Nearly all of them involve using a sharp tool to remove a small amount of tissue. If the biopsy will be on the skin or other sensitive area, numbing medicine can be applied first. A biopsy or aspirate can be performed according to any of the known methods in the art. For example, in a bone marrow aspirate, a large needle is used to enter the pelvis bone to collect bone marrow.

In some aspects, a mesenchymal stem cell can be isolated from a subject. Mesenchymal stem cells can be isolated according to any method known in the art, such as from a subject's bone marrow or peripheral blood. For example, marrow aspirate can be collected into a syringe with heparin. Cells can be washed and centrifuged on a Percoll™ density gradient. Cells, such as blood cells, liver cells, interstitial cells, macrophages, mast cells, and thymocytes, can be separated using density gradient centrifugation media, Percoll™. The cells can then be cultured in Dulbecco's modified Eagle's medium (DMEM) (low glucose) containing 10% fetal bovine serum (FBS) (Pittinger et. al., 1999, Science 284: 143-147).

### 6.8.1. Exemplary Genomic Targets

The Type II Cas proteins and gRNAs of the disclosure can be used to alter various genomic targets. In some aspects, the methods of altering a cell are methods for altering a CCR5, EMX1, Fas, FANCF, HBB, ZSCAN2, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, BCR, ATM, HBG1, HPRT, IL2RG, NF1, USH2A, RHO, BcLenh, or CTFR genomic sequence.

In some embodiments, the methods of altering a cell are methods for altering a hemoglobin subunit beta (HBB) gene. HBB mutations are associated with 3-thalassemia and SCD. Dever et al., 2016 Nature 539(7629):384-389.

In some embodiments, the methods of altering a cell are methods for altering a CCR5 gene. CCR5 has demonstrated involvement in several different disease states including, but not limited to, human immunodeficiency virus (HIV) and acquired immune deficiency syndrome (AIDS). WO 2018/119359 describes CCR5 editing by CRISPR-Cas to make loss of function CCR5 in order to provide protection against HIV infection, decrease one or more symptoms of HIV infection, halt or delay progression of HIV to AIDS, and/or decrease one or more symptoms of AIDS.

In some embodiments, the methods of altering a cell are methods for altering a PD1, B2M gene, TRAC gene, or a combination thereof. CAR-T cells having PD1, B2M and TRAC genes disrupted by CRISPR-Type II Cas have demonstrated enhanced activity in preclinical glioma models. Choi et al., 2019, Journal for ImmunoTherapy of Cancer 7:309.

In some embodiments, the methods of altering a cell are methods for altering an USH2A gene. Mutations in the USH2A gene can cause Usher syndrome type 2A, which is characterized by progressive hearing and vision loss.

In some embodiments, the methods of altering a cell are methods for altering a RHO gene. Mutations in the RHO gene can cause retinitis pigmentosa (RP).

In some embodiments, the methods of altering a cell are methods for altering a DNMT1 gene. Mutations in the DNMT1 gene can cause DNMT1-related disorder, which is a degenerative disorder of the central and peripheral nervous systems. DNMT1-related disorder is characterized by sensory impairment, loss of sweating, dementia, and hearing loss.

### 7. EXAMPLES

### 7.1. Example 1: Identification and Characterization of BNK and AIK Type II Cas Proteins

This Example describes studies performed to identify and characterize BNK and AIK Type II Cas orthologs.

### 7.1.1. Materials and Methods

### 7.1.1.1. Plasmids

A pX330-derived plasmid was used to express the Type II Cas orthologs in mammalian cells. Briefly, pX330 was modified by substituting SpCas9 and its sgRNA scaffold with the human codon-optimized coding sequence of the Type II Cas of interest and its sgRNA scaffold, generating pX-Type II Cas-AIK and pX-Type II Cas-BNK. The BNK and AIK Type II Cas coding sequences, modified by the addition of an SV5 tag at the N-terminus and two nuclear localization signals (one at the N-terminus and one at the C-terminus) and human codon-optimized, as well as the sgRNA scaffolds were obtained as synthetic fragments from either Genscript or Genewiz. Spacer sequences were cloned into the pX-Type II Cas plasmids as annealed DNA oligonucleotides containing a variable 24-nt spacer sequence using a double BsaI site present in the plasmid. The list of spacer sequences and relative cloning oligonucleotides used in the present Example is reported in Table 5.

TABLE 5
Sequences of the Oligonucleotides Used For Cloning sgRNA Spacers and Sequences of Their Relative Target Sites

| Spacer Sequences Used in Reporter Assays | | | | | Oligo Used to Clone the Spacer in pX Plasmid (**) | | | |
|---|---|---|---|---|---|---|---|---|
| | | SEQ ID | | SEQ ID | | SEQ ID | | SEQ ID |
| Name | Protospacer | NO: | Target (*) | NO: | Oligo 1 (5′ > 3′) | NO: | Oligo 2 (5′ > 3′) | NO: |
| gRNA2_ | GCCCTTCAGCTC | 80 | gatGCCCTTCAGCTCGATGCG | 81 | caccGCCCTTCAGCTCG | 82 | agacGTGAACCGCATC | 83 |
| AIK_EGFP | GATGCGGTTCAC | | GTTCACCAGGGTGTcgc | | ATGCGGTTCAC | | GAGCTGAAGGGC | |
| gRNA3_ | CCTCGCCGGACA | 84 | cgcCCTCGCCGGACACGCTG | 85 | caccGCCTCGCCGGACA | 86 | agacACAAGTTCAGCG | 87 |
| AIK_EGFP | CGCTGAACTTGT | | AACTTGTGGCCGTTTacg | | CGCTGAACTTGT | | TGTCCGGCGAGGC | |
| gRNA2_ | GGTGCGCTCCTG | 88 | gatGGTGCGCTCCTGGACGT | 89 | caccGGTGCGCTCCTGG | 90 | gaacGAAGGCTACGTC | 91 |
| BNK_EGFP | GACGTAGCCTTC | | AGCCTTCGGGCATGGcgg | | ACGTAGCCTTC | | CAGGAGCGCACC | |
| gRNA3_ | GCCCGAAGGCTA | 92 | catGCCCGAAGGCTACGTCC | 93 | caccGCCCGAAGGCTAC | 94 | gaacCGCTCCTGGACG | 95 |
| BNK_EGFP | CGTCCAGGAGCG | | AGGAGCGCACCATCTtct | | GTCCAGGAGCG | | TAGCCTTCGGGC | |
| Spacer Sequences to Target Endogenous Loci | | | | | Oligo Used to Clone the Spacer in pX Plasmid (**) | | | |
| | | SEQ ID | | SEQ ID | | | | SEQ ID |
| Name | Protospacer | NO: | Target (*) | NO: | Oligo 1 (5′ > 3′) | | Oligo 2 (5′ > 3′) | NO: |
| gRNA1_ | TGCCCCTCCCTC | 96 | ctgTGCCCCTCCCTCCCTGGC | 97 | caccGTGCCCCTCCCTC | 98 | agacACCTGGGCCAG | 99 |
| AIK_EMX1 | CCTGGCCCAGGT | | CCAGGTGAAGGTGTggt | | CCTGGCCCAGGT | | GGAGGGAGGGGCAC | |
| gRNA2_ | CCCAGTGGCTGC | 100 | aggCCCAGTGGCTGCTCTGG | 101 | caccGCCCAGTGGCTGC | 102 | agacGAGGCCCCCAG | 103 |
| AIK_EMX1 | TCTGGGGGCCTC | | GGGCCTCCTGAGTTTctc | | TCTGGGGGCCTC | | AGCAGCCACTGGGC | |
| gRNA3_ | GGGCATGGTTTC | 104 | aatGGGCATGGTTTCATAACT | 105 | caccGGGCATGGTTTCAT | 106 | agacCCTCCTAGTTAT | 107 |
| AIK_EMX1 | ATAACTAGGAGG | | AGGAGGTGGTGTTTata | | AACTAGGAGG | | GAAACCATGCCC | |
| gRNA1_ | GAAGCAGGCCAA | 108 | cacGAAGCAGGCCAATGGGG | 109 | caccGAAGCAGGCCAAT | 110 | gaacATGTCCTCCCCA | 111 |
| BNK_EMX1 | TGGGGAGGACAT | | AGGACATCGATGTCAcct | | GGGGAGGACAT | | TTGGCCTGCTTC | |
| gRNA3_ | GCCCACTGTGTC | 112 | ctgGCCCACTGTGTCCTCTTC | 113 | caccGCCCACTGTGTCC | 114 | gaacGGGCAGGAAGA | 115 |
| BNK_EMX1 | CTCTTCCTGCCC | | CTGCCCTGCCATCCcct | | TCTTCCTGCCC | | GGACACAGTGGGC | |
| gRNA1_ | CGCCTGGGCAGC | 116 | gctCCGCCTGGGCAGCCAGG | 117 | caccGCGCCTGGGCAGC | 118 | agacAGGCCAGCCCTG | 119 |
| AIK_FAS_ | CAGGGCTGGCCT | | GCTGGCCTCAGGGTGTGTT | | CAGGGCTGGCCT | | GCTGCCCAGGCGC | |
| gRNA2_ | GAAGGGAGACAA | 120 | aagAGAGAATTCCCGGAAGG | 121 | caccGAGAGAATTCCCG | 122 | agacTTGTCTCCCTTC | 123 |
| AIK_FAS_ | AGAGAATTCCCG | | GAGACAAGGCAGTTTctt | | GAAGGGAGACAA | | CGGGAATTCTCTC | |
| gRNA1_ | CCCAGCAGGAGA | 124 | aacCCCAGCAGGAGACCAAG | 125 | caccGCCCAGCAGGAGA | 126 | gaacATTTCTGCTTGGT | 127 |
| BNK_FAS | CCAAGCAGAAAT | | CAGAAATCACCATGGgag | | CCAAGCAGAAAT | | CTCCTGCTGGGC | |
| gRNA2_ | ACAAGGCAGTTTC | 128 | gagACAAGGCAGTTTCTTTTT | 129 | caccGACAAGGCAGTTT | 130 | gaacACACAGAAAAAG | 131 |
| BNK_FAS | TTTTTCTGTGT | | CTGTGTGACAATAAaaa | | CTTTTTCTGTGT | | AAACTGCCTTGTC | |
| gRNA1_ | GACAAGTGTGATC | 132 | ggtGACAAGTGTGATCACTTG | 133 | caccGACAAGTGTGATCA | 134 | agacACCACCCAAGTG | 135 |
| AIK_CCR5 | ACTTGGGTGGT | | GGTGGTGGCTGTGTttg | | CTTGGGTGGT | | ATCACACTTGTC | |
| gRNA2_ | GCAAGAGGCTCC | 136 | ccaGCAAGAGGCTCCCGAGC | 137 | caccGCAAGAGGCTCCC | 138 | agacCTTGCTCGCTCG | 139 |
| AIK_CCR5 | CGAGCGAGCAAG | | GAGCAAGCTCAGTTTaca | | GAGCGAGCAAG | | GGAGCCTCTTGC | |
| gRNA1_ | GCACAGGGCTGT | 140 | gagGCACAGGGCTGTGAGGC | 827 | caccGCACAGGGCTGTG | 828 | gaacAAGATAAGCCTC | 829 |
| BNK_CCR5 | GAGGCTTATCTT | | TTATCTTCACCATCAtga | | AGGCTTATCTT | | ACAGCCCTGTGC | |
| gRNA2_ | CTTCTTACTGTCC | 141 | ttcCTTCTTACTGTCCCCTTCT | 142 | caccCTTCTTACTGTCCC | 143 | gaacAGCCCAGAAGG | 144 |
| BNK_CCR5 | CCTTCTGGGCT | | GGGCTCACTATGCtgc | | CTTCTGGGCT | | GGACAGTAAGAAG | |
| gRNA1_ | GCATGTCAATCTC | 145 | aatGCATGTCAATCTCCCAGC | 146 | caccGCATGTCAATCTCC | 147 | agacAAAGACGCTGGG | 148 |
| AIK_FANCF | CCAGCGTCTTT | | GTCTTTATCCGTGTtcc | | CAGCGTCTTT | | AGATTGACATGC | |
| gRNA2_ | GGAAGGCCGAAG | 149 | tggGGAAGGCCGAAGCGGAG | 150 | caccGGAAGGCCGAAGC | 151 | agacCGGGACGCTCC | 152 |
| AIK_FANCF | CGGAGCGTCCCG | | CGTCCCGCCAGGTTTctc | | GGAGCGTCCCG | | GCTTCGGCCTTCC | |
| gRNA3_ | GCGCGCTACCTG | 156 | tggGCGCGCTACCTGCGCCA | 157 | caccGCGCGCTACCTGC | 158 | agacATGGATGTGGCG | 159 |
| AIK_FANCF | CGCCACATCCAT | | CATCCATCGGCGCTTtgg | | GCCACATCCAT | | CAGGTAGCGCGC | |
| gRNA1_ | GAGGGAGGGCT | 160 | caaGAGATATATCTTAGAGGG | 161 | caccGAGATATATCTTAG | 162 | agacAGCCCTCCCTCT | 163 |
| AIK_HBB | GAGATATATCTTA | | AGGGCTGAGGGTTTgaa | | AGGGAGGGCT | | AAGATATATCTC | |
| gRNA2_ | ATTGGCCAACCC | 164 | tgcTCCTGGGAGTAGATTGGC | 165 | caccGTCCTGGGAGTAG | 166 | agacGGGTTGGCCAAT | 167 |
| AIK_HBB | TCCTGGGAGTAG | | CAACCCTAGGGTGTggc | | ATTGGCCAACCC | | CTACTCCCAGGAC | |
| gRNA1_ | CATGAGGCATTTG | 168 | gcaCATGAGGCATTTGTAGG | 169 | caccGCATGAGGCATTT | 170 | agacGAGAAGCCCTAC | 171 |
| AIK_ZSCAN2 | TAGGGCTTCTC | | GCTTCTCGCCCGTGTggg | | GTAGGGCTTCTC | | AAATGCCTCATGC | |
| gRNA2_ | CATTTCCCACACT | 172 | gaaCATTTCCCACACTCGCTG | 173 | caccGCATTTCCCACACT | 174 | agacCAAATGCAGCGA | 175 |
| AIK_ZSCAN2 | CGCTGCATTTG | | CATTTGTAGGGTTTctc | | CGCTGCATTTG | | GTGTGGGAAATGC | |
| gRNA1_ | CAACATGAGAG | 176 | actTTGATTCTTACAACAACAT | 177 | caccGTTGATTCTTACAA | 178 | agacCTCTCATGTTGTT | 179 |
| AIK_Chr6 | TTGATTCTTACAA | | GAGAGAGGGGTGTtgt | | CAACATGAGAG | | GTAAGAATCAAC | |
| gRNA1_ | AAAGAAATACTAA | 180 | tagAAAGAAATACTAAGACAT | 181 | caccGAAAGAAATACTAA | 182 | agacCTCTGCATGTCT | 183 |
| AIK_ADAM | GACATGCAGAG | | GCAGAGAGGTGCTTtgc | | GACATGCAGAG | | TAGTATTTCTTTC | |
| gRNA2_ | GGGGCAGAGAGA | 184 | tggGGGGCAGAGAGAGAGAG | 185 | caccGGGGCAGAGAGAG | 186 | agacTCGCTCACTCTC | 187 |
| AIK_ADAM | GAGAGTGAGCGA | | TGAGCGAGTGAGTGTgtg | | AGAGTGAGCGA | | TCTCTCTGCCCC | |
| gRNA1_ | GATTGGCTGGGC | 188 | tgcGGGCCTTGTCCTGATTGG | 189 | caccGGGCCTTGTCCTG | 190 | lagacGCCCAGCCAATC | 191 |
| AIK_B2M | GGGCCTTGTCCT | | CTGGGCACGCGTTTaat | | ATTGGCTGGGC | | AGGACAAGGCCC | |
| gRNA2_ | GAACGCGTGGAG | 192 | ggcGCTGAGGTTTGTGAACG | 193 | caccGCTGAGGTTTGTG | 194 | agacCTCCACGCGTTC | 195 |
| AIK_B2M | GCTGAGGTTTGT | | CGTGGAGGGGCGCTTggg | | AACGCGTGGAG | | ACAAACCTCAGC | |
| gRNA1_ | GTTGGCTGAAAA | 196 | gctGTTGGCTGAAAAGGTGGT | 197 | caccGTTGGCTGAAAAG | 198 | agacACATAGACCACC | 199 |
| AIK_CXCR4 | GGTGGTCTATGT | | CTATGTTGGCGTCTgga | | GTGGTCTATGT | | TTTTCAGCCAAC | |
| gRNA2_ | GTCATCTACACAG | 200 | catGTCATCTACACAGTCAAC | 201 | caccGTCATCTACACAGT | 202 | lagacGTAGAGGTTGAC | 203 |
| AIK_CXCR4 | TCAACCTCTAC | | CTCTACAGCAGTGTcct | | CAACCTCTAC | | TGTGTAGATGAC | |
| gRNA1_ | TGTCCCAGAGCC | 204 | gggTCGGCGGTCAGGTGTCC | 205 | caccGTCGGCGGTCAGG | 206 | agacGGCTCTGGGACA | 207 |
| AIK_PD1 | TCGGCGGTCAGG | | CAGAGCCAGGGGTCTgga | | TGTCCCAGAGCC | | CCTGACCGCCGAC | |
| gRNA2_ | GTTCTTAGGTAGG | 208 | atgGTTCTTAGGTAGGTGGGG | 209 | caccGTTCTTAGGTAGGT | 210 | agacCCGCCGACCCCA | 211 |
| AIK_PD1 | TGGGGTCGGCGG | | TCGGCGGTCAGGTGTccc | | GGGGTCGGCGG | | CCTACCTAAGAAC | |
| gRNA1_ | TCGCCTGTCAAGT | 212 | tacTCGCCTGTCAAGTGGCGT | 213 | caccGTCGCCTGTCAAG | 214 | agacGGTGTCACGCCA | 215 |
| AIK_DNMT1 | GGCGTGACACC | | GACACCGGGCGTGTtcc | | TGGCGTGACACC | | CTTGACAGGCGAC | |
| gRNA2_ | GGGAGGTGGCAG | 216 | gaaGGGAGGTGGCAGGGGG | 217 | caccGGGAGGTGGCAGG | 218 | agacGCTTTCCTCCCC | 219 |
| AIK_Match8 | GGGGAGGAAAGC | | AGGAAAGCAGAGGTTTggg | | GGGAGGAAAGC | | CTGCCACCTCCC | |
| gRNA1 | GATAAGGCCGAG | 220 | atgGATAAGGCCGAGACCAC | 221 | caccGATAAGGCCGAGA | 222 | agacCTGATTGGTGGT | 223 |
| AIK_TRAC | ACCACCAATCAG | | CAATCAGAGGAGTTTtag | | CCACCAATCAG | | CTCGGCCTTATC | |
| gRNA1_ | GCCTCGGCGCTG | 224 | cagGCCTCGGCGCTGACGAT | 225 | caccGCCTCGGCGCTGA | 226 | agacCACCCAGATCGT | 227 |
| AIK_TRBC | ACGATCTGGGTG | | CTGGGTGACGGGTTTggc | | CGATCTGGGTG | | CAGCGCCGAGGC | |
| gRNA2_ | GTCAGAGGAAGC | 228 | gctGTCAGAGGAAGCTGGTCT | 229 | caccGTCAGAGGAAGCT | 230 | agacAGGCCCAGACCA | 231 |
| AIK_TRBC | TGGTCTGGGCCT | | GGGCCTGGGAGTCTgtg | | GGTCTGGGCCT | | GCTTCCTCTGAC | |
| gRNA1_ | GAGGAGGTGGTA | 232 | gggGAGGAGGTGGTAGCTGG | 233 | caccGAGGAGGTGGTAG | 234 | agacCCCAGCCCCAGC | 235 |
| AIK_ | GCTGGGGCTGGG | | GGCTGGGGGCGGTGTctg | | CTGGGGCTGGG | | TACCACCTCCTC | |
| VEGFAsite2 | | | | | | | | |
| gRNA2_ | GGAGGTGGTAGC | 236 | ggaGGAGGTGGTAGCTGGGG | 237 | caccGGAGGTGGTAGCT | 238 | agacCCCCCAGCCCCA | 239 |
| AIK_ | TGGGGCTGGGGG | | CTGGGGGCGGTGTCTgtc | | GGGGCTGGGGG | | GCTACCACCTCC | |
| VEGFAsite2 | | | | | | | | |
| gRNA1_ | GCCCATTCCCTCT | 240 | aaaGCCCATTCCCTCTTTAGC | 241 | caccGCCCATTCCCTCTT | 242 | agacGCTCTGGCTAAA | 243 |
| AIK_ | TTAGCCAGAGC | | CAGAGCCGGGGTGTgca | | TAGCCAGAGC | | GAGGGAATGGGC | |
| VEGFAsite3 | | | | | | | | |
| gRNA1_ | GAGAGAGGCTCC | 244 | ctgGAGAGAGGCTCCCATCAC | 245 | caccGAGAGAGGCTCCC | 246 | agacTCCCCCGTGATG | 247 |
| AIK_CACNA | CATCACGGGGGA | | GGGGGAGGGAGTTTgct | | ATCACGGGGGA | | GGAGCCTCTCTC | |
| gRNA1_ | GCAGCAGAAATA | 248 | cttGCAGCAGAAATAGACTAA | 249 | caccGCAGCAGAAATAG | 250 | agacATGCAATTAGTC | 251 |
| AIK_ | GACTAATTGCAT | | TTGCATGGGCGTTTccc | | ACTAATTGCAT | | TATTTCTGCTGC | |
| HEKsite3 | | | | | | | | |
| gRNA1_ | AAGTCACCATCAC | 252 | tcaAAGTCACCATCACAAGGA | 253 | caccGAAGTCACCATCAC | 254 | agacAGCGTTTCCTTG | 255 |
| AIK | AAGGAAACGCT | | AACGCTTGGTGTATtga | | AAGGAAACGCT | | TGATGGTGACTTC | |
| HEKsite4 | | | | | | | | |
| gRNA2_ | CCAGGTCAGATAA | 256 | gtcCCAGGTCAGATAAATTTT | 257 | caccGCCAGGTCAGATA | 258 | agacCTTCCTAAAATTT | 259 |
| AIK_ | ATTTTAGGAAG | | AGGAAGTGCTGTTTtcc | | AATTTTAGGAAG | | ATCTGACCTGGC | |
| HEKsite4 | | | | | | | | |
| gRNA1_ | CAGCGGAAAGGG | 260 | gtgGGCAGGGCCTGACAGCG | 261 | caccGGCAGGGCCTGAC | 262 | agacCCCTTTCCGCTG | 263 |
| AIK_Chr8 | GGCAGGGCCTGA | | GAAAGGGTGGAGCTTtat | | AGCGGAAAGGG | | TCAGGCCCTGCC | |
| gRNA2_ | AAGGGTAGAGT | 264 | tttGACCCCTAATATGAAGGGT | 265 | caccGACCCCTAATATGA | 266 | agacACTCTACCCTTC | 267 |
| AIK_Chr8 | GACCCCTAATATG | | AGAGTGAGTGTGTgtg | | AGGGTAGAGT | | ATATTAGGGGTC | |
| gRNA1_ | GGTAGCGTGGG | 268 | cttGTGGCTGTGCTTAGGTAG | 269 | caccGTGGCTGTGCTTA | 270 | agacCCCACGCTACCT | 271 |
| AIK_BCR | GTGGCTGTGCTTA | | CGTGGGATGTGTGTgtt | | GGTAGCGTGGG | | AAGCACAGCCAC | |
| gRNA2_ | CCCCTTCCCCA | 272 | accAGTTCTTGCCGTGCCCCT | 273 | caccGAGTTCTTGCCGT | 274 | agacTGGGGAAGGGG | 275 |
| AIK BCR | AGTTCTTGCCGTG | | TCCCCAGGGTGTGTggt | | GCCCCTTCCCCA | | CACGGCAAGAACTC | |

(*)The target sequences are reported with three flanking nucleotides on each side. The PAM sequence is highlighted in bold.
(**)The cloning overhang is reported in lowercase. Nucleotides in bold text represent 5′-G appended to favor transcription from canonical U6 Pol III promoters.

### 7.1.1.2. Cell Lines

HEK293T cells (obtained from ATCC) and U2OS.EGFP cells (a kind gift of Claudio Mussolino, University of Freiburg), harboring a single integrated copy of an EGFP reporter gene, were cultured in DMEM (Life Technologies) supplemented with 10% FBS (Life Technologies), 2 mM GlutaMax™ (Life Technologies) and penicillin/streptomycin (Life Technologies). All cells were incubated at 37° C. and 5% CO₂ in a humidified atmosphere. All cells tested mycoplasma negative (PlasmoTest, Invivogen).

### 7.1.1.3. Identification of Type II Cas Proteins From Metagenomic Data

154,723 bacterial and archaeal metagenome-assembled genomes (MAGs) reconstructed from the human microbiome (Pasolli, et al., 2019, Cell 176(3):649-662.e20) were screened in order to find new Type II Cas proteins. cas1, cas2 and cas9 genes were identified from the protein annotation, performed with Prokka version 1.12 (Seemann, 2014, Bioinformatics 30(14):2068-2069). CRISPR arrays were identified using MinCED version 0.4.2 (with default parameters) (Bland, et al., 2007, BMC bioinformatics 8:209). Only loci having a CRISPR array and cas1-2-9 genes at a maximum distance of 10 kbp from each other were considered. Loci containing Type II Cas proteins shorter than 950 aa were discarded. The resulting 17173 CRISPR-Type II Cas loci were filtered by selecting short proteins (less than 1100 aa) from putative unknown species. Type II Cas proteins from the same species, having similar length but slightly different sequence, were compared by multiple sequence alignment. Proteins presenting deletions in nucleasic domains were discarded. The remaining proteins were compared for sequencing coverage and the ortholog with the highest coverage was selected for each species.

7.1.1.4. tracrRNA Identification

Identification of tracrRNAs for CRISPR-Type II Cas loci of interest was performed with a method based on a work by Chyou and Brown (Chyou and Brown, 2019, RNA biology 16(4):423-434). Starting from unique direct repeats in the CRISPR array, BLAST version 2.2.31 (with parameters -task blastn-short-gapopen 2-gapextend 1-penalty-1-reward 1-evalue 1-word_size 8) (Altschul, et al., 1990, Journal of Molecular Biology 215(3):403-410) was used to identify anti-repeats within a 3000 bp window flanking the CRISPR-Type II Cas locus. A custom version of RNIE (Gardner, et al., 2011, Nucleic Acids Research 39(14):5845-5852) was used to predict Rho-independent transcription terminators (RITs) near anti-repeats. Putative tracrRNA sequences, starting with an anti-repeat and ending with either a RIT (when found) or a poly-T, were combined with directed repeats to form sgRNA scaffolds. The secondary structure of sgRNA scaffolds was predicted using RNAsubopt version 2.4.14 (with parameters -noLP-e 5) (Lorenz, et al., 2011, Algorithms for Molecular Biology 6(1):26). sgRNAs lacking the functional modules identified by (Briner, et al., 2014 Molecular Cell 56(2):333-339), namely the repeat:anti-repeat duplex, nexus and 3′ hairpin-like folds, were discarded.

### 7.1.1.5. Bacterial-based Negative Selection Assay For Type II Cas PAM Identification

The assay was performed according to the methods from Kleinstiver et al. (Kleinstiver, et al., 2015, Nature 523(7561):481-485). Briefly, electrocompetent E. coli BW25141(DE3) cells (a kind gift from David Edgell, Western University) were transformed with a BPK764-derived plasmid expressing the Type II Cas protein together with its sgRNA. Cells were then electroporated with 100 ng of a p11-LacY-wtx1 (Addgene plasmid #69056)-derived plasmid library containing the target for the sgRNA (target 2 from (Kleinstiver, et al., 2015, Nature 523(7561):481-485) was used) flanked by a randomized 8-nucleotides PAM. Cells were resuspended in 1 mL of recovery medium+IPTG 0.5 mM to induce high levels of protein expression and incubated for 1 hour at 3700 shaking. An appropriate number of cells were plated on a square LB bioassay dish containing ampicillin+chloramphenicol+IPTG 0.5 mM to guarantee around 100× coverage of the randomized PAM library. Surviving colonies, containing PAMs not recognized and cleaved by the Type II Cas protein, were harvested and the plasmid DNA was purified by maxi-prep (Macherey-Nagel). Two PCR steps (Phusion® HF DNA polymerase—Thermo Fisher Scientific) were performed to prepare the plasmid PAM library for NGS analysis: the first, using a set of forward primers and two different reverse primers, to amplify the region containing the protospacer and the PAM and the second to attach the Illumina Nextera™ DNA indexes and adapters (Table 6). PCR products were purified using Agencourt AMPure™ beads (Beckman Coulter) in a 1:0.8 ratio. The library was analyzed with a 150-bp single read sequencing, using a v2 or v3 flow cell on an Illumina MiSeq sequencer.

TABLE 6
Sequences of the Primers Used For NGS Library Preparation in the
Bacterial-based PAM Assay

| Primer | | SEQ ID |
|---|---|---|
| name | Sequence | NO: |
| F1a | TCGTCGGCAGCGTCAGATGTGTATAAGAGACAGAAAACACACCGC | 276 |
| | ATACGTACGATTTA | |
| F1b | TCGTCGGCAGCGTCAGATGTGTATAAGAGACAGTTTAATCACACCG | 277 |
| | CATACGTACGATTTA | |
| R1 | GTCTCGTGGGCTCGGAGATGTGTATAAGAGACAGCGTTCTGATTTA | 278 |
| | ATCTGTATCAGGC | |
| F2a | TCGTCGGCAGCGTCAGATGTGTATAAGAGACAGTTTTTGGTTACGC | 279 |
| | ATCTGTGCGGTATTTC | |
| F2b | TCGTCGGCAGCGTCAGATGTGTATAAGAGACAGCCCTTACGCATCT | 280 |
| | GTGCGGTATTTC | |
| F3a | TCGTCGGCAGCGTCAGATGTGTATAAGAGACAGTATCGATTTAAAT | 281 |
| | AGGCCTGACTCAC | |
| F3b | TCGTCGGCAGCGTCAGATGTGTATAAGAGACAGATTTCGATTTAAA | 282 |
| | TAGGCCTGACTCACTA | |
| R2 | GTCTCGTGGGCTCGGAGATGTGTATAAGAGACAGAATCTTCTCTCA | 283 |
| | TCCGCCAAA | |

A script adapted from Kleinstiver et al. (Kleinstiver, et al., 2015, Nature 523(7561):481-485) was used to extract 8 nt randomized PAMs from Illumina MiSeq™ reads. PAM depletion was evaluated by computing the frequency of PAM sequences in the cleaved library divided by the frequency of the same sequences in a control uncleaved library. Sequences depleted at least 10-fold were used to generate PAM sequence logos, using Logomaker version 0.8 (Tareen and Kinney, 2020, Bioinformatics 36(7):2272-2274). PAMs were also displayed using PAM heatmaps (described in Walton, et al., 2021, Nature Protocols 16(3):1511-1547), showing the fold depletion for each combination of bases at the four most informative positions in the sequence logos.

7.1.1.6. In vitro Type II Cas PAM Identification Assay

The in vitro PAM evaluation of the novel Type II Cas orthologs was performed according to the protocol from Karvelis, Young and Siksnys (Karvelis, et al., 2019, Methods in Enzymology 616:219-240). In brief: the human codon optimized version of the Type II Cas gene was ordered as a synthetic construct (Genscript) and cloned into an expression vector for in vitro transcription and translation (IVT) (pT7-N-His-GST, Thermo Fisher Scientific). The reaction was performed according to the manufacturer's protocol (1-Step Human High-Yield Mini VT Kit, Thermo Fisher Scientific). The Type II Cas-guide RNA RNP complex was assembled by combining 20 μL of the supernatant containing the soluble Type II Cas protein with 1 μL of RiboLock™ RNase Inhibitor (Thermo Fisher Scientific) and 2 μg of guide RNA (custom synthesized sgRNAs obtained from IDT). The Type II Cas-guide complex was used to digest 1 μg of the same PAM plasmid DNA library used for the bacterial assay for 1 hour at 3700.

A double stranded DNA adapter (Table 7) was ligated to the DNA ends generated by the targeted Type II Cas cleavage and the final ligation product was purified using a GeneJet™ PCR Purification Kit (Thermo Fisher Scientific).

TABLE 7
Sequences of the Two Oligonucleotides Used to Prepare the dsDNA
Adapter for the in vitro PAM

| Assay | | SEQ ID |
|---|---|---|
| Name | Sequence | NO: |
| Oligo UP | CGGCATTCCTGCTGAACCGCTCTTCCGATCT | 284 |
| Oligo BOTTOM | GATCGGAAGAGCGGTTCAGCAGGAATGCCG | 285 |

One round of a two-step PCR (Phusion® HF DNA polymerase, Thermo Fisher Scientific) was performed to enrich the sequences that were cut using a set of forward primers annealing on the adapter and a reverse primer designed on the plasmid backbone downstream of the PAM (Table 8). A second round of PCR was performed to attach the Illumina indexes and adapters. PCR products were purified using Agencourt AMPure™ beads in a 1:0.8 ratio.

TABLE 8
Sequences of the Primers Used for NGS Library Preparation in
the in vitro PAM Assay

| Primer | | SEQ ID |
|---|---|---|
| name | | NO: |
| F4a | TCGTSequenceCGGCAGCGTCAGATGTGTATAAGAGACAGCTGCTGAACCGC | 286 |
| | TCTTCCGATC | |
| F4b | TCGTCGGCAGCGTCAGATGTGTATAAGAGACAGTAAGACTGCTGAA | 287 |
| | CCGCTCTTCCGATC | |
| F4c | TCGTCGGCAGCGTCAGATGTGTATAAGAGACAGGCTAGACCTAATG | 288 |
| | TGATCTGCTGAACCGCTCTTCCGATC | |
| R3 | GTCTCGTGGGCTCGGAGATGTGTATAAGAGACAGTCTGCGTTCTGA | 289 |
| | TTTAATCTGTATCAGGC | |

The library was analyzed with a 71-bp single read sequencing, using a flow cell v2 micro, on an Illumina MiSeq™ sequencer.

PAM sequences were extracted from Illumina MiSeq™ reads and used to generate PAM sequence logos, using Logomaker version 0.8. PAM heatmaps were used to display PAM enrichment, computed dividing the frequency of PAM sequences in the cleaved library by the frequency of the same sequences in a control uncleaved library.

### 7.1.1.7. Cell Line Transfections

To perform editing studies, 200,000 U2OS.EGFP cells were nucleofected with 1 μg of px-Cas plasmid bearing a sgRNA designed to target EGFP using the 4D-Nucleofector™ X Kit (Lonza), DN100 program, according to the manufacturer's protocol. After electroporation, cells were plated in a 96-well plate. After 48 hours cells were expanded in a 24-well plate. EGFP knock-out was analysed 4 days after nucleofection using a BD FACSCanto™ (BD) flow cytometer.

Similarly, 100,000 HEK293T cells were seeded in a 24-well plate 24 hours before transfection. Cells were then transfected with 1 μg of the px-Cas plasmid expressing the variant of interest and targeting the locus of interest using the TranslT®-LT1 reagent (Mirus Bio) according to the manufacturer's protocol. Cell pellets were collected 3 day from transfection for indel analysis.

### 7.1.1.8. Evaluation of Indel Formation

Three days after transfection transfected cells were collected and DNA was extracted using the QuickExtract™ DNA Extraction Solution (Lucigen) according to the manufacturer's instructions. To amplify the target loci, PCR reactions were performed using the HOT FIREPol® polymerase (Solis BioDyne), using the oligonucleotides listed in Table 9. The amplified products were purified, sent for Sanger sequencing (EasyRun service, Microsynth) and analyzed with the TIDE web tool (shinyapps.datacurators.nl/tide/) to quantify indels. The primers used for Sanger sequencing reactions on amplicons generated with the oligonucleotides of Table 9 are reported in Table 10, associated with their respective target locus.

TABLE 9
Oligonucleotides Used to Amplify Genomic Regions and to Perform TIDE Analysis

| | | SEQ | | SEQ ID |
|---|---|---|---|---|
| Locus | For (5′→3′) | ID NO: | Rev (5′→3′) | NO: |
| EMX1 | ATTTCGGACTACCCTGAGG | 290 | GGAATCTACCACCCCAG | 291 |
| | AG | | GCTCT | |
| FAS_1 | TTAGAAAGGGCAGGAGGC | 292 | CTTGTCCAGGAGTTCCG | 293 |
| | | | CTC | |
| FAS_2 | AATTGAAGCGGAAGTCTGG | 294 | AACACTTCTCTCGCTATG | 295 |
| | G | | CC | |
| CCR5 | ATGCACAGGGTGGAACAA | 296 | CTAAGCCATGTGCACAAC | 297 |
| | GATGGA | | TCTGAC | |
| FANCF | GGCACATCTTGGGACTCAG | 298 | AGCATAGCGCCTGGCAT | 299 |
| | | | TAATAGG | |
| HBB | CAAAGAACCTCTGGGTCCA | 300 | GCATATTCTGGAGACGC | 301 |
| | AG | | AGG | |
| ZSCAN2 | GACTGTGGGCAGAGGTTC | 302 | TGTATACGGGACTTGACT | 303 |
| | AGC | | CAGACC | |
| CHR6 | ATGTCCTCATGCCGGACTG | 304 | TCCAAGAGCATACGCAC | 305 |
| | | | ACATTCC | |
| ADAMTSL1 | TAGGACTAGGCTCTTGGAG | 306 | CATAGAGTACTTAGTATG | 307 |
| | | | AGCGAGGC | |
| B2M | CCAGTCTAGTGCATGCCTT | 308 | GTTCCCATCACATGTCAC | 309 |
| | C | | | |
| CXCR4 | GGACAGGATGACAATACCA | 310 | AGAGGAGTTAGCCAAGA | 311 |
| | GGCAGGATAAGGCC | | TGTGACTTTGAAACC | |
| PD1 | ACGTCGTAAAGCCAAGGTT | 312 | CACCCTCCCTTCAACCTG | 313 |
| | AGTCC | | ACC | |
| DNMT1 | GTCTTAATTTCCACTCATAC | 314 | CGTTTTGGGCTCTGGGA | 315 |
| | AGTGGTAG | | CTCAG | |
| MATCH8 | TGTGTCGTCCATAAACGCT | 316 | CATCTTCCCTGAAATTTC | 317 |
| | GCC | | TTAAGAGGC | |
| TRAC | CTGTCCCTGAGTCCCAGT | 318 | GGCCTAGAAGAGCAGTA | 319 |
| | | | AGG | |
| TRBC | CTGACCACGTGGAGCTGA | 320 | CTTACTTACCCGAGGTAA | 321 |
| | G | | AGCC | |
| VEGFAsite2 | TGCGAGCAGCGAAAGCGA | 322 | TCCAATGCACCCAAGACA | 323 |
| | CA | | GC | |
| VEGFAsite3 | GCATACGTGGGCTCCAACA | 324 | CCGCAATGAAGGGGAAG | 325 |
| | GGT | | CTCGA | |
| CACNA | TACAGCAGGACTGTGTGG | 326 | CTTCCATCCTCCATCAGG | 327 |
| | CACG | | TCAGG | |
| HEKsite3 | TAGCTACGCCTGTGATGG | 328 | CCAGAGAAGTTGCTAGG | 329 |
| | | | ATGAAAGG | |
| HEKsite4 | AACAATTTCAGATCGCGG | 330 | GTCAGACGTCCAAAACC | 331 |
| | | | AGACTCC | |
| CHR8 | TCCTGGGTCTGAGTTTCTG | 332 | ACAACACAGATCTGCAGA | 333 |
| | AGAGG | | TCTCCG | |
| BCR | GTCAGGGCGCTCCTTCCTT | 334 | GTGTACAGGGCACCTGC | 335 |
| | C | | A | |

TABLE 10
Oligonucleotides Used for Sanger Sequencing to Perform TIDE Analysis

| gRNA name | Oligo sequence (5′ > 3′) | SEQ ID NO: |
|---|---|---|
| gRNA1_AIK_EMX1 | CTGCCATCCCCTTCTGTGAATGT | 336 |
| gRNA2_AIK_EMX1 | GAAGCGATTATGATCTCTCC | 337 |
| gRNA3_AIK_EMX1 | ATTTCGGACTACCCTGAGGAG (Oligo For EMX | 338 |
| | amplification)* | |
| gRNA1_BNK_EMX1 | CTGCCATCCCCTTCTGTGAATGT | 339 |
| gRNA3_BNK_EMX1 | GAAGCGATTATGATCTCTCC | 340 |
| gRNA1_AIK_FAS | TTAGAAAGGGCAGGAGGC (Oligo For FAS_1 | 341 |
| | amplification)* | |
| gRNA2_AIK_FAS | AATTGAAGCGGAAGTCTGGG (Oligo For FAS_2 | 342 |
| | amplification)* | |
| gRNA1_BNK_FAS | AACACTTCTCTCGCTATGCC (Oligo Rev FAS_2 | 343 |
| | amplification)* | |
| gRNA2_BNK_FAS | AATTGAAGCGGAAGTCTGGG (Oligo For FAS_2 | 344 |
| | amplification)* | |
| gRNA1_AIK_CCR5 | ACCTGTTAGAGCTACTGC | 345 |
| gRNA2_AIK_CCR5 | AGAAGAAGAGGCACAGGGC | 346 |
| gRNA1_BNK_CCR5 | CTAAGCCATGTGCACAACTCTGAC (Oligo Rev CCR5 | 347 |
| | amplification)* | |
| gRNA2_BNK_CCR5 | ATGCACAGGGTGGAACAAGATGGA (Oligo For CCR5 | 348 |
| | amplification)* | |
| gRNA1_AIK_FANCF | AGCATAGCGCCTGGCATTAATAGG (Oligo Rev FANCF | 349 |
| | amplification)* | |
| gRNA2_AIK_FANCF | GGCACATCTTGGGACTCAG (Oligo For FANCF | 350 |
| | amplification)* | |
| gRNA3_AIK_FANCF | GCCAGGCTCTCTTGGAGTGTC | 351 |
| gRNA1_AIK_HBB | GCATATTCTGGAGACGCAGG (Oligo Rev HBB | 352 |
| | amplification)* | |
| gRNA2_AIK_HBB | CTCCTTAAACCTGTCTTG | 353 |
| gRNA1_AIK_ZSCAN2 | GACTGTGGGCAGAGGTTCAGC (Oligo For ZSCAN2 | 354 |
| | amplification)* | |
| gRNA2_AIK_ZSCAN2 | GACTGTGGGCAGAGGTTCAGC (Oligo For ZSCAN2 | 355 |
| | amplification)* | |
| gRNA1_AIK_Chr6 | ATGTCCTCATGCCGGACTG | 356 |
| gRNA1_AIK_ADAM | TAGGACTAGGCTCTTGGAG | 357 |
| gRNA2_AIK_ADAM | CAACCCCACCACTGAGTTATTAGG | 358 |
| gRNA1_AIK_B2M | CCAGTCTAGTGCATGCCTTC (Oligo For B2M | 359 |
| | amplification)* | |
| gRNA2_AIK_B2M | GTTCCCATCACATGTCAC (Oligo Rev B2M amplification)* | 360 |
| gRNA1_AIK_CXCR4 | GGACAGGATGACAATACCAGGCAGGATAAGGCC (Oligo | 361 |
| | For CXCR4 amplification)* | |
| gRNA2_AIK_CXCR4 | GGACAGGATGACAATACCAGGCAGGATAAGGCC (Oligo | 362 |
| | For CXCR4 amplification)* | |
| gRNA1_AIK_PD1 | CACCCTCCCTTCAACCTGACC (Oligo Rev PD1 | 363 |
| | amplification)* | |
| gRNA2_AIK_PD1 | CACCCTCCCTTCAACCTGACC (Oligo Rev PD1 | 364 |
| | amplification)* | |
| gRNA1_AIK_DNMT1 | CGTTTTGGGCTCTGGGACTCAG (Oligo Rev DNMT1 | 365 |
| | amplification)* | |
| gRNA2_AIK_Match8 | TGTGTCGTCCATAAACGCTGCC (Oligo For Match8 | 366 |
| | amplification)* | |
| gRNA1_AIK_TRAC | GGCCTAGAAGAGCAGTAAGG (Oligo Rev TRAC | 367 |
| | amplification)* | |
| gRNA1_AIK_TRBC | CTGACCACGTGGAGCTGAG (Oligo For TRBC | 368 |
| | amplification)* | |
| gRNA2_AIK_TRBC | CTTACTTACCCGAGGTAAAGCC (Oligo Rev TRBC | 369 |
| | amplification)* | |
| gRNA1_AIK_VEGFAsite2 | TGCGAGCAGCGAAAGCGACA (Oligo For VEGFAsite2 | 370 |
| | amplification)* | |
| gRNA2_AIK_VEGFAsite2 | TGCGAGCAGCGAAAGCGACA (Oligo For VEGFAsite2 | 371 |
| | amplification)* | |
| gRNA1_AIK_VEGFAsite3 | GCATACGTGGGCTCCAACAGGT (Oligo For VEGFAsite3 | 372 |
| | amplification)* | |
| gRNA1_AIK_CACNA | TACAGCAGGACTGTGTGGCACG (Oligo For CACNA | 373 |
| | amplification)* | |
| gRNA1_AIK_HEKsite3 | TAGCTACGCCTGTGATGG (Oligo For HEKsite3 | 374 |
| | amplification)* | |
| gRNA1_AIK_HEKsite4 | AACAATTTCAGATCGCGG (Oligo For HEKsite4 | 375 |
| | amplification)* | |
| gRNA2_AIK_HEKsite4 | AGAGAAGTTGGAGTGAAGGCAGAG | 376 |
| gRNA1_AIK_Chr8 | ACAACACAGATCTGCAGATCTCCG (Oligo Rev Chr8 | 377 |
| | amplification)* | |
| gRNA2_AIK_Chr8 | TCCTGGGTCTGAGTTTCTGAGAGG (Oligo For Chr8 | 378 |
| | amplification)* | |
| gRNA1_AIK_BCR | GTCAGGGCGCTCCTTCCTTC (Oligo For BCR | 379 |
| | amplification)* | |
| gRNA2_AIK_BCR | GTGTACAGGGCACCTGCA (Oligo Rev BCR amplification)* | 380 |

*These oligonucleotides were also listed in Table 9, as indicated

### 7.1.2. Results

### 7.1.2.1. Identification of Novel Type II Cas Orthologs From Metagenomic Data

The great development of the genome editing field, with several upcoming clinical applications already tested in the first patients, and new technologies to modify the cellular DNA going beyond the introduction of double strand breaks, pushes for the discovery of new tools to edit the genetic material of cells. In particular, the discovery of new Type II Cas nucleases with smaller sizes compared to the most widely used SpCas9 and a variety of different PAM specificities is of great interest to the advancement of the field, both for industrial/applied and the basic research. These features will allow on one hand to increase the density of targetable sites in a defined genome (more PAMs) and on the other hand to provide much easier vectorization, especially in AAV vectors which suffer from limitations in cargo size, thanks to the smaller CDS size.

For these studies, a curated collection of assembled bacterial and archaeal metagenome-based genomes (Pasolli, et al., 2019, Cell 176(3):649-662.e20) was explored exploiting a custom-written bioinformatic pipeline to identify novel Type II Cas proteins with extremely low sequence homology to Type II Cas orthologs previously published and characterized. The discovered Type II Cas orthologs were filtered based on: i) the length of their coding sequence, discarding those too short (<950 aa) or too long (>1100 aa); ii) their origin from putative unknown species and iii) the presence of intact nucleasic domains. Type II Cas proteins with high sequence similarity were clustered together and the orthologs with the greater sequence representation in the original metagenomic library were selected for each cluster. Among the identified Type II Cas proteins, two were of particular interest:
AIK Type II Cas, originating from the Genus Collinsella, 1004 aa long
BNK Type II Cas, originating from an unclassified Proteobacterium, 1002 aa long

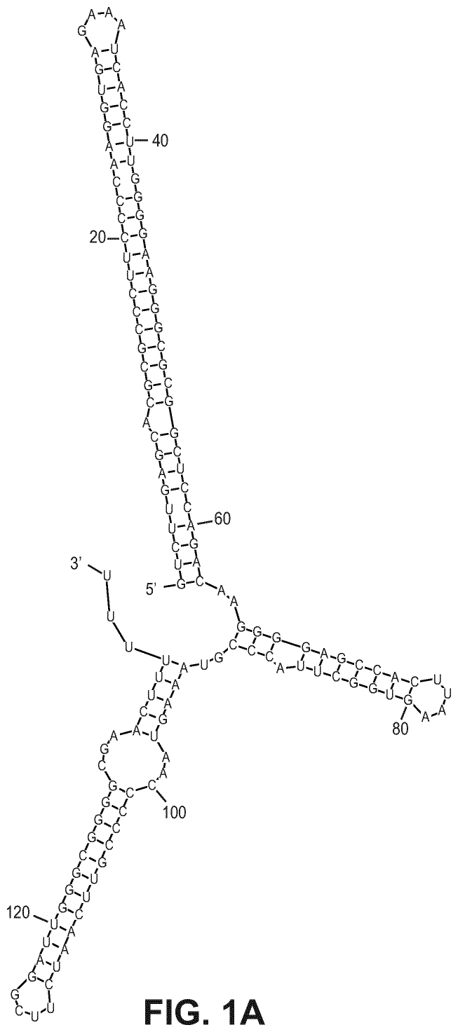

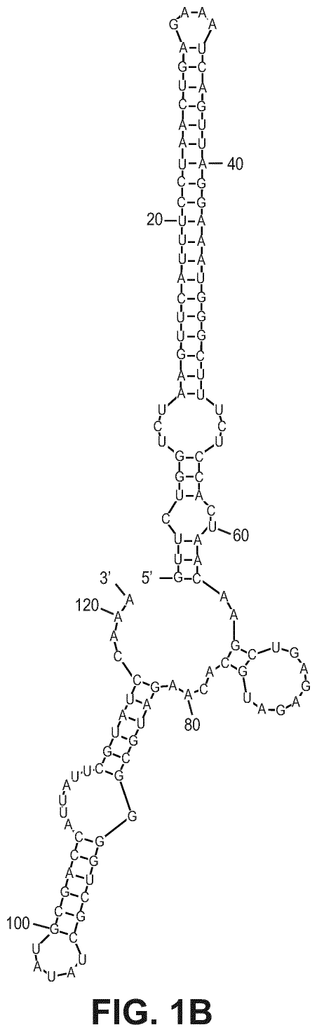

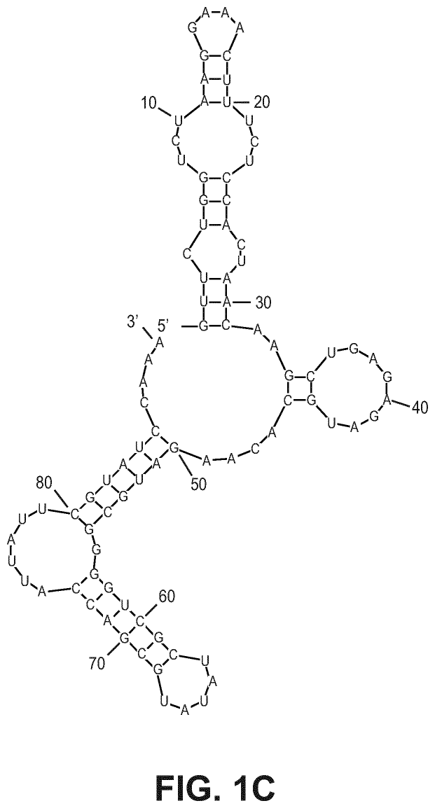

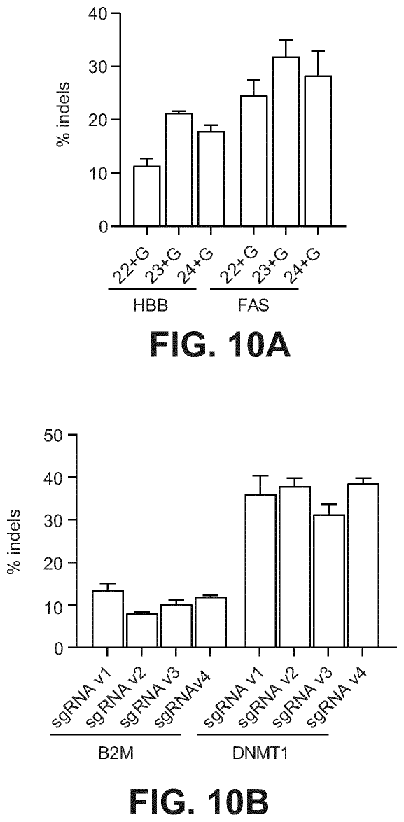

Next a search to identify the tracrRNA of these two nucleases from the same metagenomic data was performed using a custom-built bioinformatic pipeline and sgRNAs were designed for both Type II Cas variants by combining the identified tracrRNA with the corresponding crRNAs extracted from the CRISPR arrays of each of the two nucleases. The predicted hairpin structure of the sgRNA molecules for AIK Type II Cas and BNK Type II Cas are represented in FIG. 1A-B, while the sequences are reported in Table 11. The sgRNA sequence of BNK Type II Cas was further modified by the introduction of a U>A substitution to interrupt a polyU stretch which may affect negatively RNA PolIII-mediated transcription of the guide RNA (compare BNK_sgRNA_V1 with BNK_sgRNA_V2 in Table 11). In addition, an alternative design for BNK Type II Cas sgRNA, with a trimmed scaffold structure and containing the aforementioned U-A flip is reported in FIG. 10 (BNK_sgRNA_V3).

TABLE 11
Sequences of crRNAs, tracrRNAs and sgRNAs for Type II Cas Orthologs AIK and BNK

| | | SEQ ID |
|---|---|---|
| Name | Sequence | NO: |
| AIK Type II Cas | NNNNNNNNNNNNNNNNNNNNGUCUUGAGCACGCGCCCUUCCCCAAGG | 381 |
| crRNA | UGAUACGCU | |
| BNK Type II | NNNNNNNNNNNNNNNNNNNNGUUCUGGUCUAAGUUCAUUUCCUAACUG | 382 |
| Cas crRNA | AUAAAAUC | |
| AIK Type II Cas | UCACCUUGGGGAAGGGCGCGGCUCCAGACAAGGGGAGCCACUUAAGU | 383 |
| tracrRNA | GGCUUACCCGUAAAGUAACCCCCGUUCAAUCUUCGGAUUGGGGGGGG | |
| | CGAACUUUUUU | |
| BNK Type II | UCAGUUAGGAAAUGGGCUUUCUCCACUAACAAGCUGAGAGAUGCACAA | 384 |
| Cas tracrRNA | GAUGCGGGGUCGCUAUAUGCGACCAUUUUUCGUAUCCAAA | |
| AIK Type II Cas | NNNNNNNNNNNNNNNNNNNNGUCUUGAGCACGCGCCCUUCCCCAAGG | 385 |
| sgRNA_v1 | UGAGAAAUCACCUUGGGGAAGGGCGCGGCUCCAGACAAGGGGAGCCA | |
| | CUUAAGUGGCUUACCCGUAAAGUAACCCCCGUUCAAUCUUCGGAUUGG | |
| | GCGGGGCGAACUUUUUU | |
| AIK Type II Cas | NNNNNNNNNNNNNNNNNNNNGUCUUGAGCACGCGCCCUUCCGCAAGG | 386 |
| sgRNA_v2 | UGAGAAAUCACCUUGCGGAAGGGCGCGGCUCCAGACAAGCGGAGCCA | |
| | CUUAAGUGGCUUACGCGUAAAGUAACCGCCGUUCAAUCUUCGGAUUGG | |
| | GCGGCGCGAACUUUUUU | |
| AIK Type II Cas | NNNNNNNNNNNNNNNNNNNNGUCUUGAGCACGCGAAAGCGGCUCCAG | 387 |
| sgRNA_v3 | ACAAGGGGAGCCACUUAAGUGGCUUACCCGUAAAGUAACCCCCGUUCA | |
| | AUCUUCGGAUUGGGGGGGGCGAACUUUUUU | |
| AIK Type II Cas | NNNNNNNNNNNNNNNNNNNNGUCUUGAGCACGCGAAAGCGGCUCCAG | 388 |
| sgRNA_v4 | ACAAGCGGAGCCACUUAAGUGGCUUACGCGUAAAGUAACCGCCGUUCA | |
| | AUCUUCGGAUUGGGCGGCGCGAACUUUUUU | |
| BNK Type II | NNNNNNNNNNNNNNNNNNNNGUUCUGGUCUAAGUUCAUUUCCUAACUG | 389 |
| Cas sgRNA_v1 | AGAAAUCAGUUAGGAAAUGGGCUUUCUCCACUAACAAGCUGAGAGAUG | |
| | CACAAGAUGCGGGGUCGCUAUAUGCGACCAUUUUUCGUAUCCAAA | |
| BNK Type II | NNNNNNNNNNNNNNNNNNNNGUUCUGGUCUAAGUUCAUUUCCUAACUG | 390 |
| Cas sgRNA_v2 | AGAAAUCAGUUAGGAAAUGGGCUUUCUCCACUAACAAGCUGAGAGAUG | |
| | CACAAGAUGCGGGGUCGCUAUAUGCGACCAUUAUUCGUAUCCAAA | |
| BNK Type II | NNNNNNNNNNNNNNNNNNNNGUUCUGGUCUAAGGAAACUUUCUCCACU | 391 |
| Cas sgRNA_v3 | AACAAGCUGAGAGAUGCACAAGAUGCGGGGUCGCUAUAUGCGACCAUU | |
| | AUUCGUAUCCAAA | |
| BNK Type II | NNNNNNNNNNNNNNNNNNNNGUUCUGGUCUAAGUUCAUUUCCUAACUG | 392 |
| Cas sgRNA_v4 | AGAAAUCAGUUAGGAAAUGGGCUUUCUCCACUAACAAGCGAAAGCACA | |
| | AGAUGCGGGCUCGCUAUAUGCGAGCAUUAUUCGUAUCCAAA | |
| BNK Type II | NNNNNNNNNNNNNNNNNNNNGUUCUGGUCUAAGGAAACUUUCUCCACU | 393 |
| Cas sgRNA_v5 | AACAAGCGAAAGCACAAGAUGCGGGCUCGCUAUAUGCGAGCAUUAUUC | |
| | GUAUCCAAA | |

### 7.1.2.2. Determination of the PAM Specificity of the AIK and BNK Type II Cas Nucleases

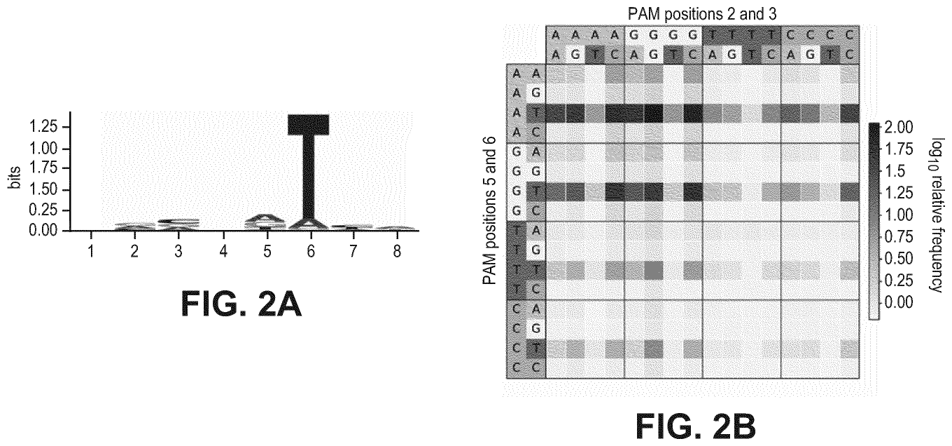

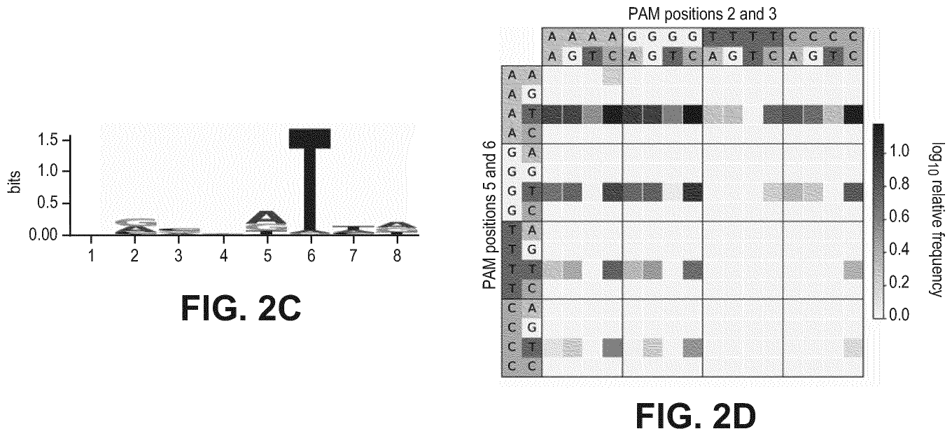

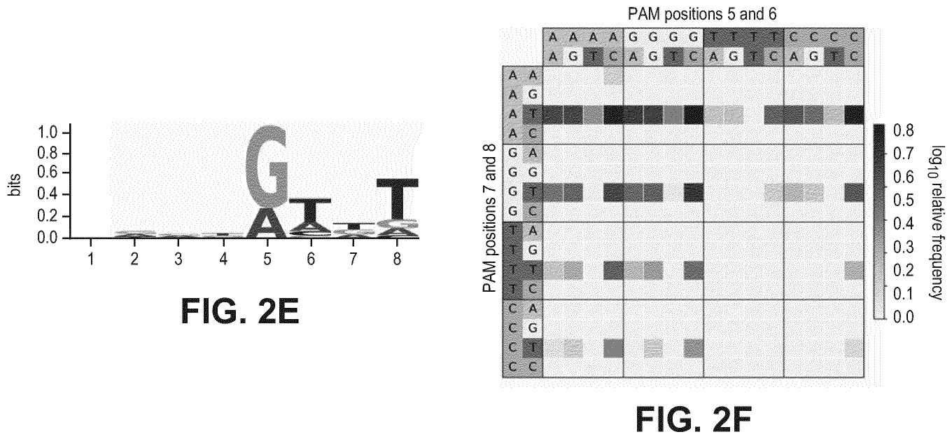

Having determined the sgRNA requirements for AIK Type II Cas and BNK Type II Cas, it was possible to proceed with the discovery of the PAM sites recognized by the two nucleases. The AIK_sgRNA_V1 and BNK_sgRNA_V1 versions of the guide RNAs were used for the PAM discovery assays. The PAM preference of BNK Type II Cas was evaluated in bacteria (E. coli) and in vitro. Both the assays indicated a 3′ NRVNRT PAM preference, cross-confirming the reliability of both methods for PAM assessment (compare FIG. 2A and FIG. 2C). AIK Type II Cas PAM preference was determined only in vitro, resulting in a preference for a 3′ N₄RHNT, N₄RYNT or N₄GYNT PAM (FIG. 2E). The visualization of PAM enrichment as heatmaps allowed a more precise evaluation of the PAMs that were better cut by the two Type II Cas (FIG. 2B,D,2F), revealing that AIK Type II Cas slightly prefers N₄GTTT and N₄GTGT PAMs, while BNK Type II Cas slightly prefers a NRCNAT PAM. This first set of studies also allowed a preliminary validation of the activity of the sgRNAs designed for the two novel CRISPR orthologs.

### 7.1.2.3. Evaluation of the Editing Activity Using an EGFP Reporter System

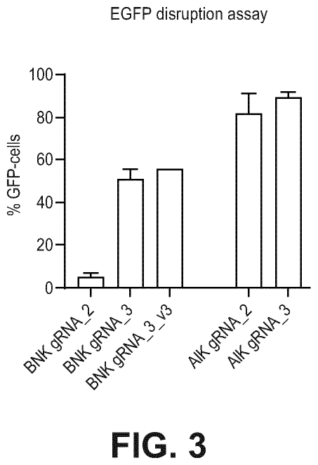

After the discovery of the PAM sequences and the sgRNAs of AIK Type II Cas and BNK Type II Cas and after obtaining preliminary information on the ability of these two CRISPR nucleases to cut a desired target in vitro and inside bacterial cells (E. coli, only for BNK Type II Cas), their ability to cleave selected targets in mammalian cells was investigated. At first, an EGFP reporter system was used as it allowed an easier readout on the editing activity, based on the loss of fluorescence of treated cells quantitatively measured by cytofluorimetry. sgRNAs targeting the EGFP coding sequence were thus designed both for AIK Type II Cas and BNK Type II Cas and evaluated in U2OS cells stably expressing a single copy of an EGFP reporter by transient electroporation. To the Inventor's surprise, as reported in FIG. 3, one out of two guides evaluated for BNK Type II Cas showed appreciable editing levels, and two out of two AIK Type II Cas sgRNA were able to strongly induce EGFP downregulation in treated cells (approximately 80% knock-out). For BNK, also the trimmed version of the sgRNA_3 (BNK gRNA_3_v3, see Table 11) was evaluated and showed comparable editing levels to the non-trimmed version. These data clearly demonstrate that both Type II Cas orthologs were able to efficiently modify genetic targets in mammalian cells and can thus be exploited to edit the mammalian genome.

### 7.1.2.4. Evaluation of the Editing Activity on a Panel of Endogenous Genomic Loci

After having evaluated the editing efficacy of the two newly discovered Type II Cas variants using an EGFP-based reporter system, their activity was measured on a more relevant panel of endogenous genomic loci by transient transfection in HEK293T cells.

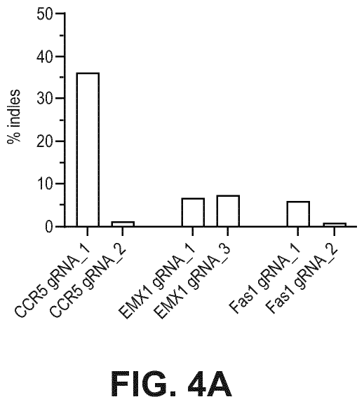

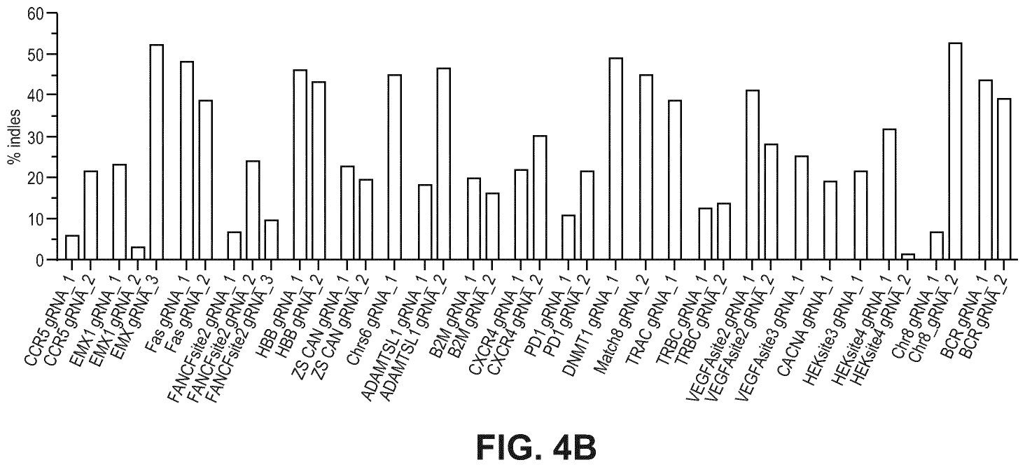

For BNK Type II Cas a panel of three genomic loci was evaluated (CCR5, EMX1 and Fas), selecting two different sgRNAs to target each locus. As shown in FIG. 4A, editing was detected at all targeted loci with at least one of the two evaluated guides. For targeting the EMX1 locus the sgRNA_v2 design was adopted, while for CCR5 and Fas the trimmed sgRNA_v3 design was used. While indel formation was particularly efficient on the CCR5 locus (up to 35%, gRNA1), only lower level modifications were measured on the other evaluated genomic targets (approximately 5% detected indels).

AIK Type II Cas was similarly evaluated on a panel of genomic target sites including the same genes evaluated for BNK Type II Cas (CCR5, EMX1, Fas) plus additional targets (FANCF, HBB, ZSCAN, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, BCR) with multiple guides designed to target the majority of the loci, except for Chr6, DNMT1, Match8, TRAC, VEGFAsite3, CACNA and HEKsite3, for which only one gRNA was evaluated. Overall, a total of 22 different sgRNAs were evaluated for activity. Among the evaluated guides, all were selected to recognize one of the best performing PAM (N₄GTNT) except for one of the guides targeting ADAMSTL1, B2M, Chr8 and FANCF for which the PAM was N₄GCTT. Good editing levels (20-50% indel formation) were measured on the vast majority the evaluated sites with at least one of the sgRNA candidates, and in many instances more guide RNAs targeting the same locus worked equally well (e.g. Fas, HBB, ZSCAN, CXCR4, BCR, B2M, VEGFAsite2), demonstrating the robustness of AIK Type II Cas genome editing activity (FIG. 4B).

### 7.2. Example 2: Identification and Characterization of HPLH and ANAB Type II Cas Proteins

This Example describes studies performed to identify and characterize HPLH and ANAB Type II Cas orthologs.

### 7.2.1. Materials and Methods

This Example describes studies performed to identify and characterize HPLH and ANAB Type II Cas orthologs.

7.2.1.1. Identification of Type II Cas Proteins from Metagenomic Data and tracrRNA Identification

Two previously uncharacterized Type II Cas proteins, HPLH Type II Cas and ANAB Type II Cas, were identified by screening metagenomic data as described in Section 7.1.1.3. tracrRNAs for the Type II Cas loci were identified as described in Section 7.1.1.4. PAM sequences were identified as described in Sections 7.1.1.5 and 7.1.1.6.

### 7.2.1.2. Plasmids

A pX330-derived plasmid was used to express Type II Cas nucleases and their relative sgRNAs in mammalian cells. Briefly, pX330 was modified by substituting the SpCas9 and its sgRNA scaffold with the human codon-optimized sequence of ANAB Cas9 (see, Table 1 D), HPLH Cas9 (see, Table 1C) and its sgRNA scaffold (either full length or trimmed), generating pX-ANABCas or pX-HPLHCas. The Type II Cas sequences, fused with a V5 tag at the N-terminus and two nuclear localization signals (one at the N-terminus and one at the C-terminus), and the sgRNA scaffolds, were obtained as synthetic fragments from either Genscript or Genewiz. Spacer sequences were cloned into the pX-Cas plasmids as annealed DNA oligonucleotides containing a variable 20 or 24 nt spacer sequence using a double BsaI site present in the plasmid. pX-AIKCas (prepared as described in Section 7.1.1.1) was also used in this Example. The list of spacer sequences used in the Example is reported in Table 12.

TABLE 12
Spacer Sequences, Their Targets, and sgRNA Oligonucleotides

| Spacer Sequences and Targets | | | | | Oligo Used to Clone the Spacer in pX Plasmid (****) | | | |
|---|---|---|---|---|---|---|---|---|
| | | SEQ | | SEQ | | SEQ | | SEQ |
| Name (*) | Protospacer | ID NO: | Target (***) | ID NO: | Oligo 1 (5′ > 3′) | ID NO: | Oligo 2 (5′ > 3′) | ID NO: |
| gRNA1_AIK_ | TGCCCCTCCCTC | 96 | ctgTGCCCCTCCCTCC | 97 | caccGTGCCCCTCCCTCCC | 98 | agacACCTGGGCCAGG | 99 |
| EMX1 | CCTGGCCCAGGT | | CTGGCCCAGGTGAAG | | TGGCCCAGGT | | GAGGGAGGGGCAC | |
| | | | GTGTggt | | | | | |
| gRNA2_AIK_ | GGGCATGGTTTC | 104 | aatGGGCATGGTTTCA | 105 | caccGGGCATGGTTTCATA | 106 | agacCCTCCTAGTTATG | 107 |
| EMX1 | ATAACTAGGAGG | | TAACTAGGAGGTGGT | | ACTAGGAGG | | AAACCATGCCC | |
| | | | GTTTata | | | | | |
| gRNA1_AIK_ | CGCCTGGGCAG | 116 | gctCCGCCTGGGCAGC | 117 | caccGCGCCTGGGCAGCC | 118 | agacAGGCCAGCCCTG | 119 |
| FAS | CCAGGGCTGGC | | CAGGGCTGGCCTCAG | | AGGGCTGGCCT | | GCTGCCCAGGCGC | |
| | CT | | GGTGTgtt | | | | | |
| gRNA2_24_ | CCTGGGCAGCCA | 394 | ccgCCTGGGCAGCCAG | 395 | caccGCCTGGGCAGCCAG | 396 | agacTGAGGCCAGCCCT | 397 |
| AIK_FAS | GGGCTGGCCTCA | | GGCTGGCCTCAGGGT | | GGCTGGCCTCA | | GGCTGCCCAGGC | |
| | | | GTGTtcc | | | | | |
| gRNA2_23_ | CTGGGCAGCCAG | 398 | cgcCTGGGCAGCCAGG | 399 | caccGCTGGGCAGCCAGG | 400 | agacTGAGGCCAGCCCT | 401 |
| AIK_FAS | GGCTGGCCTCA | | GCTGGCCTCAGGGTG | | GCTGGCCTCA | | GGCTGCCCAGC | |
| | | | TGTtcc | | | | | |
| gRNA2_22_ | TGGGCAGCCAG | 402 | gccTGGGCAGCCAGG | 403 | caccGTGGGCAGCCAGGG | 404 | agacTGAGGCCAGCCCT | 405 |
| AIK_FAS | GGCTGGCCTCA | | GCTGGCCTCAGGGTG | | CTGGCCTCA | | GGCTGCCCAC | |
| | | | TGTtcc | | | | | |
| gRNA1_AIK_ | GCAAGAGGCTCC | 136 | ccaGCAAGAGGCTCCC | 137 | caccGCAAGAGGCTCCCGA | 138 | agacCTTGCTCGCTCGG | 139 |
| CCR5 | CGAGCGAGCAAG | | GAGCGAGCAAGCTCA | | GCGAGCAAG | | GAGCCTCTTGC | |
| | | | GTTTaca | | | | | |
| gRNA1_AIK_ | GGAAGGCCGAA | 830 | tggGGAAGGCCGAAGC | 153 | caccGGAAGGCCGAAGCG | 154 | agacCGGGACGCTCCG | 155 |
| FANCF | GCGGAGCGTCC | | GGAGCGTCCCGCCAG | | GAGCGTCCCG | | CTTCGGCCTTCC | |
| | CG | | GTTTctc | | | | | |
| gRNA2_AIK_ | GCGCGCTACCTG | 156 | tggGCGCGCTACCTGC | 157 | caccGCGCGCTACCTGCG | 158 | agacATGGATGTGGCGC | 159 |
| FANCF | CGCCACATCCAT | | GCCACATCCATCGGC | | CCACATCCAT | | AGGTAGCGCGC | |
| | | | GCTTtgg | | | | | |
| gRNA1_AIK_ | GAGATATATCTTA | 160 | caaGAGATATATCTTAG | 161 | caccGAGATATATCTTAGA | 162 | agacAGCCCTCCCTCTA | 163 |
| HBB | GAGGGAGGGCT | | AGGGAGGGCTGAGG | | GGGAGGGCT | | AGATATATCTC | |
| | | | GTTTgaa | | | | | |
| gRNA2_24_ | TCTCCTCAGGAG | 406 | actTCTCCTCAGGAGTC | 407 | caccGTCTCCTCAGGAGTC | 408 | agacTGGTGCACCTGAC | 409 |
| AIK_HBB | TCAGGTGCACCA | | AGGTGCACCATGGTG | | AGGTGCACCA | | TCCTGAGGAGAC | |
| | | | TCTgtt | | | | | |
| gRNA2_23_ | CTCCTCAGGAGT | 410 | cttCTCCTCAGGAGTCA | 411 | caccGCTCCTCAGGAGTCA | 412 | agacTGGTGCACCTGAC | 413 |
| AIK_HBB | CAGGTGCACCA | | GGTGCACCATGGTGT | | GGTGCACCA | | TCCTGAGGAGC | |
| | | | CTgtt | | | | | |
| gRNA2_22_ | TCCTCAGGAGTC | 414 | ttcTCCTCAGGAGTCAG | 415 | caccGTCCTCAGGAGTCAG | 416 | agacTGGTGCACCTGAC | 417 |
| AIK_HBB | AGGTGCACCA | | GTGCACCATGGTGTC | | GTGCACCA | | TCCTGAGGAC | |
| | | | Tgtt | | | | | |
| gRNA1_AIK_ | CATGAGGCATTT | 168 | gcaCATGAGGCATTTG | 169 | caccGCATGAGGCATTTGT | 170 | agacGAGAAGCCCTACA | 171 |
| ZSCAN2 | GTAGGGCTTCTC | | TAGGGCTTCTCGCCC | | AGGGCTTCTC | | AATGCCTCATGC | |
| | | | GTGTggg | | | | | |
| gRNA2_AIK_ | GGCTTCTCCACC | 794 | tagGGCTTCTCCACCAT | 795 | caccGGCTTCTCCACCATG | 796 | agacGAGAACCCACATG | 797 |
| ZSCAN2 | ATGTGGGTTCTC | | GTGGGTTCTCCGGTG | | TGGGTTCTC | | GTGGAGAAGCC | |
| | | | TGTggc | | | | | |
| gRNA1_AIK_ | TTGATTCTTACAA | 176 | actTTGATTCTTACAAC | 177 | caccGTTGATTCTTACAACA | 178 | agacCTCTCATGTTGTT | 179 |
| Chr6 | CAACATGAGAG | | AACATGAGAGAGGGG | | ACATGAGAG | | GTAAGAATCAAC | |
| | | | TGTtgt | | | | | |
| gRNA1_AIK_ | GGGGCAGAGAG | 798 | tggGGGGCAGAGAGAG | 799 | caccGGGGCAGAGAGAGA | 800 | agacTCGCTCACTCTCT | 801 |
| ADAM | AGAGAGTGAGCG | | AGAGTGAGCGAGTGA | | GAGTGAGCGA | | CTCTCTGCCCC | |
| | A | | GTGTgtg | | | | | |
| gRNA2_AIK_ | AAAGAAATACTAA | 802 | tagAAAGAAATACTAAG | 803 | caccGAAAGAAATACTAAG | 804 | agacCTCTGCATGTCTT | 805 |
| ADAM | GACATGCAGAG | | ACATGCAGAGAGGTG | | ACATGCAGAG | | AGTATTTCTTTC | |
| | | | CTTtgc | | | | | |
| gRNA1_AIK_ | GGGCCTTGTCCT | 188 | tgcGGGCCTTGTCCTG | 189 | caccGGGCCTTGTCCTGAT | 190 | agacGCCCAGCCAATCA | 191 |
| B2M | GATTGGCTGGGC | | ATTGGCTGGGCACGC | | TGGCTGGGC | | GGACAAGGCCC | |
| | | | GTTTaat | | | | | |
| gRNA2_AIK_ | GCTGAGGTTTGT | 192 | ggcGCTGAGGTTTGTG | 193 | caccGCTGAGGTTTGTGAA | 194 | agacCTCCACGCGTTCA | 195 |
| B2M | GAACGCGTGGAG | | AACGCGTGGAGGGGC | | CGCGTGGAG | | CAAACCTCAGC | |
| | | | GCTTggg | | | | | |
| gRNA1_AIK_ | GTCATCTACACA | 806 | catGTCATCTACACAGT | 807 | caccGTCATCTACACAGTC | 808 | agacGTAGAGGTTGACT | 809 |
| CXCR4 | GTCAACCTCTAC | | CAACCTCTACAGCAG | | AACCTCTAC | | GTGTAGATGAC | |
| | | | TGTcct | | | | | |
| gRNA2_AIK_ | GTTGGCTGAAAA | 810 | gctGTTGGCTGAAAAG | 811 | caccGTTGGCTGAAAAGGT | 812 | agacACATAGACCACCT | 813 |
| CXCR4 | GGTGGTCTATGT | | GTGGTCTATGTTGGC | | GGTCTATGT | | TTTCAGCCAAC | |
| | | | GTCTgga | | | | | |
| gRNA1_AIK_ | GTTCTTAGGTAG | 814 | atgGTTCTTAGGTAGGT | 815 | caccGTTCTTAGGTAGGTG | 816 | agacCCGCCGACCCCA | 817 |
| PD1 | GTGGGGTCGGC | | GGGGTCGGCGGTCA | | GGGTCGGCGG | | CCTACCTAAGAAC | |
| | GG | | GGTGTccc | | | | | |
| gRNA2_AIK_ | TCGGCGGTCAGG | 818 | gggTCGGCGGTCAGGT | 819 | caccGTCGGCGGTCAGGT | 820 | agacGGCTCTGGGACAC | 821 |
| PD1 | TGTCCCAGAGCC | | GTCCCAGAGCCAGGG | | GTCCCAGAGCC | | CTGACCGCCGAC | |
| | | | GTCTgga | | | | | |
| gRNA1_AIK_ | TCGCCTGTCAAG | 212 | tacTCGCCTGTCAAGT | 213 | caccGTCGCCTGTCAAGTG | 214 | agacGGTGTCACGCCAC | 215 |
| DNMT1 | TGGCGTGACACC | | GGCGTGACACCGGGC | | GCGTGACACC | | TTGACAGGCGAC | |
| | | | GTGTtcc | | | | | |
| gRNA1_AIK_ | GGGAGGTGGCA | 216 | gaaGGGAGGTGGCAG | 217 | caccGGGAGGTGGCAGGG | 218 | agacGCTTTCCTCCCCC | 219 |
| Match8 | GGGGGAGGAAA | | GGGGAGGAAAGCAGA | | GGAGGAAAGC | | TGCCACCTCCC | |
| | GC | | GGTTTggg | | | | | |
| gRNA1_AIK_ | GATAAGGCCGAG | 220 | atgGATAAGGCCGAGA | 221 | caccGATAAGGCCGAGACC | 222 | agacCTGATTGGTGGTC | 223 |
| TRAC | ACCACCAATCAG | | CCACCAATCAGAGGA | | ACCAATCAG | | TCGGCCTTATC | |
| | | | GTTTtag | | | | | |
| gRNA1_AIK_ | GCCTCGGCGCTG | 224 | cagGCCTCGGCGCTGA | 225 | caccGCCTCGGCGCTGAC | 226 | agacCACCCAGATCGTC | 227 |
| TRBC | ACGATCTGGGTG | | CGATCTGGGTGACGG | | GATCTGGGTG | | AGCGCCGAGGC | |
| | | | GTTTggc | | | | | |
| gRNA2_AIK_ | GTCAGAGGAAGC | 228 | gctGTCAGAGGAAGCT | 229 | caccGTCAGAGGAAGCTGG | 230 | agacAGGCCCAGACCA | 231 |
| TRBC | TGGTCTGGGCCT | | GGTCTGGGCCTGGGA | | TCTGGGCCT | | GCTTCCTCTGAC | |
| | | | GTCTgtg | | | | | |
| gRNA1_AIK_ | GAGGAGGTGGTA | 232 | gggGAGGAGGTGGTA | 233 | caccGAGGAGGTGGTAGCT | 234 | agacCCCAGCCCCAGCT | 235 |
| VEGFAsite2 | GCTGGGGCTGG | | GCTGGGGCTGGGGG | | GGGGCTGGG | | ACCACCTCCTC | |
| | G | | CGGTGTctg | | | | | |
| gRNA2_AIK_ | GGAGGTGGTAGC | 236 | ggaGGAGGTGGTAGCT | 237 | caccGGAGGTGGTAGCTG | 238 | agacCCCCCAGCCCCA | 239 |
| VEGFAsite2 | TGGGGCTGGGG | | GGGGCTGGGGGCGG | | GGGCTGGGGG | | GCTACCACCTCC | |
| | G | | TGTCTgtc | | | | | |
| gRNA1_AIK_ | GCCCATTCCCTC | 240 | aaaGCCCATTCCCTCT | 241 | caccGCCCATTCCCTCTTT | 242 | agacGCTCTGGCTAAAG | 243 |
| VEGFAsite3 | TTTAGCCAGAGC | | TTAGCCAGAGCCGGG | | AGCCAGAGC | | AGGGAATGGGC | |
| | | | GTGTgca | | | | | |
| gRNA1_AIK_ | GAGAGAGGCTCC | 244 | ctgGAGAGAGGCTCCC | 245 | caccGAGAGAGGCTCCCAT | 246 | agacTCCCCCGTGATGG | 247 |
| CACNA2D4 | CATCACGGGGGA | | ATCACGGGGGAGGGA | | CACGGGGGA | | GAGCCTCTCTC | |
| | | | GTTTgct | | | | | |
| gRNA1_AIK_ | GCAGCAGAAATA | 248 | cttGCAGCAGAAATAGA | 249 | caccGCAGCAGAAATAGAC | 250 | agacATGCAATTAGTCT | 251 |
| HEKsite3 | GACTAATTGCAT | | CTAATTGCATGGGCG | | TAATTGCAT | | ATTTCTGCTGC | |
| | | | TTTccc | | | | | |
| gRNA1_AIK_ | CCAGGTCAGATA | 256 | gtcCCAGGTCAGATAAA | 257 | caccGCCAGGTCAGATAAA | 258 | agacCTTCCTAAAATTTA | 259 |
| HEKsite4(**) | AATTTTAGGAAG | | TTTTAGGAAGTGCTGT | | TTTTAGGAAG | | TCTGACCTGGC | |
| | | | TTtcc | | | | | |
| gRNA2_AIK_ | AAGTCACCATCA | 252 | tcaAAGTCACCATCACA | 253 | caccGAAGTCACCATCACA | 254 | agacAGCGTTTCCTTGT | 255 |
| HEKsite4 | CAAGGAAACGCT | | AGGAAACGCTTGGTG | | AGGAAACGCT | | GATGGTGACTTC | |
| | | | TATtga | | | | | |
| gRNA1_AIK_ | GACCCCTAATAT | 264 | tttGACCCCTAATATGA | 265 | caccGACCCCTAATATGAA | 266 | agacACTCTACCCTTCA | 267 |
| Chr8 | GAAGGGTAGAGT | | AGGGTAGAGTGAGTG | | GGGTAGAGT | | TATTAGGGGTC | |
| | | | TGTgtg | | | | | |
| gRNA2_AIK_ | GGCAGGGCCTG | 260 | gtgGGCAGGGCCTGAC | 261 | caccGGCAGGGCCTGACA | 262 | agacCCCTTTCCGCTGT | 263 |
| Chr8 | ACAGCGGAAAGG | | AGCGGAAAGGGTGGA | | GCGGAAAGGG | | CAGGCCCTGCC | |
| | G | | GCTTtat | | | | | |
| gRNA1_AIK_ | GTGGCTGTGCTT | 268 | cttGTGGCTGTGCTTAG | 269 | caccGTGGCTGTGCTTAGG | 270 | agacCCCACGCTACCTA | 271 |
| BCR | AGGTAGCGTGGG | | GTAGCGTGGGATGTG | | TAGCGTGGG | | AGCACAGCCAC | |
| | | | TGTgtt | | | | | |
| gRNA2_AIK_ | AGTTCTTGCCGT | 272 | accAGTTCTTGCCGTG | 273 | caccGAGTTCTTGCCGTGC | 274 | agacTGGGGAAGGGGC | 275 |
| BCR | GCCCCTTCCCCA | | CCCCTTCCCCAGGGT | | CCCTTCCCCA | | ACGGCAAGAACTC | |
| | | | GTGTggt | | | | | |
| gRNA1_AIK_ | AGAGCTCTTGGT | 418 | gatAGAGCTCTTGGTA | 419 | caccGAGAGCTCTTGGTAC | 420 | agacATAACTTCAGGTA | 421 |
| HEKsite 1 | ACCTGAAGTTAT | | CCTGAAGTTATAGGG | | CTGAAGTTAT | | CCAAGAGCTCTC | |
| | | | GTTTagt | | | | | |
| gRNA1_AIK_ | GAGGATACCAGG | 422 | ataGAGGATACCAGGA | 423 | caccGAGGATACCAGGACT | 424 | agacGACAAAAGAAGTC | 425 |
| HBG1 | ACTTCTTTTGTC | | CTTCTTTTGTCAGCCG | | TCTTTTGTC | | CTGGTATCCTC | |
| | | | TTTttt | | | | | |
| gRNA2_AIK_ | GAAATGACCCAT | 426 | tgtGAAATGACCCATGG | 427 | caccGAAATGACCCATGGC | 428 | agacAGTCCAGACGCCA | 429 |
| HBG1 | GGCGTCTGGACT | | CGTCTGGACTAGGAG | | GTCTGGACT | | TGGGTCATTTC | |
| | | | CTTatt | | | | | |
| gRNA1_AIK_ | GCTTGCATTGTA | 430 | aatGCTTGCATTGTATG | 431 | caccGCTTGCATTGTATGT | 432 | agacAATAGCCAGACAT | 433 |
| HPRT | TGTCTGGCTATT | | TCTGGCTATTCTGTGT | | CTGGCTATT | | ACAATGCAAGC | |
| | | | TTtta | | | | | |
| gRNA2_AIK_ | ATCATTATGCTGA | 434 | ctaATCATTATGCTGAG | 435 | caccGATCATTATGCTGAG | 436 | agacTTTCCAAATCCTCA | 437 |
| HPRT | GGATTTGGAAA | | GATTTGGAAAGGGTG | | GATTTGGAAA | | GCATAATGATC | |
| | | | TTTatt | | | | | |
| gRNA1_AIK_ | GCACCTAATCTC | 438 | gcaGCACCTAATCTCC | 439 | caccGCACCTAATCTCCTA | 440 | agacTAAGTCCTCTAGG | 441 |
| IL2RG | CTAGAGGACTTA | | TAGAGGACTTAGCCC | | GAGGACTTA | | AGATTAGGTGC | |
| | | | GTGTcac | | | | | |
| gRNA2_AIK_ | GAGGCAGGAGG | 442 | gctGAGGCAGGAGGAT | 443 | caccGAGGCAGGAGGATC | 444 | agacGGCCTCTAGTGAT | 445 |
| IL2RG | ATCACTAGAGGC | | CACTAGAGGCCAGGA | | ACTAGAGGCC | | CCTCCTGCCTC | |
| | C | | GTTTgag | | | | | |
| gRNA2_AIK_ | GTTAGGAACTTT | 446 | gtaGTTAGGAACTTTAT | 447 | caccGTTAGGAACTTTATTG | 448 | agacGTTCCAGCCAATA | 449 |
| ATM | ATTGGCTGGAAC | | TGGCTGGAACTGGAG | | GCTGGAAC | | AAGTTCCTAAC | |
| | | | TTTcct | | | | | |
| gRNA2_AIK_ | GTTGAGGGGTCT | 450 | gtgGTTGAGGGGTCTC | 451 | caccGTTGAGGGGTCTCCT | 452 | agacCCACCACAAGGAG | 453 |
| NF1 | CCTTGTGGTGG | | CTTGTGGTGGAGGAG | | TGTGGTGG | | ACCCCTCAAC | |
| | | | TCTcct | | | | | |
| gRNA2_AIK_ | GAAAGGTTTTAC | 454 | gcaGAAAGGTTTTACTA | 455 | caccGAAAGGTTTTACTATT | 456 | agacAATTGTTGAATAGT | 457 |
| USH2A | TATTCAACAATT | | TTCAACAATTAGGAGT | | CAACAATT | | AAAACCTTTC | |
| | | | GTcca | | | | | |
| gRNA1_AIK_ | GGAATGAAATAA | 458 | atgGGAATGAAATAATT | 459 | caccGGAATGAAATAATTT | 460 | agacTGGCATACAAATT | 461 |
| BCLenh | TTTGTATGCCA | | TGTATGCCATGCCGT | | GTATGCCA | | ATTTCATTCC | |
| | | | GTgga | | | | | |
| gRNA1_AIK_ | GCCCTGTAAAGG | 462 | ctgGCCCTGTAAAGGA | 463 | caccGCCCTGTAAAGGAAA | 464 | agacTGTTCCAGTTTCC | 465 |
| HEKsite2 | AAACTGGAACA | | AACTGGAACACAAAG | | CTGGAACA | | TTTACAGGGC | |
| | | | CATaga | | | | | |
| gRNA5_AIK_ | GAGTATTCCATG | 466 | agtGAGTATTCCATGTC | 467 | caccGAGTATTCCATGTCC | 468 | agacTACACAATAGGAC | 469 |
| CFTR | TCCTATTGTGTA | | CTATTGTGTAGATTGT | | TATTGTGTA | | ATGGAATACTC | |
| | | | GTttt | | | | | |
| gRNA8_AIK_ | TGGAAAGTGAGT | 470 | cctTGGAAAGTGAGTAT | 471 | caccGTGGAAAGTGAGTAT | 472 | agacGGACATGGAATAC | 473 |
| CFTR | ATTCCATGTCC | | TCCATGTCCTATTGTG | | TCCATGTCC | | TCACTTTCCAC | |
| | | | Taga | | | | | |
| gRNA2_AIK_ | GAGTGAAGGCAG | 474 | ttgGAGTGAAGGCAGA | 475 | caccGAGTGAAGGCAGAGA | 476 | agacTTAACCCCTCTCT | 477 |
| HEKsite4.2 | AGAGGGGTTA | | GAGGGGTTAAGGTAG | | GGGGTTAA | | GCCTTCACTC | |
| | | | CATata | | | | | |
| gRNA2_ | TGGTTTCATAACT | 478 | gcaTGGTTTCATAACTA | 479 | caccGTGGTTTCATAACTA | 480 | aaacCCTCCTAGTTATG | 481 |
| SpCas9_EMX1 | AGGAGG | | GGAGGTGGtgt | | GGAGG | | AAACCAC | |
| gRNA2_ | GGCAGCCAGGG | 482 | ctgGGCAGCCAGGGCT | 483 | caccGGCAGCCAGGGCTG | 484 | aaacTGAGGCCAGCCCT | 485 |
| SpCas9_FAS | CTGGCCTCA | | GGCCTCAGGGtgt | | GCCTCA | | GGCTGCC | |
| gRNA2_ | GCTACCTGCGCC | 486 | cgcGCTACCTGCGCCA | 487 | caccGCTACCTGCGCCACA | 488 | aaacATGGATGTGGCGC | 489 |
| SpCas9_FANCF | ACATCCAT | | CATCCATCGGcgc | | TCCAT | | AGGTAGC | |
| gRNA2_ | TCAGGAGTCAGG | 490 | tccTCAGGAGTCAGGT | 491 | caccGTCAGGAGTCAGGTG | 492 | aaacTGGTGCACCTGAC | 493 |
| SpCas9_HBB | TGCACCA | | GCACCATGGtgt | | CACCA | | TCCTGAC | |
| gRNA2_ | CTCCACCATGTG | 494 | cttCTCCACCATGTGGG | 495 | caccGCTCCACCATGTGGG | 496 | aaacGAGAACCCACATG | 497 |
| SpCas9_ZSCAN2 | GGTTCTC | | TTCTCCGGtgt | | TTCTC | | GTGGAGC | |
| gRNA2_ | TCTTACAACAACA | 498 | gatTCTTACAACAACAT | 499 | caccGTCTTACAACAACAT | 500 | aaacCTCTCATGTTGTT | 501 |
| SpCas9_CHR6 | TGAGAG | | GAGAGAGGggt | | GAGAG | | GTAAGAC | |
| gRNA2_ | AATACTAAGACAT | 502 | agaAATACTAAGACAT | 503 | caccGAATACTAAGACATG | 504 | aaacCTCTGCATGTCTT | 505 |
| SpCas9_ADAM | GCAGAG | | GCAGAGAGGtgc | | CAGAG | | AGTATTC | |
| gRNA2_ | GGTTTGTGAACG | 506 | tgaGGTTTGTGAACGC | 507 | caccGGGTTTGTGAACGCG | 508 | aaacCTCCACGCGTTCA | 509 |
| SpCas9_B2M | CGTGGAG | | GTGGAGGGGcgc | | TGGAG | | CAAACCC | |
| gRNA2_ | GCTGAAAAGGTG | 510 | ttgGCTGAAAAGGTGGT | 511 | caccGCTGAAAAGGTGGTC | 512 | aaacACATAGACCACCT | 513 |
| SpCas9_CXCR4 | GTCTATGT | | CTATGTTGGcgt | | TATGT | | TTTCAGC | |
| gRNA2_ | GGTCAGGTGTCC | 514 | ggcGGTCAGGTGTCCC | 515 | caccGGGTCAGGTGTCCCA | 516 | aaacGGCTCTGGGACAC | 517 |
| SpCas9_PD1 | CAGAGCC | | AGAGCCAGGggt | | GAGCC | | CTGACCC | |
| gRNA2_ | TGTCAAGTGGCG | 518 | gccTGTCAAGTGGCGT | 519 | caccGTGTCAAGTGGCGTG | 520 | aaacGGTGTCACGCCAC | 521 |
| SpCas9_DNMT1 | TGACACC | | GACACCGGGcgt | | ACACC | | TTGACAC | |
| gRNA2_ | GGCCGAGACCAC | 522 | taaGGCCGAGACCACC | 523 | caccGGGCCGAGACCACC | 524 | aaacCTGATTGGTGGTC | 525 |
| SpCas9_TRAC | CAATCAG | | AATCAGAGGagt | | AATCAG | | TCGGCCC | |
| gRNA2_ | GAGGAAGCTGGT | 526 | tcaGAGGAAGCTGGTC | 527 | caccGAGGAAGCTGGTCTG | 528 | aaacAGGCCCAGACCA | 529 |
| SpCas9_TRBC | CTGGGCCT | | TGGGCCTGGGagt | | GGCCT | | GCTTCCTC | |
| gRNA2_ | GTGGTAGCTGGG | 530 | gagGTGGTAGCTGGG | 531 | caccGTGGTAGCTGGGGCT | 532 | aaacCCCCCAGCCCCA | 533 |
| SpCas9_ | GCTGGGGG | | GCTGGGGGCGGtgt | | GGGGG | | GCTACCAC | |
| VEGFAsite2 | | | | | | | | |
| gRNA2_ | TTCCCTCTTTAGC | 534 | ccaTTCCCTCTTTAGCC | 535 | caccGTTCCCTCTTTAGCC | 536 | aaacGCTCTGGCTAAAG | 537 |
| SpCas9_ | CAGAGC | | AGAGCCGGggt | | AGAGC | | AGGGAAC | |
| VEGFAsite3 | | | | | | | | |
| gRNA2_ | GAGGCTCCCATC | 538 | agaGAGGCTCCCATCA | 539 | caccGAGGCTCCCATCACG | 540 | aaacTCCCCCGTGATGG | 541 |
| SpCas9_ | ACGGGGGA | | CGGGGGAGGGagt | | GGGGA | | GAGCCTC | |
| CACNAD24 | | | | | | | | |
| gRNA2_ | AGAAATAGACTA | 542 | agcAGAAATAGACTAAT | 543 | caccGAGAAATAGACTAAT | 544 | aaacATGCAATTAGTCT | 545 |
| SpCas9_ | ATTGCAT | | TGCATGGGcgt | | TGCAT | | ATTTCTC | |
| HEKsite3 | | | | | | | | |
| gRNA2_ | ACCATCACAAGG | 546 | gtcACCATCACAAGGAA | 547 | caccGACCATCACAAGGAA | 548 | aaacAGCGTTTCCTTGT | 549 |
| SpCas9_ | AAACGCT | | ACGCTTGGtgt | | ACGCT | | GATGGTC | |
| HEKsite4 | | | | | | | | |
| gRNA2_ | GGGCCTGACAGC | 550 | gcaGGGCCTGACAGC | 551 | caccGGGCCTGACAGCGG | 552 | aaacCCCTTTCCGCTGT | 553 |
| SpCas9_Chr8 | GGAAAGGG | | GGAAAGGGTGGagc | | AAAGGG | | CAGGCCC | |
| gRNA2_ | TTGCCGTGCCCC | 554 | ttcTTGCCGTGCCCCTT | 555 | caccGTTGCCGTGCCCCTT | 556 | AAACTGGGGAAGGGG | 557 |
| SpCas9_BCR | TTCCCCA | | CCCCAGGGtgt | | CCCCA | | CACGGCAAC | |
| gRNA2_ | TCTTGGTACCTG | 558 | agcTCTTGGTACCTGA | 559 | caccGTCTTGGTACCTGAA | 560 | aaacATAACTTCAGGTA | 561 |
| SpCas9_ | AAGTTAT | | AGTTATAGGggt | | GTTAT | | CCAAGAC | |
| HEKsite 1 | | | | | | | | |
| gRNA2_ | GACCCATGGCGT | 562 | aatGACCCATGGCGTC | 563 | caccGGACCCATGGCGTCT | 564 | aaacAGTCCAGACGCCA | 565 |
| SpCas9_HBG1 | CTGGACT | | TGGACTAGGagc | | GGACT | | TGGGTCC | |
| gRNA2_ | TATGCTGAGGAT | 566 | catTATGCTGAGGATTT | 567 | caccGTATGCTGAGGATTT | 568 | aaacTTTCCAAATCCTCA | 569 |
| SpCas9_HPRT | TTGGAAA | | GGAAAGGGtgt | | GGAAA | | GCATAC | |
| gRNA2_ | AGGAGGATCACT | 570 | ggcAGGAGGATCACTA | 571 | caccGAGGAGGATCACTAG | 572 | aaacGGCCTCTAGTGAT | 573 |
| SpCas9_IL2RG | AGAGGCC | | GAGGCCAGGagt | | AGGCC | | CCTCCTC | |
| gRNA1_ | TTATCACAGGCT | 574 | cttTTATCACAGGCTCC | 575 | caccGTTATCACAGGCTCC | 576 | aaacTTCCTGGAGCCTG | 577 |
| SpCas9- | CCAGGAA | | AGGAAGGgtt | | AGGAA | | TGATAAC | |
| NG_BCLenh | | | | | | | | |
| gRNA1_ | GAACACAAAGCA | 578 | ctgGAACACAAAGCATA | 579 | caccGAACACAAAGCATAG | 580 | aaacGCAGTCTATGCTT | 581 |
| SpCas9- | TAGACTGC | | GACTGCGGggc | | ACTGC | | TGTGTTC | |
| NG_HEKsite2 | | | | | | | | |
| gRNA1_ | GTGAGTATTCCA | 582 | aaaGTGAGTATTCCAT | 583 | caccGTGAGTATTCCATGT | 584 | aaacATAGGACATGGAA | 585 |
| SpCas9-NG_CFTR | TGTCCTAT | | GTCCTATTGtgt | | CCTAT | | TACTCAC | |
| gRNA2_ | GAAGGCAGAGAG | 586 | agtGAAGGCAGAGAGG | 587 | caccGGAAGGCAGAGAGG | 588 | aaacTTAACCCCTCTCT | 589 |
| SpCas9- | GGGTTAA | | GGTTAAGGtag | | GGTTAA | | GCCTTCC | |
| NG_HEKsite4.2 | | | | | | | | |
| gRNA2_ | GAACTTTATTGG | 590 | tagGAACTTTATTGGCT | 591 | caccGAACTTTATTGGCTG | 592 | caacCCAGTTCCAGCCA | 593 |
| Nme2Cas9_ATM | CTGGAACTGG | | GGAACTGGAGTTTCCt | | GAACTGG | | ATAAAGTTC | |
| | | | ct | | | | | |
| gRNA2_ | GCTACTTGTGAA | 594 | tatGCTACTTGTGAAAC | 595 | caccGCTACTTGTGAAACA | 596 | caacGTTCAAGATGTTT | 597 |
| Nme2Cas9_CHR6 | ACATCTTGAAC | | ATCTTGAACAACACCc | | TCTTGAAC | | CACAAGTAGC | |
| | | | ct | | | | | |
| gRNA2_ | GAGGGGTCTCCT | 598 | gttGAGGGGTCTCCTTG | 599 | caccGAGGGGTCTCCTTGT | 600 | caacCCTCCACCACAAG | 601 |
| Nme2Cas9_NF1 | TGTGGTGGAGG | | TGGTGGAGGAGTCTC | | GGTGGAGG | | GAGACCCCTC | |
| | | | Cttc | | | | | |
| gRNA2_ | GTTACTCGCCTG | 602 | tctGTTACTCGCCTGTC | 603 | caccGTTACTCGCCTGTCA | 604 | caacACGCCACTTGACA | 605 |
| Nme2Cas9_DNMT1 | TCAAGTGGCGT | | AAGTGGCGTGACACC | | AGTGGCGT | | GGCGAGTAAC | |
| | | | ggg | | | | | |
| gRNA2_ | GTTTTACTATTCA | 606 | aagGTTTTACTATTCAA | 607 | caccGTTTTACTATTCAACA | 608 | caacCCTAATTGTTGAAT | 609 |
| Nme2Cas9_USH2A | ACAATTAGG | | CAATTAGGAGTGTCCa | | ATTAGG | | AGTAAAAC | |
| | | | ag | | | | | |
| gRNA2_ | GAGTCAATGCAG | 610 | cctGAGTCAATGCAGAT | 611 | caccGAGTCAATGCAGATA | 612 | caacAAGAGCTCTATCT | 613 |
| Nme2Cas9_ | ATAGAGCTCTT | | AGAGCTCTTGGTACCt | | GAGCTCTT | | GCATTGACTC | |
| HEKsite 1 | | | ga | | | | | |
| gRNA2_ | GGCACCAGTTCT | 614 | cctGGCACCAGTTCTTG | 615 | caccGGCACCAGTTCTTGC | 616 | caacGGGGCACGGCAA | 617 |
| Nme2Cas9_BCR | TGCCGTGCCCC | | CCGTGCCCCTTCCCC | | CGTGCCCC | | GAACTGGTGCC | |
| | | | agg | | | | | |
| gRNA2_ | GGGCTGTCAGAG | 618 | gctGGGCTGTCAGAGG | 619 | caccGGGCTGTCAGAGGAA | 620 | caacGACCAGCTTCCTC | 621 |
| Nme2Cas9_TRBC | GAAGCTGGTC | | AAGCTGGTCTGGGCCt | | GCTGGTC | | TGACAGCCC | |
| | | | gg | | | | | |
| gRNA2_ | GGTGGCAATGGA | 622 | tttGGTGGCAATGGATA | 623 | caccGGTGGCAATGGATAA | 624 | caacCTCGGCCTTATCC | 625 |
| Nme2Cas9_TRAC | TAAGGCCGAG | | AGGCCGAGACCACCa | | GGCCGAG | | ATTGCCACC | |
| | | | at | | | | | |
| gRNA2_ | GGAACTTTATTG | 626 | ttaGGAACTTTATTGGC | 627 | caccGGAACTTTATTGGCT | 628 | aaacGTTCCAGCCAATA | 629 |
| SaCas9_ATM | GCTGGAAC | | TGGAACTGGAGTttc | | GGAAC | | AAGTTCC | |
| gRNA2_ | GATTCTTACAACA | 630 | tttGATTCTTACAACAAC | 631 | caccGATTCTTACAACAACA | 632 | aaacCTCTCATGTTGTT | 633 |
| SaCas9_CHR6 | ACATGAGAG | | ATGAGAGAGGGGTgtt | | TGAGAG | | GTAAGAATC | |
| gRNA2_ | GAGGGGTCTCCT | 634 | gttGAGGGGTCTCCTTG | 635 | caccGAGGGGTCTCCTTGT | 636 | aaacCCACCACAAGGAG | 637 |
| SaCas9_NF1 | TGTGGTGG | | TGGTGGAGGAGTctc | | GGTGG | | ACCCCTC | |
| gRNA2_ | GTGACACCGGGC | 638 | ggcGTGACACCGGGC | 639 | caccGTGACACCGGGCGT | 640 | aaacGGGAACACGCCC | 641 |
| SaCas9_DNMT1 | GTGTTCCC | | GTGTTCCCCAGAGTga | | GTTCCC | | GGTGTCAC | |
| | | | c | | | | | |
| gRNA2_ | GGTTTTACTATTC | 642 | aaaGGTTTTACTATTCA | 643 | caccGGTTTTACTATTCAAC | 644 | aaacAATTGTTGAATAGT | 645 |
| SaCas9_USH2A | AACAATT | | ACAATTAGGAGTgtc | | AATT | | AAAACC | |
| gRNA2_ | GCTCTTGGTACC | 646 | agaGCTCTTGGTACCT | 647 | caccGCTCTTGGTACCTGA | 648 | aaacATAACTTCAGGTA | 649 |
| SaCas9_ | TGAAGTTAT | | GAAGTTATAGGGGTtta | | AGTTAT | | CCAAGAGC | |
| HEKsite1 | | | | | | | | |
| gRNA2_ | GTTCTTGCCGTG | 650 | ggtGTTCTTGCCGTGC | 651 | caccGTTCTTGCCGTGCCC | 652 | aaacGGGAAGGGGCAC | 653 |
| SaCas9_BCR | CCCCTTCCC | | CCCTTCCCCAGGGTgt | | CTTCCC | | GGCAAGAAC | |
| | | | g | | | | | |
| gRNA2_ | GAGGAAGCTGGT | 654 | tcaGAGGAAGCTGGTC | 655 | caccGAGGAAGCTGGTCTG | 656 | aaacAGGCCCAGACCA | 657 |
| SaCas9_TRBC | CTGGGCCT | | TGGGCCTGGGAGTctg | | GGCCT | | GCTTCCTC | |
| gRNA2_ | AGGCCGAGACCA | 658 | ataAGGCCGAGACCAC | 659 | caccGAGGCCGAGACCAC | 660 | aaacCTGATTGGTGGTC | 661 |
| SaCas9_TRAC | CCAATCAG | | CAATCAGAGGAGTttt | | CAATCAG | | TCGGCCTC | |
| AIK sgRHO-1 | TCTACGTGCCCT | 662 | actTCTACGTGCCCTTC | 663 | caccGTCTACGTGCCCTTC | 664 | agacCGCATTGGAGAAG | 665 |
| | TCTCCAATGCG | | TCCAATGCGACGGGT | | TCCAATGCG | | GGCACGTAGAC | |
| | | | GTggt | | | | | |
| AIK sgRHO-2 | CTCGAAGGGGCT | 666 | gtaCTCGAAGGGGCTG | 667 | caccGCTCGAAGGGGCTG | 668 | agacGGTGTGGTACGCA | 669 |
| | GCGTACCACACC | | CGTACCACACCCGTC | | CGTACCACACC | | GCCCCTTCGAGC | |
| | | | GCATtgg | | | | | |
| AIK sgRHO-3 | GGTAGTACTGTG | 670 | ccaGGTAGTACTGTGG | 671 | caccGGTAGTACTGTGGGT | 672 | agacCCTTCGAGTACCC | 673 |
| | GGTACTCGAAGG | | GTACTCGAAGGGGCT | | ACTCGAAGG | | ACAGTACTACC | |
| | | | GCGTacc | | | | | |
| AIK sgRHO-4 | ACGATCAGCAGA | 674 | agcACGATCAGCAGAA | 675 | caccGACGATCAGCAGAAA | 676 | agacCGCCTACATGTTT | 677 |
| | AACATGTAGGCG | | ACATGTAGGCGGCCA | | CATGTAGGCG | | CTGCTGATCGTC | |
| | | | GCATgga | | | | | |
| AIK sgRHO-5 | GGCAGTTCTCCA | 678 | catGGCAGTTCTCCAT | 679 | caccGGCAGTTCTCCATGC | 680 | agacAGGCGGCCAGCA | 681 |
| | TGCTGGCCGCCT | | GCTGGCCGCCTACAT | | TGGCCGCCT | | TGGAGAACTGCC | |
| | | | GTTTctg | | | | | |
| AIK sgRHO-6 | GCCTACATGTTT | 682 | gccGCCTACATGTTTCT | 683 | caccGCCTACATGTTTCTG | 684 | agacCACGATCAGCAGA | 685 |
| | CTGCTGATCGTG | | GCTGATCGTGCTGGG | | CTGATCGTG | | AACATGTAGGC | |
| | | | CTTccc | | | | | |
| AIK sgRHO-7 | GCTTCTTGTGCT | 686 | gcaGCTTCTTGTGCTG | 687 | caccGCTTCTTGTGCTGGA | 688 | agacCGTCACCGTCCAG | 689 |
| | GGACGGTGACG | | GACGGTGACGTAGAG | | CGGTGACG | | CACAAGAAGC | |
| | | | CGTgag | | | | | |
| AIK sgRHO-8 | GCAGGATGTAGT | 690 | tgaGCAGGATGTAGTT | 691 | caccGCAGGATGTAGTTGA | 692 | agacCACGCCTCTCAAC | 693 |
| | TGAGAGGCGTG | | GAGAGGCGTGCGCAG | | GAGGCGTG | | TACATCCTGC | |
| | | | CTTctt | | | | | |
| AIK sgRHO-9 | GCTAGGTTGAGC | 694 | acgGCTAGGTTGAGCA | 695 | caccGCTAGGTTGAGCAGG | 696 | agacCAACTACATCCTG | 697 |
| | AGGATGTAGTTG | | GGATGTAGTTGAGAG | | ATGTAGTTG | | CTCAACCTAGC | |
| | | | GCGTgcg | | | | | |
| AIK sgRHO- | GTGGCTGACCTC | 698 | gccGTGGCTGACCTCT | 699 | caccGTGGCTGACCTCTTC | 700 | agacTAGGACCATGAAG | 701 |
| 10 | TTCATGGTCCTA | | TCATGGTCCTAGGTG | | ATGGTCCTA | | AGGTCAGCCAC | |
| | | | GCTTcac | | | | | |
| AIK sgRHO- | GCTTCACCAGCA | 702 | gtgGCTTCACCAGCAC | 703 | caccGCTTCACCAGCACCC | 704 | agacAGGTGTAGAGGGT | 705 |
| 11 | CCCTCTACACCT | | CCTCTACACCTCTCTG | | TCTACACCT | | GCTGGTGAAGC | |
| | | | CATgga | | | | | |
| AIK sgRHO- | AATTGCATCCTG | 706 | ccaAATTGCATCCTGT | 707 | caccGAATTGCATCCTGTG | 708 | agacTCTTCGGGCCCAC | 709 |
| 12 | TGGGCCCGAAGA | | GGGCCCGAAGACGAA | | GGCCCGAAGA | | AGGATGCAATTC | |
| | | | GTATcca | | | | | |
| AIK sgRHO- | TTGGAGGGCTTC | 710 | aatTTGGAGGGCTTCTT | 711 | caccGTTGGAGGGCTTCTT | 712 | agacCAGGGTGGCAAA | 713 |
| 13 | TTTGCCACCCTG | | TGCCACCCTGGGCGG | | TGCCACCCTG | | GAAGCCCTCCAAC | |
| | | | TATgag | | | | | |
| AIK sgRHO- | CCCGAAGACGAA | 714 | gggCCCGAAGACGAAG | 715 | caccGCCCGAAGACGAAGT | 716 | agacCTGCATGGATACT | 717 |
| 14 | GTATCCATGCAG | | TATCCATGCAGAGAG | | ATCCATGCAG | | TCGTCTTCGGGC | |
| | | | GTGTaga | | | | | |
| AIK sgRHO- | CCAGGGTGGCAA | 718 | cgcCCAGGGTGGCAAA | 719 | caccGCCAGGGTGGCAAA | 720 | agacTGGAGGGCTTCTT | 721 |
| 15 | AGAAGCCCTCCA | | GAAGCCCTCCAAATT | | GAAGCCCTCCA | | TGCCACCCTGGC | |
| | | | GCATcct | | | | | |
| AIK sgRHO- | TTCGGGCCCACA | 722 | gtcTTCGGGCCCACAG | 723 | caccGTTCGGGCCCACAG | 724 | agacCAAATTGCATCCT | 725 |
| 16 | GGATGCAATTTG | | GATGCAATTTGGAGG | | GATGCAATTTG | | GTGGGCCCGAAC | |
| | | | GCTTctt | | | | | |
| AIK sgRHO- | CCTCTACACCTC | 726 | cacCCTCTACACCTCT | 727 | caccGCCTCTACACCTCTC | 728 | agacTATCCATGCAGAG | 729 |
| 17 | TCTGCATGGATA | | CTGCATGGATACTTCG | | TGCATGGATA | | AGGTGTAGAGGC | |
| | | | TCTtcg | | | | | |
| gRNA3_AIK_ | CCTCGCCGGACA | 84 | cgcCCTCGCCGGACAC | 85 | caccGCCTCGCCGGACAC | 86 | agacACAAGTTCAGCGT | 87 |
| EGFP | CGCTGAACTTGT | | GCTGAACTTGTGGCC | | GCTGAACTTGT | | GTCCGGCGAGGC | |
| | | | GTTTacg | | | | | |
| gRNA_ | GGGCACGGGCA | 730 | ccaGGGCACGGGCAG | 731 | caccGGGCACGGGCAGCT | 732 | gaacCCGGCAAGCTGC | 733 |
| SpCas9_EGFP | GCTTGCCGG | | CTTGCCGGTGGtgc | | TGCCGG | | CCGTGCCC | |
| gRNA_HPLH_ | GCCCATCCTGGT | 734 | ggtGCCCATCCTGGTC | 735 | caccGCCCATCCTGGTCGA | 736 | taacCCGTCCAGCTCGA | 737 |
| EGFP | CGAGCTGGACG | | GAGCTGGACGGCGAC | | GCTGGACGG | | CCAGGATGGGC | |
| | G | | GTAAcga | | | | | |
| gRNA_ANAB_ | GACGGCAACTAC | 738 | gacGACGGCAACTACA | 739 | caccGACGGCAACTACAAG | 740 | agacGCGCGGGTCTTGT | 741 |
| GFP | AAGACCCGCGC | | AGACCCGCGCCGAGG | | ACCCGCGC | | AGTTGCCGTC | |
| | | | TGAagt | | | | | |
| gRNA_ANAB_ | ATGTCTGTTACTC | 742 | tccATGTCTGTTACTCG | 743 | caccGATGTCTGTTACTCG | 744 | agacCTTGACAGGCGAG | 745 |
| DNMT1 | GCCTGTCAAG | | CCTGTCAAGTGGCGT | | CCTGTCAAG | | TAACAGACATC | |
| | | | GAcac | | | | | |
| gRNA_ANAB_ | AAAGCTGTGGGA | 746 | tgaAAAGCTGTGGGAA | 747 | caccGAAAGCTGTGGGAAA | 748 | agacGCGACCCGATTTC | 749 |
| HEKsite1 | AATCGGGTCGC | | ATCGGGTCGCTGGAG | | TCGGGTCGC | | CCACAGCTTTC | |
| | | | GAAggg | | | | | |
| gRNA_HPLH_ | GCCCGGTGTCAC | 750 | cacGCCCGGTGTCACG | 751 | caccGCCCGGTGTCACGC | 752 | taacCTGTCAAGTGGCG | 753 |
| DNMT1g1 | GCCACTTGACAG | | CCACTTGACAGGCGA | | CACTTGACAG | | TGACACCGGGC | |
| | | | GTAAcag | | | | | |
| gRNA_HPLH_ | GAGCCAAATTCA | 754 | gctGAGCCAAATTCACC | 755 | caccGAGCCAAATTCACCG | 756 | taacACTCCTGCTCGGT | 757 |
| DNMT1g2 | CCGAGCAGGAGT | | GAGCAGGAGTGAGGG | | AGCAGGAGT | | GAATTTGGCTC | |
| | | | AAAcgg | | | | | |
| gRNA_HPLH_ | GGGAAAGACCCA | 758 | agtGGGAAAGACCCAG | 759 | caccGGGAAAGACCCAGCA | 760 | taacACCCACGGATGCT | 761 |
| HEKsite1 | GCATCCGTGGGT | | CATCCGTGGGTCGCT | | TCCGTGGGT | | GGGTCTTTCCC | |
| | | | GAAAagc | | | | | |

(*)As for the guides used for the comparison of AIK Type II Cas with SpCas9, Nme2Cas9 and SaCas9, gRNA1 indicates guides NOT overlapping with SpCas9/SaCas9/Nme2Cas9 guides, while gRNA2 indicates overlapping guides. If gRNA2 is not indicated, gRNA1 is overlapping with SpCas9/SaCas9/Nme2Cas9. Note that guide names do not necessarily correspond to the guide names in Example 1.
(**)This guide was the same used for the evaluation of AIK Type II Cas as ABE in the HEKsite4.1 locus.
(***)The target sequences are reported with three flanking nucleotides on each side. The PAM sequence is highlighted in bold.
(****)The cloning overhang is reported in lowercase. Nucleotides highlighted in bold represent 5′-G appended to favor transcription from canonical U6 Pol III promoters.

### 7.2.1.1. Cell Lines

HEK293T cells (obtained from ATCC), U2OS-EGFP cells harboring a single integrated copy of an EGFP reporter gene and HEK293-RHO-EGFP cells stably expressing a RHO-EGFP minigene construct were cultured in DMEM (Life Technologies) supplemented with 10% FBS (Life Technologies), 2 mM GlutaMax (Life Technologies) and penicillin/streptomycin (Life Technologies). HEK293-RHO-EGFP cells were obtained by stable transfection of HEK293 cells with a RHO-EGFP reporter construct, obtained by cloning a fragment of the RHO gene up to exon 2 (retaining introns 1 and 2) fused to part of RHO cDNA containing exons 3-5 in frame with the EGFP coding sequence into a CMV-driven expression plasmid. Cells were pool-selected with 5 μg/ml Hygromycin (Invivogen) and single clones were subsequently isolated and expanded. All cells were incubated at 37° C. and 5% CO₂ in a humidified atmosphere. All cells tested mycoplasma negative (PlasmoTest™, Invivogen).

### 7.2.1.2. PAM Identification

PAM sequences of HPLH and ANABType II Cas proteins were identified as described in Sections 7.1.1.5. and 7.1.1.6.

### 7.2.1.3. Cell Line Transfections

For EGFP disruption assays, U2OS-EGFP cells were nucleofected with pX-Cas plasmid expressing the nuclease of interest as described in Section 7.1.1.7.

For editing analyses of endogenous genomic loci, HEK293T cells were transfected with pX-Cas plasmids expressing the nuclease of interest as described in Section 7.1.1.7.

### 7.2.1.4. Evaluation of Editing Activity

EGFP knock-out was analyzed four days after nucleofection using a BD FACSCanto™ (BD) flow cytometer. For the evaluation of indel formation at genomic loci cells, were collected three days after transfection and DNA was extracted using the QuickExtract™ DNA Extraction Solution (Lucigen) according to the manufacturer's instructions. To amplify the target loci, PCR reactions were performed using the HOT FIREPol® polymerase (Solis BioDyne), using the oligonucleotides listed in Table 13. The amplified products were purified, Sanger sequenced (EasyRun service, Microsynth) and analyzed with the TIDE web tool (shinyapps.datacurators.nl/tide/) to quantify indels or with the EditR web tool (baseeditr.com) to quantify base editing events.

TABLE 13
Oligonucleotides Used to Amplify Genomic Regions and to Perform TIDE Analysis.

| | | SEQ ID | | SEQ ID |
|---|---|---|---|---|
| Locus | For (5′→3′) | NO: | Rev (5′→3′) | NO: |
| EMX1 | ATTTCGGACTACCCTGA | 290 | GGAATCTACCACCCCAGGCT | 291 |
| | GGAG | | CT | |
| FAS_1 | TTAGAAAGGGCAGGAG | 292 | CTTGTCCAGGAGTTCCGCTC | 293 |
| | GC | | | |
| FAS 2 | AATTGAAGCGGAAGTCT | 294 | AACACTTCTCTCGCTATGCC | 295 |
| | GGG | | | |
| CCR5 | ATGCACAGGGTGGAAC | 296 | CTAAGCCATGTGCACAACTC | 297 |
| | AAGATGGA | | TGAC | |
| FANCF | GGCACATCTTGGGACTC | 298 | AGCATAGCGCCTGGCATTAA | 299 |
| | AG | | TAGG | |
| HBB | CAAAGAACCTCTGGGTC | 300 | GCATATTCTGGAGACGCAGG | 301 |
| | CAAG | | | |
| ZSCAN2 | GACTGTGGGCAGAGGT | 302 | TGTATACGGGACTTGACTCA | 303 |
| | TCAGC | | GACC | |
| CHR6 | ATGTCCTCATGCCGGAC | 304 | TCCAAGAGCATACGCACACA | 305 |
| | TG | | TTCC | |
| ADAMTSL1 | TAGGACTAGGCTCTTGG | 306 | CATAGAGTACTTAGTATGAG | 307 |
| | AG | | CGAGGC | |
| B2M | CCAGTCTAGTGCATGCC | 308 | GTTCCCATCACATGTCAC | 309 |
| | TTC | | | |
| CXCR4 | GGACAGGATGACAATAC | 310 | AGAGGAGTTAGCCAAGATGT | 311 |
| | CAGGCAGGATAAGGCC | | GACTTTGAAACC | |
| PD1 | ACGTCGTAAAGCCAAG | 312 | CACCCTCCCTTCAACCTGAC | 313 |
| | GTTAGTCC | | C | |
| DNMT1 | GTCTTAATTTCCACTCAT | 314 | CGTTTTGGGCTCTGGGACTC | 315 |
| | ACAGTGGTAG | | AG | |
| MATCH8 | TGTGTCGTCCATAAACG | 316 | CATCTTCCCTGAAATTTCTTA | 317 |
| | CTGCC | | AGAGGC | |
| TRAC | CTGTCCCTGAGTCCCAG | 318 | GGCCTAGAAGAGCAGTAAG | 319 |
| | T | | G | |
| TRBC | CTGACCACGTGGAGCT | 320 | CTTACTTACCCGAGGTAAAG | 321 |
| | GAG | | CC | |
| VEGFAsite2 | TGCGAGCAGCGAAAGC | 322 | TCCAATGCACCCAAGACAGC | 323 |
| | GACA | | | |
| VEGFAsite3 | GCATACGTGGGCTCCA | 324 | CCGCAATGAAGGGGAAGCT | 325 |
| | ACAGGT | | CGA | |
| CACNA2D4 | TACAGCAGGACTGTGTG | 326 | CTTCCATCCTCCATCAGGTC | 327 |
| | GCACG | | AGG | |
| HEKsite1 | GAAGGATAGAGGGTGG | 762 | TGGAGTGCAATGGCGTGAC | 763 |
| | GAGAGG | | | |
| HEKsite3 | TAGCTACGCCTGTGATG | 328 | CCAGAGAAGTTGCTAGGATG | 329 |
| | G | | AAAGG | |
| HEKsite4 | AACAATTTCAGATCGCG | 330 | GTCAGACGTCCAAAACCAGA | 331 |
| | G | | CTCC | |
| CHR8 | TCCTGGGTCTGAGTTTC | 332 | ACAACACAGATCTGCAGATC | 333 |
| | TGAGAGG | | TCCG | |
| BCR | GTCAGGGCGCTCCTTC | 334 | GTGTACAGGGCACCTGCA | 335 |
| | CTTC | | | |
| ATM | CTAAGGGGTCTGACACA | 764 | GTGGCTACAAGACATTTCCT | 765 |
| | GACTG | | CC | |
| HBG1 | GCCTGTGAGATTGACAA | 766 | TACTGCGCTGAAACTGTGGC | 767 |
| | GAACAG | | | |
| HPRT 1 | ACAGTTACTAATATCAT | 768 | GGCTGAAAGGAGAGAACT | 769 |
| | CTTACACC | | | |
| HPRT 2 | CAGCAGCTGTTCTGAGT | 770 | CCCTTGACCCAGAAATTCCA | 771 |
| | ACTTG | | C | |
| IL2RG | CTGGTTTGGATTAGATC | 772 | GTTCCAAGTGCAATTCATG | 773 |
| | AGAGG | | | |
| NF1 | GCAGTACTGCAAGCATC | 774 | GCTCCAAGATGGCCAACTAG | 775 |
| | CTG | | C | |
| USH2A | TCCACATCCCTCCCTTT | 776 | CCAGAGTAGAAGGCAGCTA | 777 |
| | CATG | | GC | |
| RHO | CAGTGATAGAGATCTCC | 778 | GAGATAGATGCGGGCTTCCA | 779 |
| | CTATC | | | |
| BCLenh | GGACTTGGGAGTTATCT | 780 | GAGGCAAGTCAGTTGGGAA | 781 |
| | GTAG | | C | |
| HEKsite2 | CACTGCCATTCTACCAA | 782 | CTAAAACATCCAACCTTGATA | 783 |
| | CAATAGAGG | | GAACACC | |
| CFTR | TGAGTTTTGCCACATTG | 784 | AGAGCATGCACCCTTAACCT | 785 |
| | GCCAG | | CA | |

### 7.2.2. Results

### 7.2.2.1. Identification of a HPLH and ANAB Type II Cas Orthologs

In this Example, a similar approach to Example 1 was employed to identify small Type II Cas orthologs between 950 aa and 1100 aa. Based on the integrity of the deriving locus a group, two additional Type II Cas nucleases with reduced molecular weights, HPLH Type II Cas and ANAB Type II Cas were identified.

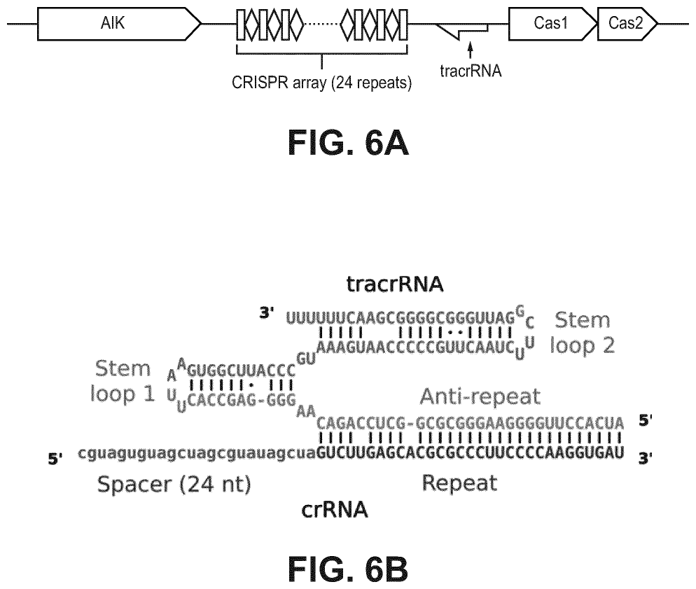

Notably, ANAB Type II Cas exhibits high sequence homology to AIK Type II Cas protein characterized in Example 1, as they are approximately 94% identical in their amino acid sequences. A schematic representation of the AIK Type II Cas bacterial genomic locus is reported in FIG. 6A. This locus includes the cas1, cas2 and cas9 genes and a CRISPR array composed of 23 spacer-direct repeat units. The domain structure of the newly identified nucleases, as inferred by multiple sequence alignment with Cas9 proteins with known structure, is reported in Table 2.

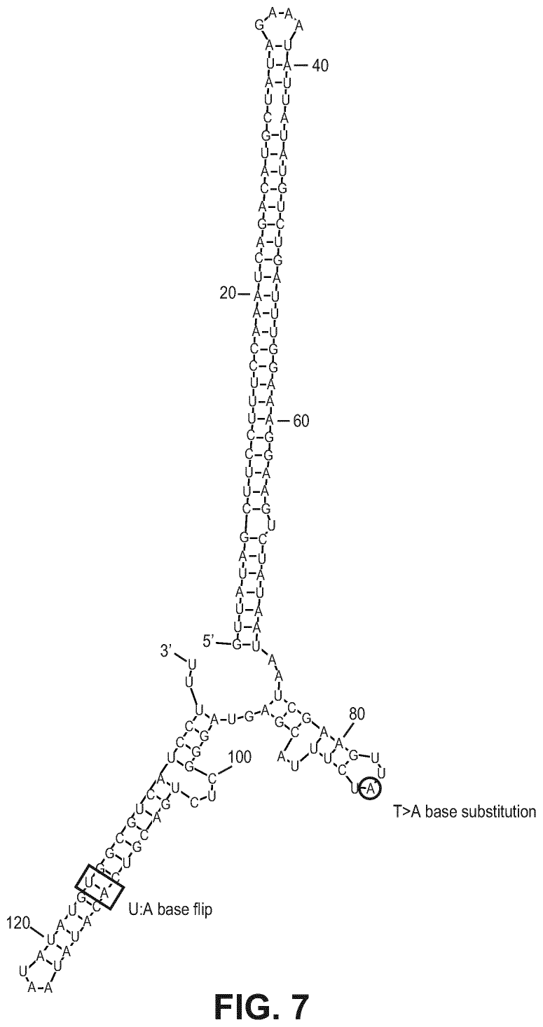

Remarkably, ANAB Type II Cas and AIK Type II Cas share the exact tracrRNA sequence (see, FIG. 6B). The identification of the tracrRNAs allowed the construction of exemplary sgRNAs for each nuclease, reported in Table 40 and Table 4D. Schematic representation of the exemplary sgRNAs are shown in FIG. 1A and FIG. 5B for ANAB Type II Cas (as well as AIK Type II Cas) and FIG. 7 for HPLH Type II Cas. When generating the HPLH Type II Cas sgRNA the 3′-end of the crRNA and the 5′-end of the tracrRNA were trimmed to improve the folding. In addition, a U:A base flip was introduced in the last stem-loop, together a T>A base substitution in the second loop to interrupt a T stretch and favor Pol III-mediated transcription (see FIG. 7).

### 7.2.2.2. Determination of the PAM Specificity of the ANAB and HPLH Type II Cas Nucleases

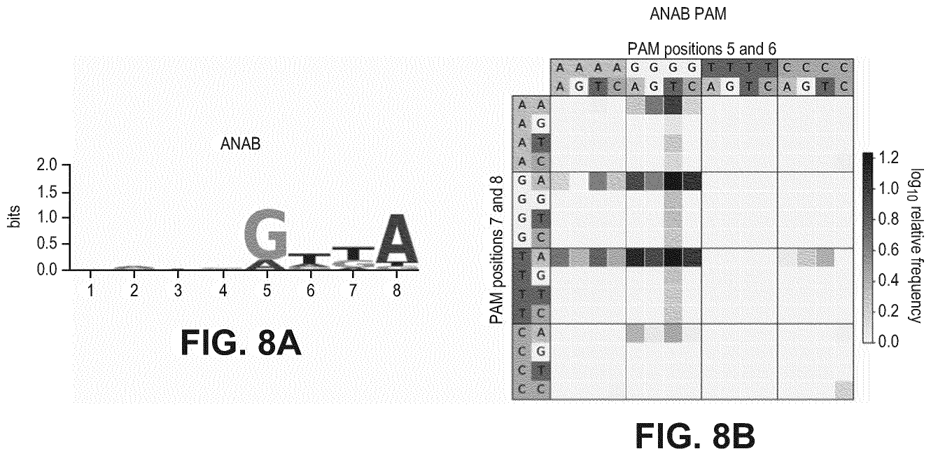

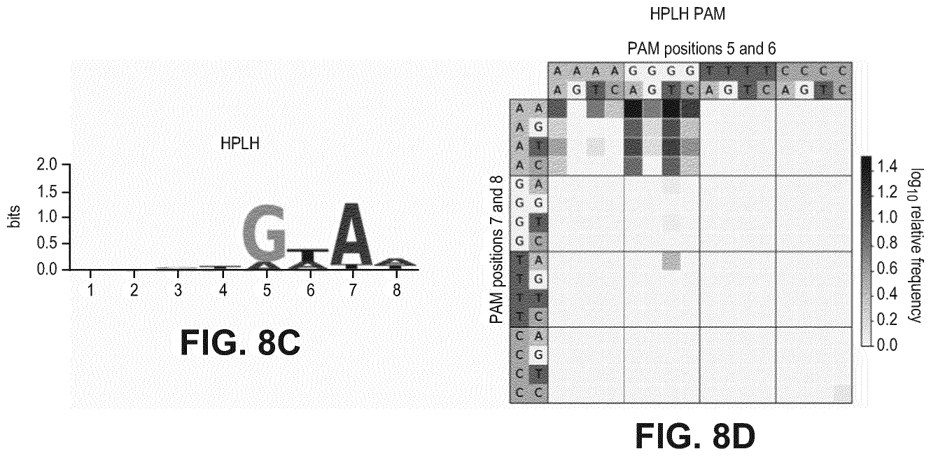

The PAM preferences of ANAB and HPLH nucleases were determined using an in vitro cleavage assay followed by NGS. The PAM sequence of ANAB Type II Cas corresponds to 5′-N₄RNKA-3′ where R=G or A and K=G or T (FIGS. 8A-8B). The PAM sequence for HPLH Type II Cas was determined to be 5′-N₄GWAN-3′, where W=T or A (FIGS. 8C-8D). To comprehensively visualize the PAM recognition profile, the relative frequency of all 256 four-nucleotide PAMs were plotted as a heatmap, showing additional preferences:
ANAB Type II Cas shows a preference for G in position 5 and a non-G nucleotide in position 6 thus resulting in a preferred 5′-N₄GHKA-3′ (where H=A, C or T and K=G or T), with the optimal PAM being 5′-N₄GTKA-3′ (Table 3D).
HPLH Type II Cas shows a preference for A in position 8 resulting in the optimal PAM 5′-N₄GWAA-3′ (where W=T or A). In addition, good levels of cleavage were observed also with a 5′-N₄GNAA-3′ PAM (Table 3C).

### 7.2.2.3. Evaluation of the Editing Activity of the Novel Type II Cas Proteins Using an EGFP Reporter System

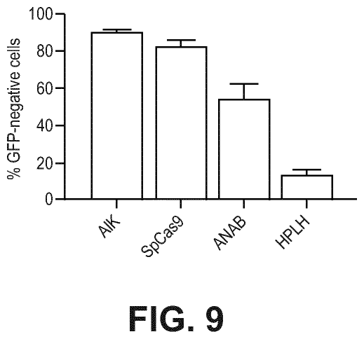

The editing activity of ANAB Type II Cas and HPLH Type II Cas was first evaluated through an EGFP disruption assay and compared to the editing activity of AIK Type II Cas (FIG. 9). Briefly, the highest editing activity was registered with AIK Type II Cas, with nearly 80% of cells being EGFP-negative. ANAB Type II Cas showed intermediate levels of EGFP knock-out (approximately 50%), whereas HPLH Type II Cas showed the least editing producing about 15% of EGFP-negative cells (FIG. 9).

Since AIK Type II Cas showed the most promising results in the initial studies among the identified nucleases, and ANAB Type II Cas shares identical guide RNA requirements, a process of sgRNA optimization in terms of spacer length and sgRNA scaffold was undertaken. A slight preference for 23-24 nt long spacers was revealed by comparing the editing activity of AIK Type II Cas on two genomic loci (HBB and FAS) using spacers going from 22 to 24 nt, (FIG. 10A). In addition, several alternative scaffold designs including modifications such as stem-loop trimming and specific base substitutions (FIG. 5B and sequences in Table 4B) were evaluated in parallel by targeting the DNMT1 B2M and DNMT1 loci without showing any significant difference in editing efficacy (FIG. 10B). Since having more compact sgRNAs is generally an advantage when packaging the nuclease into viral vectors (e.g., AAV vectors), the AIK Type II Cas trimmed sgRNAv4 was chosen alongside the full-length sgRNAv1 scaffold in subsequent studies.

### 7.2.2.4. Evaluation of Editing Activity on a Panel of Endogenous Genomic Loci

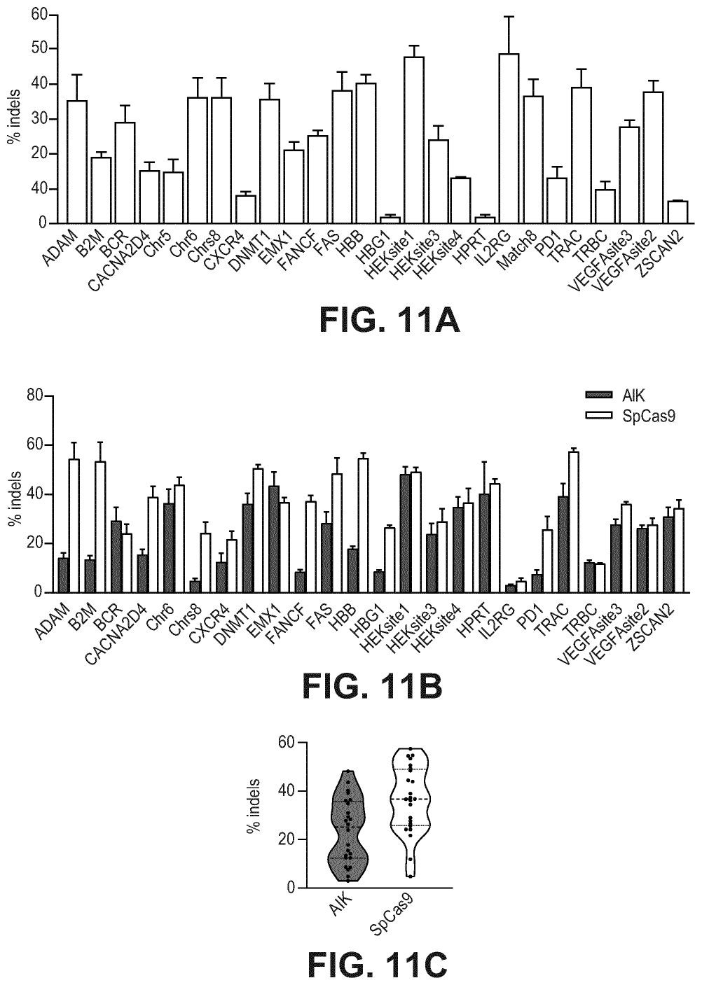

To evaluate editing activities of the ANAB and HPLH Type II Cas proteins and compare it to the activity of AIK Type II Cas, first the AIK Type II Cas activity was measured against a panel of 26 endogenous genomic loci, displaying up to 55% indels at specific sites (HEKsite1 and IL2RG) and variable efficacy throughout the targeted loci (FIG. 11A). To compare its activity with the commonly used SpCas9, a set of genomic targets (n=24) with overlapping spacer sequences was selected. Both AIK Type II Cas and SpCas9 produced comparable percentages of indels in the majority of the evaluated sites, with the former showing slightly lower editing activity (median difference 8.75%, FIG. 11B-C).

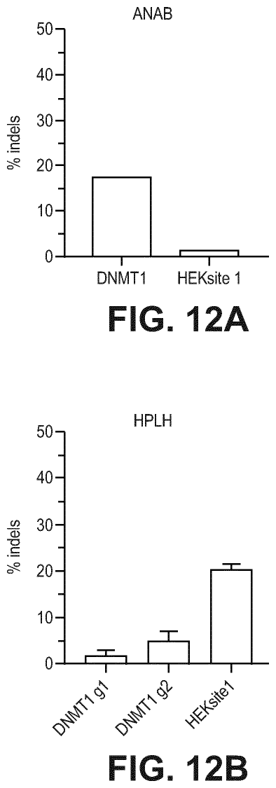

Next, the activities of ANAB Type II Cas and HPLH Type II Cas were evaluated on a panel of endogenous genomic loci. For both Type II Cas proteins, appreciable levels of editing (>10% indel) were measured in at least one evaluated site (DNMT1 for ANAB Type II Cas and HEKsite1 for HPLH Type II Cas), while lower percentages of indel formation were detected on the rest of the targets (FIG. 12A for ANAB and FIG. 12B for HPLH).

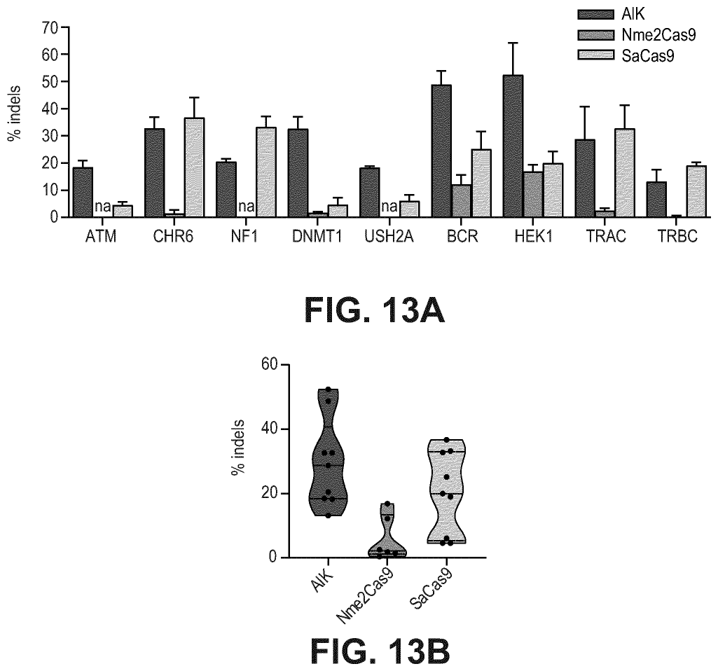

The reduced molecular weight of the Type II Cas proteins described herein is an attractive feature for size compatibility with AAV vectors. Currently, very few Type II Cas proteins with appreciable editing efficacy can be accommodated in these vectors, the two most notable of which are SaCas9 and Nme2Cas9. To comparatively analyze the editing efficacies of AIK Type II Cas with SaCa9 and Nme2Cas9 we identified 9 genomic loci (only six loci were evaluated for Nme2Cas9) with overlapping PAM sequences and measured indel formation. While Nme2Cas9 showed overall low activity throughout the analyzed loci (FIG. 13A), AIK Type II Cas and SaCas9 displayed comparable efficiency even though AIK Type II Cas generated more indels in the majority of the analyzed targets (5 out of 9, FIG. 13A). Overall, AIK Type II Cas was more active than both Nme2Cas9 and SaCas9 (e.g., 12.2% increase in the median editing activity when compared to SaCas9, FIG. 13B).

### 7.3. Example 3: Further Characterization of AIK Type II Cas Activity

This Example describes studies performed to further characterize the AIK Type II Cas ortholog.

### 7.3.1. Methods

### 7.3.1.1. Plasmids, Cell Lines, and Cell Transfections

Preparation of AIK Type II Cas plasmid constructs was described in detail in Section 7.1.1.1. Base editor constructs were made with a nickase version of AIK Type II Cas containing the D23A mutation, which was fused to the adenosine deaminase moiety contained in the adenine base editor ABE8e (Richter, 2020, Nature Biotechnology 38:883-891), generating pCMV-AIKABE8e. The ABE8e-AIK fusion comprised the amino acid sequence:

(SEQ ID NO: 793)
MKRTADGSEFESPKKKRKVSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIGRVVFGVRNSKRGAAGSLMN
VLNYPGMNHRVEITEGILADECAALLCDFYRMPRQVFNAQKKAQSSINSGGSSGGSSGSETPGTSESAT
PESSGGSSGGSEITINREIGKLGLPRHLVLGMAPGIASCGFALIDTANREILDLGVRLFDSPTHPKTGQSLA
VIRRGFRSTRRNIDRTQARLKHCLQILKAYGLIPQDATKEYFHTTKGDKQPLKLRVDGLDRLLNDREWAL
VLYSLCKRRGYIPHGEGNQDKSSEGGKVLSALAANKEAIAETSCRTVGEWLAQQPQSRNRGGNYDKCV
THAQLIEETHILFDAQRSFGSKYASPEFEAAYIEVCDWERSRKDFDRRTYDLVGHCSYFPTEKRAARCTL
TSELVSAYGALGNITIIHDDGTSRALSATERDECIAILFSCEPIRGNKDCAVKFGALRKALDLSSGDYFKGV
PAADEKTREVYKPKGWRVLRNTLNAANPILLQRLRDDRNLADAVMEAVAYSSALPVLQEQLQGLPLSEA
EIEALCRLPYSSKALNGYGNRSKKALDMLLDCLEEPEVLNLTQAENDCGLLGLRIAGTQLERSDRLMPYE
TWIERTGRTNNNPVVIRAMSQMRKVVNAICRKWGVPNEIHVELDRELRLPQRAKDEIAKANKKNEKNRE
RIAGQIAELRGCTADEVTGKQIEKYRLWEEQECFDLYTGAKIEVDRLISDDTYTQIDHILPFSRTGENSRNN
KVLVLAKSNQDKREQTPYEWMSHDGAPSWDAFERRVQENQKLSRRKKNFLLEKDLDTKEGEFLARSFT
DTAYMSREVCAYLADCLLFPDDGAKAHVVPTTGRATAWLRRRWGLNFGSNGEKDRSDDRHHATDACVI
AACSRSLVIKTARINQETHWSITRGMNETQRRDAIMKALESVMPWETFANEVRAAHDFVVPTRFVPRKG
KGELFEQTVYRYAGVNAQGKDIARKASSDKDIVMGNAVVSADEKSVIKVSEMLCLRLWHDPEAKKGQGA
WYADPVYKADIPALKDGTYVPRIAKQKYGRKVWKAVPNSALTQKPLEIYLGDLIKVGDKLGRYNGYNIAT
ANWSFVDALTKKEIAFPSVGMLSNELQPIIIRESILDNSGGSKRTADGSEFEPKKKRKV.

pCMV-NGABE8e, in which SpCas9-NG (Nishimasu, 2018, Science, 361(6408):1259-1262) is fused to the same adenosine deaminase, was used as a control. sgRNAs were expressed using dedicated pUC-derived plasmids, containing a U6-driven expression cassette for either the AIK Type II Cas sgRNAv4 or the SpCas9 sgRNA when using pCMV-NGABE8e.

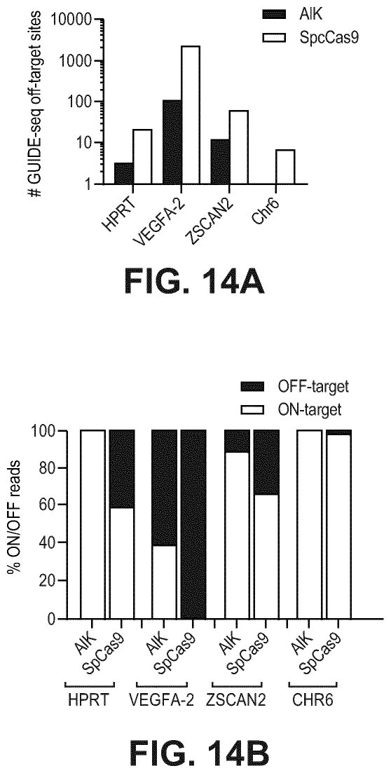

The AAV-EFS-AIK and AAV-EFS-ABE8e-AIK plasmids were designed as shown in FIG. 14A and synthesized by Vectorbuilder.

Cell lines and cell maintenance protocols used were as previously described in Section 7.1.1.2. Transfections of cell lines were carried out as described in Section 7.1.1.7. For editing analyses of endogenous genomic loci, 100,000 HEK293T cells were seeded in a 24-well plate 24 hours before transfection. Cells were then transfected with 1 μg of the pX-Cas plasmid expressing the nuclease of interest using the TranslT®-LT1 reagent (Mirus Bio) according to the manufacturer's protocol. Cell pellets were collected three days after transfection for indel evaluation. For base editing studies, cells were co-transfected with 750 ng of pCMV-ABE8e and 250 ng of pUC-sgRNA.

### 7.3.1.2. AAV Transductions

For AAV-DJ production, 107 AAVpro-293T cells (Takara) were seeded in P150 dishes in DMEM supplemented with 10% FBS, Pen/Strep and 2 mM Glutamine 24 hours before transfection. The next day, cells were transfected with pHelper, pAAV ITR-expression, and pAAV Rep-Cap plasmids using branched PEI (Sigma-Aldrich) in three P150 dishes for each vector production.

One day post transfection, the medium was replaced with OptiPro™ (LifeTechnologies) supplemented with Pen/Strep. Three days post-transfection, media and cells were collected, centrifuged and processed separately. Cells were washed and lysed with an acidic citrate buffer (55 mM citric acid, 55 mM sodium citrate, 800 mM NaCl, pH4.2, as described in Kimura et al., 2019. Sci Rep (9):13601). The lysates were cleared by centrifugation, and the pH was neutralized using 1 M HEPES buffer. The product was then treated with DNasel and RNaseA (both from ThermoFisher) and then mixed with the collected medium and NaCl (final concentration 500 mM). AAVs were precipitated with polyethylene glycol (PEG) 8000 (final concentration 8% v/v) overnight at 4° C. The precipitated AAVs were collected by centrifugation and resuspended in TNE Buffer (100 mM Tris-CI, pH 8.0, 150 mM NaCl, 20 mM EDTA) followed by 1:1 chloroform extraction. AAVs were collected and brought to a final volume of 1 ml and stored at 4° C.

For AAV transduction studies, 10⁵ cells were transduced in 24-well plates with 50 μl of the AAV productions and collected 6 days post-transduction for editing analysis and 10 days post-transduction for FACS analysis.

### 7.3.1.3. Off-target Evaluation

GUIDE-seq studies were performed as previously described (Casini et al., 2018, Nature Biotechnology. 36:265-271). Briefly, 2×10⁵ HEK293T cells were transfected using Lipofectamine 3000 (Invitrogen) with 1 μg of the all-in-one pX-AIKCas plasmid, encoding AIK Type II Cas and its sgRNA, and 10 pmol of the bait dsODN. Scramble sgRNA was used as negative control. The day after transfection, cells were detached and put under selection with 1 μg/ml puromycin. Two days after transfection, cells were collected, and genomic DNA extracted using NucleoSpin™ Tissue Kit (Macherey-Nagel) following manufacturer's instructions. Using a Covaris S200 sonicator, genomic DNA was sheared to an average length of 500 bp. End-repair reaction was performed using the NEBNext® Ultra™ End Repair/dA Tailing Module and adaptor ligation using NEBNext® Ultra™ Ligation Module, as described by Nobles et al. (Nobles et al., 2019, Genome Biology (20):14). Amplification steps for library preparation were performed following the original GUIDE-seq protocol from Tsai et al. (Tsai et al., 2015, Nature Biotechnology (33):187-197). After quantification, libraries were sequenced on an Illumina Miseq platform (v2 chemistry—300 cycles).

### 7.3.2. Results

### 7.3.2.1. Evaluation of AIK Type II Cas Off-target Activity

To evaluate the target specificity of AIK Type II Cas, a comparative off-target analysis with SpCas9 was performed through a whole-genome off-target detection method, GUIDE-seq. To this aim, a panel of four genomic loci (HPRT, VEGFA site 2, ZSCAN2 and Chr6) where both nucleases displayed similar on-target editing efficacy using overlapping spacer sequences was selected (FIG. 11B). In all examined loci, AIK Type II Cas produced far fewer off-target cleavages than SpCas9 (FIG. 14A) and these off-targets were less prone to be cut than the on-target site, as determined by the distribution of the GUIDE-seq reads (FIG. 14B). The superior performance of AIK Type II Cas was particularly striking at the VEGFA site2 where AIK Type II Cas showed at least 10 times fewer unwanted cleavages (FIG. 14A). At this specific gold standard site, SpCas9 barely discriminated between the on-target and the off-target, producing 1950 off-target cleavages, while the off-target cleavages by AIK Type II Cas were limited to 101 (FIG. 14A). In addition, SpCas9 was associated with many off-target sites with greater accumulation of GUIDE-seq reads than the desired on-target indicating an extreme lack of specificity, in contrast to the observations with AIK Type II Cas.

### 7.3.2.2. Evaluating the Efficacy of AIK Type II Cas as an Adenine Base Editor

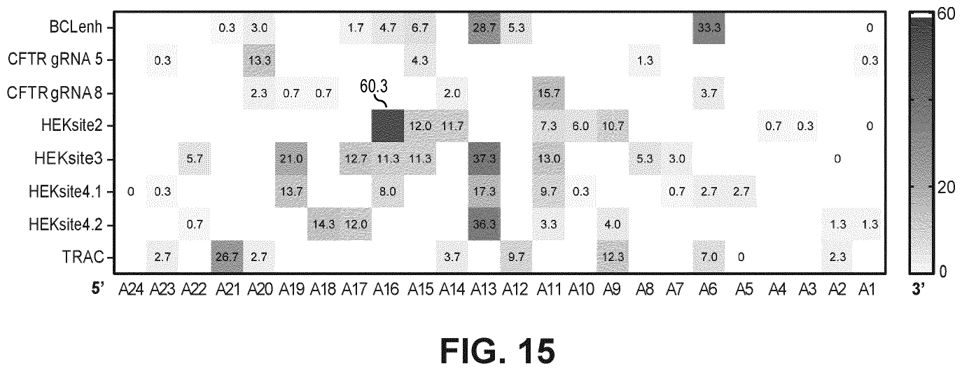

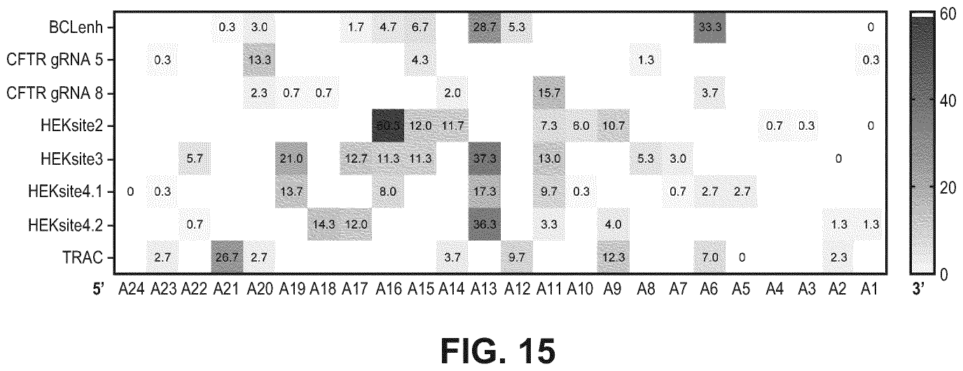

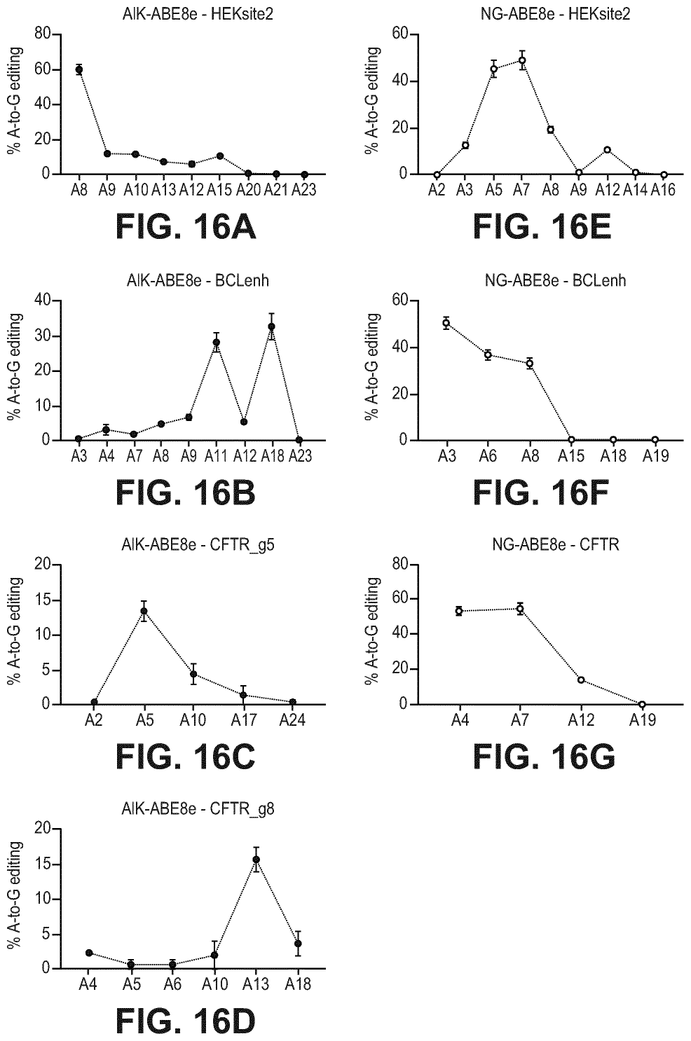

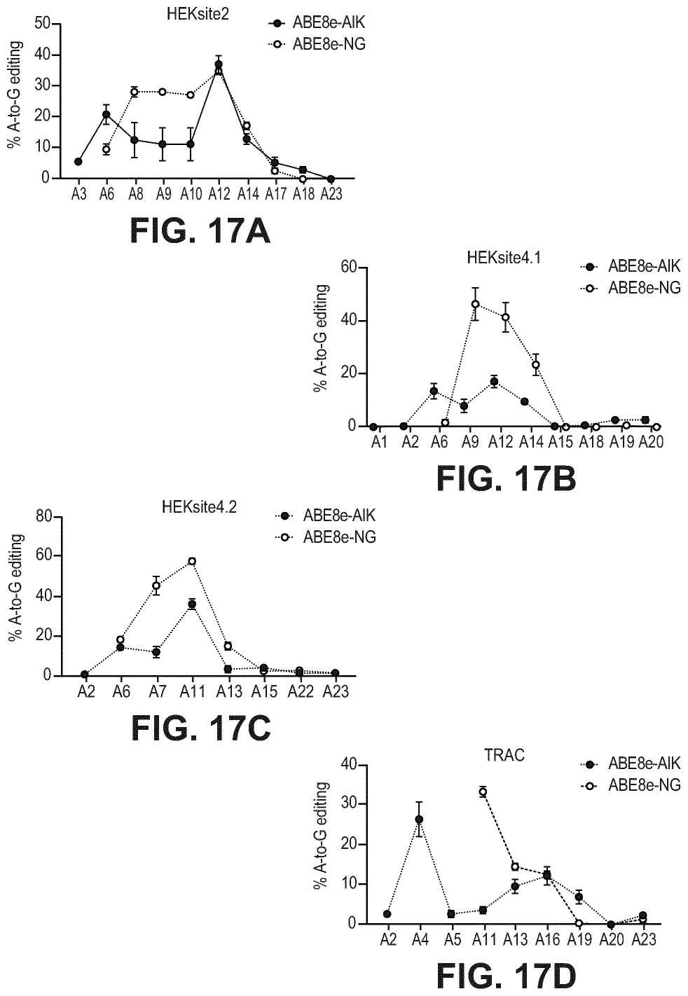

AIK Type II Cas was then evaluated in base-editing applications by fusing its nickase version (mutated at the D23 residue of the RuvC-I domain) with an engineered adenosine deaminase, ABE8e-AIKCas9 (Richter et al., 2020. Nature Biotechnology, (38):883-891). In each of the eight evaluated loci percentages of A to G transition ranging from −15% to 60% were detected depending on the target (FIG. 15). To further analyze the editing window and efficacy of ABE8e-AIKCas9, a comparative analysis was performed with ABE8e-NGCas9 (Nishimasu et al., 2018, Science 361(6408):1259-1262), both on neighboring (FIGS. 16A-16G) and matched sites (FIGS. 17A-D), observing that the main A to G transition occurs at different positions from the PAM between the two base editors, possibly due to different protein structures. Notably, even though the editing windows differ, the percentages of A to G transitions are similar between the two orthologs, thus confirming that adenine base editors based on AIK Type II Cas have similar editing power as those based on SpCas9 (FIGS. 16A-17D).

### 7.3.2.3. Delivery of AIK Type II Cas and ABE8e-AIK Using Single AAV Vectors

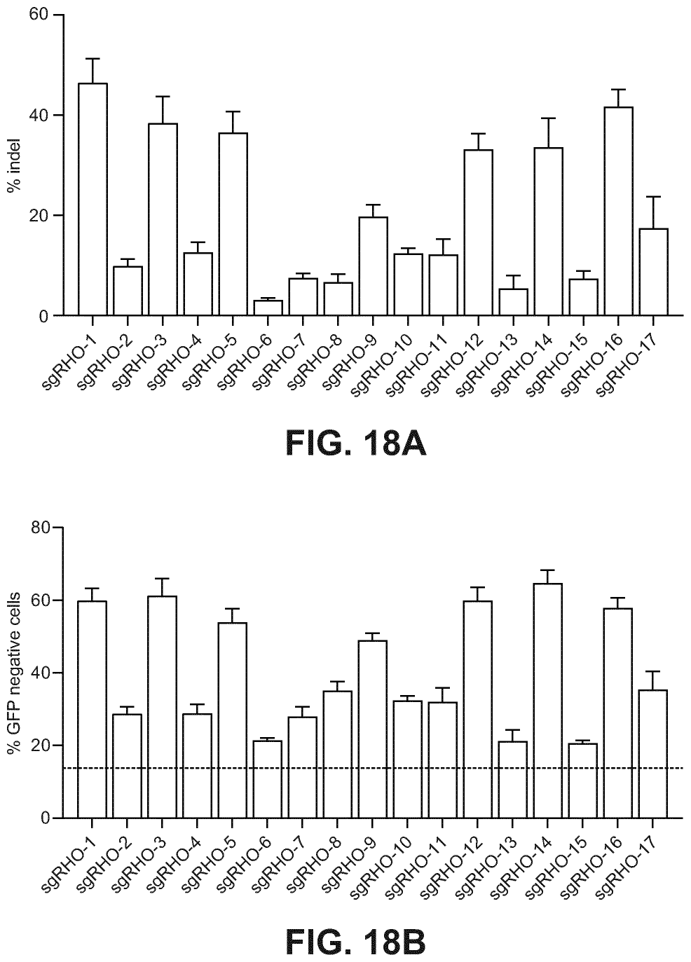

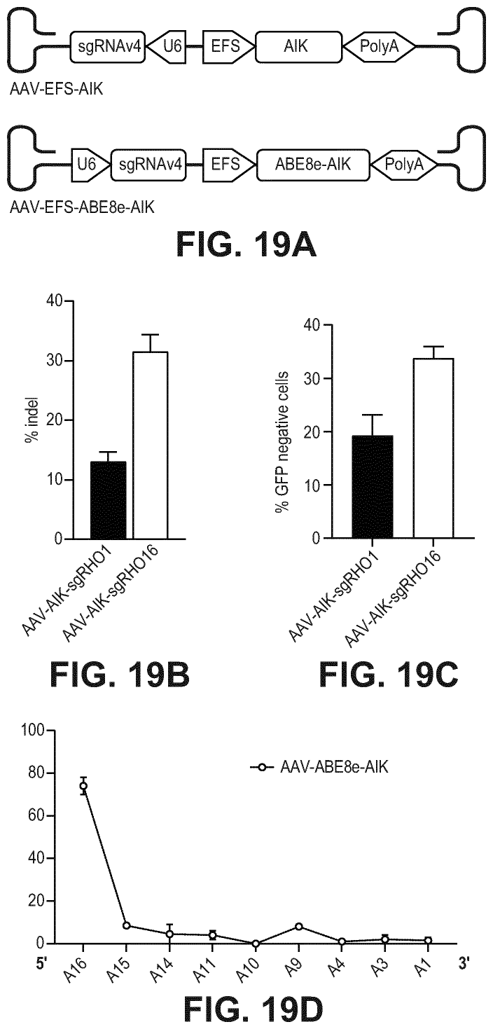

Given the promising properties of AIK Type II Cas for clinical development, its delivery as a nuclease or base editor through a single AAV including the sgRNA (schematically shown in FIG. 19A) was evaluated. AIK Type II Cas nuclease was evaluated against the RHO gene since this is a target with therapeutic potential. A panel of guides targeting the first exon of the human RHO gene were evaluated for their cleavage activity by transient transfection in HEK293 cells that stably express a RHO-EGFP reporter gene (FIG. 18A). Moreover, to confirm indel formation and gene KO, downregulation of RHO-EGFP was also measured by FACS analysis in the same treated cells (FIG. 18B). By incorporating the best performing guides (sgRHO-1 and sgRHO-16, which displayed up to 50% editing efficacy) in the AAV vectors, up to 30% indels were obtained after transduction of the HEK293 RHO-EGFP reporter cells (FIG. 19B). This was paralleled by a corresponding decrease in the percentage of RHO-EGFP positive cells (FIG. 14C).

Next, to evaluate the possibility of delivering the compact AIK Type II Cas-based adenine base editor (ABE8e-AIK) together with its sgRNA using a single AAV vector, HEK293T cells were transduced with the all-in-one AAV particle targeting HEKsite2 showing up to 80% of A to G transitions (FIG. 19D), thus having a similar base editing efficacy to the one observed through plasmid transfection (˜60%; FIG. 16A). Therefore, AIK Type II Cas is fully compatible with AAV delivery as demonstrated by complete conservation of the editing efficacy for both indels and deamination, obtained by transient transfect of plasmids. These results demonstrate the great potential of AIK Type II Cas and the other Type II Cas proteins described herein for clinical exploitation.

7.4. Example 4: “Super trimmed” sgRNA scaffold

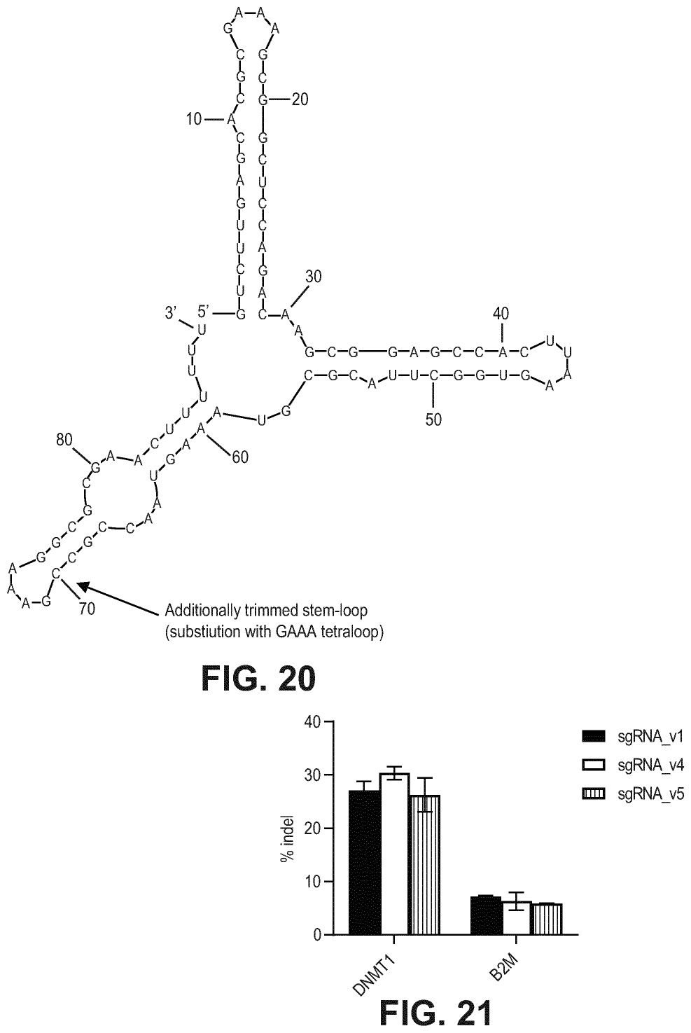

A “super trimmed” scaffold based on the AIK Type II Cas sgRNA_v4 scaffold was designed. The scaffold, AIK Type II Cas sgRNA_v5, includes the features of the v4 scaffold but includes an additionally trimmed stem-loop (FIG. 20). Indel formation at the DNMT1 and B2M loci was evaluated as in Example 1 using wild-type AIK Type II Cas and gRNAs having the AIK Type II Cas sgRNA_v1, sgRNA_v4, or sgRNA_v5 scaffold with six 3′ uracils (SEQ ID NO:26, SEQ ID NO:29, and SEQ ID NO:823, respectively). Results are shown in FIG. 21.

### 8. SPECIFIC EMBODIMENTS

The present disclosure is exemplified by the specific embodiments below.

1. A Type II Cas protein comprising an amino acid sequence having at least 50% sequence identity to:
(a) the amino acid sequence of a RuvC-I domain of a reference protein sequence;
(b) the amino acid sequence of a RuvC-II domain of a reference protein sequence;
(c) the amino acid sequence of a RuvC-III domain of a reference protein sequence;
(d) the amino acid sequence of a BH domain of a reference protein sequence;
(e) the amino acid sequence of a REC domain of a reference protein sequence;
(f) the amino acid sequence of a HNH domain of a reference protein sequence;
(g) the amino acid sequence of a WED domain of a reference protein sequence;
(h) the amino acid sequence of a PID domain of a reference protein sequence; or
(i) the amino acid sequence of the full length of a reference protein sequence;
wherein the reference protein sequence is SEQ ID NO:1, SEQ ID NO:2, SEQ ID NO:7, SEQ ID NO:8, SEQ ID NO:30, SEQ ID NO:31, SEQ ID NO:34, or SEQ ID NO:35.

2. The Type II Cas protein of embodiment 1, wherein the amino acid sequence of the Type II Cas protein comprises an amino acid sequence that is at least 50% identical to the amino acid sequence of the RuvC-I domain of the reference protein sequence.

3. The Type II Cas protein of embodiment 1, wherein the amino acid sequence of the Type II Cas protein comprises an amino acid sequence that is at least 55% identical to the amino acid sequence of the RuvC-I domain of the reference protein sequence.

4. The Type II Cas protein of embodiment 1, wherein the amino acid sequence of the Type II Cas protein comprises an amino acid sequence that is at least 60% identical to the amino acid sequence of the RuvC-I domain of the reference protein sequence.

5. The Type II Cas protein of embodiment 1, wherein the amino acid sequence of the Type II Cas protein comprises an amino acid sequence that is at least 65% identical to the amino acid sequence of the RuvC-I domain of the reference protein sequence.

6. The Type II Cas protein of embodiment 1, wherein the amino acid sequence of the Type II Cas protein comprises an amino acid sequence that is at least 70% identical to the amino acid sequence of the RuvC-I domain of the reference protein sequence.

7. The Type II Cas protein of embodiment 1, wherein the amino acid sequence of the Type II Cas protein comprises an amino acid sequence that is at least 75% identical to the amino acid sequence of the RuvC-I domain of the reference protein sequence.

8. The Type II Cas protein of embodiment 1, wherein the amino acid sequence of the Type II Cas protein comprises an amino acid sequence that is at least 80% identical to the amino acid sequence of the RuvC-I domain of the reference protein sequence.

9. The Type II Cas protein of embodiment 1, wherein the amino acid sequence of the Type II Cas protein comprises an amino acid sequence that is at least 85% identical to the amino acid sequence of the RuvC-I domain of the reference protein sequence.

10. The Type II Cas protein of embodiment 1, wherein the amino acid sequence of the Type II Cas protein comprises an amino acid sequence that is at least 90% identical to the amino acid sequence of the RuvC-I domain of the reference protein sequence.

11. The Type II Cas protein of embodiment 1, wherein the amino acid sequence of the Type II Cas protein comprises an amino acid sequence that is at least 95% identical to the amino acid sequence of the RuvC-I domain of the reference protein sequence.

12. The Type II Cas protein of embodiment 1, wherein the amino acid sequence of the Type II Cas protein comprises an amino acid sequence that is at least 96% identical to the amino acid sequence of the RuvC-I domain of the reference protein sequence.

13. The Type II Cas protein of embodiment 1, wherein the amino acid sequence of the Type II Cas protein comprises an amino acid sequence that is at least 97% identical to the amino acid sequence of the RuvC-I domain of the reference protein sequence.

14. The Type II Cas protein of embodiment 1, wherein the amino acid sequence of the Type II Cas protein comprises an amino acid sequence that is at least 98% identical to the amino acid sequence of the RuvC-I domain of the reference protein sequence.

15. The Type II Cas protein of embodiment 1, wherein the amino acid sequence of the Type II Cas protein comprises an amino acid sequence that is at least 99% identical to the amino acid sequence of the RuvC-I domain of the reference protein sequence.

16. The Type II Cas protein of embodiment 1, wherein the amino acid sequence of the Type II Cas protein comprises an amino acid sequence that is identical to the amino acid sequence of the RuvC-I domain of the reference protein sequence.

17. The Type II Cas protein of any one of embodiments 1 to 16, wherein the amino acid sequence of the Type II Cas protein comprises an amino acid sequence that is at least 50% identical to the amino acid sequence of the RuvC-II domain of the reference protein sequence.

18. The Type II Cas protein of any one of embodiments 1 to 16, wherein the amino acid sequence of the Type II Cas protein comprises an amino acid sequence that is at least 55% identical to the amino acid sequence of the RuvC-II domain of the reference protein sequence.

19. The Type II Cas protein of any one of embodiments 1 to 16, wherein the amino acid sequence of the Type II Cas protein comprises an amino acid sequence that is at least 60% identical to the amino acid sequence of the RuvC-II domain of the reference protein sequence.

20. The Type II Cas protein of any one of embodiments 1 to 16, wherein the amino acid sequence of the Type II Cas protein comprises an amino acid sequence that is at least 65% identical to the amino acid sequence of the RuvC-II domain of the reference protein sequence.

21. The Type II Cas protein of any one of embodiments 1 to 16, wherein the amino acid sequence of the Type II Cas protein comprises an amino acid sequence that is at least 70% identical to the amino acid sequence of the RuvC-II domain of the reference protein sequence.

22. The Type II Cas protein of any one of embodiments 1 to 16, wherein the amino acid sequence of the Type II Cas protein comprises an amino acid sequence that is at least 75% identical to the amino acid sequence of the RuvC-II domain of the reference protein sequence.

23. The Type II Cas protein of any one of embodiments 1 to 16, wherein the amino acid sequence of the Type II Cas protein comprises an amino acid sequence that is at least 80% identical to the amino acid sequence of the RuvC-II domain of the reference protein sequence.

24. The Type II Cas protein of any one of embodiments 1 to 16, wherein the amino acid sequence of the Type II Cas protein comprises an amino acid sequence that is at least 85% identical to the amino acid sequence of the RuvC-II domain of the reference protein sequence.

25. The Type II Cas protein of any one of embodiments 1 to 16, wherein the amino acid sequence of the Type II Cas protein comprises an amino acid sequence that is at least 90% identical to the amino acid sequence of the RuvC-II domain of the reference protein sequence.

26. The Type II Cas protein of any one of embodiments 1 to 16, wherein the amino acid sequence of the Type II Cas protein comprises an amino acid sequence that is at least 95% identical to the amino acid sequence of the RuvC-II domain of the reference protein sequence.

27. The Type II Cas protein of any one of embodiments 1 to 16, wherein the amino acid sequence of the Type II Cas protein comprises an amino acid sequence that is at least 96% identical to the amino acid sequence of the RuvC-II domain of the reference protein sequence.

28. The Type II Cas protein of any one of embodiments 1 to 16, wherein the amino acid sequence of the Type II Cas protein comprises an amino acid sequence that is at least 97% identical to the amino acid sequence of the RuvC-II domain of the reference protein sequence.

29. The Type II Cas protein of any one of embodiments 1 to 16, wherein the amino acid sequence of the Type II Cas protein comprises an amino acid sequence that is at least 98% identical to the amino acid sequence of the RuvC-II domain of the reference protein sequence.

30. The Type II Cas protein of any one of embodiments 1 to 16, wherein the amino acid sequence of the Type II Cas protein comprises an amino acid sequence that is at least 99% identical to the amino acid sequence of the RuvC-II domain of the reference protein sequence.

31. The Type II Cas protein of any one of embodiments 1 to 16, wherein the amino acid sequence of the Type II Cas protein comprises an amino acid sequence that is identical to the amino acid sequence of the RuvC-II domain of the reference protein sequence.

32. The Type II Cas protein of any one of embodiments 1 to 31, wherein the amino acid sequence of the Type II Cas protein comprises an amino acid sequence that is at least 50% identical to the amino acid sequence of the RuvC-III domain of the reference protein sequence.

33. The Type II Cas protein of any one of embodiments 1 to 31, wherein the amino acid sequence of the Type II Cas protein comprises an amino acid sequence that is at least 55% identical to the amino acid sequence of the RuvC-III domain of the reference protein sequence.

34. The Type II Cas protein of any one of embodiments 1 to 31, wherein the amino acid sequence of the Type II Cas protein comprises an amino acid sequence that is at least 60% identical to the amino acid sequence of the RuvC-III domain of the reference protein sequence.

35. The Type II Cas protein of any one of embodiments 1 to 31, wherein the amino acid sequence of the Type II Cas protein comprises an amino acid sequence that is at least 65% identical to the amino acid sequence of the RuvC-III domain of the reference protein sequence.

36. The Type II Cas protein of any one of embodiments 1 to 31, wherein the amino acid sequence of the Type II Cas protein comprises an amino acid sequence that is at least 70% identical to the amino acid sequence of the RuvC-III domain of the reference protein sequence.

37. The Type II Cas protein of any one of embodiments 1 to 31, wherein the amino acid sequence of the Type II Cas protein comprises an amino acid sequence that is at least 75% identical to the amino acid sequence of the RuvC-III domain of the reference protein sequence.

38. The Type II Cas protein of any one of embodiments 1 to 31, wherein the amino acid sequence of the Type II Cas protein comprises an amino acid sequence that is at least 80% identical to the amino acid sequence of the RuvC-III domain of the reference protein sequence.

39. The Type II Cas protein of any one of embodiments 1 to 31, wherein the amino acid sequence of the Type II Cas protein comprises an amino acid sequence that is at least 85% identical to the amino acid sequence of the RuvC-III domain of the reference protein sequence.

40. The Type II Cas protein of any one of embodiments 1 to 31, wherein the amino acid sequence of the Type II Cas protein comprises an amino acid sequence that is at least 90% identical to the amino acid sequence of the RuvC-III domain of the reference protein sequence.

41. The Type II Cas protein of any one of embodiments 1 to 31, wherein the amino acid sequence of the Type II Cas protein comprises an amino acid sequence that is at least 95% identical to the amino acid sequence of the RuvC-III domain of the reference protein sequence.

42. The Type II Cas protein of any one of embodiments 1 to 31, wherein the amino acid sequence of the Type II Cas protein comprises an amino acid sequence that is at least 96% identical to the amino acid sequence of the RuvC-III domain of the reference protein sequence.

43. The Type II Cas protein of any one of embodiments 1 to 31, wherein the amino acid sequence of the Type II Cas protein comprises an amino acid sequence that is at least 97% identical to the amino acid sequence of the RuvC-III domain of the reference protein sequence.

44. The Type II Cas protein of any one of embodiments 1 to 31, wherein the amino acid sequence of the Type II Cas protein comprises an amino acid sequence that is at least 98% identical to the amino acid sequence of the RuvC-III domain of the reference protein sequence.

45. The Type II Cas protein of any one of embodiments 1 to 31, wherein the amino acid sequence of the Type II Cas protein comprises an amino acid sequence that is at least 99% identical to the amino acid sequence of the RuvC-III domain of the reference protein sequence.

46. The Type II Cas protein of any one of embodiments 1 to 31, wherein the amino acid sequence of the Type II Cas protein comprises an amino acid sequence that is identical to the amino acid sequence of the RuvC-III domain of the reference protein sequence.

47. The Type II Cas protein of any one of embodiments 1 to 46, wherein the amino acid sequence of the Type II Cas protein comprises an amino acid sequence that is at least 50% identical to the amino acid sequence of the BH domain of the reference protein sequence.

48. The Type II Cas protein of any one of embodiments 1 to 46, wherein the amino acid sequence of the Type II Cas protein comprises an amino acid sequence that is at least 55% identical to the amino acid sequence of the BH domain of the reference protein sequence.

49. The Type II Cas protein of any one of embodiments 1 to 46, wherein the amino acid sequence of the Type II Cas protein comprises an amino acid sequence that is at least 60% identical to the amino acid sequence of the BH domain of the reference protein sequence.

50. The Type II Cas protein of any one of embodiments 1 to 46, wherein the amino acid sequence of the Type II Cas protein comprises an amino acid sequence that is at least 65% identical to the amino acid sequence of the BH domain of the reference protein sequence.

51. The Type II Cas protein of any one of embodiments 1 to 46, wherein the amino acid sequence of the Type II Cas protein comprises an amino acid sequence that is at least 70% identical to the amino acid sequence of the BH domain of the reference protein sequence.

52. The Type II Cas protein of any one of embodiments 1 to 46, wherein the amino acid sequence of the Type II Cas protein comprises an amino acid sequence that is at least 75% identical to the amino acid sequence of the BH domain of the reference protein sequence.

53. The Type II Cas protein of any one of embodiments 1 to 46, wherein the amino acid sequence of the Type II Cas protein comprises an amino acid sequence that is at least 80% identical to the amino acid sequence of the BH domain of the reference protein sequence.

54. The Type II Cas protein of any one of embodiments 1 to 46, wherein the amino acid sequence of the Type II Cas protein comprises an amino acid sequence that is at least 85% identical to the amino acid sequence of the BH domain of the reference protein sequence.

55. The Type II Cas protein of any one of embodiments 1 to 46, wherein the amino acid sequence of the Type II Cas protein comprises an amino acid sequence that is at least 90% identical to the amino acid sequence of the BH domain of the reference protein sequence.

56. The Type II Cas protein of any one of embodiments 1 to 46, wherein the amino acid sequence of the Type II Cas protein comprises an amino acid sequence that is at least 95% identical to the amino acid sequence of the BH domain of the reference protein sequence.

57. The Type II Cas protein of any one of embodiments 1 to 46, wherein the amino acid sequence of the Type II Cas protein comprises an amino acid sequence that is at least 96% identical to the amino acid sequence of the BH domain of the reference protein sequence.

58. The Type II Cas protein of any one of embodiments 1 to 46, wherein the amino acid sequence of the Type II Cas protein comprises an amino acid sequence that is at least 97% identical to the amino acid sequence of the BH domain of the reference protein sequence.

59. The Type II Cas protein of any one of embodiments 1 to 46, wherein the amino acid sequence of the Type II Cas protein comprises an amino acid sequence that is at least 98% identical to the amino acid sequence of the BH domain of the reference protein sequence.

60. The Type II Cas protein of any one of embodiments 1 to 46, wherein the amino acid sequence of the Type II Cas protein comprises an amino acid sequence that is at least 99% identical to the amino acid sequence of the BH domain of the reference protein sequence.

61. The Type II Cas protein of any one of embodiments 1 to 46, wherein the amino acid sequence of the Type II Cas protein comprises an amino acid sequence that is identical to the amino acid sequence of the BH domain of the reference protein sequence.

62. The Type II Cas protein of any one of embodiments 1 to 61, wherein the amino acid sequence of the Type II Cas protein comprises an amino acid sequence that is at least 50% identical to the amino acid sequence of the REC domain of the reference protein sequence.

63. The Type II Cas protein of any one of embodiments 1 to 61, wherein the amino acid sequence of the Type II Cas protein comprises an amino acid sequence that is at least 55% identical to the amino acid sequence of the REC domain of the reference protein sequence.

64. The Type II Cas protein of any one of embodiments 1 to 61, wherein the amino acid sequence of the Type II Cas protein comprises an amino acid sequence that is at least 60% identical to the amino acid sequence of the REC domain of the reference protein sequence.

65. The Type II Cas protein of any one of embodiments 1 to 61, wherein the amino acid sequence of the Type II Cas protein comprises an amino acid sequence that is at least 65% identical to the amino acid sequence of the REC domain of the reference protein sequence.

66. The Type II Cas protein of any one of embodiments 1 to 61, wherein the amino acid sequence of the Type II Cas protein comprises an amino acid sequence that is at least 70% identical to the amino acid sequence of the REC domain of the reference protein sequence.

67. The Type II Cas protein of any one of embodiments 1 to 61, wherein the amino acid sequence of the Type II Cas protein comprises an amino acid sequence that is at least 75% identical to the amino acid sequence of the REC domain of the reference protein sequence.

68. The Type II Cas protein of any one of embodiments 1 to 61, wherein the amino acid sequence of the Type II Cas protein comprises an amino acid sequence that is at least 80% identical to the amino acid sequence of the REC domain of the reference protein sequence.

69. The Type II Cas protein of any one of embodiments 1 to 61, wherein the amino acid sequence of the Type II Cas protein comprises an amino acid sequence that is at least 85% identical to the amino acid sequence of the REC domain of the reference protein sequence.

70. The Type II Cas protein of any one of embodiments 1 to 61, wherein the amino acid sequence of the Type II Cas protein comprises an amino acid sequence that is at least 90% identical to the amino acid sequence of the REC domain of the reference protein sequence.

71. The Type II Cas protein of any one of embodiments 1 to 61, wherein the amino acid sequence of the Type II Cas protein comprises an amino acid sequence that is at least 95% identical to the amino acid sequence of the REC domain of the reference protein sequence.

72. The Type II Cas protein of any one of embodiments 1 to 61, wherein the amino acid sequence of the Type II Cas protein comprises an amino acid sequence that is at least 96% identical to the amino acid sequence of the REC domain of the reference protein sequence.

73. The Type II Cas protein of any one of embodiments 1 to 61, wherein the amino acid sequence of the Type II Cas protein comprises an amino acid sequence that is at least 97% identical to the amino acid sequence of the REC domain of the reference protein sequence.

74. The Type II Cas protein of any one of embodiments 1 to 61, wherein the amino acid sequence of the Type II Cas protein comprises an amino acid sequence that is at least 98% identical to the amino acid sequence of the REC domain of the reference protein sequence.

75. The Type II Cas protein of any one of embodiments 1 to 61, wherein the amino acid sequence of the Type II Cas protein comprises an amino acid sequence that is at least 99% identical to the amino acid sequence of the REC domain of the reference protein sequence.

76. The Type II Cas protein of any one of embodiments 1 to 61, wherein the amino acid sequence of the Type II Cas protein comprises an amino acid sequence that is identical to the amino acid sequence of the REC domain of the reference protein sequence.

77. The Type II Cas protein of any one of embodiments 1 to 76, wherein the amino acid sequence of the Type II Cas protein comprises an amino acid sequence that is at least 50% identical to the amino acid sequence of the HNH domain of the reference protein sequence.

78. The Type II Cas protein of any one of embodiments 1 to 76, wherein the amino acid sequence of the Type II Cas protein comprises an amino acid sequence that is at least 55% identical to the amino acid sequence of the HNH domain of the reference protein sequence.

79. The Type II Cas protein of any one of embodiments 1 to 76, wherein the amino acid sequence of the Type II Cas protein comprises an amino acid sequence that is at least 60% identical to the amino acid sequence of the HNH domain of the reference protein sequence.

80. The Type II Cas protein of any one of embodiments 1 to 76, wherein the amino acid sequence of the Type II Cas protein comprises an amino acid sequence that is at least 65% identical to the amino acid sequence of the HNH domain of the reference protein sequence.

81. The Type II Cas protein of any one of embodiments 1 to 76, wherein the amino acid sequence of the Type II Cas protein comprises an amino acid sequence that is at least 70% identical to the amino acid sequence of the HNH domain of the reference protein sequence.

82. The Type II Cas protein of any one of embodiments 1 to 76, wherein the amino acid sequence of the Type II Cas protein comprises an amino acid sequence that is at least 75% identical to the amino acid sequence of the HNH domain of the reference protein sequence.

83. The Type II Cas protein of any one of embodiments 1 to 76, wherein the amino acid sequence of the Type II Cas protein comprises an amino acid sequence that is at least 80% identical to the amino acid sequence of the HNH domain of the reference protein sequence.

84. The Type II Cas protein of any one of embodiments 1 to 76, wherein the amino acid sequence of the Type II Cas protein comprises an amino acid sequence that is at least 85% identical to the amino acid sequence of the HNH domain of the reference protein sequence.

85. The Type II Cas protein of any one of embodiments 1 to 76, wherein the amino acid sequence of the Type II Cas protein comprises an amino acid sequence that is at least 90% identical to the amino acid sequence of the HNH domain of the reference protein sequence.

86. The Type II Cas protein of any one of embodiments 1 to 76, wherein the amino acid sequence of the Type II Cas protein comprises an amino acid sequence that is at least 95% identical to the amino acid sequence of the HNH domain of the reference protein sequence.

87. The Type II Cas protein of any one of embodiments 1 to 76, wherein the amino acid sequence of the Type II Cas protein comprises an amino acid sequence that is at least 96% identical to the amino acid sequence of the HNH domain of the reference protein sequence.

88. The Type II Cas protein of any one of embodiments 1 to 76, wherein the amino acid sequence of the Type II Cas protein comprises an amino acid sequence that is at least 97% identical to the amino acid sequence of the HNH domain of the reference protein sequence.

89. The Type II Cas protein of any one of embodiments 1 to 76, wherein the amino acid sequence of the Type II Cas protein comprises an amino acid sequence that is at least 98% identical to the amino acid sequence of the HNH domain of the reference protein sequence.

90. The Type II Cas protein of any one of embodiments 1 to 76, wherein the amino acid sequence of the Type II Cas protein comprises an amino acid sequence that is at least 99% identical to the amino acid sequence of the HNH domain of the reference protein sequence.

91. The Type II Cas protein of any one of embodiments 1 to 76, wherein the amino acid sequence of the Type II Cas protein comprises an amino acid sequence that is identical to the amino acid sequence of the HNH domain of the reference protein sequence.

92. The Type II Cas protein of any one of embodiments 1 to 91, wherein the amino acid sequence of the Type II Cas protein comprises an amino acid sequence that is at least 50% identical to the amino acid sequence of the WED domain of the reference protein sequence.

93. The Type II Cas protein of any one of embodiments 1 to 91, wherein the amino acid sequence of the Type II Cas protein comprises an amino acid sequence that is at least 55% identical to the amino acid sequence of the WED domain of the reference protein sequence.

94. The Type II Cas protein of any one of embodiments 1 to 91, wherein the amino acid sequence of the Type II Cas protein comprises an amino acid sequence that is at least 60% identical to the amino acid sequence of the WED domain of the reference protein sequence.

95. The Type II Cas protein of any one of embodiments 1 to 91, wherein the amino acid sequence of the Type II Cas protein comprises an amino acid sequence that is at least 65% identical to the amino acid sequence of the WED domain of the reference protein sequence.

96. The Type II Cas protein of any one of embodiments 1 to 91, wherein the amino acid sequence of the Type II Cas protein comprises an amino acid sequence that is at least 70% identical to the amino acid sequence of the WED domain of the reference protein sequence.

97. The Type II Cas protein of any one of embodiments 1 to 91, wherein the amino acid sequence of the Type II Cas protein comprises an amino acid sequence that is at least 75% identical to the amino acid sequence of the WED domain of the reference protein sequence.

98. The Type II Cas protein of any one of embodiments 1 to 91, wherein the amino acid sequence of the Type II Cas protein comprises an amino acid sequence that is at least 80% identical to the amino acid sequence of the WED domain of the reference protein sequence.

99. The Type II Cas protein of any one of embodiments 1 to 91, wherein the amino acid sequence of the Type II Cas protein comprises an amino acid sequence that is at least 85% identical to the amino acid sequence of the WED domain of the reference protein sequence.

100. The Type II Cas protein of any one of embodiments 1 to 91, wherein the amino acid sequence of the Type II Cas protein comprises an amino acid sequence that is at least 90% identical to the amino acid sequence of the WED domain of the reference protein sequence.

101. The Type II Cas protein of any one of embodiments 1 to 91, wherein the amino acid sequence of the Type II Cas protein comprises an amino acid sequence that is at least 95% identical to the amino acid sequence of the WED domain of the reference protein sequence.

102. The Type II Cas protein of any one of embodiments 1 to 91, wherein the amino acid sequence of the Type II Cas protein comprises an amino acid sequence that is at least 96% identical to the amino acid sequence of the WED domain of the reference protein sequence.

103. The Type II Cas protein of any one of embodiments 1 to 91, wherein the amino acid sequence of the Type II Cas protein comprises an amino acid sequence that is at least 97% identical to the amino acid sequence of the WED domain of the reference protein sequence.

104. The Type II Cas protein of any one of embodiments 1 to 91, wherein the amino acid sequence of the Type II Cas protein comprises an amino acid sequence that is at least 98% identical to the amino acid sequence of the WED domain of the reference protein sequence.

105. The Type II Cas protein of any one of embodiments 1 to 91, wherein the amino acid sequence of the Type II Cas protein comprises an amino acid sequence that is at least 99% identical to the amino acid sequence of the WED domain of the reference protein sequence.

106. The Type II Cas protein of any one of embodiments 1 to 91, wherein the amino acid sequence of the Type II Cas protein comprises an amino acid sequence that is identical to the amino acid sequence of the WED domain of the reference protein sequence.

107. The Type II Cas protein of any one of embodiments 1 to 106, wherein the amino acid sequence of the Type II Cas protein comprises an amino acid sequence that is at least 50% identical to the amino acid sequence of the PID domain of the reference protein sequence.

108. The Type II Cas protein of any one of embodiments 1 to 106, wherein the amino acid sequence of the Type II Cas protein comprises an amino acid sequence that is at least 55% identical to the amino acid sequence of the PID domain of the reference protein sequence.

109. The Type II Cas protein of any one of embodiments 1 to 106, wherein the amino acid sequence of the Type II Cas protein comprises an amino acid sequence that is at least 60% identical to the amino acid sequence of the PID domain of the reference protein sequence.

110. The Type II Cas protein of any one of embodiments 1 to 106, wherein the amino acid sequence of the Type II Cas protein comprises an amino acid sequence that is at least 65% identical to the amino acid sequence of the PID domain of the reference protein sequence.

111. The Type II Cas protein of any one of embodiments 1 to 106, wherein the amino acid sequence of the Type II Cas protein comprises an amino acid sequence that is at least 70% identical to the amino acid sequence of the PID domain of the reference protein sequence.

112. The Type II Cas protein of any one of embodiments 1 to 106, wherein the amino acid sequence of the Type II Cas protein comprises an amino acid sequence that is at least 75% identical to the amino acid sequence of the PID domain of the reference protein sequence.

113. The Type II Cas protein of any one of embodiments 1 to 106, wherein the amino acid sequence of the Type II Cas protein comprises an amino acid sequence that is at least 80% identical to the amino acid sequence of the PID domain of the reference protein sequence.

114. The Type II Cas protein of any one of embodiments 1 to 106, wherein the amino acid sequence of the Type II Cas protein comprises an amino acid sequence that is at least 85% identical to the amino acid sequence of the PID domain of the reference protein sequence.

115. The Type II Cas protein of any one of embodiments 1 to 106, wherein the amino acid sequence of the Type II Cas protein comprises an amino acid sequence that is at least 90% identical to the amino acid sequence of the PID domain of the reference protein sequence.

116. The Type II Cas protein of any one of embodiments 1 to 106, wherein the amino acid sequence of the Type II Cas protein comprises an amino acid sequence that is at least 95% identical to the amino acid sequence of the PID domain of the reference protein sequence.

117. The Type II Cas protein of any one of embodiments 1 to 106, wherein the amino acid sequence of the Type II Cas protein comprises an amino acid sequence that is at least 96% identical to the amino acid sequence of the PID domain of the reference protein sequence.

118. The Type II Cas protein of any one of embodiments 1 to 106, wherein the amino acid sequence of the Type II Cas protein comprises an amino acid sequence that is at least 97% identical to the amino acid sequence of the PID domain of the reference protein sequence.

119. The Type II Cas protein of any one of embodiments 1 to 106, wherein the amino acid sequence of the Type II Cas protein comprises an amino acid sequence that is at least 98% identical to the amino acid sequence of the PID domain of the reference protein sequence.

120. The Type II Cas protein of any one of embodiments 1 to 106, wherein the amino acid sequence of the Type II Cas protein comprises an amino acid sequence that is at least 99% identical to the amino acid sequence of the PID domain of the reference protein sequence.

121. The Type II Cas protein of any one of embodiments 1 to 106, wherein the amino acid sequence of the Type II Cas protein comprises an amino acid sequence that is identical to the amino acid sequence of the PID domain of the reference protein sequence.

122. The Type II Cas protein of embodiment 1, wherein the amino acid sequence of the Type II Cas protein comprises an amino acid sequence that is at least 55% identical to the full length of the reference protein sequence.

123. The Type II Cas protein of embodiment 1, wherein the amino acid sequence of the Type II Cas protein comprises an amino acid sequence that is at least 60% identical to the full length of the reference protein sequence.

124. The Type II Cas protein of embodiment 1, wherein the amino acid sequence of the Type II Cas protein comprises an amino acid sequence that is at least 65% identical to the full length of the reference protein sequence.

125. The Type II Cas protein of embodiment 1, wherein the amino acid sequence of the Type II Cas protein comprises an amino acid sequence that is at least 70% identical to the full length of the reference protein sequence.

126. The Type II Cas protein of embodiment 1, wherein the amino acid sequence of the Type II Cas protein comprises an amino acid sequence that is at least 75% identical to the full length of the reference protein sequence.

127. The Type II Cas protein of embodiment 1, wherein the amino acid sequence of the Type II Cas protein comprises an amino acid sequence that is at least 80% identical to the full length of the reference protein sequence.

128. The Type II Cas protein of embodiment 1, wherein the amino acid sequence of the Type II Cas protein comprises an amino acid sequence that is at least 85% identical to the full length of the reference protein sequence.

129. The Type II Cas protein of embodiment 1, wherein the amino acid sequence of the Type II Cas protein comprises an amino acid sequence that is at least 90% identical to the full length of the reference protein sequence.

130. The Type II Cas protein of embodiment 1, wherein the amino acid sequence of the Type II Cas protein comprises an amino acid sequence that is at least 95% identical to the full length of the reference protein sequence.

131. The Type II Cas protein of embodiment 1, wherein the amino acid sequence of the Type II Cas protein comprises an amino acid sequence that is at least 96% identical to the full length of the reference protein sequence.

132. The Type II Cas protein of embodiment 1, wherein the amino acid sequence of the Type II Cas protein comprises an amino acid sequence that is at least 97% identical to the full length of the reference protein sequence.

133. The Type II Cas protein of embodiment 1, wherein the amino acid sequence of the Type II Cas protein comprises an amino acid sequence that is at least 98% identical to the full length of the reference protein sequence.

134. The Type II Cas protein of embodiment 1, wherein the amino acid sequence of the Type II Cas protein comprises an amino acid sequence that is at least 99% identical to the full length of the reference protein sequence.

135. The Type II Cas protein of embodiment 1, wherein the amino acid sequence of the Type II Cas protein comprises an amino acid sequence that is identical to the full length of the reference protein sequence.

136. The Type II Cas protein of any one of embodiments 1 to 134, which is a chimeric Type II Cas protein.

137. The Type II Cas protein of any one of embodiments 1 to 136, which is a fusion protein.

138. The Type II Cas protein of embodiment 137, which comprises one or more nuclear localization signals.

139. The Type II Cas protein of embodiment 138, which comprises two or more nuclear localization signals.

140. The Type II Cas protein of embodiment 138 or embodiment 139, which comprises an N-terminal nuclear localization signal.

141. The Type II Cas protein of any one of embodiments 138 to 140, which comprises a C-terminal nuclear localization signal.

142. The Type II Cas protein of any one of embodiments 138 to 141, which comprises an N-terminal nuclear localization signal and a C-terminal nuclear localization signal.

143. The Type II Cas protein of any one of embodiments 138 to 142, wherein the amino acid sequence of one or more of the nuclear localization signals comprises the amino acid sequence KRTADGSEFESPKKKRKV (SEQ ID NO:38), PKKKRKV (SEQ ID NO:39), PKKKRRV (SEQ ID NO:40), KRPAATKKAGQAKKKK (SEQ ID NO:41), YGRKKRRQRRR (SEQ ID NO:42), RKKRRQRRR (SEQ ID NO:43), PAAKRVKLD (SEQ ID NO:44), RQRRNELKRSP (SEQ ID NO:45), VSRKRPRP (SEQ ID NO:46), PPKKARED (SEQ ID NO:47), PQPKKKPL (SEQ ID NO:48), SALIKKKKKMAP (SEQ ID NO:49), PKQKKRK (SEQ ID NO:50), RKLKKKIKKL (SEQ ID NO:51), REKKKFLKRR (SEQ ID NO:52), KRKGDEVDGVDEVAKKKSKK (SEQ ID NO:53), RKCLQAGMNLEARKTKK (SEQ ID NO:54), NQSSNFGPMKGGNFGGRSSGPYGGGGQYFAKPRNQGGY (SEQ ID NO:55), or RMRIZFKNKGKDTAELRRRRVEVSVELRKAKKDEQILKRRNV (SEQ ID NO:56).

144. The Type II Cas protein of embodiment 143, wherein the amino acid sequence of one or more of the nuclear localization signals comprises the amino acid sequence KRTADGSEFESPKKKRKV (SEQ ID NO:38).

145. The Type II Cas protein of embodiment 143, wherein the amino acid sequence of one or more of the nuclear localization signals comprises the amino acid sequence PKKKRKV (SEQ ID NO:39).

146. The Type II Cas protein of embodiment 143, wherein the amino acid sequence of one or more of the nuclear localization signals comprises the amino acid sequence PKKKRRV (SEQ ID NO:40).

147. The Type II Cas protein of embodiment 143, wherein the amino acid sequence of one or more of the nuclear localization signals comprises the amino acid sequence KRPAATKKAGQAKKKK (SEQ ID NO:41).

148. The Type II Cas protein of embodiment 143, wherein the amino acid sequence of one or more of the nuclear localization signals comprises the amino acid sequence YGRKKRRQRRR (SEQ ID NO:42).

149. The Type II Cas protein of embodiment 143, wherein the amino acid sequence of one or more of the nuclear localization signals comprises the amino acid sequence RKKRRQRRR (SEQ ID NO:43).

150. The Type II Cas protein of embodiment 143, wherein the amino acid sequence of one or more of the nuclear localization signals comprises the amino acid sequence PAAKRVKLD (SEQ ID NO:44).

151. The Type II Cas protein of embodiment 143, wherein the amino acid sequence of one or more of the nuclear localization signals comprises the amino acid sequence RQRRNELKRSP (SEQ ID NO:45).

152. The Type II Cas protein of embodiment 143, wherein the amino acid sequence of one or more of the nuclear localization signals comprises the amino acid sequence VSRKRPRP (SEQ ID NO:46).

153. The Type II Cas protein of embodiment 143, wherein the amino acid sequence of one or more of the nuclear localization signals comprises the amino acid sequence PPKKARED (SEQ ID NO:47).

154. The Type II Cas protein of embodiment 143, wherein the amino acid sequence of one or more of the nuclear localization signals comprises the amino acid sequence PQPKKKPL (SEQ ID NO:48).

155. The Type II Cas protein of embodiment 143, wherein the amino acid sequence of one or more of the nuclear localization signals comprises the amino acid sequence SALIKKKKKMAP (SEQ ID NO:49).

156. The Type II Cas protein of embodiment 143, wherein the amino acid sequence of one or more of the nuclear localization signals comprises the amino acid sequence PKQKKRK (SEQ ID NO:50).

157. The Type II Cas protein of embodiment 143, wherein the amino acid sequence of one or more of the nuclear localization signals comprises the amino acid sequence RKLKKKIKKL (SEQ ID NO:51).

158. The Type II Cas protein of embodiment 143, wherein the amino acid sequence of one or more of the nuclear localization signals comprises the amino acid sequence REKKKFLKRR (SEQ ID NO:52).

159. The Type II Cas protein of embodiment 143, wherein the amino acid sequence of one or more of the nuclear localization signals comprises the amino acid sequence KRKGDEVDGVDEVAKKKSKK (SEQ ID NO:53).

160. The Type II Cas protein of embodiment 143, wherein the amino acid sequence of one or more of the nuclear localization signals comprises the amino acid sequence RKCLQAGMNLEARKTKK (SEQ ID NO:54).

161. The Type II Cas protein of embodiment 143, wherein the amino acid sequence of one or more of the nuclear localization signals comprises the amino acid sequence NQSSNFGPMKGGNFGGRSSGPYGGGGQYFAKPRNQGGY (SEQ ID NO:55).

162. The Type II Cas protein of embodiment 143, wherein the amino acid sequence of one or more of the nuclear localization signals comprises the amino acid sequence RMRIZFKNKGKDTAELRRRRVEVSVELRKAKKDEQILKRRNV (SEQ ID NO:56).

163. The Type II Cas protein of any one of embodiments 138 to 162, wherein the amino acid sequence of each nuclear localization signal is the same.

164. The Type II Cas protein of any one of embodiments 136 to 163, which comprises a fusion partner which is a DNA, RNA or protein modification enzyme, optionally wherein the DNA, RNA or protein modification enzyme is an adenosine deaminase, a cytidine deaminase, a reverse transcriptase, a guanosyl transferase, a DNA methyltransferase, a RNA methyltransferase, a DNA demethylase, a RNA demethylase, a dioxygenase, a polyadenylate polymerase, a pseudouridine synthase, an acetyltransferase, a deacetylase, a ubiquitin-ligase, a deubiquitinase, a kinase, a phosphatase, a NEDD8-ligase, a de-NEDDylase, a SUMO-ligase, a deSUMOylase, a histone deacetylase, a histone acetyltransferase, a histone methyltransferase, or a histone demethylase.

165. The Type II Cas protein of any one of embodiments 136 to 164, which comprises a means for deaminating adenosine, optionally wherein the means for deaminating adenosine is an adenosine deaminase.

166. The Type II Cas protein of any one of embodiments 136 to 164, which comprises a fusion partner which is an adenosine deaminase, optionally wherein the amino acid sequence of the adenosine deaminase comprises an amino acid sequence having at least 50%, at least 60%, at least 70%, at least 80%, at least 90%, at least 95%, at least 97%, at least 98%, at least 99%, or 100% sequence identity with SEQ ID NO:792, optionally wherein the adenosine deaminase is the adenosine deaminase moiety contained in the adenine base editor ABE8e.

167. The Type II Cas protein of any one of embodiments 136 to 164, which comprises a means for deaminating cytidine, optionally wherein the means for deaminating cytidine is a cytodine deaminase.

168. The Type II Cas protein of any one of embodiments 136 to 164, which comprises a fusion partner which is a cytodine deaminase.

169. The Type II Cas protein of any one of embodiments 136 to 164, which comprises a means for synthesizing DNA from a single-stranded template, optionally wherein the means for synthesizing DNA from a single-stranded template is a reverse transcriptase.

170. The Type II Cas protein of any one of embodiments 136 to 164, which comprises a fusion partner which is a reverse transcriptase.

171. The Type II Cas protein of any one of embodiments 136 to 170, which comprises a tag.

172. The Type II Cas protein of embodiment 171, wherein the tag is a SV5 tag, optionally wherein the SV5 tag comprises the amino acid sequence GKPIPNPLLGLDST (SEQ ID NO:57).

173. The Type II Cas protein of any one of embodiments 1 to 172, wherein the reference protein sequence is SEQ ID NO:1.

174. The Type II Cas protein of embodiment 173, whose amino acid sequence comprises the amino acid sequence of SEQ ID NO:1.

175. The Type II Cas protein of any one of embodiments 1 to 172, wherein the reference protein sequence is SEQ ID NO:2.

176. The Type II Cas protein of any one of embodiments 173 to 175, whose amino acid sequence comprises the amino acid sequence of SEQ ID NO:2.

177. The Type II Cas protein of embodiment 173 or embodiment 174, whose amino acid sequence comprises the amino acid sequence of SEQ ID NO:3.

178. The Type II Cas protein of any one of embodiments 1 to 172, wherein the reference protein sequence is SEQ ID NO:7.

179. The Type II Cas protein of embodiment 178, whose amino acid sequence comprises the amino acid sequence of SEQ ID NO:7.

180. The Type II Cas protein of any one of embodiments 1 to 172, wherein the reference protein sequence is SEQ ID NO:8.

181. The Type II Cas protein of any one of embodiments 178 to 180, whose amino acid sequence comprises the amino acid sequence of SEQ ID NO:8.

182. The Type II Cas protein of embodiment 178 or embodiment 179, whose amino acid sequence comprises the amino acid sequence of SEQ ID NO:9.

183. The Type II Cas protein of any one of embodiments 1 to 172, wherein the reference protein sequence is SEQ ID NO:30.

184. The Type II Cas protein of embodiment 183, whose amino acid sequence comprises the amino acid sequence of SEQ ID NO:30.

185. The Type II Cas protein of any one of embodiments 1 to 172, wherein the reference protein sequence is SEQ ID NO:31.

186. The Type II Cas protein of any one of embodiments 183 to 185, whose amino acid sequence comprises the amino acid sequence of SEQ ID NO:31.

187. The Type II Cas protein of embodiment 183 or embodiment 184, whose amino acid sequence comprises the amino acid sequence of SEQ ID NO:786.

188. The Type II Cas protein of any one of embodiments 1 to 172, wherein the reference protein sequence is SEQ ID NO:34.

189. The Type II Cas protein of embodiment 188, whose amino acid sequence comprises the amino acid sequence of SEQ ID NO:34.

190. The Type II Cas protein of any one of embodiments 1 to 172, wherein the reference protein sequence is SEQ ID NO:35.

191. The Type II Cas protein of any one of embodiments 188 to 190, whose amino acid sequence comprises the amino acid sequence of SEQ ID NO:35.

192. The Type II Cas protein of embodiment 188 or embodiment 189, whose amino acid sequence comprises the amino acid sequence of SEQ ID NO:787.

193. A Type II Cas protein whose amino acid sequence is identical to a Type II Cas protein of any one of embodiments 1 to 192 except for one or more amino acid substitutions relative to the reference sequence that provide nickase activity.

194. The Type II Cas of embodiment 193, wherein the one or more amino acid substitutions relative to the reference sequence that provide nickase activity comprise a D23A mutation, wherein the position of the D23A substitution is defined with respect to the amino acid numbering of SEQ ID NO:8.

195. A gRNA comprising a spacer and a sgRNA scaffold, wherein:
(a) the spacer is positioned 5′ to the sgRNA scaffold; and
(b) the nucleotide sequence of the sgRNA scaffold comprises a nucleotide sequence that is at least 50% identical to a reference scaffold sequence, wherein the reference scaffold sequence is SEQ ID NO:15, SEQ ID NO:16, SEQ ID NO:17, SEQ ID NO:18, SEQ ID NO:19, SEQ ID NO:22, SEQ ID NO:23, SEQ ID NO:24, SEQ ID NO:25, SEQ ID NO:75, or SEQ ID NO:822.

196. A gRNA comprising a means for binding a target mammalian genomic sequence and a sgRNA scaffold, optionally wherein the means for binding a target mammalian genomic sequence is a spacer, wherein:
(a) the means for binding a target genomic sequence is positioned 5′ to the sgRNA scaffold; and
(b) the nucleotide sequence of the sgRNA scaffold comprises a nucleotide sequence that is at least 50% identical to a reference scaffold sequence, wherein the reference scaffold sequence is SEQ ID NO:15, SEQ ID NO:16, SEQ ID NO:17, SEQ ID NO:18, SEQ ID NO:19, SEQ ID NO:22, SEQ ID NO:23, SEQ ID NO:24, SEQ ID NO:25, SEQ ID NO:75, or SEQ ID NO:822.

197. The gRNA of embodiment 195 or embodiment 196, wherein the sgRNA scaffold comprises one or more G:C couples not present in the reference scaffold sequence.

198. The gRNA of any one of embodiments 195 to 196, wherein the sgRNA scaffold comprises one or more U to A substitutions relative to the reference scaffold sequence.

199. The gRNA of any one of embodiments 195 to 198, wherein the sgRNA scaffold comprises one or more trimmed stem loop sequences in place of one or more longer stem loop sequences in the reference scaffold sequence.

200. The gRNA of embodiment 199, wherein the trimmed stem loop sequence comprises a GAAA tetraloop in place of a longer stem loop sequence in the reference scaffold sequence.

201. The gRNA of any one of embodiments 195 to 200, wherein the sgRNA scaffold comprises one or more trimmed loop sequences in place of one or more longer loop sequences in the reference scaffold sequence.

202. The gRNA of embodiment 201, wherein the sgRNA scaffold comprises a GAAA tetraloop in place of a longer loop sequence in the reference scaffold sequence.

203. The gRNA of any one of embodiments 195 to 202, wherein the sgRNA scaffold comprises a nucleotide sequence that is at least 55% identical to the reference scaffold sequence.

204. The gRNA of embodiment 203, wherein the sgRNA scaffold comprises a nucleotide sequence that is at least 60% identical to the reference scaffold sequence.

205. The gRNA of embodiment 203, wherein the sgRNA scaffold comprises a nucleotide sequence that is at least 65% identical to the reference scaffold sequence.

206. The gRNA of embodiment 203, wherein the sgRNA scaffold comprises a nucleotide sequence that is at least 70% identical to the reference scaffold sequence.

207. The gRNA of embodiment 203, wherein the sgRNA scaffold comprises a nucleotide sequence that is at least 75% identical to the reference scaffold sequence.

208. The gRNA of embodiment 203, wherein the sgRNA scaffold comprises a nucleotide sequence that is at least 80% identical to the reference scaffold sequence.

209. The gRNA of embodiment 203, wherein the sgRNA scaffold comprises a nucleotide sequence that is at least 85% identical to the reference scaffold sequence.

210. The gRNA of embodiment 203, wherein the sgRNA scaffold comprises a nucleotide sequence that is at least 90% identical to the reference scaffold sequence.

211. The gRNA of embodiment 203, wherein the sgRNA scaffold comprises a nucleotide sequence that is at least 95% identical to the reference scaffold sequence.

212. The gRNA of embodiment 203, wherein the sgRNA scaffold comprises a nucleotide sequence that is at least 96% identical to the reference scaffold sequence.

213. The gRNA of embodiment 203, wherein the sgRNA scaffold comprises a nucleotide sequence that is at least 97% identical to the reference scaffold sequence.

214. The gRNA of embodiment 203, wherein the sgRNA scaffold comprises a nucleotide sequence that is at least 98% identical to the reference scaffold sequence.

215. The gRNA of embodiment 203, wherein the sgRNA scaffold comprises a nucleotide sequence that is at least 99% identical to the reference scaffold sequence.

216. The gRNA of embodiment 203, wherein the sgRNA scaffold comprises a nucleotide sequence that has no more than 5 nucleotide mismatches with the reference scaffold sequence.

217. The gRNA of embodiment 203, wherein the sgRNA scaffold comprises a nucleotide sequence that has no more than 4 nucleotide mismatches with the reference scaffold sequence.

218. The gRNA of embodiment 203, wherein the sgRNA scaffold comprises a nucleotide sequence that has no more than 3 nucleotide mismatches with the reference scaffold sequence.

219. The gRNA of embodiment 203, wherein the sgRNA scaffold comprises a nucleotide sequence that has no more than 2 nucleotide mismatches with the reference scaffold sequence.

220. The gRNA of embodiment 203, wherein the sgRNA scaffold comprises a nucleotide sequence that has no more than 1 nucleotide mismatches with the reference scaffold sequence.

221. The gRNA of embodiment 195 or embodiment 196, wherein the sgRNA scaffold comprises a nucleotide sequence that is 100% identical to the reference scaffold sequence.

222. The gRNA of any one of embodiments 195 to 221, wherein the reference scaffold sequence is SEQ ID NO:15, SEQ ID NO:16, SEQ ID NO:17, SEQ ID NO:18, or SEQ ID NO:19.

223. The gRNA of embodiment 222, wherein the reference scaffold sequence is SEQ ID NO:15.

224. The gRNA of embodiment 222, wherein the reference scaffold sequence is SEQ ID NO:16.

225. The gRNA of embodiment 222, wherein the reference scaffold sequence is SEQ ID NO:17.

226. The gRNA of embodiment 222, wherein the reference scaffold sequence is SEQ ID NO:18.

227. The gRNA of embodiment 222, wherein the reference scaffold sequence is SEQ ID NO:19.

228. The gRNA of any one of embodiments 195 to 221, wherein the reference scaffold sequence is SEQ ID NO:22, SEQ ID NO:23, SEQ ID NO:24, SEQ ID NO:25, or SEQ ID NO:822.

229. The gRNA of embodiment 228, wherein the reference scaffold sequence is SEQ ID NO:22.

230. The gRNA of embodiment 228, wherein the reference scaffold sequence is SEQ ID NO:23.

231. The gRNA of embodiment 228, wherein the reference scaffold sequence is SEQ ID NO:24.

232. The gRNA of embodiment 228, wherein the reference scaffold sequence is SEQ ID NO:25.

233. The gRNA of embodiment 228, wherein the reference scaffold sequence is SEQ ID NO:822.

234. The gRNA of embodiment 195 or embodiment 196, wherein the nucleotide sequence of the sgRNA scaffold comprises the nucleotide sequence of SEQ ID NO:26.

235. The gRNA of embodiment 195 or embodiment 196, wherein the nucleotide sequence of the sgRNA scaffold comprises the nucleotide sequence of SEQ ID NO:27.

236. The gRNA of embodiment 195 or embodiment 196, wherein the nucleotide sequence of the sgRNA scaffold comprises the nucleotide sequence of SEQ ID NO:28.

237. The gRNA of embodiment 195 or embodiment 196, wherein the nucleotide sequence of the sgRNA scaffold comprises the nucleotide sequence of SEQ ID NO:29.

238. The gRNA of embodiment 195 or embodiment 196, wherein the nucleotide sequence of the sgRNA scaffold comprises the nucleotide sequence of SEQ ID NO:823.

239. The gRNA of any one of embodiments 195 to 221, wherein the reference scaffold sequence is SEQ ID NO:75.

240. The gRNA of any one of embodiments 195 to 239, wherein the sgRNA scaffold comprises 1 to 8 uracils at its 3′ end.

241. The gRNA of embodiment 240, wherein the sgRNA scaffold comprises 1 uracil at its 3′ end.

242. The gRNA of embodiment 240, wherein the sgRNA scaffold comprises 2 uracils at its 3′ end.

243. The gRNA of embodiment 240, wherein the sgRNA scaffold comprises 3 uracils at its 3′ end.

244. The gRNA of embodiment 240, wherein the sgRNA scaffold comprises 4 uracils at its 3′ end.

245. The gRNA of embodiment 240, wherein the sgRNA scaffold comprises 5 uracils at its 3′ end.

246. The gRNA of embodiment 240, wherein the sgRNA scaffold comprises 6 uracils at its 3′ end.

247. The gRNA of embodiment 240, wherein the sgRNA scaffold comprises 7 uracils at its 3′ end.

248. The gRNA of embodiment 240, wherein the sgRNA scaffold comprises 8 uracils at its 3′ end.

249. The gRNA of any one of embodiments 195 to 248, wherein the nucleotide sequence of the spacer is partially or fully complementary to a target mammalian genomic sequence.

250. A gRNA comprising (i) a crRNA comprising a spacer and a crRNA scaffold, wherein the spacer is 5′ to the crRNA scaffold, and (ii) a tracrRNA, wherein the nucleotide sequence of the spacer is partially or fully complementary to a target mammalian genomic sequence and the nucleotide sequence of the crRNA scaffold comprises the nucleotide sequence of SEQ ID NO:13, SEQ ID NO:20, SEQ ID NO:788, or SEQ ID NO:790.

251. A gRNA comprising (i) a crRNA comprising a means for binding a target mammalian genomic sequence (which is optionally a spacer) and a crRNA scaffold, wherein the means for binding a target mammalian genomic sequence is 5′ to the crRNA scaffold, and (ii) a tracrRNA, wherein the nucleotide sequence of the crRNA scaffold comprises the nucleotide sequence of SEQ ID NO:13, SEQ ID NO:20, SEQ ID NO:788, or SEQ ID NO:790.

252. The gRNA of embodiment 250 or 251, wherein the nucleotide sequence of the crRNA scaffold comprises the nucleotide sequence of SEQ ID NO:13.

253. The gRNA of any one of embodiments 250 to 252, wherein the nucleotide sequence of the tracrRNA comprises the nucleotide sequence of SEQ ID NO:14.

254. The gRNA of embodiment 250 or 251, wherein the nucleotide sequence of the crRNA scaffold comprises the nucleotide sequence of SEQ ID NO:20.

255. The gRNA of embodiment 250, embodiment 251, or embodiment 254, wherein the nucleotide sequence of the tracrRNA comprises the nucleotide sequence of SEQ ID NO:21.

256. The gRNA of embodiment 250 or 251, wherein the nucleotide sequence of the crRNA scaffold comprises the nucleotide sequence of SEQ ID NO:788.

257. The gRNA of embodiment 250, embodiment 251, or embodiment 256, wherein the nucleotide sequence of the tracrRNA comprises the nucleotide sequence of SEQ ID NO:789.

258. The gRNA of embodiment 250 or 251, wherein the nucleotide sequence of the crRNA scaffold comprises the nucleotide sequence of SEQ ID NO:790.

259. The gRNA of embodiment 250, embodiment 251, or embodiment 258, wherein the nucleotide sequence of the tracrRNA comprises the nucleotide sequence of SEQ ID NO:791.

260. The gRNA of any one of embodiments 250 to 259, wherein the gRNA comprises separate crRNA and tracrRNA molecules.

261. The gRNA of any one of embodiments 250 to 259, wherein the gRNA is a single guide RNA (sgRNA).

262. The gRNA of any one of embodiments 249 to 261, wherein the target mammalian genomic sequence is a human genomic sequence.

263. The gRNA of embodiment 262, wherein the target mammalian genomic sequence is a CCR5, EMX1, Fas, FANCF, HBB, ZSCAN2, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, BCR, ATM, HBG1, HPRT, IL2RG, NF1, USH2A, RHO, BcLenh, or CTFR genomic sequence.

264. The gRNA of embodiment 262, wherein the target mammalian genomic sequence is a CCR5, EMX1, Fas, FANCF, HBB, ZSCAN, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, or BCR genomic sequence.

265. The gRNA of any one of embodiments 249 to 264, wherein the target mammalian genomic sequence is upstream of a protospacer adjacent motif (PAM) sequence in the non-target strand recognized by a Type II Cas protein, optionally wherein the Type II Cas protein is a Type II Cas protein according to any one of embodiments 1 to 194.

266. The gRNA of embodiment 265, wherein the PAM sequence is NRVNRT.

267. The gRNA of embodiment 266, wherein the PAM sequence is NRCNAT.

268. The gRNA of embodiment 265, wherein the PAM sequence is N₄RHNT.

269. The gRNA of embodiment 268, wherein the PAM sequence is N₄RYNT.

270. The gRNA of embodiment 268, wherein the PAM sequence is N₄GYNT.

271. The gRNA of embodiment 268, wherein the PAM sequence is N₄GTNT.

272. The gRNA of embodiment 268, wherein the PAM sequence is N₄GTTT.

273. The gRNA of embodiment 268, wherein the PAM sequence is N₄GTGT.

274. The gRNA of embodiment 268, wherein the PAM sequence is N₄GCTT.

275. The gRNA of embodiment 268, wherein the PAM sequence is N₄GWAN.

276. The gRNA of embodiment 268, wherein the PAM sequence is N₄GWAA.

277. The gRNA of embodiment 268, wherein the PAM sequence is N₄GNAA.

278. The gRNA of embodiment 268, wherein the PAM sequence is N₄RNKA.

279. The gRNA of embodiment 268, wherein the PAM sequence is N₄GHKA.

280. The gRNA of any one of embodiments 195 to 279, wherein the spacer is 15 to 30 nucleotides in length.

281. The gRNA of embodiment 280, wherein the spacer is 15 to 25 nucleotides in length.

282. The gRNA of embodiment 280, wherein the spacer is 16 to 24 nucleotides in length.

283. The gRNA of embodiment 280, wherein the spacer is 17 to 23 nucleotides in length.

284. The gRNA of embodiment 280, wherein the spacer is 18 to 22 nucleotides in length.

285. The gRNA of embodiment 280, wherein the spacer is 19 to 21 nucleotides in length.

286. The gRNA of embodiment 280, wherein the spacer is 18 to 30 nucleotides in length.

287. The gRNA of embodiment 280, wherein the spacer is 20 to 28 nucleotides in length.

288. The gRNA of embodiment 280, wherein the spacer is 22 to 26 nucleotides in length.

289. The gRNA of embodiment 280, wherein the spacer is 23 to 25 nucleotides in length.

290. The gRNA of embodiment 280, wherein the spacer is 20 nucleotides in length.

291. The gRNA of embodiment 280, wherein the spacer is 21 nucleotides in length.

292. The gRNA of embodiment 280, wherein the spacer is 22 nucleotides in length.

293. The gRNA of embodiment 280, wherein the spacer is 23 nucleotides in length.

294. The gRNA of embodiment 280, wherein the spacer is 24 nucleotides in length.

295. The gRNA of embodiment 280, wherein the spacer is 25 nucleotides in length.

296. The gRNA of embodiment 280, wherein the spacer is 26 nucleotides in length.

297. The gRNA of embodiment 280, wherein the spacer is 27 nucleotides in length.

298. The gRNA of embodiment 280, wherein the spacer is 28 nucleotides in length.

299. A system comprising the Type II Cas protein of any one of embodiments 1 to 194 and a guide RNA (gRNA) comprising a spacer sequence, optionally wherein the gRNA is a gRNA according to any one of embodiments 195 to 298.

300. A system comprising the Type II Cas protein of any one of embodiments 1 to 194 and a means for targeting the Type II Cas protein to a target genomic sequence, optionally wherein the means for targeting the Type II Cas protein to a target genomic sequence is a guide RNA (gRNA) molecule, optionally as described in in any one of embodiments 195 to 298, optionally wherein the gRNA molecule comprises a spacer partially or fully complementary to a target mammalian genomic sequence.

301. The system of embodiment 299, wherein the spacer sequence is partially or fully complementary to a target mammalian genomic sequence.

302. The system of any one of embodiments 299 to 301, wherein the target mammalian genomic sequence is a human genomic sequence.

303. The system of embodiment 302, wherein the target mammalian genomic sequence is a CCR5, EMX1, Fas, FANCF, HBB, ZSCAN2, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, BCR, ATM, HBG1, HPRT, IL2RG, NF1, USH2A, RHO, BcLenh, or CTFR genomic sequence.

304. The system of embodiment 302, wherein the target mammalian genomic sequence is a CCR5, EMX1, Fas, FANCF, HBB, ZSCAN, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, or BCR genomic sequence.

305. The system of any one of embodiments 300 to 303, wherein the target mammalian genomic sequence is upstream of a protospacer adjacent motif (PAM) sequence in the non-target strand recognized by the Type II Cas protein.

306. The system of embodiment 305, wherein the reference protein sequence is SEQ ID NO:1 or SEQ ID NO:2 and wherein the PAM sequence is NRVNRT.

307. The system of embodiment 306, wherein the PAM sequence is NRCNAT.

308. The system of embodiment 305, wherein the reference protein sequence is SEQ ID NO:7 or SEQ ID NO:8 and the PAM sequence is N₄RHNT.

309. The system of embodiment 308, wherein the PAM sequence is N₄RYNT.

310. The system of embodiment 308, wherein the PAM sequence is N₄GYNT.

311. The system of embodiment 308, wherein the PAM sequence is N₄GTNT.

312. The system of embodiment 308, wherein the PAM sequence is N₄GTTT.

313. The system of embodiment 308, wherein the PAM sequence is N₄GTGT.

314. The system of embodiment 308, wherein the PAM sequence is N₄GCTT.

315. The system of embodiment 305, wherein the reference protein sequence is SEQ ID NO:30 or SEQ ID NO:31 and the PAM sequence is N₄GWAN.

316. The system of embodiment 305, wherein the reference protein sequence is SEQ ID NO:30 or SEQ ID NO:31 and the PAM sequence is N₄GWAA.

317. The system of embodiment 305, wherein the reference protein sequence is SEQ ID NO:34 or SEQ ID NO:35 and the PAM sequence is N₄RNKA.

318. The system of embodiment 305, wherein the reference protein sequence is SEQ ID NO:34 or SEQ ID NO:35 and the PAM sequence is N₄GHKA.

319. The system of any one of embodiments 299 to 318, wherein the gRNA comprises a crRNA sequence and a tracrRNA sequence.

320. The system of embodiment 319, wherein the reference protein sequence is SEQ ID NO:1 or SEQ ID NO:2 and wherein the crRNA sequence comprises the spacer sequence 5′ to the nucleotide sequence of SEQ ID NO:13.

321. The system of embodiment 319 or embodiment 320, wherein the reference protein sequence is SEQ ID NO:1 or SEQ ID NO:2 and wherein the tracrRNA sequence comprises the nucleotide sequence of SEQ ID NO:14.

322. The system of embodiment 319, wherein the reference protein sequence is SEQ ID NO:7 or SEQ ID NO:8 and wherein the crRNA sequence comprises the spacer sequence 5′ to the nucleotide sequence of SEQ ID NO:20.

323. The system of embodiment 319 or embodiment 322, wherein the reference protein sequence is SEQ ID NO:7 or SEQ ID NO:8 and wherein the tracrRNA sequence comprises the nucleotide sequence of SEQ ID NO:21.

324. The system of embodiment 319, wherein the reference protein sequence is SEQ ID NO:30 or SEQ ID NO:31 and wherein the crRNA sequence comprises the spacer sequence 5′ to the nucleotide sequence of SEQ ID NO:788.

325. The system of embodiment 319 or embodiment 324, wherein the reference protein sequence is SEQ ID NO:30 or SEQ ID NO:31 and wherein the tracrRNA sequence comprises the nucleotide sequence of SEQ ID NO:789.

326. The system of embodiment 319, wherein the reference protein sequence is SEQ ID NO:34 or SEQ ID NO:35 and wherein the crRNA sequence comprises the spacer sequence 5′ to the nucleotide sequence of SEQ ID NO:790.

327. The system of embodiment 319 or embodiment 326, wherein the reference protein sequence is SEQ ID NO:34 or SEQ ID NO:35 and wherein the tracrRNA sequence comprises the nucleotide sequence of SEQ ID NO:791.

328. The system of any one of embodiments 315 to 327, wherein the gRNA comprises separate crRNA and tracrRNA molecules.

329. The system of any one of embodiments 299 to 327, wherein the gRNA is a single guide RNA (sgRNA) comprising the spacer and a sgRNA scaffold, wherein the spacer is positioned 5′ to the sgRNA scaffold.

330. The system of embodiment 329, wherein the nucleotide sequence of the sgRNA scaffold comprises a nucleotide sequence that is at least 50% identical to a reference scaffold sequence.

331. The system of embodiment 330, wherein the sgRNA scaffold comprises one or more G:C couples not present in the reference scaffold sequence.

332. The system of embodiment 330 or embodiment 331, wherein the sgRNA scaffold comprises one or more U to A substitutions relative to the reference scaffold sequence

333. The system of any one of embodiments 330 to 332, wherein the sgRNA scaffold comprises one or more trimmed stem loop sequences in place of one or more longer stem loop structures in the reference scaffold sequence.

334. The system of embodiment 333, wherein the trimmed stem loop sequence comprises a GAAA tetraloop in place of a longer stem loop sequence in the reference scaffold sequence.

335. The system of any one of embodiments 330 to 334, wherein the sgRNA scaffold comprises one or more trimmed loop sequences in place of one or more longer loop sequences in the reference scaffold sequence.

336. The system of embodiment 335, wherein the sgRNA scaffold comprises a GAAA tetraloop in place of a longer loop sequence in the reference scaffold sequence.

337. The system of any one of embodiments 330 to 336, wherein the sgRNA scaffold comprises a nucleotide sequence that is at least 55% identical to the reference scaffold sequence.

338. The system of embodiment 337, wherein the sgRNA scaffold comprises a nucleotide sequence that is at least 60% identical to the reference scaffold sequence.

339. The system of embodiment 337, wherein the sgRNA scaffold comprises a nucleotide sequence that is at least 65% identical to the reference scaffold sequence.

340. The system of embodiment 337, wherein the sgRNA scaffold comprises a nucleotide sequence that is at least 70% identical to the reference scaffold sequence.

341. The system of embodiment 337, wherein the sgRNA scaffold comprises a nucleotide sequence that is at least 75% identical to the reference scaffold sequence.

342. The system of embodiment 337, wherein the sgRNA scaffold comprises a nucleotide sequence that is at least 80% identical to the reference scaffold sequence.

343. The system of embodiment 337, wherein the sgRNA scaffold comprises a nucleotide sequence that is at least 85% identical to the reference scaffold sequence.

344. The system of embodiment 337, wherein the sgRNA scaffold comprises a nucleotide sequence that is at least 90% identical to the reference scaffold sequence.

345. The system of embodiment 337, wherein the sgRNA scaffold comprises a nucleotide sequence that is at least 95% identical to the reference scaffold sequence.

346. The system of embodiment 337, wherein the sgRNA scaffold comprises a nucleotide sequence that is at least 96% identical to the reference scaffold sequence.

347. The system of embodiment 337, wherein the sgRNA scaffold comprises a nucleotide sequence that is at least 97% identical to the reference scaffold sequence.

348. The system of embodiment 337, wherein the sgRNA scaffold comprises a nucleotide sequence that is at least 98% identical to the reference scaffold sequence.

349. The system of embodiment 330, wherein the sgRNA scaffold comprises a nucleotide sequence that is at least 99% identical to the reference scaffold sequence.

350. The system of embodiment 337, wherein the sgRNA scaffold comprises a nucleotide sequence that has no more than 5 nucleotide mismatches with the reference scaffold sequence.

351. The system of embodiment 337, wherein the sgRNA scaffold comprises a nucleotide sequence that has no more than 4 nucleotide mismatches with the reference scaffold sequence.

352. The system of embodiment 337, wherein the sgRNA scaffold comprises a nucleotide sequence that has no more than 3 nucleotide mismatches with the reference scaffold sequence.

353. The system of embodiment 337, wherein the sgRNA scaffold comprises a nucleotide sequence that has no more than 2 nucleotide mismatches with the reference scaffold sequence.

354. The system of embodiment 337, wherein the sgRNA scaffold comprises a nucleotide sequence that has no more than 1 nucleotide mismatches with the reference scaffold sequence.

355. The system of any one of embodiments 329 to 354, wherein the reference protein sequence is SEQ ID NO:1 or SEQ ID NO:2 and the reference scaffold sequence is SEQ ID NO:15.

356. The system of any one of embodiments 329 to 354, wherein the reference protein sequence is SEQ ID NO:1 or SEQ ID NO:2 and the reference scaffold sequence is SEQ ID NO:16.

357. The system of any one of embodiments 329 to 354, wherein the reference protein sequence is SEQ ID NO:1 or SEQ ID NO:2 and the reference scaffold sequence is SEQ ID NO:17.

358. The system of any one of embodiments 329 to 354, wherein the reference protein sequence is SEQ ID NO:1 or SEQ ID NO:2 and the reference scaffold sequence is SEQ ID NO:18.

359. The system of any one of embodiments 329 to 354, wherein the reference protein sequence is SEQ ID NO:1 or SEQ ID NO:2 and the reference scaffold sequence is SEQ ID NO:19.

360. The system of any one of embodiments 329 to 354, wherein the reference protein sequence is SEQ ID NO:7 or SEQ ID NO:8 and the reference scaffold sequence is SEQ ID NO:22.

361. The system of any one of embodiments 329 to 354, wherein the reference protein sequence is SEQ ID NO:7 or SEQ ID NO:8 and the reference scaffold sequence is SEQ ID NO:23.

362. The system of any one of embodiments 329 to 354, wherein the reference protein sequence is SEQ ID NO:7 or SEQ ID NO:8 and the reference scaffold sequence is SEQ ID NO:24.

363. The system of any one of embodiments 329 to 354, wherein the reference protein sequence is SEQ ID NO:7 or SEQ ID NO:8 and the reference scaffold sequence is SEQ ID NO:25.

364. The system of any one of embodiments 329 to 354, wherein the reference protein sequence is SEQ ID NO:7 or SEQ ID NO:8 and the reference scaffold sequence is SEQ ID NO:822.

365. The system of any one of embodiments 329 to 354, wherein the reference protein sequence is SEQ ID NO:30 or SEQ ID NO:31 and the reference scaffold sequence is SEQ ID NO:75.

366. The system of any one of embodiments 329 to 354, wherein the reference protein sequence is SEQ ID NO:34 or SEQ ID NO:35 and the reference scaffold sequence is SEQ ID NO:76.

367. The system of any one of embodiments 329 to 354, wherein the reference protein sequence is SEQ ID NO:34 or SEQ ID NO:35 and the reference scaffold sequence is SEQ ID NO:77.

368. The system of any one of embodiments 329 to 354, wherein the reference protein sequence is SEQ ID NO:34 or SEQ ID NO:35 and the reference scaffold sequence is SEQ ID NO:78.

369. The system of any one of embodiments 329 to 354, wherein the reference protein sequence is SEQ ID NO:34 or SEQ ID NO:35 and the reference scaffold sequence is SEQ ID NO:79.

370. The system of any one of embodiments 329 to 354, wherein the reference protein sequence is SEQ ID NO:34 or SEQ ID NO:35 and the reference scaffold sequence is SEQ ID NO:822.

371. The system of any one of embodiments 329 to 370, wherein the sgRNA scaffold comprises 1 to 8 uracils at its 3′ end.

372. The system of embodiment 371, wherein the sgRNA scaffold comprises 1 uracil at its 3′ end.

373. The system of embodiment 371, wherein the sgRNA scaffold comprises 2 uracils at its 3′ end.

374. The system of embodiment 371, wherein the sgRNA scaffold comprises 3 uracils at its 3′ end.

375. The system of embodiment 371, wherein the sgRNA scaffold comprises 4 uracils at its 3′ end.

376. The system of embodiment 371, wherein the sgRNA scaffold comprises 5 uracils at its 3′ end.

377. The system of embodiment 371, wherein the sgRNA scaffold comprises 6 uracils at its 3′ end.

378. The system of embodiment 371, wherein the sgRNA scaffold comprises 7 uracils at its 3′ end.

379. The system of embodiment 371, wherein the sgRNA scaffold comprises 8 uracils at its 3′ end.

380. The system of any one of embodiments 299 to 379, wherein the spacer is 15 to 30 nucleotides in length.

381. The system of embodiment 380, wherein the spacer is 15 to 25 nucleotides in length.

382. The system of embodiment 380, wherein the spacer is 16 to 24 nucleotides in length.

383. The system of embodiment 380, wherein the spacer is 17 to 23 nucleotides in length.

384. The system of embodiment 380, wherein the spacer is 18 to 22 nucleotides in length.

385. The system of embodiment 380, wherein the spacer is 19 to 21 nucleotides in length.

386. The system of embodiment 380, wherein the spacer is 18 to 30 nucleotides in length.

387. The system of embodiment 380, wherein the spacer is 20 to 28 nucleotides in length.

388. The system of embodiment 380, wherein the spacer is 22 to 26 nucleotides in length.

389. The system of embodiment 380, wherein the spacer is 23 to 25 nucleotides in length.

390. The system of embodiment 380, wherein the spacer is 20 nucleotides in length.

391. The system of embodiment 380, wherein the spacer is 21 nucleotides in length.

392. The system of embodiment 380, wherein the spacer is 22 nucleotides in length.

393. The system of embodiment 380, wherein the spacer is 23 nucleotides in length.

394. The system of embodiment 380, wherein the spacer is 24 nucleotides in length.

395. The system of embodiment 380, wherein the spacer is 25 nucleotides in length.

396. The system of embodiment 380, wherein the spacer is 26 nucleotides in length.

397. The system of embodiment 380, wherein the spacer is 27 nucleotides in length.

398. The system of embodiment 380, wherein the spacer is 28 nucleotides in length.

399. The system of any one of embodiments 299 to 398, which is a ribonucleoprotein (RNP) comprising the Type II Cas protein complexed to the gRNA or means for targeting the Type II Cas protein to a target genomic sequence.

400. A nucleic acid encoding the Type II Cas protein of any one of embodiments 1 to 194, optionally wherein the nucleotide sequence encoding the Type II Cas protein is operably linked to a promoter that is heterologous to the Type II Cas protein.

401. The nucleic acid of embodiment 400, wherein the nucleotide sequence encoding the Type II Cas protein is codon optimized for expression in human cells.

402. The nucleic acid of embodiment 401, wherein when the reference protein sequence is SEQ ID NO:1 or SEQ ID NO:2, the nucleotide sequence encoding the Type II Cas protein comprises a nucleotide sequences that is at least 80%, at least 85%, at least 90%, at least 95%, at least 96%, at least 97%, at least 98%, at least 99%, or 100% identical to the nucleotide sequence of SEQ ID NO:5 or SEQ ID NO:6.

403. The nucleic acid of embodiment 401, wherein when the reference protein sequence is SEQ ID NO:7 or SEQ ID NO:8, the nucleotide sequence encoding the Type II Cas protein comprises a nucleotide sequences that is at least 80%, at least 85%, at least 90%, at least 95%, at least 96%, at least 97%, at least 98%, at least 99%, or 100% identical to the nucleotide sequence of SEQ ID NO: 11 or SEQ ID NO:12.

404. The nucleic acid of embodiment 401, wherein when the reference protein sequence is SEQ ID NO:30 or SEQ ID NO:31, the nucleotide sequence encoding the Type II Cas protein comprises a nucleotide sequences that is at least 80%, at least 85%, at least 90%, at least 95%, at least 96%, at least 97%, at least 98%, at least 99%, or 100% identical to the nucleotide sequence of SEQ ID NO:32 or SEQ ID NO:33.

405. The nucleic acid of embodiment 401, wherein when the reference protein sequence is SEQ ID NO:34 or SEQ ID NO:35, the nucleotide sequence encoding the Type II Cas protein comprises a nucleotide sequences that is at least 80%, at least 85%, at least 90%, at least 95%, at least 96%, at least 97%, at least 98%, at least 99%, or 100% identical to the nucleotide sequence of SEQ ID NO:36 or SEQ ID NO:37.

406. The nucleic acid of any one of embodiments embodiment 400 to 405, which is a plasmid.

407. The nucleic acid of any one of embodiments embodiment 400 to 405, which is a viral genome.

408. The nucleic acid of embodiment 407, wherein the viral genome is an adeno-associated virus (AAV) genome.

409. The nucleic acid of embodiment 408, wherein the AAV genome is an AAV2, AAV5, AAV7m8, AAV8, AAV9, AAVrh8r, or AAVrh10 genome.

410. The nucleic acid of embodiment 409, wherein the AAV genome is an AAV2 genome.

411. The nucleic acid of embodiment 409, wherein the AAV genome is an AAV5 genome.

412. The nucleic acid of embodiment 409, wherein the AAV genome is an AAV7m8 genome.

413. The nucleic acid of embodiment 409, wherein the AAV genome is an AAV8 genome.

414. The nucleic acid of embodiment 409, wherein the AAV genome is an AAV9 genome.

415. The nucleic acid of embodiment 409, wherein the AAV genome is an AAVrh8r genome.

416. The nucleic acid of embodiment 409, wherein the AAV genome is an AAVrh10 genome.

417. The nucleic acid of any one of embodiments 400 to 416, further encoding a gRNA, optionally wherein the gRNA is a gRNA according to any one of embodiments 195 to 298.

418. A nucleic acid encoding the gRNA of any one of embodiments 195 to 298.

419. The nucleic acid of embodiment 418, which is a plasmid.

420. The nucleic acid of embodiment 418, which is a viral genome.

421. The nucleic acid of embodiment 420, wherein the viral genome is an adeno-associated virus (AAV) genome.

422. The nucleic acid of embodiment 421, wherein the AAV genome is a AAV2, AAV5, AAV7m8, AAV8, AAV9, AAVrh8r, or AAVrh10 genome.

423. The nucleic acid of embodiment 422, wherein the AAV genome is an AAV2 genome.

424. The nucleic acid of embodiment 422, wherein the AAV genome is an AAV5 genome.

425. The nucleic acid of embodiment 422, wherein the AAV genome is an AAV7m8 genome.

426. The nucleic acid of embodiment 422, wherein the AAV genome is an AAV8 genome.

427. The nucleic acid of embodiment 422, wherein the AAV genome is an AAV9 genome.

428. The nucleic acid of embodiment 422, wherein the AAV genome is an AAVrh8r genome.

429. The nucleic acid of embodiment 422, wherein the AAV genome is an AAVrh10 genome.

430. The nucleic acid of any one of embodiments 418 to 429, further encoding a Type II Cas protein, optionally wherein the Type II Cas protein is a Type II Cas protein according to any one of embodiments 1 to 194.

431. A nucleic acid encoding the Type II Cas protein and gRNA of the system of any one of embodiments 299 to 399.

432. The nucleic acid of embodiment 431, wherein the nucleotide sequence encoding the Type II Cas protein is codon optimized for expression in human cells.

433. The nucleic acid of embodiment 431 or embodiment 432, which is a plasmid.

434. The nucleic acid of embodiment 431 or embodiment 432, which is a viral genome.

435. The nucleic acid of embodiment 434, wherein the viral genome is an adeno-associated virus (AAV) genome.

436. The nucleic acid of embodiment 435, wherein the AAV genome is a AAV2, AAV5, AAV7m8, AAV8, AAV9, AAVrh8r, or AAVrh10 genome.

437. The nucleic acid of embodiment 436, wherein the AAV genome is an AAV2 genome.

438. The nucleic acid of embodiment 436, wherein the AAV genome is an AAV5 genome.

439. The nucleic acid of embodiment 436, wherein the AAV genome is an AAV7m8 genome.

440. The nucleic acid of embodiment 436, wherein the AAV genome is an AAV8 genome.

441. The nucleic acid of embodiment 436, wherein the AAV genome is an AAV9 genome.

442. The nucleic acid of embodiment 436, wherein the AAV genome is an AAVrh8r genome.

443. The nucleic acid of embodiment 436, wherein the AAV genome is an AAVrh10 genome.

444. A plurality of nucleic acids comprising separate nucleic acids encoding the Type II Cas protein and gRNA of the system of any one of embodiments 299 to 399.

445. The plurality of nucleic acid of embodiment 444, wherein the separate nucleic acids encoding the Type II Cas protein and gRNA are plasmids.

446. The plurality of nucleic acids of embodiment 444, wherein the separate nucleic acids encoding the Type II Cas protein and gRNA are viral genomes.

447. The plurality of nucleic acids of embodiment 446, wherein the viral genomes are adeno-associated virus (AAV) genomes.

448. The plurality of nucleic acids of embodiment 447, wherein the AAV genomes the encoding the Type II Cas protein and gRNA are independently an AAV2, AAV5, AAV7m8, AAV8, AAV9, AAVrh8r, or AAVrh10 genome.

449. A Type II Cas protein according to any one of embodiments 1 to 194, a gRNA according to any one of embodiments 195 to 298, a system according to of any one of embodiments 299 to 399, a nucleic acid according to any one of embodiments 400 to 443, or a plurality of nucleic acids according to of any one of embodiments 444 to 448 for use in a method of editing a human genomic sequence.

450. The Type II Cas protein, gRNA, system, nucleic acid, or a plurality of nucleic acids for use according to embodiment 449, wherein the human genomic sequence is a CCR5, EMX1, Fas, FANCF, HBB, ZSCAN2, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, BCR, ATM, HBG1, HPRT, IL2RG, NF1, USH2A, RHO, BcLenh, or CTFR genomic sequence.

451. The Type II Cas protein, gRNA, system, nucleic acid, or a plurality of nucleic acids for use according to embodiment 449, wherein the human genomic sequence is a CCR5, EMX1, Fas, FANCF, HBB, ZSCAN, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, or BCR genomic sequence.

452. A particle comprising a Type II Cas protein according to any one of embodiments 1 to 194, a gRNA according to any one of embodiments 195 to 298, a system according to of any one of embodiments 299 to 399, a nucleic acid according to any one of embodiments 400 to 443, or a plurality of nucleic acids according to of any one of embodiments 444 to 448.

453. The particle of embodiment 452, which is a lipid nanoparticle, a vesicle, a gold nanoparticle, a viral-like particle (VLP) or a viral particle.

454. The particle of embodiment 453, which is a lipid nanoparticle.

455. The particle of embodiment 453, which is a vesicle.

456. The particle of embodiment 453, which is a gold nanoparticle.

457. The particle of embodiment 453, which is a viral-like particle (VLP).

458. The particle of embodiment 453, which is a viral particle.

459. The particle of embodiment 457, which is an adeno-associated virus (AAV) particle.

460. The particle of embodiment 459, wherein the AAV particle is an AAV2, AAV5, AAV7m8, AAV8, AAV9, AAVrh8r, or AAVrh10 particle.

461. The particle of embodiment 460, wherein the AAV particle is an AAV2 particle.

462. The particle of embodiment 460, wherein the AAV particle is an AAV5 particle.

463. The particle of embodiment 460, wherein the AAV particle is an AAV7m8 particle.

464. The particle of embodiment 460, wherein the AAV particle is an AAV8 particle.

465. The particle of embodiment 460, wherein the AAV particle is an AAV9 particle.

466. The particle of embodiment 460, wherein the AAV particle is an AAVrh8r particle.

467. The particle of embodiment 460, wherein the AAV particle is an AAVrh10 particle.

468. A pharmaceutical composition comprising a Type II Cas protein according to any one of embodiments 1 to 194, a gRNA according to any one of embodiments 195 to 298, a system according to of any one of embodiments 299 to 399, a nucleic acid according to any one of embodiments 400 to 443, or a plurality of nucleic acids according to of any one of embodiments 444 to 448, or a particle according to any one of embodiments 452 to 467 and at least one pharmaceutically acceptable excipient.

469. A cell comprising a Type II Cas protein according to any one of embodiments 1 to 194, a gRNA according to any one of embodiments 195 to 298, a system according to of any one of embodiments 299 to 399, a nucleic acid according to any one of embodiments 400 to 443, or a plurality of nucleic acids according to of any one of embodiments 444 to 448, or a particle according to any one of embodiments 452 to 467.

470. The cell of embodiment 469, which is a human cell.

471. The cell of embodiment 469 or embodiment 470, wherein the cell is a hematopoietic progenitor cell.

472. The cell of any one of embodiments 469 to 471, which is a stem cell.

473. The cell of embodiment 472, wherein the stem cell is a hematopoietic stem cell (HSC), a pluripotent stem cell, or an induced pluripotent stem cell (iPS).

474. The cell of embodiment 473, wherein the stem cell is an embryonic stem cell.

475. The cell of any one of embodiments 469 to 474, which is an ex vivo cell.

476. A population of cells according to any one embodiments 469 to 475.

477. A method for altering a cell, the method comprising contacting the cell with a Type II Cas protein according to any one of embodiments 1 to 194, a gRNA according to any one of embodiments 195 to 298, a system according to of any one of embodiments 299 to 399, a nucleic acid according to any one of embodiments 400 to 443, or a plurality of nucleic acids according to of any one of embodiments 444 to 448, a particle according to any one of embodiments 452 to 467, or a pharmaceutical composition according to embodiment 468.

478. The method of embodiment 477, which comprises contacting the cell with the Type II Cas protein of any one of embodiments 1 to 194.

479. The method of embodiment 477, which comprises contacting the cell with the gRNA of any one of embodiments 195 to 298.

480. The method of embodiment 477, which comprises contacting the cell with the system of any one of embodiments 299 to 399.

481. The method of embodiment 480, which comprises electroporation of the cell prior to contacting the cell with the system.

482. The method of embodiment 480, which comprises lipid-mediated delivery of the system to the cell, optionally wherein the lipid-mediated delivery is cationic lipid-mediated delivery.

483. The method of embodiment 480, which comprises polymer-mediated delivery of the system to the cell.

484. The method of embodiment 480, which comprises delivery of the system to the cell by lipofection.

485. The method of embodiment 480, which comprises delivery of the system to the cell by nucleofection.

486. The method of embodiment 477, which comprises contacting the cell with the nucleic acid of any one of embodiments 400 to 443.

487. The method of embodiment 477, which comprises contacting the cell with the plurality of nucleic acids of any one of embodiments 444 to 448.

488. The method of embodiment 477, which comprises contacting the cell with the particle of any one of embodiments 452 to 467.

489. The method of embodiment 477, which comprises contacting the cell with the pharmaceutical composition of embodiment 468.

490. The method of any one of embodiments 477 to 489, wherein the contacting alters a CCR5, EMX1, Fas, FANCF, HBB, ZSCAN2, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, BCR, ATM, HBG1, HPRT, IL2RG, NF1, USH2A, RHO, BcLenh, or CTFR genomic sequence 491. The method of any one of embodiments 477 to 489, wherein the contacting alters a CCR5, EMX1, Fas, FANCF, HBB, ZSCAN, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, or BCR genomic sequence.

492. The method of any one of embodiments 477 to 490, wherein the cell is a human cell.

493. The method of any one of embodiments 477 to 492, wherein the cell is a hematopoietic progenitor cell.

494. The method of any one of embodiments 477 to 493, wherein the cell is a stem cell.

495. The method of embodiment 494, wherein the stem cell is a hematopoietic stem cell (HSC), a pluripotent stem cell, or an induced pluripotent stem cell (iPS).

496. The method of embodiment 495, wherein the stem cell is an embryonic stem cell.

497. The method of any one of embodiments 477 to 496, wherein the contacting is in vitro.

498. The method of embodiment 497, further comprising transplanting the cell to a subject.

499. The method of any one of embodiments 477 to 496, wherein the contacting is in vivo in a subject.

500. A cell or population of cells produced by the method of any one of embodiments 477 to 497.

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

1. A Type II Cas protein comprising an amino acid sequence having at least 50% sequence identity to:
(a) the amino acid sequence of a RuvC-I domain of a reference protein sequence;
(b) the amino acid sequence of a RuvC-II domain of a reference protein sequence;
(c) the amino acid sequence of a RuvC-III domain of a reference protein sequence;
(d) the amino acid sequence of a BH domain of a reference protein sequence;
(e) the amino acid sequence of a REC domain of a reference protein sequence;
(f) the amino acid sequence of a HNH domain of a reference protein sequence;
(g) the amino acid sequence of a WED domain of a reference protein sequence;
(h) the amino acid sequence of a PID domain of a reference protein sequence; or
(i) the amino acid sequence of the full length of a reference protein sequence;
wherein the reference protein sequence is SEQ ID NO:1, SEQ ID NO:2, SEQ ID NO:7, SEQ ID NO:8, SEQ ID NO:30, SEQ ID NO:31, SEQ ID NO:34, or SEQ ID NO:35.

2-9. (canceled)

10. The Type II Cas protein of claim 1, wherein the amino acid sequence of the Type II Cas protein comprises an amino acid sequence that is at least 55% identical to the full length of the reference protein sequence.

11. The Type II Cas protein of claim 1, which is a chimeric Type II Cas protein.

12. The Type II Cas protein of claim 1, which is a fusion protein.

13. The Type II Cas protein of claim 12, which comprises one or more nuclear localization signals.

14-16. (canceled)

17. The Type II Cas protein of claim 12, which comprises: (a) a fusion partner which is an adenosine deaminase; (b) a fusion partner which is a cytodine deaminase; or (c) a fusion partner which is a reverse transcriptase.

18. (canceled)

19. The Type II Cas protein of claim 1, wherein (a) the reference protein sequence is SEQ ID NO:1 and the amino acid sequence of the Type II Cas protein comprises the amino acid sequence of SEQ ID NO:1, SEQ ID NO:2, or SEQ ID NO:3; (b) the reference protein sequence is SEQ ID NO:2 and the amino acid sequence of the Type II Cas protein comprises the amino acid sequence of SEQ ID NO:2; (c) the reference protein sequence is SEQ ID NO:7 and the amino acid sequence of the Type II Cas protein comprises the amino acid sequence of SEQ ID NO:7, SEQ ID NO:8, or SEQ ID NO:9; (d) the reference protein sequence is SEQ ID NO:8 and the amino acid sequence of the Type II Cas protein comprises the amino acid sequence of SEQ ID NO:8; (e) the reference protein sequence is SEQ ID NO:30 and the amino acid sequence of the Type II Cas protein comprises the amino acid sequence of SEQ ID NO:30, SEQ ID NO:31, or SEQ ID NO:786; (f) the reference protein sequence is SEQ ID NO:31 and amino acid sequence of the Type II Cas protein comprises the amino acid sequence of SEQ ID NO:31; (g) the reference protein sequence is SEQ ID NO:34 and the amino acid sequence of the Type II Cas protein comprises the amino acid sequence of SEQ ID NO: 34, SEQ ID NO:35 or SEQ ID NO:787; or (h) the reference protein sequence is SEQ ID NO:35 and the amino acid sequence of the Type II Cas protein comprises the amino acid sequence of SEQ ID NO:35.

20. A Type II Cas protein whose amino acid sequence is identical to a Type II Cas protein of claim 1 except for one or more amino acid substitutions relative to the reference sequence that provide nickase activity, optionally wherein the one or more amino acid substitutions relative to the reference sequence that provide nickase activity comprise a D23A mutation, wherein the position of the D23A substitution is defined with respect to the amino acid numbering of SEQ ID NO:8.

21. A gRNA comprising a spacer and a sgRNA scaffold, wherein:
(a) the spacer is positioned 5′ to the sgRNA scaffold; and
(b) the nucleotide sequence of the sgRNA scaffold comprises a nucleotide sequence that is at least 50% identical to a reference scaffold sequence, wherein the reference scaffold sequence is SEQ ID NO:15, SEQ ID NO:16, SEQ ID NO:17, SEQ ID NO:18, SEQ ID NO:19, SEQ ID NO:22, SEQ ID NO:23, SEQ ID NO:24, SEQ ID NO:25, SEQ ID NO:75, or SEQ ID NO:822.

22-27. (canceled)

28. The gRNA of claim 21, wherein the nucleotide sequence of the sgRNA scaffold comprises the nucleotide sequence of SEQ ID NO:26, SEQ ID NO:27, SEQ ID NO:28, SEQ ID NO:29, or SEQ ID NO:823.

29-30. (canceled)

31. A gRNA comprising (i) a crRNA comprising a spacer and a crRNA scaffold, wherein the spacer is 5′ to the crRNA scaffold, and (ii) a tracrRNA, wherein the nucleotide sequence of the spacer is partially or fully complementary to a target mammalian genomic sequence and the nucleotide sequence of the crRNA scaffold comprises the nucleotide sequence of SEQ ID NO:13, SEQ ID NO:20, SEQ ID NO:788, or SEQ ID NO:790.

32-37. (canceled)

38. A system comprising the Type II Cas protein of claim 1 and a guide RNA (gRNA) comprising a spacer sequence.

39-44. (canceled)

45. A nucleic acid encoding the Type II Cas protein of claim 1.

46-47. (canceled)

48. A nucleic acid encoding the gRNA of claim 21.

49. A nucleic acid encoding the Type II Cas protein and gRNA of the system of claim 38.

50. A plurality of nucleic acids comprising separate nucleic acids encoding the Type II Cas protein and gRNA of the system of claim 38.

51. (canceled)

52. A particle comprising the Type II Cas protein of claim 1.

53. A pharmaceutical composition comprising the Type II Cas protein of claim 1 and at least one pharmaceutically acceptable excipient.

54. A human cell comprising the Type II Cas protein of claim 1.

55. A population of cells according to claim 54.

56. A method for altering a cell, the method comprising contacting the cell with the Type II Cas protein of claim 1.

57. A cell or population of cells produced by the method of claim 56.
